(19) United States
(12) Patent Application Publication
(10) Pub. No.: US 2007/0232527 A1
(43) Pub. Date: Oct. 4, 2007

(54) Medicaments and methods combining a HCV protease inhibitor and an AKR competitor

(76) Inventors: Anima Ghosal, Edison, NJ (US); Narendra S. Kishnani, East Brunswick, NJ (US); Kevin B. Alton, Cedar Knolls, NJ (US); Ronald E. White, Cranbury, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(21) Appl. No.: 11/502,562

(22) Filed: Aug. 10, 2006

Related U.S. Application Data
(63) Continuation-in-part of application No. 11/443,647, filed on May 31, 2006.
(60) Provisional application No. 60/686,924, filed on Jun. 2, 2005.

(51) Int. Cl. A61K 38/12; A61K 38/04

(52) U.S. Cl. 514/9; 514/18; 530/317; 530/331

(57) ABSTRACT

Disclosed are medicaments, pharmaceutical compositions, pharmaceutical kits, and methods based on combinations of a hepatitis C virus (HCV) protease inhibitor and an aldo-keto reductase (AKR) competitor, for concurrent or consecutive administration in treating, preventing, or ameliorating one or more symptoms of HCV, treating disorders associated with HCV, or inhibiting cathepsin activity in a subject.

### REFERENCE TO RELATED APPLICATION

This application is a continuation-in-part of U.S. patent application Ser. No. 11/443,647, filed May 31, 2006 which claims the benefit of priority to U.S. Provisional Patent Application 60/686,924 filed Jun. 2, 2005, the entire disclosure of each of the priority applications hereby incorporated by reference.

### FIELD OF THE INVENTION

The present invention relates to medicaments, pharmaceutical compositions, pharmaceutical kits, and methods based on combinations of a hepatitis C virus (HCV) protease inhibitor and an aldo-keto reductase (AKR) competitor, for concurrent or consecutive administration treating, preventing, or ameliorating one or more symptoms of HCV, treating disorders associated with HCV, or inhibiting cathepsin activity in a subject.

### BACKGROUND OF THE INVENTION

HCV has been implicated in cirrhosis of the liver and in induction of hepatocellular carcinoma. The prognosis for patients suffering from HCV infection is currently poor. HCV infection is more difficult to treat than other forms of hepatitis due to the lack of immunity or remission associated with HCV infection. Current data indicates a less than 50% survival rate at four years post cirrhosis diagnosis. Patients diagnosed with localized resectable hepatocellular carcinoma have a five-year survival rate of 10-30%, whereas those with localized unresectable hepatocellular carcinoma have a five-year survival rate of less than 1%.

Current therapies for hepatitis C include interferon-α (INF_{α}) and combination therapy with ribavirin and interferon. See, e.g., Berenguer and Wright, Proc Assoc Am Physicians, 110(2):98-112 (1998). These therapies suffer from a low sustained response rate and frequent side effects. See, e.g., Hoofnagle and di Bisceglie, N Engl J Med, 336(5):347-356 (1997). Currently, no vaccine is available for HCV infection.

HCV is a (+)-sense single-stranded RNA virus that has been implicated as the major causative agent in non-A, non-B hepatitis (NANBH), particularly in blood-associated NANBH (BB-NANBH) (see, International Patent Application Publication No. WO 89/04669 and European Patent Application Publication No. EP 381 216). NANBH is to be distinguished from other types of viral-induced liver disease, such as hepatitis A virus (HAV), hepatitis B virus (HBV), delta hepatitis virus (HDV), cytomegalovirus (CMV) and Epstein-Barr virus (EBV), as well as from other forms of liver disease such as alcoholism and primary biliar cirrhosis.

Recently, a HCV protease necessary for polypeptide processing and viral replication has been identified, cloned and expressed; (see, e.g., U.S. Pat. No. 5,712,145). This approximately 3000 amino acid polyprotein contains, from the amino terminus to the carboxy terminus, a nucleocapsid protein (C), envelope proteins (E1 and E2) and several non-structural proteins (NS1, 2, 3, 4a, 5a and 5b). NS3 is an approximately 68 kda protein, encoded by approximately 1893 nucleotides of the HCV genome, and has two distinct domains: (a) a serine protease domain consisting of approximately 200 of the N-terminal amino acids; and (b) an RNA-dependent ATPase domain at the C-terminus of the protein. The NS3 protease is considered a member of the chymotrypsin family because of similarities in protein sequence, overall three-dimensional structure and mechanism of catalysis. Other chymotrypsin-like enzymes are elastase, factor Xa, thrombin, trypsin, plasmin, urokinase, tPA and PSA. The HCV NS3 serine protease is responsible for proteolysis of the polypeptide (polyprotein) at the NS3/NS4a, NS4a/NS4b, NS4b/NS5a and NS5a/NS5b junctions and is thus responsible for generating five viral proteins during viral replication. This has made the HCV NS3 serine protease an attractive target for antiviral chemotherapy.

It has been determined that the NS4a protein, an approximately 6 kda polypeptide, is a co-factor for the serine protease activity of NS3. Autocleavage of the NS3/NS4a junction by the NS3/NS4a serine protease occurs intramolecularly (i.e., cis) while the other cleavage sites are processed intermolecularly (i.e., trans).

Analysis of the natural cleavage sites for HCV protease revealed the presence of cysteine at P1 and serine at P1′ and that these residues are strictly conserved in the NS4a/NS4b, NS4b/NS5a and NS5a/NS5b junctions. The NS3/NS4a junction contains a threonine at P1 and a serine at P1′. The Cys→Thr substitution at NS3/NS4a is postulated to account for the requirement of cis rather than trans processing at this junction. See, e.g., Pizzi et al., Proc Natl Acad Sci (USA), 91(3):888-892 (1994), Failla et al., Fold Des, 1(1):35-42 (1996), Wang et al., J Virol, 78(2):700-709 (2004). The NS3/NS4a cleavage site is also more tolerant of mutagenesis than the other sites. See, e.g., Kolykhalov et al., J Virol, 68(11):7525-7533 (1994). It has also been found that acidic residues in the region upstream of the cleavage site are required for efficient cleavage. See, e.g., Komoda et al., J Virol, 68(11):7351-7357 (1994).

Inhibitors of HCV protease that have been reported include antioxidants (see, International Patent Application Publication No. WO 98/14181), certain peptides and peptide analogs (see, International Patent Application Publication No. WO 98/17679, Landro et. al., Biochemistry, 36(31):9340-9348 (1997), Ingallinelia et al., Biochemistry, 37(25):8906-8914 (1998), Llinás-Brunet et al., Bioorg Med Chem Lett, 8(13):1713-1718 (1998)), inhibitors based on the 70-amino acid polypeptide eglin c (Martin et al., Biochemistry, 37(33):11459-11468 (1998), inhibitors affinity selected from human pancreatic secretory trypsin inhibitor (hPST1-C3) and minibody repertoires (MBip) (Dimasi et. al., J Virol, 71(10):7461-7469 (1997)), cVHE2 (a “camelized” variable domain antibody fragment) (Martin et al., Protein Eng, 10(5):607-614 (1997), and α1-antichymotrypsin (ACT) (Elzouki et. al., J Hepat, 27(1):42-48 (1997)). A ribozyme designed to selectively destroy hepatitis C virus RNA has recently been disclosed (see, BioWorld Today, 9(217):4 (Nov. 10, 1998)).

Reference is also made to the PCT Publications, No. WO 98/17679, published Apr. 30, 1998 (Vertex Pharmaceuticals Incorporated); WO 98/22496, published May 28, 1998 (F. Hoffmann-La Roche AG); and WO 99/07734, published Feb. 18, 1999 (Boehringer Ingelheim Canada Ltd.).

Pending and copending U.S. patent applications, Ser. No. 60/194,607, filed Apr. 5, 2000 (corresponding to U.S. Publication No. 2002/010781), and Ser. No. 60/198,204, filed Apr. 19, 2000 (corresponding to U.S. Publication No. 2002/0016294), Ser. No. 60/220,110, filed Jul. 21, 2000 (corresponding to U.S. Publication No. 2002/0102235), Ser. No. 60/220,109, filed Jul. 21, 2000 (corresponding to U.S. Publication No. 2003/0036501), Ser. No. 60/220,107, filed Jul. 21, 2000 (corresponding to U.S. Publication No. 2002/0160962), Ser. No. 60/254,869, filed Dec. 12, 2000 (corresponding to U.S. Publication No. 2002/0147139), Ser. No. 60/220,101, filed Jul. 21, 2000 (corresponding to U.S. Publication No. 2002/0068702), Ser. No. 60/568,721 filed May 6, 2004 (corresponding to WO 2005/107745), and WO 2003/062265, disclose various types of peptides and/or other compounds as NS-3 serine protease inhibitors of hepatitis C virus.

There is a need for new treatments and therapies for HCV infection to treat, prevent or ameliorate of one or more symptoms of HCV, methods for modulating the activity of serine proteases, particularly the HCV NS3/NS4a serine protease, and for methods of modulating the processing of the HCV polypeptide.

Another aspect of the present invention is directed to inhibiting cathepsin activity. Cathepsins (Cats) belong to the papain superfamily of lysosomal cysteine proteases. Cathepsins are involved in the normal proteolysis and turnover of target proteins and tissues as well as in initiating proteolytic cascades by proenzyme activation and in participating in MHC class II molecule expression. Baldwin, Proc Natl Acad Sci, 90(14):6796-6800 (1993); Mizuochi, Immunol Lett, 43(3):189-193 (1994).

However, aberrant cathepsin expression has also been implicated in several serious human disease states. Cathepsins have been shown to be abundantly expressed in cancer cells, including breast, lung, prostate, glioblastoma and head/neck cancer cells, (Kos and Lah, Oncol Rep, 5(6):1349-1361 (1998); Yan et al., Biol Chem, 379(2): 113-123 (1998); Mort and Buttle, Int J Biochem Cell Biol, 29(5): 715-720 (1997); Friedrich et al., Eur J Cancer, 35(1):138-144 (1999)) and are associated with poor treatment outcome of patients with breast cancer, lung cancer, brain tumor and head/neck cancer. Kos and Lah, supra. Additionally, aberrant expression of cathepsin is evident in several inflammatory disease states, including rheumatoid arthritis and osteoarthritis. Keyszer et al., Arthritis Rheum, 38(7):976-984 (1995).

The molecular mechanisms of cathepsin activity are not completely understood. Recently, it was shown that forced expression of cathepsin B rescued cells from serum deprivation-induced apoptotic death (Shibata et al., Biochem Biophys Res Commun, 251(1):199-203 (1998)) and that treatment of cells with antisense oligonucleotides of cathepsin B induced apoptosis. Isahara et al., Neuroscience, 91(1):233-249 (1999). These reports suggest an anti-apoptotic role for the cathepsins that is contrary to earlier reports that cathepsins are mediators of apoptosis. Roberts et al., Gastroenterology, 113(5):1714-1726 (1997); Jones et al., Am J Physiol, 275(4Pt1):G723-730 (1998).

Cathepsin K is a member of the family of enzymes which are part of the papain superfamily of cysteine proteases. Cathepsins B, H, L, N and S have been described in the literature. Recently, cathepsin K polypeptide and the cDNA encoding such polypeptide were disclosed in U.S. Pat. No. 5,501,969 (called cathepsin O therein). Cathepsin K has been recently expressed, purified, and characterized. Bossard et. al., J Biol Chem, 271(21):12517-12524 (1996); Drake et al., J Biol Chem, 271(21):12511-12516 (1996); Bromme et al., J. Biol. Chem., 271 (4):2126-2132 (1996).

Cathepsin K has been variously denoted as cathepsin O, cathepsin X or cathepsin O2 in the literature. The designation cathepsin K is considered to be the more appropriate one (name assigned by Nomenclature Committee of the International Union of Biochemistry and Molecular Biology).

Cathepsins of the papain superfamily of cysteine proteases function in the normal physiological process of protein degradation in animals, including humans, e.g., in the degradation of connective tissue. However, elevated levels of these enzymes in the body can result in pathological conditions leading to disease. Thus, cathepsins have been implicated in various disease states, including but not limited to, infections by pneumocystis carinii, trypsanoma cruzi, trypsanoma brucei brucei, and Crithidia fusiculata; as well as in schistosomiasis malaria, tumor metastasis, metachromatic leukodystrophy, muscular dystrophy, amytrophy, and the like. See International Publication Number WO 94/04172, published on Mar. 3, 1994, and references cited therein. See also European Patent Application EP 0 603 873 A1, and references cited therein. Two bacterial cysteine proteases from P. gingivallis, called gingipains, have been implicated in the pathogenesis of gingivitis. Potempa et al., Perspectives in Drug Discovery and Design, 2:445-458 (1994).

Cathepsin K is believed to play a causative role in diseases of excessive bone or cartilage loss. Bone is composed of a protein matrix in which spindle- or plate-shaped crystals of hydroxyapatite are incorporated. Type I Collagen represents the major structural protein of bone comprising approximately 90% of the structural protein. The remaining 10% of matrix is composed of a number of non-collagenous proteins, including osteocalcin, proteoglycans, osteopontin, osteonectin, thrombospondin, fibronectin, and bone sialoprotein. Skeletal bone undergoes remodeling at discrete foci throughout life. These foci, or remodeling units, undergo a cycle consisting of a bone resorption phase followed by a phase of bone replacement. Bone resorption is carried out by osteoclasts, which are multinuclear cells of hematopoietic lineage. In several disease states, such as osteoporosis and Paget's disease, the normal balance between bone resorption and formation is disrupted, and there is a net loss of bone at each cycle. Ultimately, this leads to weakening of the bone and may result in increased fracture risk with minimal trauma.

The abundant selective expression of cathepsin K in osteoclasts strongly suggests that this enzyme is essential for bone resorption. Thus, selective inhibition of cathepsin K may provide an effective treatment for diseases of excessive bone loss, including, but not limited to, osteoporosis, gingival diseases such as gingivitis and periodontitis, Paget's disease, hypercalcemia of malignancy, and metabolic bone disease. Cathepsin K levels have also been demonstrated to be elevated in chondroclasts of osteoarthritic synovium. Thus, selective inhibition of cathepsin K may also be useful for treating diseases of excessive cartilage or matrix degradation, including, but not limited to, osteoarthritis and rheumatoid arthritis. Metastatic neoplastic cells also typically express high levels of proteolytic enzymes that degrade the surrounding matrix. Thus, selective inhibition of cathepsin K may also be useful for treating certain neoplastic diseases.

There are reports in the literature of the expression of Cathepsin B and L antigen and that activity is associated with early colorectal cancer progression. Troy et al., Eur J Cancer, 40(10):1610-1616 (2004). The findings suggest that cysteine proteases play an important role in colorectal cancer progression.

Cathepsin L has been shown to be an important protein mediating the malignancy of gliomas and it has been suggested that its inhibition may diminish their invasion and lead to increased tumor cell apoptosis by reducing apoptotic threshold. Levicar et al., Cancer Gene Ther, 10(2):141-151 (2003).

Katunuma et al., Arch Biochem Biophys, 397(2):305-311 (2002) reports on antihypercalcemic and antimetastatic effects of CLIK-148 in vivo, which is a specific inhibitor of cathepsin L. This reference also reports that CLIK-148 treatment reduced distant bone metastasis to the femur and tibia of melanoma A375 tumors implanted into the left ventricle of the heart.

Rousselet et al., Cancer Res, 64(1): 146-151 (2004) reports that anti-cathepsin L single chain variable fragment (ScFv) could be used to inhibit the tumorigenic and metastatic phenotype of human melanoma, depending on procathepsin L secretion, and the possible use of anti-cathepsin L ScFv as a molecular tool in a therapeutic cellular approach.

Colella and Casey, Biotech Histochem, 78(2):101-108 (2003) reports that the cysteine proteinases cathepsin L and B participate in the invasive ability of the PC3 prostrate cancer cell line, and the potential of using cystein protease inhibitiors such as cystatins as anti-metastatic agents.

Krueger et al., Cancer Gene Ther, 8(7):522-528 (2001) reports that in human osteosarcoma cell line MNNG/HOS, cathepsin L influences cellular malignancy by promoting migration and basement membrane degradation.

Frohlich et al., Arch Dermatol Res, 295(10):411-421 (2004) reports that cathepsins B and L are involved in invasion of basal cell carcinoma (BCC) cells.

U.S. Provisional Patent Application Ser. No. 60/673,294, entitled “Compounds for Inhibiting Cathepsin Activity,” filed Apr. 20, 2005, discloses various types of peptides and/or other compounds as inhibitors of cathepsin.

Cathepsins therefore are attractive targets for the discovery of novel chemotherapeutics and methods of treatment effective against a variety of diseases. There is a need for compounds and combinations useful in the inhibition of cathepsin activity and in the treatment of these disorders.

It would also be desirable to modify the pharmacokinetic behavior of HCV treatments and cathepsin inhibitors to enhance the efficacy and duration of action thereof.

### SUMMARY OF THE INVENTION

The present invention provides medicaments, pharmaceutical compositions, pharmaceutical kits, and methods based on combinations comprising, separately or together: (a) at least one AKR competitor; and (b) at least one compound of Formula I to XXVII below, for concurrent or consecutive administration in treating, preventing, or ameliorating one or more symptoms of HCV, treating disorders associated with HCV, or inhibiting cathepsin activity in a subject.

The present invention also provides medicaments, pharmaceutical compositions, pharmaceutical kits, and methods based on combinations comprising, separately or together: (a) at least one aldo-keto reductase (AKR) competitor; and (b) at least one HCV protease inhibitor, or a mixture of two or more thereof for concurrent or consecutive administration in treating or ameliorating one or more symptoms of HCV, or disorders associated with HCV in a subject in need thereof.

The present invention also provides medicaments, pharmaceutical compositions, pharmaceutical kits, and methods based on combinations comprising, separately or together: (a) at least one AKR competitor; and (b) at least one compound of Formula I to XXVII below, or a mixture of two or more thereof for concurrent or consecutive administration in treating or ameliorating one or more symptoms of HCV, or disorders associated with HCV in a subject in need thereof.

In one embodiment, the “at least one compound” is a compound of structural

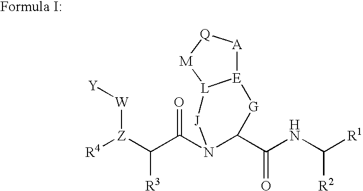

or a pharmaceutically acceptable salt, solvate or ester thereof; wherein in Formula I:

Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;

X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;

X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;

R¹ is COR⁵, wherein R⁵ is COR⁷ wherein R⁷ is NHR⁹, wherein R⁹ is selected from the group consisting of H, alkyl, aryl, heteroalkyl, heteroaryl, cycloalkyl, cycloalkyl, arylalkyl, heteroarylalkyl, [CH(R¹′)]ₚCOOR¹¹, [CH(R¹′)]ₚCONR¹²R¹³, [CH(R¹′)]ₚSO₂R¹¹, [CH(R¹′)]ₚCOR¹¹, [CH(R¹′) ]ₚCH(OH)R¹¹, CH(R¹′)CONHCH(R²)COOR¹¹, CH(R¹′)CONHCH(R²′)CONR¹²R¹³, CH (R¹′)CONHCH(R²)R′, CH(R¹′)CONHCH(R²′)CONHCH(R³)COOR¹¹, CH(R¹′)CONHCH (R²′)CONHCH(R³)CONR¹²R¹³, CH(R^{1′})CONHCH(R²′)CONHCH(R³′)CONHCH(R⁴′)COOR¹¹, CH(R¹′)CONHCH(R²)CONHCH(R³)CONHCH(R⁴)CONR¹²R¹³, CH(R¹′)CONHCH(R²′)CONHCH(R³′)CONHCH(R⁴′)CONHCH(R⁵′)COOR¹¹ and CH(R¹′)CONHCH (R²′)CONHCH(R³′)CONHCH(R⁴′)CONHCH(R⁵′) CONR¹²R¹³, wherein R¹′, R²′, R³′, R⁴′, R⁵′, R¹¹, R¹², R¹³, and R′ are independently selected from the group consisting of H, alkyl, aryl, heteroalkyl, heteroaryl, cycloalkyl, alkyl-aryl, alkyl-heteroaryl, aryl-alkyl and heteroaralkyl;

Z is selected from O, N, CH or CR;

W maybe present or absent, and if W is present, W is selected from C═O, C═S, C(═N—CN), or SO₂;

Q maybe present or absent, and when Q is present, Q is CH, N, P, (CH₂)ₚ, (CHR)ₚ, (CRR′)ₚ, O, NR, S, or SO₂; and when Q is absent, M may be present or absent; when Q and M are absent, A is directly linked to L;

A is O, CH₂, (CHR)ₚ, (CHR—CHR′) p, (CRR′) p, NR, S, SO₂ or a bond;

E is CH, N, CR, or a double bond towards A, L or G;

G may be present or absent, and when G is present, G is (CH₂)ₚ, (CHR)ₚ, or (CRR′)ₚ; and when G is absent, J is present and E is directly connected to the carbon atom in Formula I as G is linked to;

J maybe present or absent, and when J is present, J is (CH₂)ₚ, (CHR)ₚ, or (CRR′)ₚ, SO₂, NH, NR or O; and when J is absent, G is present and E is directly linked to N shown in Formula I as linked to J;

L may be present or absent, and when L is present, L is CH, CR, O, S or NR; and when L is absent, then M may be present or absent; and if M is present with L being absent, then M is directly and independently linked to E, and J is directly and independently linked to E;

M may be present or absent, and when M is present, M is O, NR, S, SO₂, (CH₂) p, (CHR)ₚ (CHR—CHR′)ₚ, or (CRR′) p;

p is a number from 0 to 6; and

R, R′, R², R³ and R⁴ are independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen; (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;

wherein said alkyl, heteroalkyl, alkenyl, heteroalkenyl, aryl, heteroaryl, cycloalkyl and heterocycloalkyl moieties may be optionally and chemically-suitably substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;

further wherein said unit N-C-G-E-L-J-N represents a five-membered or six-membered cyclic ring structure with the proviso that when said unit N-C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of the cyclic ring.

In another embodiment, the “at least one compound” is a compound of structural Formula II: 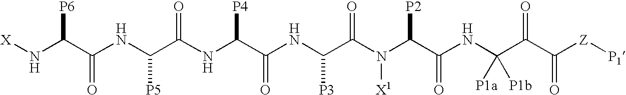
or a pharmaceutically acceptable salt, solvate or ester thereof;
wherein in Formula II:

Z is NH;

X is alkylsulfonyl, heterocyclylsulfonyl, heterocyclylalkylsulfonyl, arylsulfonyl, heteroarylsulfonyl, alkylcarbonyl, heterocyclylcarbonyl, heterocyclylalkylcarbonyl, arylcarbonyl, heteroarylcarbonyl, alkoxycarbonyl, heterocyclyloxycarbonyl, aryloxycarbonyl, heteroaryloxycarbonyl, alkyaminocarbonyl, heterocyclylaminocarbonyl, arylaminocarbonyl, or heteroarylaminocarbonyl moiety, with the proviso that X may be additionally optionally substituted with R¹² or R¹³;

X¹: is H; C₁-C₄ straight chain alkyl; C₁-C₄ branched alkyl or; CH₂-aryl (substituted or unsubstituted);

R¹² is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that R¹² may be additionally optionally substituted with R¹³.

R¹³ is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro moiety, with the proviso that the alkyl, alkoxy, and aryl may be additionally optionally substituted with moieties independently selected from R¹³.

P1a, P1b, P2, P3, P4, P5, and P6 are independently: H; C1-C10 straight or branched chain alkyl; C2-C10 straight or branched chain alkenyl; C3-C8 cycloalkyl, C3-C8 heterocyclic; (cycloalkyl)alkyl or (heterocyclyl)alkyl, wherein said cycloalkyl is made up of 3 to 8 carbon atoms, and zero to 6 oxygen, nitrogen, sulfur, or phosphorus atoms, and said alkyl is of 1 to 6 carbon atoms; aryl, heteroaryl, arylalkyl, or heteroarylalkyl, wherein said alkyl is of 1 to 6 carbon atoms;

wherein said alkyl, alkenyl, cycloalkyl, heterocyclyl; (cycloalkyl)alkyl and (heterocyclyl)alkyl moieties may be optionally substituted with R¹³, and further wherein said P1a and P1b may optionally be joined to each other to form a spirocyclic or spiroheterocyclic ring, with said spirocyclic or spiroheterocyclic ring containing zero to six oxygen, nitrogen, sulfur, or phosphorus atoms, and may be additionally optionally substituted with R¹³; and

P1′ is H, alkyl, alkenyl, alkynyl, cycloalkyl, cycloalkyl-alkyl, heterocyclyl, heterocyclyl-alkyl, aryl, aryl-alkyl, heteroaryl, or heteroaryl-alkyl; with the proviso that said P1′ may be additionally optionally substituted with R

In another embodiment, the “at least one compound” is a compound of structural Formula III: 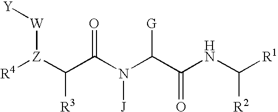
or a pharmaceutically acceptable salt, solvate or ester thereof;
wherein in Formula III:

G is carbonyl;

J and Y may be the same or different and are independently selected from the group consisting of the moieties: H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe additionally optionally substituted with X¹¹ or X¹²;

X¹¹ is selected from the group consisting of alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that X¹¹ may be additionally optionally substituted with X¹²;

X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;

R¹ is COR⁵ or C(OR)₂, wherein R⁵ is selected from the group consisting of H, OH, OR⁸, NR⁹R¹⁰, CF₃, C₂F₅, C₃F₇, CF₂R⁶, R⁶ and COR⁷ wherein R⁷ is selected from the group consisting of H, OH, OR⁸, CHR⁹R¹⁰, and NR⁹R¹⁰, wherein R⁶, R⁸, R⁹ and R¹⁰ may be the same or different and are independently selected from the group consisting of H, alkyl, aryl, heteroalkyl, heteroaryl, cycloalkyl, cycloalkyl, arylalkyl, heteroarylalkyl, CH(R¹′)COOR¹¹, CH(R¹′)CONR¹²R¹³, CH(R¹′)CONHCH(R²)COOR¹¹, CH(R¹′)CONHCH(R²)CONR¹²R¹³, CH(R¹′)CONHCH(R²)R′, CH(R¹′)CONHCH(R²)C ONHCH(R³)COOR¹¹, CH(R¹′)CONHCH(R²′)CONHCH(R³′)CONR¹²R¹³, CH(R¹′)CONHCH(R²)CONHCH(R³)CONHCH(R⁴)COOR¹¹, CH(R¹)CONHCH(R²′)C ONHCH(R³)CONHCH(R⁴)CONR¹²R¹³, CH(R¹′)CONHCH(R²)CONHCH(R³)CONH CH(R⁴)CONHCH(R⁵)COOR¹¹, and CH(R¹′)CONHCH(R²)CONHCH(R³′)CONHCH(R⁴′)CONHCH(R⁵′) CONR¹²R¹³, wherein R¹′, R², R³, R⁴, R⁵, R¹¹, R¹², R¹³, and R′ may be the same or different and are independently selected from a group consisting of H, alkyl, aryl, heteroalkyl, heteroaryl, cycloalkyl, alkyl-aryl, alkyl-heteroaryl, aryl-alkyl and heteroaralkyl;

Z is selected from O, N, or CH;

W maybe present or absent, and if W is present, W is selected from C═O, C═S, or SO₂; and

R, R′, R², R³ and R⁴ are independently selected from the group consisting of H; C1-C10 alkyl; C2-C10 alkenyl; C3-C8 cycloalkyl; C3-C8 heterocycloalkyl, alkoxy, aryloxy, alkylthio, arylthio, amino, amido, ester, carboxylic acid, carbamate, urea, ketone, aldehyde, cyano, nitro; oxygen, nitrogen, sulfur, or phosphorus atoms (with said oxygen, nitrogen, sulfur, or phosphorus atoms numbering zero to six); (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;

wherein said alkyl, heteroalkyl, alkenyl, heteroalkenyl, aryl, heteroaryl, cycloalkyl and heterocycloalkyl moieties may be optionally substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamide, sulfoxide, sulfone, sulfonylurea, hydrazide, and hydroxamate.

In another embodiment, the “at least one compound” is a compound of structural Formula IV: 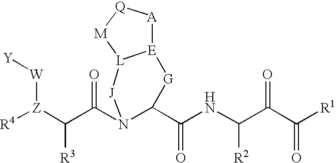
or a pharmaceutically acceptable salt, solvate or ester thereof;
wherein in Formula IV:
Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;

R¹ is selected from the following structures:

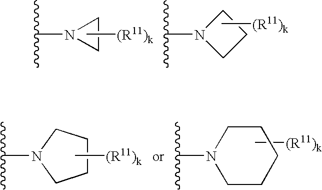

wherein k is a number from 0 to 5, which can be the same or different, R¹¹ denotes optional substituents, with each of said substituents being independently selected from the group consisting of alkyl, alkenyl, alkynyl, aryl, cycloalkyl, alkyl-aryl, heteroalkyl, heteroaryl, aryl-heteroaryl, alkyl-heteroaryl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino, heterocycloalkylamino, hydroxy, thio, alkylthio, arylthio, amino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, and nitro, with the proviso that R¹¹ (when R¹¹ ≠H) maybe optionally substituted with X¹¹ or X¹²;

Z is selected from O, N, CH or CR;

W may be present or absent, and if W is present, W is selected from C═O, C═S, C(═N—CN), or S(O₂);

Q may be present or absent, and when Q is present, Q is CH, N, P, (CH₂)ₚ, (CHR)ₚ, (CRR′)ₚ, O, N(R), S, or S(O₂); and when Q is absent, M may be present or absent; when Q and M are absent, A is directly linked to L;

A is O, CH₂, (CHR)ₚ, (CHR—CHR′)ₚ, (CRR′)ₚ, N(R), S, S(O₂) or a bond;

E is CH, N, CR, or a double bond towards A, L or G;

G may be present or absent, and when G is present, G is (CH₂)ₚ, (CHR)ₚ, or (CRR′)ₚ; and when G is absent, J is present and E is directly connected to the carbon atom in Formula I as G is linked to;

J may be present or absent, and when J is present, J is (CH₂)ₚ, (CHR)ₚ, or (CRR′)ₚ, S(O₂), NH, N(R) or 0; and when J is absent, G is present and E is directly linked to N shown in Formula I as linked to J;

L may be present or absent, and when L is present, L is CH, C(R), O, S or N(R); and when L is absent, then M may be present or absent; and if M is present with L being absent, then M is directly and independently linked to E, and J is directly and independently linked to E;

M may be present or absent, and when M is present, M is O, N(R), S, S(O₂), (CH₂)ₚ, (CHR)ₚ (CHR—CHR′)ₚ, or (CRR′) p;

p is a number from 0 to 6; and

R, R′, R², R³ and R⁴ can be the same or different, each being independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen, (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;

wherein said alkyl, heteroalkyl, alkenyl, heteroalkenyl, aryl, heteroaryl, cycloalkyl and heterocycloalkyl moieties may be optionally substituted, with said term “substituted” referring to substitution with one or more moieties which can be the same or different, each being independently selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;

further wherein said unit N-C-G-E-L-J-N represents a five-membered cyclic ring structure or six-membered cyclic ring structure with the proviso that when said unit N-C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of said five-membered cyclic ring.

In another embodiment, the “at least one compound” is a compound of structural Formula V: 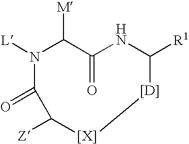
or a pharmaceutically acceptable salt, solvate or ester thereof;
wherein in Formula V:
(1) R¹ is —C(O)R⁵ or —B(OR)₂;
(2) R⁵ is H, —OH, —OR⁸, —NR⁹R¹⁰, —C(O)OR⁸, —C(O)NR⁹R¹⁰, —CF₃, —C₂F₅, C₃F₇, —CF₂R⁶, —R⁶, —C(O)R⁷ or NR⁷SO₂R⁸;
(3) R⁷ is H, —OH, —OR⁸, or —CHR⁹R¹⁰;
(4) R⁶, R⁸, R⁹ and R¹⁰ are independently selected from the group consisting of H: alkyl, alkenyl, aryl, heteroalkyl, heteroaryl, cycloalkyl, arylalkyl, heteroarylalkyl, R¹⁴, —CH(R¹′)CH(R¹′)C(O)OR¹¹, [CH(R¹′)]ₚC(O)OR¹¹, —[CH(R¹′)]ₚC(O)NR¹²R¹³, [CH(R¹′)]ₚS(O₂)R¹¹, —[CH(R¹)]ₚC(O)R¹¹, —[CH(R¹′)]ₚS(O₂)NR¹²R¹³, —CH(R¹′)C(O)N(H)CH(R²′)(R′), CH(R¹′)CH(R¹′)C(O)NR¹²R¹³, —CH(R¹′)CH(R¹)S(O₂)R¹¹, —CH(R¹′)CH(R¹′)S(O₂)NR¹²R¹³, —CH(R¹′)CH(R¹′)C(C)R¹¹, —[CH(R¹)]ₚCH(OH)R¹¹, —CH(R¹)C(O)N(H)CH(R²′)C(O)OR¹¹, C(O)N(H)CH(R²′)C(O)OR¹¹, —C(O)N(H)CH(R²′)C(O)R¹¹, CH(R¹′)C(O)N(H)CH(R²′) C(O)NR¹²R¹³, —CH(R¹′)C(O)N(H)CH(R²′)R′, CH(R¹′)C(O)N(H)CH(R²′)C(O)N(H) CH(R³′)C(O)OR¹¹, CH(R¹′)C(O)N(H)CH(R²′)C(O)CH(R³′)NR¹²R¹³, CH(R¹)C(O)N(H)C H(R²′)C(O)N(H)CH(R²′)(O)NR¹²R¹³CH(R¹′)C(O)N(H)CH(R²′)C(O)N(H)CH(R³′)C(O)N(H)CH(R⁴′)C(O)OR¹¹, H(R¹′)C(O)N(H)CH(R²′)C(O)N(H)CH(R³′)C(O)N(H)CH(R⁴′)C(O)NR¹²R¹³, CH(R¹)C(O)N(H)CH(R²′)C(O)N(H)CH(R³′)C(O)N(H)CH(R⁴′)C(O)N(H)CH(R⁵′)C(O)OR¹¹, and CH(R¹)C(O)N(H)CH(R²′)C(O)N(H)CH(R³′)C(O)N(H)CH(R⁴′)C(O)N(H)CH(R⁵′) C(O)NR¹²R¹³;
wherein R¹, R²′, R³, R⁴′, R⁵′, R¹¹, R¹² and R¹³ can be the same or different, each being independently selected from the group consisting of: H, halogen, alkyl, aryl, heteroalkyl, heteroaryl, cycloalkyl, alkoxy, aryloxy, alkenyl, alkynyl, alkyl-aryl, alkyl-heteroaryl, heterocycloalkyl, aryl-alkyl and heteroaralkyl; or
R¹² and R¹³ are linked together wherein the combination is cycloalkyl, heterocycloalkyl, ary or heteroaryl;
R¹⁴ is present or not and if present is selected from the group consisting of: H, alkyl, aryl, heteroalkyl, heteroaryl, cycloalkyl, alkyl-aryl, allyl, alkyl-heteroaryl, alkoxy, aryl-alkyl, alkenyl, alkynyl and heteroaralkyl;
(5) R and R′ are present or not and if present can be the same or different, each being independently selected from the group consisting of: H, OH, C₁-C₁₀ alkyl, C₂-C₁₀ alkenyl, C₃-C₈ cycloalkyl, C₃-C₈ heterocycloalkyl, alkoxy, aryloxy, alkylthio, arylthio, alkylamino, arylamino, amino, amido, arylthioamino, arylcarbonylamino, arylaminocarboxy, alkylaminocarboxy, heteroalkyl, alkenyl, alkynyl, (aryl)alkyl, heteroarylalkyl, ester, carboxylic acid, carbamate, urea, ketone, aldehyde, cyano, nitro, halogen, (cycloalkyl)alkyl, aryl, heteroaryl, (alkyl)aryl, alkylheteroaryl, alkyl-heteroaryl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms;
(6) L′ is H, OH, alkyl, heteroalkyl, aryl, heteroaryl, cycloalkyl, or heterocyclyl;
(7) M′ is H, alkyl, heteroalkyl, aryl, heteroaryl, cycloalkyl, arylalkyl, heterocyclyl or an amino acid side chain; or L′ and M′ are linked together to form a ring structure wherein the portion of structural Formula 1 represented by: 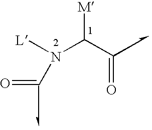
and wherein structural Formula 2 is represented by: 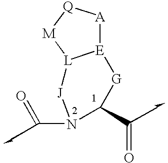
wherein in Formula 2:
E is present or absent and if present is C, CH, N or C(R);
J is present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;
p is a number from 0 to 6;
L is present or absent, and when L is present, L is C(H) or C(R); when L is absent,
M is present or absent; if M is present with L being absent, then M is directly and independently linked to E, and J is directly and independently linked to E;
G is present or absent, and when G is present, G is (CH₂)ₚ, (CHR)ₚ, (CHR—CHR′)ₚ or (CRR′)ₚ; when G is absent, J is present and E is directly connected to the carbon atom marked position 1;
Q is present or absent, and when Q is present, Q is NR, PR, (CR═CR), (CH₂)ₚ, (CHR)ₚ, (CRR′)ₚ, (CHR—CHR′)ₚ, O, NR, S, SO, or SO₂; when Q is absent, M is (i) either directly linked to A or (ii) an independent substituent on L, said independent substituent bing selected from —OR, —CH(R)(R′), S(O)₀₋₂R or —NRR′ or (iii) absent; when both Q and M are absent, A is either directly linked to L, or A is an independent substituent on E, said independent substituent bing selected from —OR, —CH(R)(R′), S(O)₀₋₂R or —NRR′ or A is absent;
A is present or absent and if present A is O, O(R), (CH₂)ₚ, (CHR)ₚ, (CHR—CHR′)ₚ, (CRR′)ₚ, N(R), NRR′, S, S(O₂), —OR, CH(R)(R′) or NRR′; or A is linked to M to form an alicyclic, aliphatic or heteroalicyclic bridge;
M is present or absent, and when M is present, M is halogen, 0, OR, N(R), S, S(O₂), (CH₂)ₚ, (CHR)ₚ(CHR—CHR′)ₚ, or (CRR′)ₚ; or M is linked to A to form an alicyclic, aliphatic or heteroalicyclic bridge;
(8) Z′ is represented by the structural Formula 3: 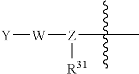
wherein in Formula 3:
Y is selected from the group consisting of: H, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, heteroalkyl-heterocycloalkyl, cycloalkyloxy, alkylamino, arylamino, alkyl-arylamino, arylamino, heteroarylamino, cycloalkylamino and heterocycloalkylamino, and Y is unsubstituted or optionally substituted with one or two substituents which are the same or different and are independently selected from X¹¹ or X¹²; X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹¹ is unsubstituted or optionally substituted with one or more of X¹² moieties which are the same or different and are independently selected;
X¹² is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, sulfonylurea, cycloalkylsulfonamido, heteroaryl-cycloalkylsulfonamido, heteroaryl-sulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;
Z is O, N, C(H) or C(R);
R³¹ is H, hydroxyl, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, cycloalkyloxy, alkylamino, arylamino, alkyl-arylamino, arylamino, heteroarylamino, cycloalkylamino or heterocycloalkylamino, and R³¹ is unsubstituted or optionally substituted with one or two substituents which are the same or different and are independently selected from X¹³ or X¹⁴;
X¹³ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹³ is unsubstituted or optionally substituted with one or more of X¹⁴ moieties which are the same or different and are independently selected;
X¹⁴ is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, cycloalkylsulfonamido, heteroaryl-cycloalkylsulfonamido, heteroarylsulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;
W may be present or absent, and if W is present, W is C(═O), C(═S), C(═N—CN), or S(O₂);

(9) X is represented by structural Formula 4: 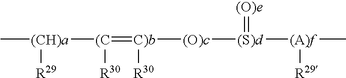
wherein in Formula 4:
a is 2, 3, 4, 5, 6, 7, 8 or 9;
b, c, d, e and f are 0, 1, 2, 3, 4 or 5;
A is C, N, S or O;
R²⁹ and R²⁹′ are independently present or absent and if present can be the same or different, each being independently one or two substituents independently selected from the group consisting of: H, halo, alkyl, aryl, cycloalkyl, cycloalkylamino, cycloalkylaminocarbonyl, cyano, hydroxy, alkoxy, alkylthio, amino, —NH(alkyl), —NH(cycloalkyl), —N(alkyl)₂, carboxyl, C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or
R²⁹ and R²⁹′ are linked together such that the combination is an aliphatic or heteroaliphatic chain of 0 to 6 carbons;
R³⁰ is present or absent and if present is one or two substituents independently selected from the group consisting of: H, alkyl, aryl, heteroaryl and cylcoalkyl;

(10) D is represented by structural Formula 5: 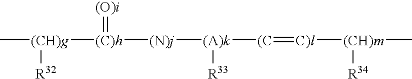
wherein in Formula 5:
R³², R³³ and R³⁴ are present or absent and if present are independently one or two substituents independently selected from the group consisting of: H, halo, alkyl, aryl, cycloalkyl, cycloalkylamino, spiroalkyl, cycloalkylaminocarbonyl, cyano, hydroxy, alkoxy, alkylthio, amino, —NH(alkyl), —NH(cycloalkyl), —N(alkyl)₂, carboxyl, —C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or R³² and R³⁴ are linked together such that the combination forms a portion of a cycloalkyl group;
g is 1, 2, 3, 4, 5, 6, 7, 8 or 9;
h, i, j, k, l and m are 0, 1, 2, 3, 4 or 5; and
A is C, N, S or O,

(11) provided that when structural Formula 2:

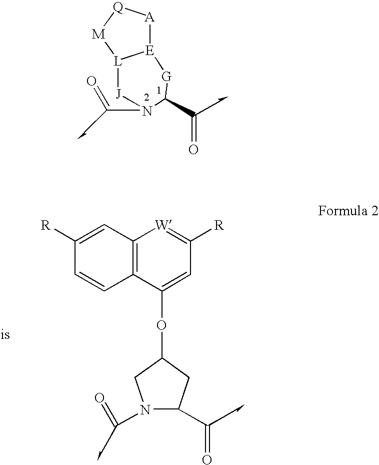

and

W′ is CH or N, both the following conditional exclusions (i) and (ii) apply: conditional exclusion (i): Z′ is not —NH—R³⁶, wherein R³⁶ is H, C_{6 or 10} aryl, heteroaryl, —C(O)—R³⁷, —C(O)—OR³⁷ or —C(O)—NHR³⁷, wherein R³⁷ is C₁₋₆ alkyl or C₃₋₆cycloalkyl;

and

conditional exclusion (ii): R¹ is not —C(O)OH, a pharmaceutically acceptable salt of —C(O)OH, an ester of —C(O)OH or —C(O)NHR³⁸ wherein R³⁸ is selected from the group consisting of C₁₋₈alkyl, C₃₋₆cycloalkyl, C_{6 to 10} aryl or C₇₋₁₆ aralkyl.

In another embodiment, the “at least one compound” is a compound of structural Formula VI: 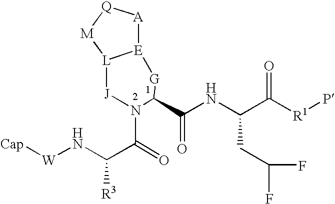
or a pharmaceutically acceptable salt, solvate or ester thereof;
wherein in Formula VI:

Cap is H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arlylalkyloxy or heterocyclylamino, wherein each of said alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arlylalkyloxy or heterocyclylamino can be unsubstituted or optionally independently substituted with one or two substituents which can be the same or different and are independently selected from X¹ and X²;

P′ is —NHR;

X¹ is alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl, or heteroarylalkyl, and X¹ can be unsubstituted or optionally independently substituted with one or more of X² moieties which can be the same or different and are independently selected;

X² is hydroxy, alkyl, aryl, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, keto, ester or nitro, wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl and heteroarylalkyl;

W may be present or absent, and when W is present W is C(═O), C(═S), C(═NH), C(═N—OH), C(═N—CN), S(O) or S(O₂);

Q maybe present or absent, and when Q is present, Q is N(R), P(R), CR═CR′, (CH₂)ₚ, (CHR)ₚ, (CRR′)ₚ, (CHR—CHR′)ₚ, O, S, S(O) or S(O₂); when Q is absent, M is (i) either directly linked to A or (ii) M is an independent substituent on L and A is an independent substituent on E, with said independent substituent being selected from —OR, —CH(R), S(O)₀₋₂R or —NRR′; when both Q and M are absent, A is either directly linked to L, or A is an independent substituent on E, selected from —OR, CH(R)(R′), —S(O)₀₋₂R or —NRR′;

A is present or absent and if present A is —O—, —O(R) CH₂—, —(CHR)ₚ—, —(CHR—CHR′)ₚ—, (CRR′)ₚ, N(R), NRR′, S, or S(O₂), and when Q is absent, A is —OR, —CH(R)(R′) or —NRR′; and when A is absent, either Q and E are connected by a bond or Q is an independent substituent on M;

E is present or absent and if present E is CH, N, C(R);

G may be present or absent, and when G is present, G is (CH₂)ₚ, (CHR)ₚ, or (CRR′)ₚ; when G is absent, J is present and E is directly connected to the carbon atom marked position 1;

J may be present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;

L may be present or absent, and when L is present, L is CH, N, or CR; when L is absent, M is present or absent; if M is present with L being absent, then M is directly and independently linked to E, and J is directly and independently linked to E;

M may be present or absent, and when M is present, M is O, N(R), S, S(O₂), (CH₂)ₚ, (CHR)ₚ, (CHR—CHR′)ₚ, or (CRR′)ₚ;

p is a number from 0 to 6;

R, R′ and R³ can be the same or different, each being independently selected from the group consisting of: H, C₁-C₁₀ alkyl, C₂-C₁₀ alkenyl, C₃-C₈ cycloalkyl, C₃-C₈ heterocyclyl, alkoxy, aryloxy, alkylthio, arylthio, amino, amido, arylthioamino, arylcarbonylamino, arylaminocarboxy, alkylaminocarboxy, heteroalkyl, heteroalkenyl, alkenyl, alkynyl, aryl-alkyl, heteroarylalkyl, ester, carboxylic acid, carbamate, urea, ketone, aldehyde, cyano, nitro, halogen, (cycloalkyl)alkyl, aryl, heteroaryl, alkyl-aryl, alkylheteroaryl, alkyl-heteroaryl and (heterocyclyl)alkyl;

R and R′ in (CRR′) can be linked together such that the combination forms a cycloalkyl or heterocyclyl moiety; and

R¹ is carbonyl.

In another embodiment, the “at least one compound” is a compound of structural Formula VII:

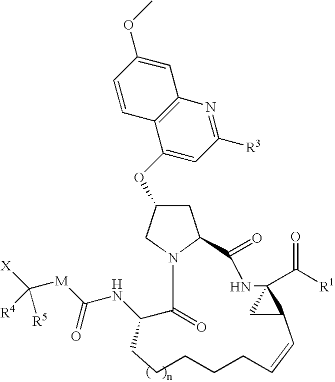

or a pharmaceutically acceptable salt, solvate or ester thereof;

wherein in Formula VIII:

M is O, N(H), or CH₂;

n is 0-4;

R¹ is —OR⁶, —NR⁶R⁷ or

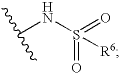

where R⁵ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 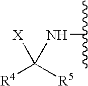
is represented by 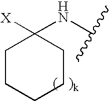
where k is 0 to 2;
X is selected from the group consisting of: and

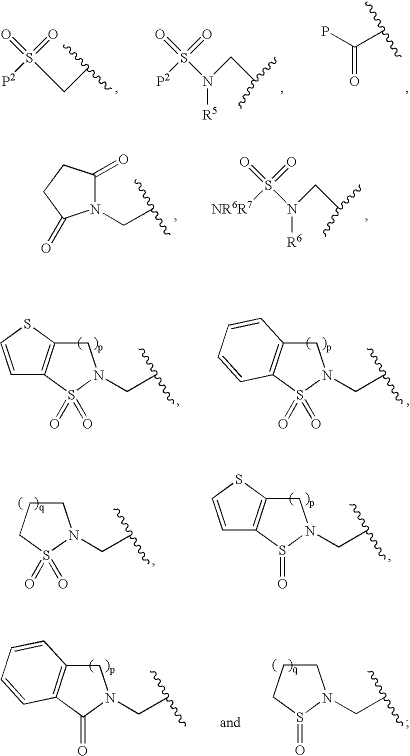

where p is 1 to 2, q is 1-3 and P² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 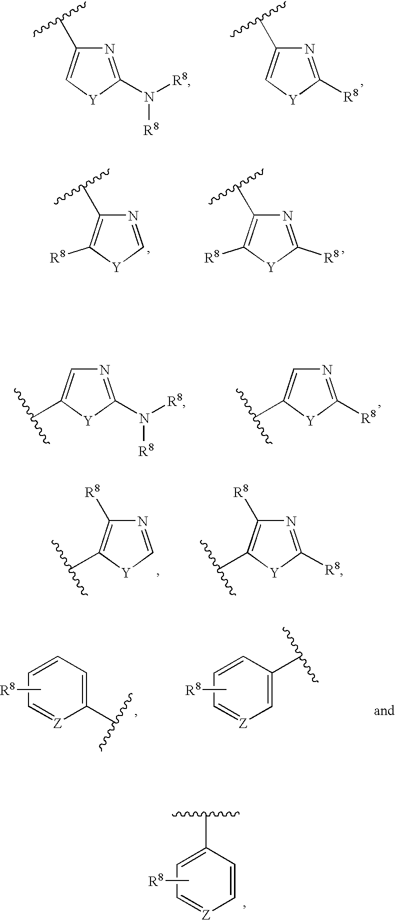
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy.

In another embodiment, the “at least one compound” is a compound of structural Formula VIII: 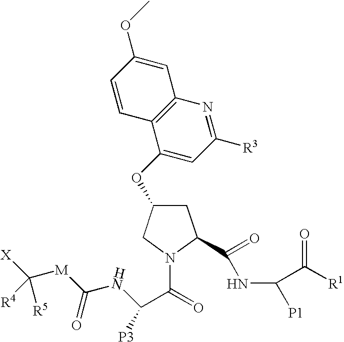
or a pharmaceutically acceptable salt, solvate or ester thereof;
wherein in Formula VIII:

M is O, N(H), or CH₂;

R¹ is —C(O)NHR⁶, where R⁶ is hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino or alkylamino;

P₁ is selected from the group consisting of alkyl, alkenyl, alkynyl, cycloalkyl haloalkyl;

P₃ is selected from the group consisting of alkyl, cycloalkyl, aryl and cycloalkyl fused with aryl;

R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 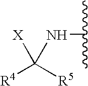
is represented by 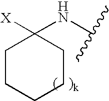
where k is 0 to 2;

X is selected from the group consisting of:

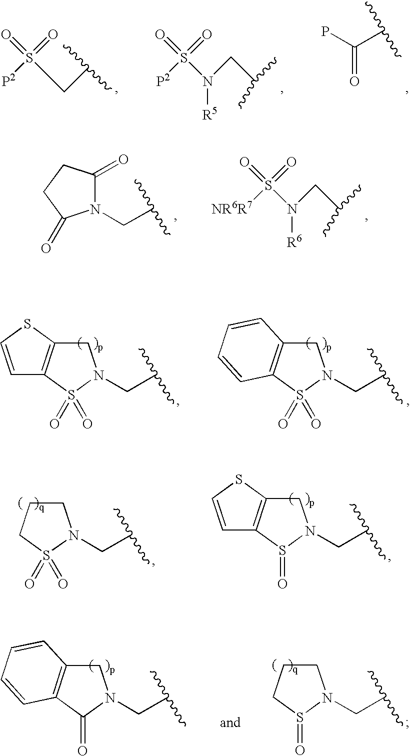

where p is 1 to 2, q is 1 to 3 and P² is alkyl, aryl, heteroaryl, heteroalkyl, cycloalkyl, dialkylamino, alkylamino, arylamino or cycloalkylamino; and

R³ is selected from the group consisting of: aryl, heterocyclyl, heteroaryl, 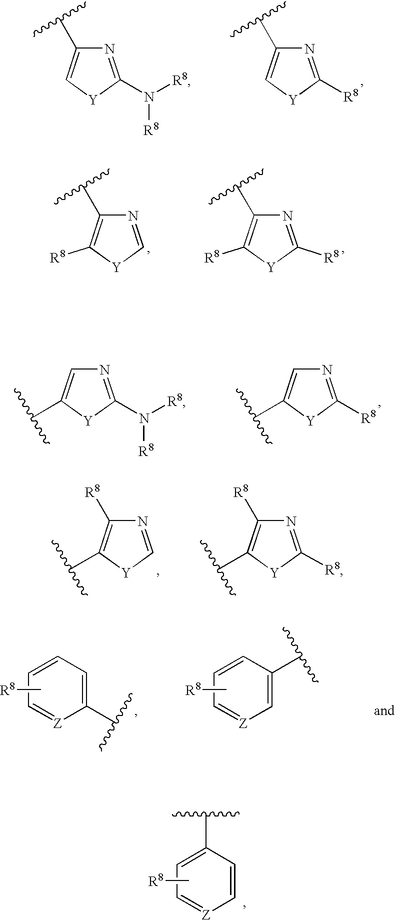
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy.

In another embodiment, the “at least one compound” is a compound of structural Formula IX: 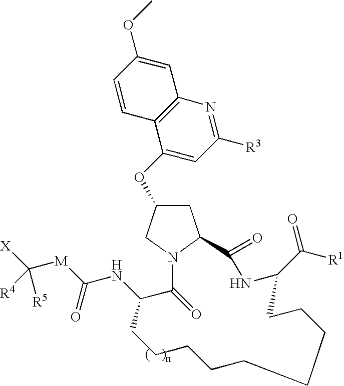
or a pharmaceutically acceptable salt, solvate or ester thereof;
wherein in Formula IX:

M is O, N(H), or CH₂;

n is 0-4;

R¹ is —OR⁶NR⁶R⁷ or

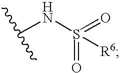

where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 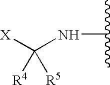
is represented by 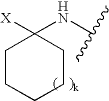
where k is 0 to 2;
X is selected from the group consisting of:

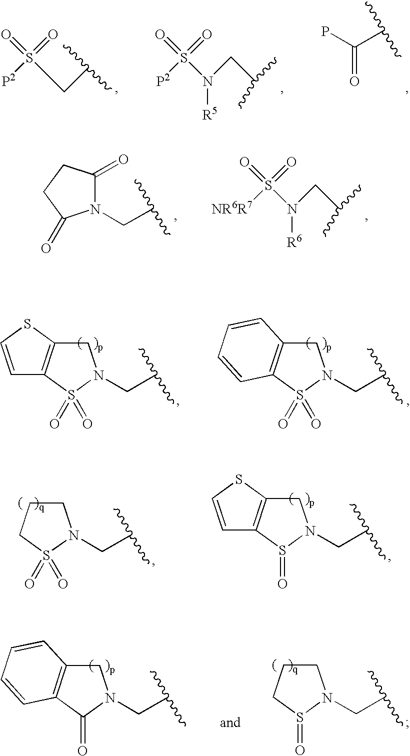

where p is 1 to 2, q is 1 to 3 and P² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl,

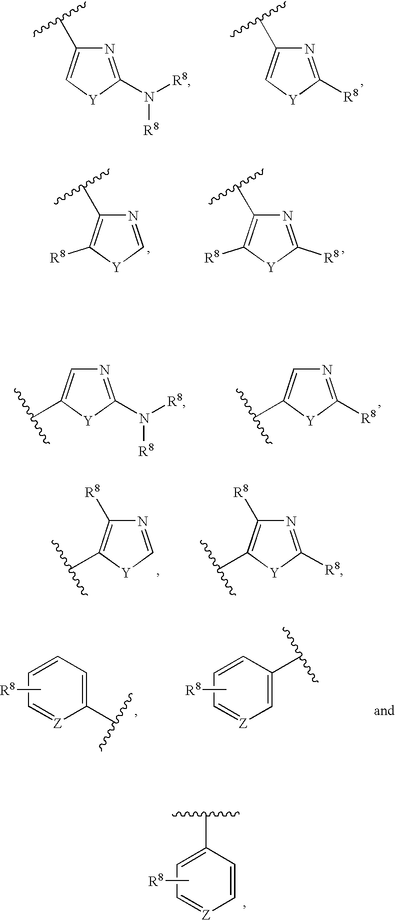

where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy.

In another embodiment, the “at least one compound” is a compound of structural Formula X: 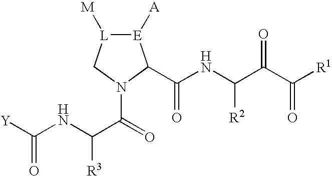
or a pharmaceutically acceptable salt, solvate or ester thereof;
wherein in Formula X:

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

A and M can be the same or different, each being independently selected from R, OR, NHR, NRR′, SR, SO₂R, and halo; or A and M are connected to each other such that the moiety: 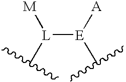
shown above in Formula I forms either a three, four, six, seven or eight-membered cycloalkyl, a four to eight-membered heterocyclyl, a six to ten-membered aryl, or a five to ten-membered heteroaryl;

E is C(H) or C(R);

L is C(H), C(R), CH₂C(R), or C(R)CH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;

and Y is selected from the following moieties: 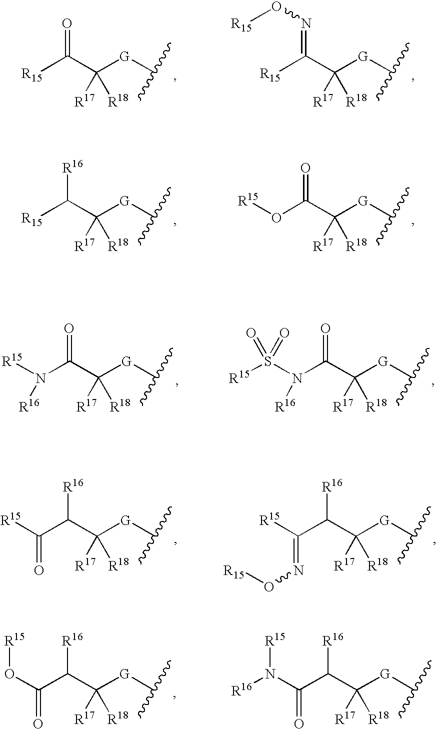
wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁵ and R¹⁶ are connected to each other to form a four to eight-membered cycloalkyl, heteroaryl or heterocyclyl structure, and likewise, independently R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In one embodiment, the “at least one compound” is a compound of structural Formula XI: 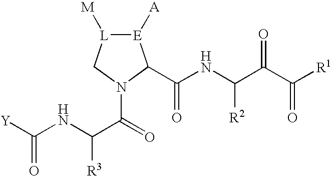
or a pharmaceutically acceptable salt, solvate or ester thereof;
wherein in Formula XI:

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

A and M can be the same or different, each being independently selected from R, NR⁹R¹⁰, SR, SO₂R, and halo; or A and M are connected to each other (in other words, A-E-L-M taken together) such that the moiety: 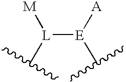
shown above in Formula I forms either a three, four, six, seven or eight-membered cycloalkyl, a four to eight-membered heterocyclyl, a six to ten-membered aryl, or a five to ten-membered heteroaryl;

E is C(H) or C(R);

L is C(H), C(R), CH₂C(R), or C(R)CH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NR⁹R¹⁰ forms a four to eight-membered heterocyclyl;

Y is selected from the following moieties:

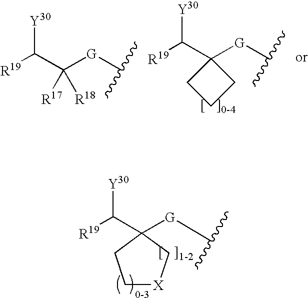

wherein Y³⁰ and Y³¹ are selected from

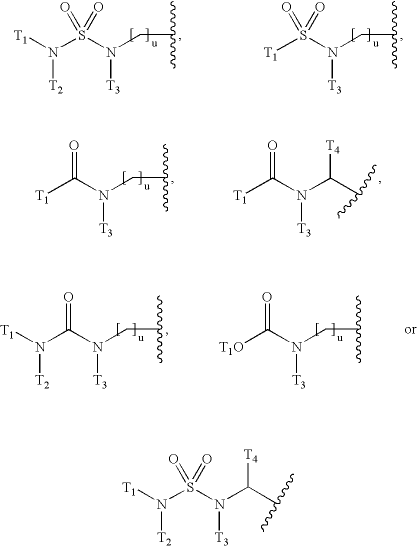

where u is a number 0-6;

X is selected from O, NR¹⁵, NC(O)R¹⁶, S, S(O) and SO₂;

G is NH or O; and

R¹⁵, R¹⁶, R¹⁷, R¹⁸, R¹⁹, T₁, T₂, T₃ and T₄ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the “at least one compound” is a compound of structural Formula XII: 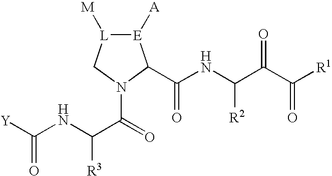
or a pharmaceutically acceptable salt, solvate or ester thereof;
wherein in Formula XII:

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

A and M can be the same or different, each being independently selected from R, OR, NHR, NRR′, SR, SO₂R, and halo; or A and M are connected to each other such that the moiety: 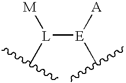
shown above in Formula I forms either a three, four, six, seven or eight-membered cycloalkyl, a four to eight-membered heterocyclyl, a six to ten-membered aryl, or a five to ten-membered heteroaryl;

E is C(H) or C(R);

L is C(H), C(R), CH₂C(R), or C(R)CH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;

and Y is selected from the following moieties: 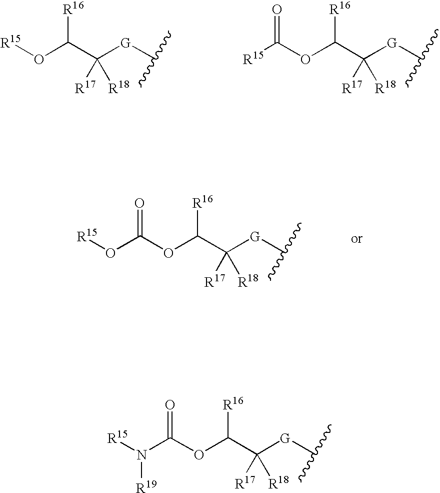
wherein G is NH or O; and R¹⁵, R¹⁶, R¹⁷, R¹⁸, and R¹⁹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, (i) either R¹⁵ and R¹⁶ are connected to each other to form a four to eight-membered cyclic structure, or R¹⁵ and R¹⁹ are connected to each other to form a four to eight-membered cyclic structure, and (ii) likewise, independently, R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro.

In another embodiment, the “at least one compound” is a compound of structural Formula XIII: 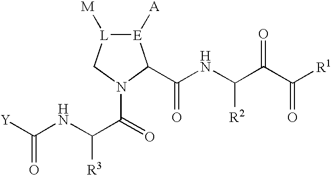
or a pharmaceutically acceptable salt, solvate or ester thereof;
wherein in Formula XIII:

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

A and M can be the same or different, each being independently selected from R, OR, NHR, NRR′, SR, SO₂R, and halo; or A and M are connected to each other (in other words, A-E-L-M taken together) such that the moiety: 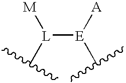
shown above in Formula I forms either a three, four, six, seven or eight-membered cycloalkyl, a four to eight-membered heterocyclyl, a six to ten-membered aryl, or a five to ten-membered heteroaryl;

E is C(H) or C(R);

L is C(H), C(R), CH₂C(R), or C(R)CH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl; and Y is selected from the following moieties:

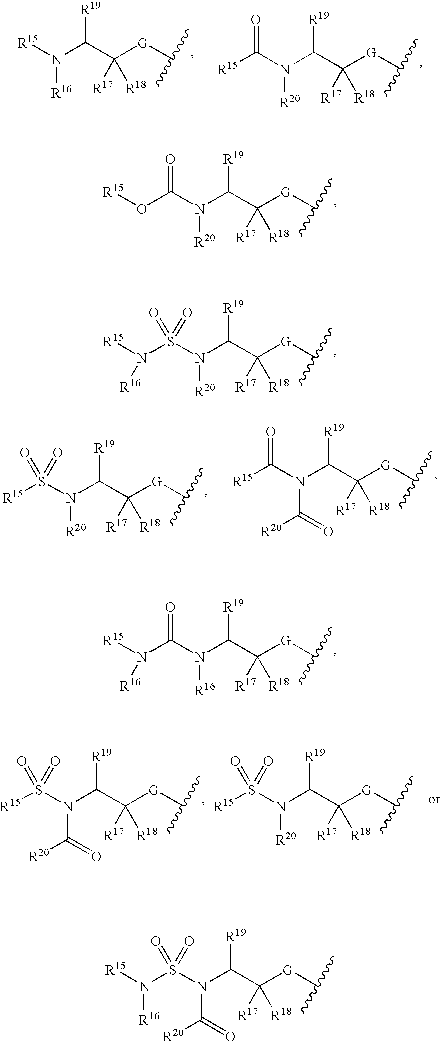

wherein G is NH or O, and R¹⁵, R¹⁶, R¹⁷, R¹⁸, R¹⁹ and R²⁰ can be the same or different, each being independently selected from the group consisting of H, C₁-C₁₀ alkyl, C₁-C₁₀ heteroalkyl, C₂-C₁₀ alkenyl, C₂-C₁₀ heteroalkenyl, C₂-C₁₀ alkynyl, C₂-C₁₀ heteroalkynyl, C₃-C₈ cycloalkyl, C₃-C₈ heterocyclyl, aryl, heteroaryl, or alternately: (i) either R¹⁵ and R¹⁶ can be connected to each other to form a four to eight-membered cycloalkyl or heterocyclyl, or R¹⁵ and R¹⁹ are connected to each other to form a five to eight-membered cycloalkyl or heterocyclyl, or R¹⁵ and R²⁰ are connected to each other to form a five to eight-membered cycloalkyl or heterocyclyl, and (ii) likewise, independently, R¹⁷ and R¹⁸ are connected to each other to form a three to eight-membered cycloalkyl or heterocyclyl,

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the “at least one compound” is a compound of structural Formula XIV: 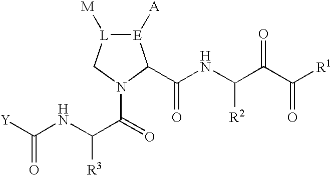
or a pharmaceutically acceptable salt, solvate or ester thereof;
wherein in Formula XIV:

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

A and M can be the same or different, each being independently selected from R, OR, NHR, NRR′, SR, SO₂R, and halo;
or A and M are connected to each other such that the moiety: 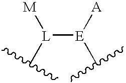
shown above in Formula I forms either a three, four, six, seven or eight-membered cycloalkyl, a four to eight-membered heterocyclyl, a six to ten-membered aryl, or a five to ten-membered heteroaryl;

E is C(H) or C═;

L is C(H), C═, CH₂C═, or C═CH₂;

R, R′, R², and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately R and R′ in NRR′ are connected to each other such that NRR′ forms a four to eight-membered heterocyclyl;

and Y is selected from the following moieties:

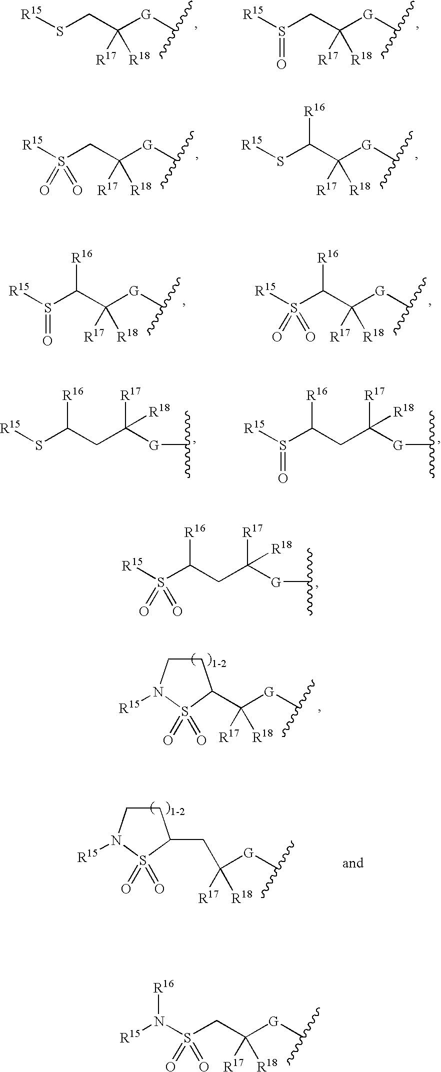

wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, and heteroaryl, or alternately, (i) R¹⁵ and R¹⁶ are connected to each other to form a four to eight-membered cyclic structure, and (ii) likewise, independently R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro.

In another embodiment, the “at least one compound” is a compound of structural Formula XV: 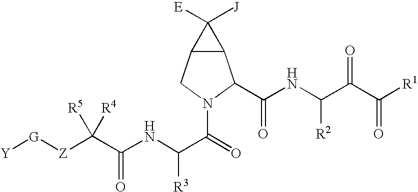
or a pharmaceutically acceptable salt, solvate or ester thereof;
wherein in Formula XV:

R¹ is NHR⁹, wherein R⁹ is H, alkyl-, aryl-, heteroalkyl-, heteroaryl-, cycloalkyl-5, cycloalkyl-, arylalkyl-, or heteroarylalkyl;

E and J can be the same or different, each being independently selected from the group consisting of R, OR, NHR, NRR⁷, SR, halo, and S(O₂)R, or E and J can be directly connected to each other to form either a three to eight-membered cycloalkyl, or a three to eight-membered heterocyclyl moiety;

Z is N(H), N®, or O, with the proviso that when Z is O, G is present or absent and if G is present with Z being 0, then G is C(═O);

G maybe present or absent, and if G is present, G is C(═O) or S(O₂), and

when G is absent, Z is directly connected to Y;

Y is selected from the group consisting of:

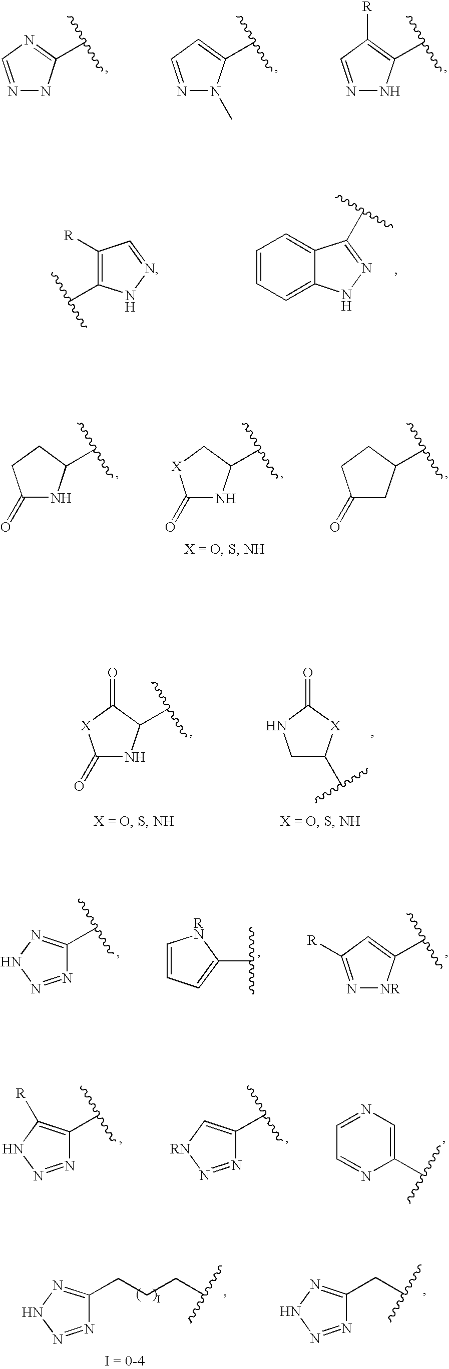

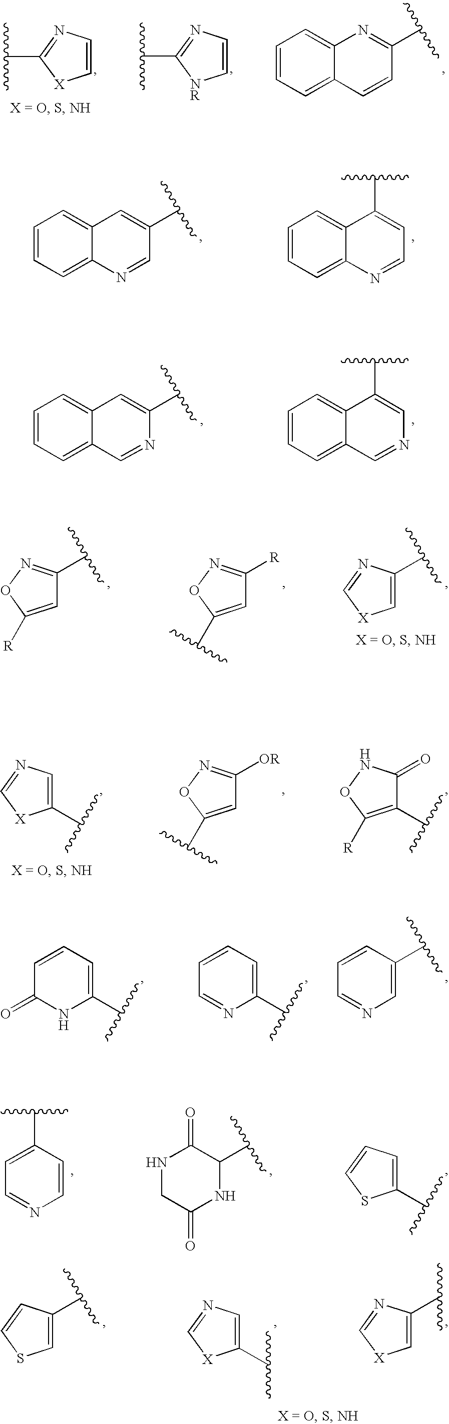

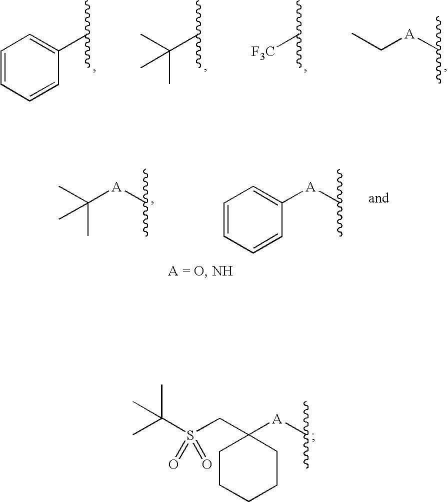

R, R⁷, R², R³, R⁴ and R⁵ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-, wherein each of said heteroalkyl, heteroaryl and heterocyclyl independently has one to six oxygen, nitrogen, sulfur, or phosphorus atoms;

wherein each of said alkyl, heteroalkyl, alkenyl, alkynyl, aryl, heteroaryl, cycloalkyl and heterocyclyl moieties can be unsubstituted or optionally independently substituted with one or more moieties selected from the group consisting of alkyl, alkenyl, alkynyl, aryl, aralkyl, cycloalkyl, heterocyclyl, halo, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate.

In another embodiment, the “at least one compound” is a compound of structural Formula XVI: 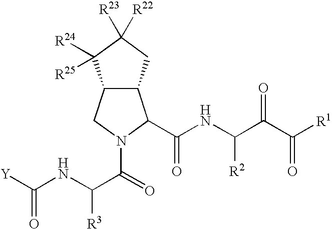
or a pharmaceutically acceptable salt, solvate or ester thereof;
wherein in Formula XVI:

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;

Y is selected from the following moieties:

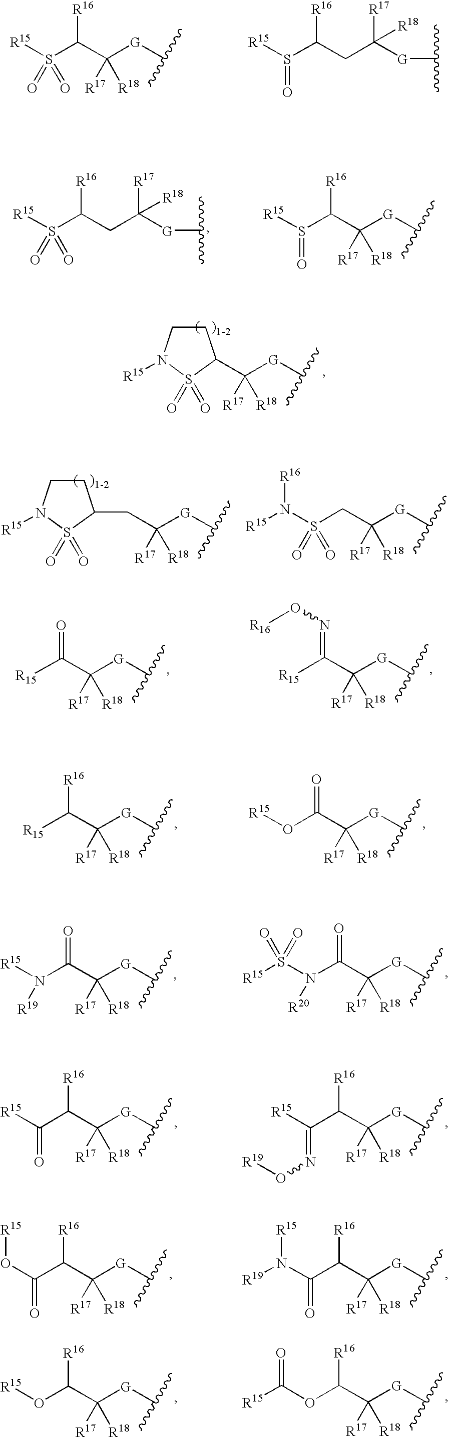

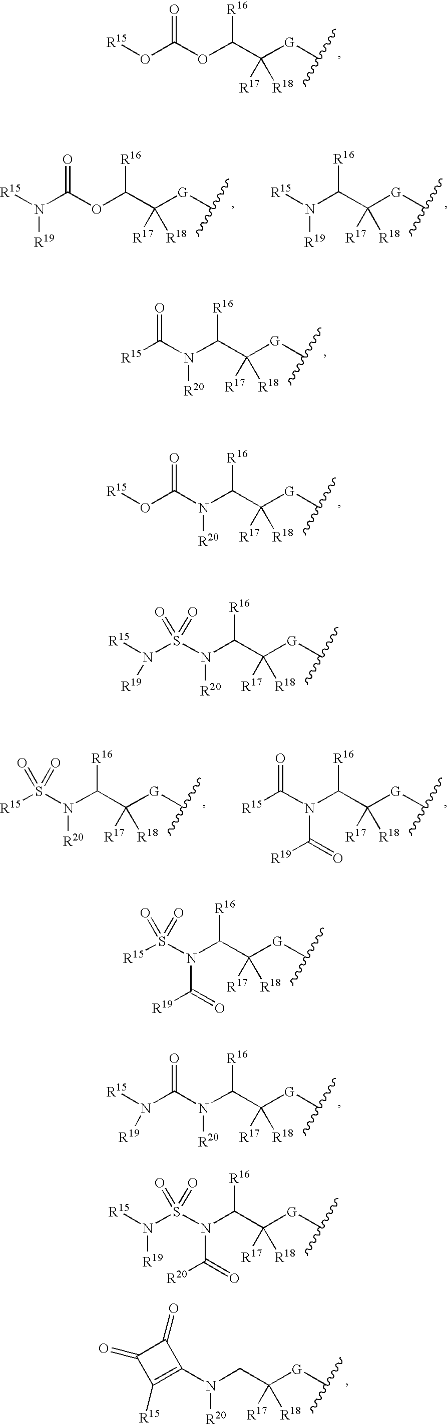

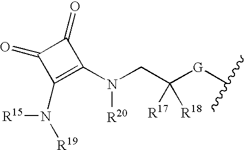

wherein G is NH or O; and R¹⁵, R¹⁶, R¹⁷, R¹⁸, R¹⁹, R²⁰, R²¹, R²², R²³, R²⁴ and R²⁵ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; (iv) likewise independently R¹⁵ and R²⁰ are connected to each other to form a four to eight-membered heterocyclyl; (v) likewise independently R²² and R²³ are connected to each other to form a three to eight-membered cycloalkyl or a four to eight-membered heterocyclyl; and (vi) likewise independently R²⁴ and R²⁵ are connected to each other to form a three to eight-membered cycloalkyl or a four to eight-membered heterocyclyl;

wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the “at least one compound” is a compound of structural Formula XVII: 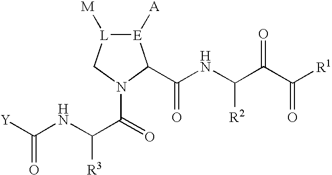
or a pharmaceutically acceptable salt, solvate or ester thereof;
wherein in Formula XVII:

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

A and M can be the same or different, each being independently selected from R, OR, NHR, NRR′, SR, SO₂R, and halo; or A and M are connected to each other such that the moiety: 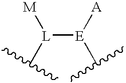
shown above in Formula I forms either a three, four, six, seven or eight-membered cycloalkyl, a four to eight-membered heterocyclyl, a six to ten-membered aryl, or a five to ten-membered heteroaryl;

E is C(H) or C═;

L is C(H), C═, CH₂C═, or C═CH₂;

R, R′, R², and R³ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;

Y is selected from the following moieties:

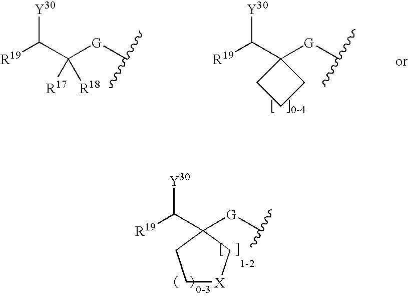

wherein Y³⁰ is selected from

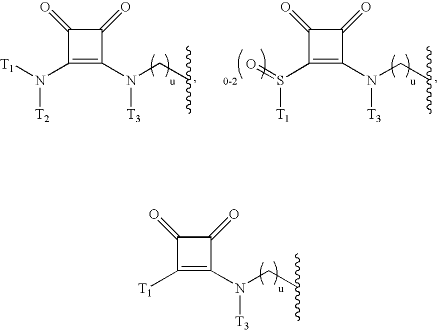

where u is a number 0-1;

X is selected from O, NR¹⁵, NC(O)R¹⁶, S, S(O) and SO₂;

G is NH or O; and

R¹⁵, R¹⁶, R¹⁷, R¹⁸, R¹⁹, T₁, T₂, and T₃ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;

wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the “at least one compound” is a compound of structural Formula XVIII: 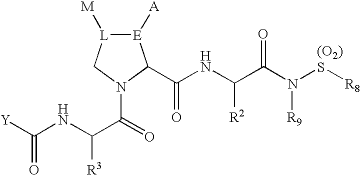
or a pharmaceutically acceptable salt, solvate or ester thereof;
wherein in Formula XVIII:
R⁸ is selected from the group consisting of alkyl-, aryl-, heteroalkyl-, heteroaryl-, cycloalkyl-, heterocyclyl-, arylalkyl-, heteroarylalkyl-, and heterocyclylalkyl;
R⁹ is selected from the group consisting of H, alkyl, alkenyl, alkynyl, aryl and cycloalkyl;
A and M can be the same or different, each being independently selected from R, OR, N(H)R, N(RR′), SR, S(O₂)R, and halo; or A and M are connected to each other (in other words, A-E-L-M taken together) such that the moiety:

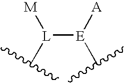

shown above in Formula I forms either a three, four, five, six, seven or eight-membered cycloalkyl, a four to eight-membered heterocyclyl, a six to ten-membered aryl, or a five to ten-membered heteroaryl;

E is C(H) or C(R);

L is C(H), C(R), CH₂C(R), or C(R)CH₂;

R and R′ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in N(RR′) are connected to each other such that N(RR′) forms a four to eight-membered heterocyclyl; R² and R³ can be the same or different, each being independently selected from the group consisting of H, alkyl, heteroalkyl, alkenyl, heteroalkenyl, alkynyl, heteroalkynyl, cycloalkyl, spiro-linked cycloalkyl, heterocyclyl, aryl, arylalkyl, heteroaryl, and heteroarylalkyl;

Y is selected from the following moieties:

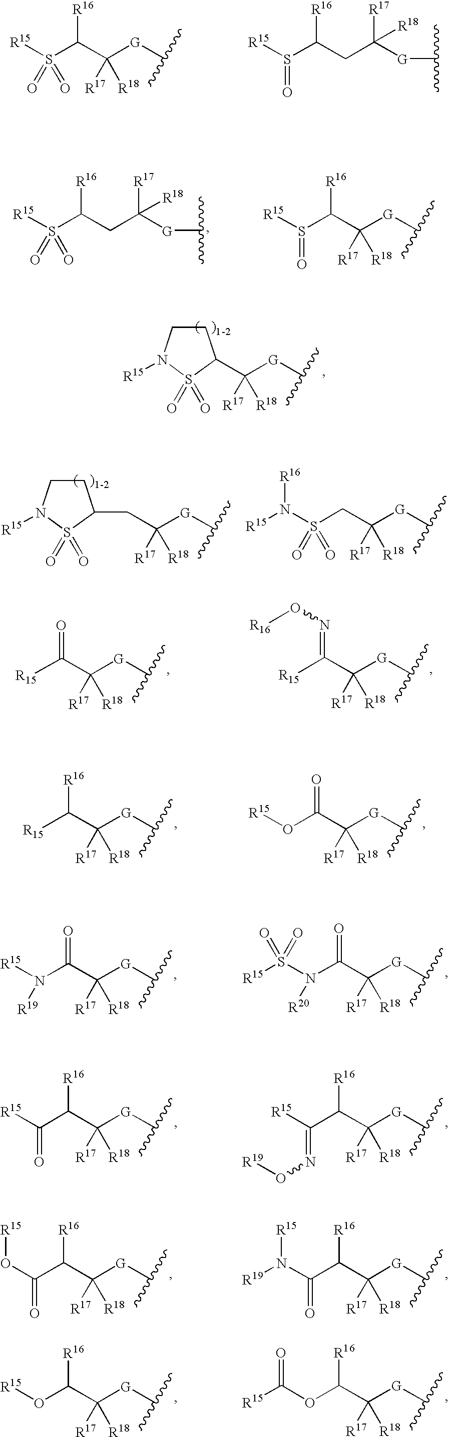

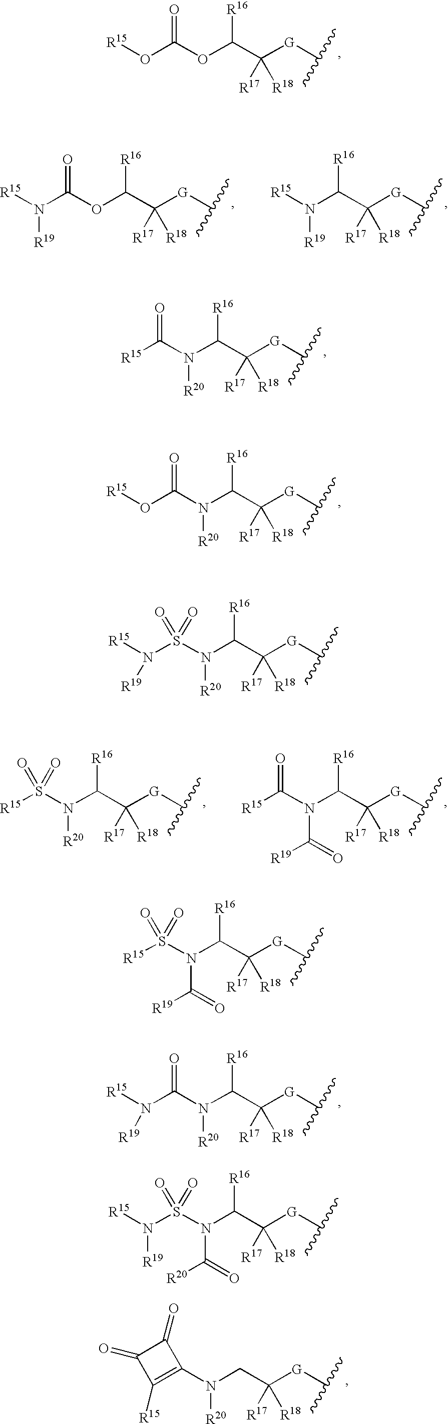

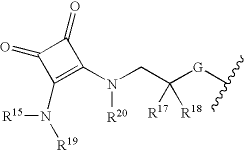

wherein G is NH or O; and R¹⁵, R¹⁶, R¹⁷, R¹⁸, R¹⁹ and R²⁰ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl; wherein each of said alkyl, aryl, heteroaryl, cycloalkyl, spiro-linked cycloalkyl, and heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, alkenyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the “at least one compound” is a compound of structural Formula XIX: 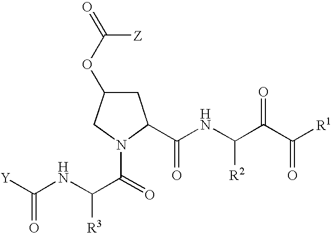
or a pharmaceutically acceptable salt, solvate or ester thereof;
wherein in Formula XIX:

Z is selected from the group consisting of a heterocyclyl moiety, N(H)(alkyl), —N(alkyl)₂, —N(H)(cycloalkyl), —N(cycloalkyl)₂, —N(H)(aryl, —N(aryl)₂, —N(H)(heterocyclyl), —N(heterocyclyl)₂, —N(H)(heteroaryl), and —N(heteroaryl)₂;

R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;

R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;

Y is selected from the following moieties:

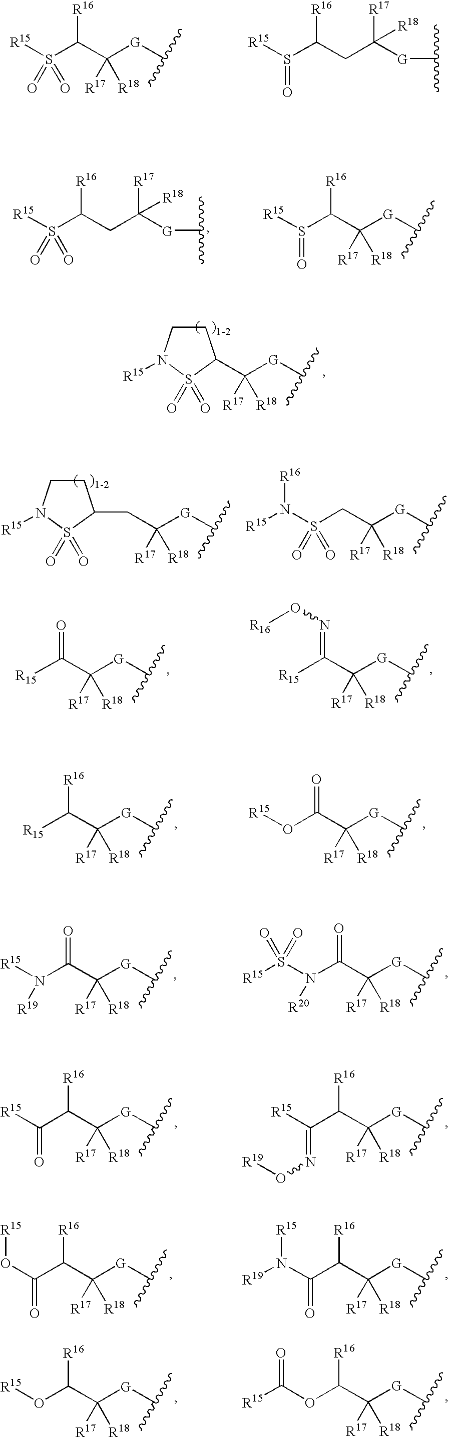

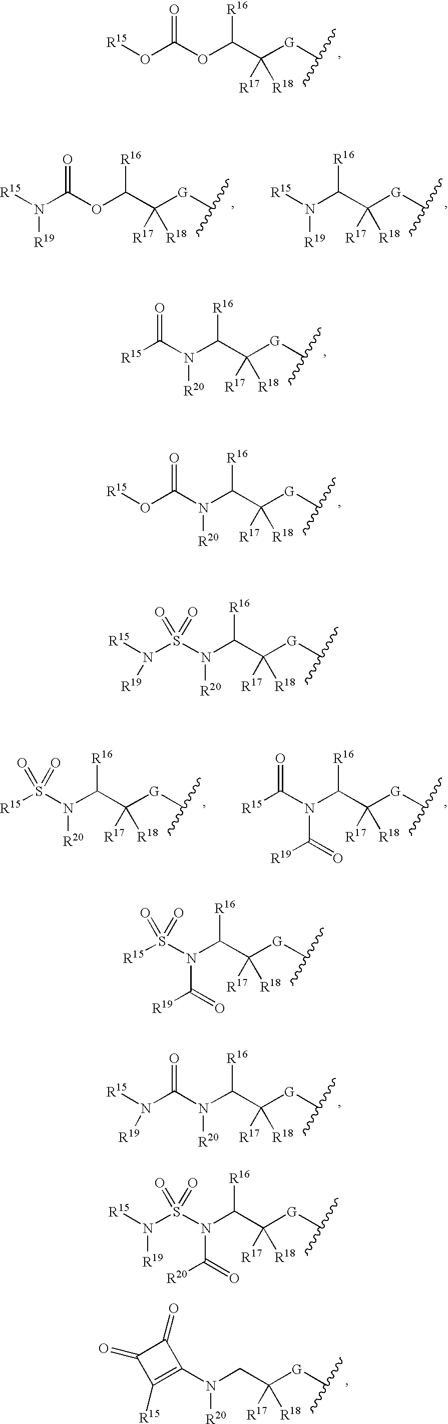

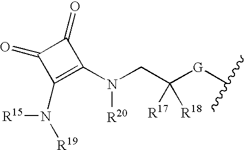

wherein G is NH or O; and R¹⁵, R¹⁶, R¹⁷, R¹⁸, R¹⁹, R²⁰ and R²¹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl; wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro.

In another embodiment, the “at least one compound” is a compound of structural Formula XX: 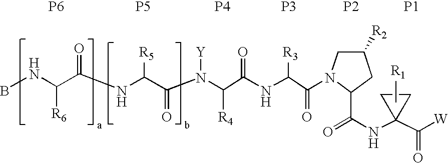
or a pharmaceutically acceptable salt, solvate or ester thereof;
wherein in Formula XX:
a is 0 or 1; b is 0 or 1; Y is H or C₁₋₆alkyl;
B is H, an acyl derivative of formula R₇—C(O)— or a sulfonyl of formula R₇—SO₂ wherein
R7 is (i) C₁₋₁₀ alkyl optionally substituted with carboxyl, C₁₋₆ alkanoyloxy or C₁₋₆ alkoxy;
(ii) C₃₋₇ cycloalkyl optionally substituted with carboxyl, (C₁₋₆ alkoxy)carbonyl or phenylmethoxycarbonyl;
(iii) C₆ or C₁₀ aryl or C₇₋₁₆ aralkyl optionally substituted with C₁₋₆alkyl, hydroxy, or amino optionally substituted with C₁₋₆ alkyl; or
(iv) Het optionally substituted with C₁₋₆ alkyl, hydroxy, amino optionally substituted with C₁₋₆ alkyl, or amido optionally substituted with C₁₋₆ alkyl;
   R₆, when present, is C₁₋₆ alkyl substituted with carboxyl;
   R₅, when present, is C₁₋₆ alkyl optionally substituted with carboxyl;
   R₄ is C₁₋₁₀ alkyl, C₃₋₇ cycloalkyl or C₄₋₁₀ (alkylcycloalkyl);
   R₃ is C₁₋₁₀ alkyl, C₃₋₇ cycloalkyl or C₄₋₁₀ (alkylcycloalkyl);
   R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkyl cycloalkyl) being optionally mono-, di- or tri-substituted with R₂₁, or R₂₀ is a C₆ or C₁₀ aryl or C₇₋₁₆ aralkyl optionally mono-, di- or tri-substituted with R₂₁,
   or R₂₀ is Het or (lower alkyl)-Het optionally mono-, di- or tri-substituted with R₂₁, wherein each R₂₁ is independently C₁₋₆ alkyl; C₁₋₆alkoxy; amino optionally mono- or di-substituted with C₁₋₆ alkyl; sulfonyl; NO₂; OH; SH; halo; haloalkyl; amido optionally mono-substituted with C₁₋₆ alkyl, C₆ or C₁₀ aryl, C₇₋₁₆ aralkyl, Het or (lower alkyl)-Het; carboxyl; carboxy(lower alkyl); C₆ or C₁₀ aryl, C₇₋₁₆ aralkyl or Het, said aryl, aralkyl or Het being optionally substituted with R₂₂;
   wherein R₂₂ is C₁₋₆alkyl; C₁₋₆ alkoxy; amino optionally mono- or di-substituted with C₁₋₆ alkyl; sulfonyl; NO₂; OH; SH; halo; haloalkyl; carboxyl; amide or (lower alkyl)amide;
   R₁ is C₁₋₆ alkyl or C₂₋₆ alkenyl optionally substituted with halogen; and
   W is hydroxy or a N-substituted amino.

In the above-shown structure of the compound of Formula XX, the terms P6, P5, P4, P3, P2 and P1 denote the respective amino acid moieties as is conventionally known to those skilled in the art.

In another embodiment, the “at least one compound” is a compound of structural Formula XXI: 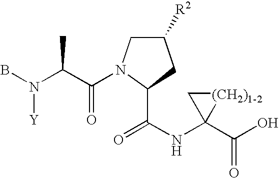
or a pharmaceutically acceptable salt, solvate or ester thereof;
wherein in Formula XXI:
B is H, a C₆ or C₁₀ aryl, C₇₋₁₆ aralkyl; Het or (lower alkyl)- Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide;
or B is an acyl derivative of formula R₄—C(O)—; a carboxyl of formula R₄—O—C(O)—; an amide of formula R₄—N(R₅)—C(O)—; a thioamide of formula R₄—N(R₅)—C(S)—; or a sulfonyl of formula R₄—SO₂ wherein

R₄ is (i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;

(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;

(iii) amino optionally mono- or di-substituted with C₁₋₆ alkyl; amido; or (lower alkyl)amide;

(iv) C₆ or C₁₀ aryl or C₇₋₁₆ aralkyl, all optionally substituted with C₁₋₆ alkyl, hydroxy, amido, (lower alkyl)amide, or amino optionally mono- or di-substituted with C₁₋₆ alkyl; or

(v) Het or (lower alkyl)-Het, both optionally substituted with C₁₋₆ alkyl, hydroxy, amido, (lower alkyl) amide, or amino optionally mono- or di-substituted with C₁₋₆alkyl;

R₅ is H or C₁₋₆alkyl;

with the proviso that when R₄ is an amide or a thioamide, R₄ is not (ii) a cycloalkoxy;

Y is H or C₁₋₆ alkyl;

R₃ is C₁₋₈ alkyl, C₃₋₇ cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;

R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkylcycloalkyl), all of which being optionally mono-, di- or tri-substituted with R₂₁, or R₂₀ is a C₆ or C₁₀ aryl or C₇₋₁₄ aralkyl, all optionally mono-, di- or tri-substituted with R₂₁,

or R₂₀ is Het or (lower alkyl)-Het, both optionally mono-, di- or tri-substituted with R₂₁,

wherein each R₂₁ is independently C₁₋₆ alkyl; C₁₋₆ alkoxy; lower thioalkyl; sulfonyl; NO₂; OH; SH; halo; haloalkyl; amino optionally mono- or di-substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;

wherein R₂₂ is C₁₋₆ alkyl; C₃₋₇ cycloalkyl; C₁₋₆ alkoxy; amino optionally mono- or di-substituted with C₁₋₆ alkyl; sulfonyl; (lower alkyl)sulfonyl; NO₂; OH; SH; halo; haloalkyl; carboxyl; amide; (lower alkyl)amide; or Het optionally substituted with C₁₋₆ alkyl;

R¹ is H; C₁₋₆ alkyl, C₃₋₇ cycloalkyl, C₂₋₆ alkenyl, or C₂₋₆ alkynyl, all optionally substituted with halogen.

In another embodiment, the “at least one compound” is a compound of structural Formula XXII: 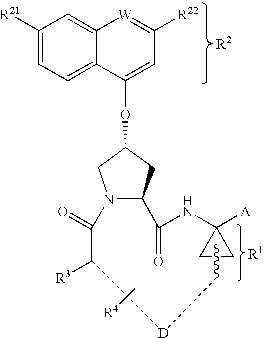
or a pharmaceutically acceptable salt, solvate or ester thereof;
wherein in Formula X)(XII:
W is CH or N,
R²¹ is H, halo, C₁₋₆ alkyl, C₃₋₆ cycloalkyl, C₁₋₆ haloalkyl, C₁₋₆ alkoxy, C₃₋₆ cycloalkoxy, hydroxy, or N(R²³)₂, wherein each R²³ is independently H, C₁₋₆ alkyl or C₃₋₆ cycloalkyl;
R²² is H, halo, C₁₋₆ alkyl, C₃₋₆ cycloalkyl, C₁ haloalkyl, C₁₋₆ thioalkyl, C₁₋₆ alkoxy, C₃₋₆ cycloalkoxy, C₂₋₇ alkoxyalkyl, C₃₋₆ cycloalkyl, C₆ or 10 aryl or Het, wherein Het is a five-, six-, or seven-membered saturated or unsaturated heterocycle containing from one to four heteroatoms selected from nitrogen, oxygen and sulfur; said cycloalkyl, aryl or Het being substituted with R²⁴, wherein R²⁴ is H, halo, C₁₋₆ alkyl, C₃₋₆ cycloalkyl, C₁₋₆ alkoxy, C₃₋₆ cycloalkoxy, NO₂, N(R²⁵)₂, NH—C(O)—R²⁵ or NH—C(O)—NH—R²⁵, wherein each R²⁵ is independently: H, C₁₋₆ alkyl or C₃₋₆ cycloalkyl;
or R²⁴ is NH—C(O)—OR²⁶ wherein R²⁶ is C₁₋₆ alkyl or C₃₋₆ cycloalkyl;
R³ is hydroxy, NH₂, or a group of formula —NH—R³¹, wherein R³¹ is C_{6 or 10} aryl, heteroaryl, —C(O)—R³², —C(O)—NHR³² or —C(O)—OR³², wherein R³² is C₁₋₆ alkyl or C₃₋₆ cycloalkyl;
D is a 5 to 10-atom saturated or unsaturated alkylene chain optionally containing one to three heteroatoms independently selected from: O, S, or N—R⁴¹, wherein R⁴¹ is H, C₁₋₆ alkyl, C₃₋₆ cycloalkyl or —C(O)—R⁴², wherein R⁴² is C₁₋₆ alkyl, C₃₋₆ cycloalkyl or C_{6 or 10} aryl; R⁴ is H or from one to three substituents at any carbon atom of said chain D, said substituent independently selected from the group consisting of: C₁₋₆ alkyl, C₁₋₆ haloalkyl, C₁₋₆ alkoxy, hydroxy, halo, amino, oxo, thio and C1-6 thioalkyl, and
A is an amide of formula —C(O)—NH—R⁵, wherein R⁵¹S selected from the group consisting of: C₁₋₈ alkyl, C₃₋₆ cycloalkyl, C_{6 or 10} aryl and C₇₋₁₆ aralkyl;
or A is a carboxylic acid.

In another embodiment, the “at least one compound” is a compound of structural Formula XXIII: 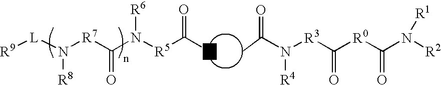
or a pharmaceutically acceptable salt, solvate or ester thereof;
wherein in Formula XXIII:
R⁰ is a bond or difluoromethylene;
R¹ is hydrogen;
R² and R⁹ are each independently optionally substituted aliphatic group, optionally substituted cyclic group or optionally substituted aromatic group;
R³, R⁵ and R⁷ are each independently:

optionally substituted (1,1- or 1,2-)cycloalkylene; or

optionally substituted (1,1- or 1,2-) heterocyclylene; or

methylene or ethylene), substituted with one substituent selected from the group consisting of an optionally substituted aliphatic group, an optionally substituted cyclic group or an optionally substituted aromatic group, and wherein the methylene or ethylene is further optionally substituted with an aliphatic group substituent; or;
R⁴, R6, R8 and R¹⁰ are each independently hydrogen or optionally substituted aliphatic group; 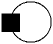
is substituted monocyclic azaheterocyclyl or optionally substituted multicyclic azaheterocyclyl, or optionally substituted multicyclic azaheterocyclenyl wherein the unsaturatation is in the ring distal to the ring bearing the R⁹—L-(N(R⁸)—R⁷—C(O)—)ₙN(R⁶)—R⁵—C(O)—N moiety and to which the —C(O)—N(R⁴)—R³—C(O)C(O)NR²R¹ moiety is attached; L is —C(O)—, —OC(O)—, —NR¹⁰C(O)—, —S(O)₂—, or —NR¹⁰S(O)₂—; and n is 0 or 1,
provided
when 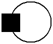
is substituted 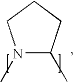
then L is —OC(O)— and R⁹ is optionally substituted aliphatic; or at least one of R³, R⁵ and R⁷ is ethylene, substituted with one substituent selected from the group consisting of an optionally substituted aliphatic group, an optionally substituted cyclic group or an optionally substituted aromatic group and wherein the ethylene is further optionally substituted with an aliphatic group substituent; or R⁴ is optionally substituted aliphatic.

In another embodiment, the “at least one compound” is a compound of structural Formula XXIV: 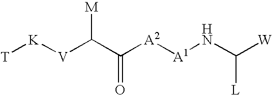
or a pharmaceutically acceptable salt, solvate or ester thereof;
wherein in Formula XXIV:
W is:

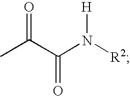

m is 0 or 1;

R² is hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroaralkyl; wherein any R² carbon atom is optionally substituted with J;

J is alkyl, aryl, aralkyl, alkoxy, aryloxy, aralkoxy, cycloalkyl, cycloalkoxy, heterocyclyl, heterocyclyloxy, heterocyclylalkyl, keto, hydroxy, amino, alkylamino, alkanoylamino, aroylamino, aralkanoylamino, carboxy, carboxyalkyl, carboxamidoalkyl, halo, cyano, nitro, formyl, acyl, sulfonyl, or sulfonamido and is optionally substituted with 1-3 J¹ groups;

J¹ is alkyl, aryl, aralkyl, alkoxy, aryloxy, heterocyclyl, heterocyclyloxy, keto, hydroxy, amino, alkanoylamino, aroylamino, carboxy, carboxyalkyl, carboxamidoalkyl, halo, cyano, nitro, formyl, sulfonyl, or sulfonamido;

L is alkyl, alkenyl, or alkynyl, wherein any hydrogen is optionally substituted with halogen, and wherein any hydrogen or halogen atom bound to any terminal carbon atom is optionally substituted with sulfhydryl or hydroxy;

A¹ is a bond;

R⁴ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;

R⁵ and R⁶ are independently hydrogen, alkyl, alkenyl, aryl, aralkyl, aralkenyl, cycloalkyl, cycloalkylalkyl, cycloalkenyl, heterocyclyl, heterocyclylalkyl, heteroaryl, or heteroaralkyl, and is optionally substituted with 1-3 J groups;

X is a bond, —C(H)(R7)—, -0-, —S—, or —N(R8)—;

R⁷ is hydrogen, alkyl, alkenyl, aryl, aralkyl, heterocyclyl, heterocyclylalkyl, heteroaryl, or heteroaralkyl, and is optionally substituted with 1-3 J groups;

R⁸ is hydrogen alkyl, aryl, aralkyl, heterocyclyl, heterocyclylalkyl, heteroaryl, heteroaralkyl, aralkanoyl, heterocyclanoyl, heteroaralkanoyl, —C(O)R¹⁴, —S0₂R¹⁴, or carboxamido, and is optionally substituted with 1-3 J groups; or R⁸ and Z, together with the atoms to which they are bound, form a nitrogen containing mono- or bicyclic ring system optionally substituted with 1-3 J groups;

R¹⁴ is alkyl, aryl, aralkyl, heterocyclyl, heterocyclyalkyl, heteroaryl, or heteroaralkyl;

Y is a bond, —CH₂—, —C(O)—, —C(O)C(O)—, —S(O)—, —S(O)₂—, or —S(O)(NR⁷)—,

wherein R⁷ is as defined above;

Z is alkyl, aryl, aralkyl, cycloalkyl, cycloalkylalkyl, heterocyclyl, heterocyclylalkyl, heteroaryl, heteroaralkyl, —OR², or —N(R²)₂, wherein any carbon atom is optionally substituted with J, wherein R² is as defined above;

A² is a bond or

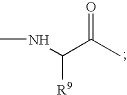

R⁹ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;

M is alkyl, cycloalkyl, aryl, aralkyl, heterocyclyl, heterocyclylalkyl, heteroaryl, or heteroaralkyl, optionally substituted by 1-3 J groups, wherein any alkyl carbon atom may be replaced by a heteroatom;

V is a bond, —CH₂—, —C(H)(R¹¹)—, -0-, —S—, or —N(R¹¹)—;

R¹¹ is hydrogen or C₁₋₃ alkyl;

K is a bond, -0-, —S—, —C(O)—, —S(O)—, —S(O)₂—, or —S(O)(NR¹¹)—, wherein R¹¹ is as defined above;

T is —R¹², -alkyl-R², -alkenyl-R², alkynyl-R¹², —OR¹², —N(R¹²)₂, —C(O)R¹², —C(═NOalkyl)R¹², or

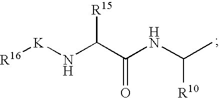

R¹² is hydrogen, aryl, heteroaryl, cycloalkyl, heterocyclyl, cycloalkylidenyl, or heterocycloalkylidenyl, and is optionally substituted with 1-3 J groups, or a first R¹² and a second R¹², together with the nitrogen to which they are bound, form a mono- or bicyclic ring system optionally substituted by 1-3 J groups;

R¹⁰ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 hydrogens J groups;

R¹⁵ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups; and

R¹⁶ is hydrogen, alkyl, aryl, heteroaryl, cycloalkyl, or heterocyclyl.

In another embodiment, the “at least one compound” is a compound of structural Formula XXV: 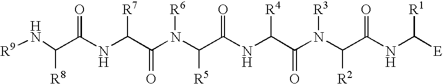
or a pharmaceutically acceptable salt, solvate or ester thereof;
wherein in Formula XXV:

E represents CHO or B(OH)₂;

R¹ represents lower alkyl, halo-lower alkyl, cyano-lower alkyl, lower alkylthio-lower alkyl, aryl-lower alkylthio-lower alkyl, aryl-lower alkyl, heteroaryllower alkyl, lower alkenyl or lower alkynyl;

R² represents lower alkyl, hydroxy-lower alkyl, carboxy-lower alkyl, aryl-lower alkyl, aminocarbonyl-lower alkyl or lower cycloalkyl-lower alkyl; and

R³ represents hydrogen or lower alkyl;

or R² and R³ together represent di- or trimethylene optionally substituted by hydroxy;

R⁴ represents lower alkyl, hydroxy-lower alkyl, lower cycloalkyl-lower alkyl, carboxy-lower alkyl, aryllower alkyl, lower alkylthio-lower alkyl, cyano-lower alkylthio-lower alkyl, aryl-lower alkylthio-lower alkyl, lower alkenyl, aryl or lower cycloalkyl;

R⁵ represents lower alkyl, hydroxy-lower alkyl, lower alkylthio-lower alkyl, aryl-lower alkyl, aryl-lower alkylthio-lower alkyl, cyano-lower alkylthio-lower alkyl or lower cycloalkyl;

R⁶ represents hydrogen or lower alkyl;

R⁷ represent lower alkyl, hydroxydower alkyl, carboxylower alkyl, aryl-lower alkyl, lower cycloalkyl-lower alkyl or lower cycloalkyl;

R⁸ represents lower alkyl, hydroxy-lower alkyl, carboxylower alkyl or aryl-lower alkyl; and

R⁹ represents lower alkylcarbonyl, carboxy-lower alkylcarbonyl, arylcarbonyl, lower alkylsulphonyl, arylsulphonyl, lower alkoxycarbonyl or aryl-lower alkoxycarbonyl.

In another embodiment, the “at least one compound” is a compound of structural Formula XXVI: 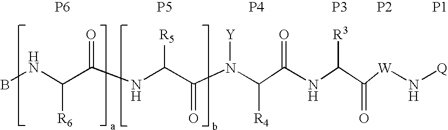
or a pharmaceutically acceptable salt, solvate or ester thereof;
wherein in Formula XXVI:

B is an acyl derivative of formula R₁₁—C(O)— wherein R₁₁ is Cl-10 alkyl optionally substituted with carboxyl; or R₁₁ is C₆ or C₁₀ aryl or C₇₋₁₆ aralkyl optionally substituted with a C₁₋₆ alkyl;

a is 0 or 1;

R₆, when present, is carboxy(lower)alkyl;

b is 0 or 1;

R₅, when present, is C₁₋₆ alkyl, or carboxy(lower)alkyl;

Y is H or C₁₋₆ alkyl;

R₄ is C₁₋₁₀ alkyl; C₃₋₁₀ cycloalkyl;

R₃ is C₁₋₁₀ alkyl; C₃₋₁₀ cycloalkyl;

W is a group of formula:

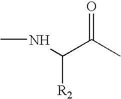

wherein R₂ is C₁₋₁₀ alkyl or C₃₋₇ cycloalkyl optionally substituted with carboxyl; C₆ or C₁₀ aryl; or C₇₋₁₆ aralkyl; or

W is a group of formula:

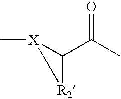

wherein X is CH or N; and

R₂′ is C₃₋₄ alkylene that joins X to form a 5- or 6-membered ring, said ring optionally substituted with OH; SH; NH2; carboxyl; R₁₂; OR₁₂, SR₁₂, NHR₁₂ or NR₁₂R₁₂′ wherein R₁₂ and R₁₂′ are independently:

cyclic C₃₋₁₆ alkyl or acyclic C₁₋₁₆ alkyl or cyclic C₃₋₁₆ alkenyl or acyclic C₂₋₁₆ alkenyl, said alkyl or alkenyl optionally substituted with NH₂, OH, SH, halo, or carboxyl; said alkyl or alkenyl optionally containing at least one heteroatom selected independently from the group consisting of: 0, S, and N; or

R₁₂ and R₁₂′ are independently C₆ or C₁₀ aryl or C₇₋₁₆ aralkyl optionally substituted with C₁₋₆alkyl, NH₂, OH, SH, halo, carboxyl or carboxy(lower)alkyl; said aryl or aralkyl optionally containing at least one heteroatom selected independently from the group consisting of: 0, S, and N;

said cyclic alkyl, cyclic alkenyl, aryl or aralkyl being optionally fused with a second 5-, 6-, or 7-membered ring to form a cyclic system or heterocycle, said second ring being optionally substituted with NH₂. OH, SH, halo, carboxyl or carboxy(lower)alkyl; C₆ or C₁₀ aryl, or heterocycle; said second ring optionally containing at least one heteroatom selected independently from the group consisting of: 0, S, and N;

Q is a group of the formula:

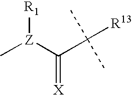

wherein Z is CH;

X is 0 or S;

R₁ is H, C₁₋₆ alkyl or C₁₋₆ alkenyl both optionally substituted with thio or halo; and

R₁₃ is C0—NH—R₁₄ wherein R₁₄ is hydrogen, cyclic C₃₋₁₀ alkyl or acyclic C₁₋₁₀ alkyl or cyclic C₃₋₁₀ alkenyl or acyclic C₂₋₁₀ alkenyl, said alkyl or alkenyl optionally substituted with NH₂, OH, SH, halo or carboxyl; said alkyl or alkenyl optionally containing at least one heteroatom selected independently from the group consisting of: 0, S, and N; or

R₁₄ is C₆ or C₁₀ aryl or C₇₋₁₆ aralkyl optionally substituted with C₁₋₆ alkyl, NH₂, OH, SH, halo, carboxyl or carboxy(lower)alkyl or substituted with a further C₃₋₇ cycloalkyl, C₆ or C₁₀ aryl, or heterocycle; said aryl or aralkyl optionally containing at least one heteroatom selected independently from the group consisting of: 0, S, and N;

said cyclic alkyl, cyclic alkenyl, aryl or aralkyl being optionally fused with a second 5-, 6-, or 7-membered ring to form a cyclic system or heterocycle, said second ring being optionally substituted with NH₂, OH, SH, halo, carboxyl or carboxy(lower)alkyl or substituted with a further C₃₋₇ cycloalkyl, C₆ or C₁₀ aryl, or heterocycle; said second ring optionally containing at least one heteroatom selected independently from the group consisting of: 0, S, and N;

with the proviso that when Z is CH, then R₁₃ is not an α-amino acid or an ester thereof;

Q is a phosphonate group of the formula:

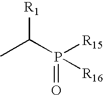

wherein R₁₅ and R₁₆ are independently C₆₋₂₀ aryloxy; and R₁ is as defined above.

In the above-shown structure of the compound of Formula XXVI, the terms P6, P5, P4, P3, P2 and P1 denote the respective amino acid moieties as is conventionally known to those skilled in the art. Thus, the actual structure of the compound of Formula XXVI is:

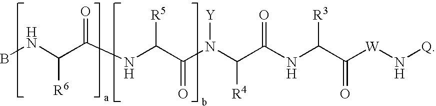

In another embodiment, the “at least one compound” is a compound of structural Formula XXVII: 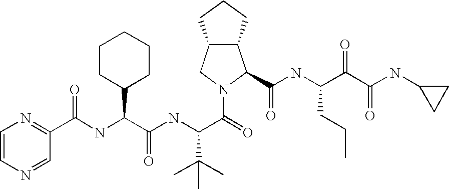
or a pharmaceutically acceptable salt, solvate or ester thereof.

In another embodiment, the “at least one compound” is selected from the group consisting of: 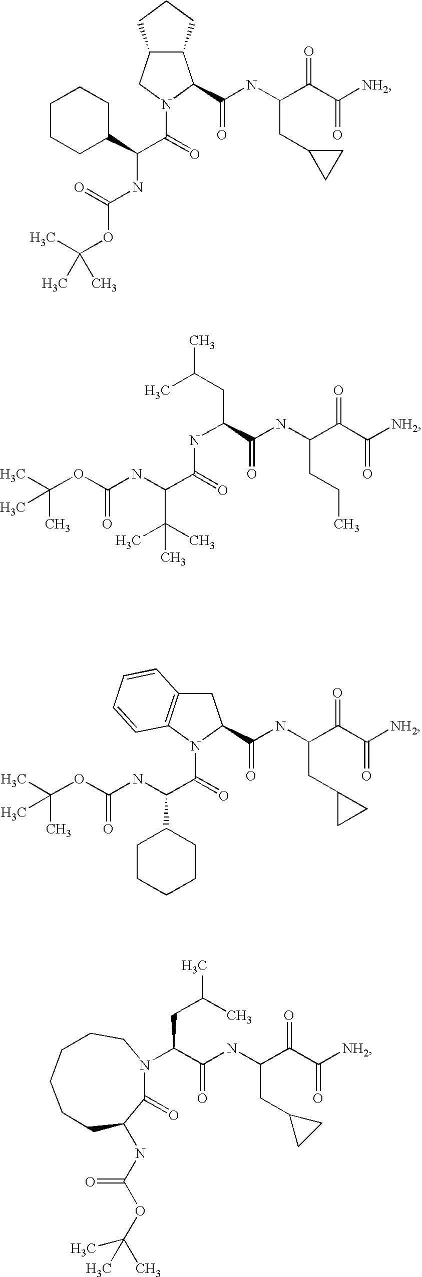 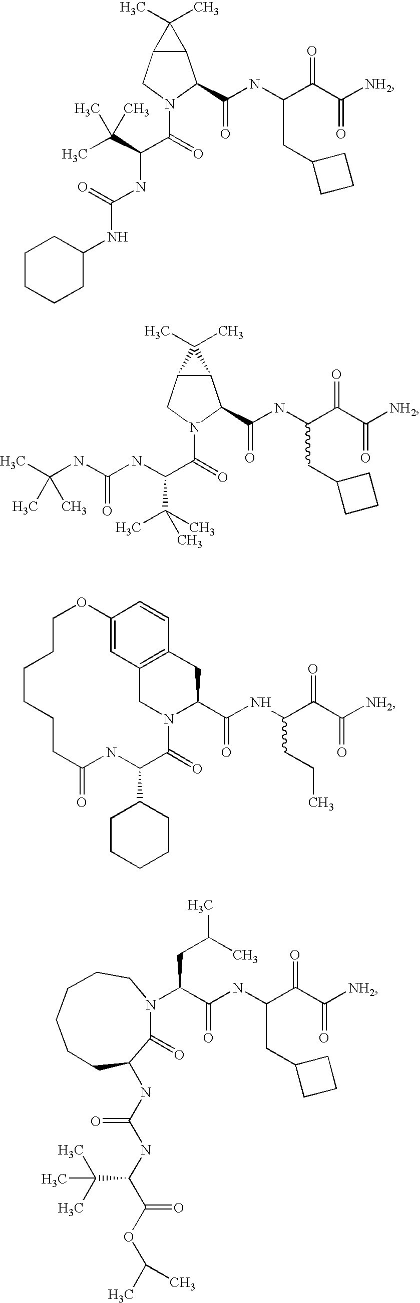 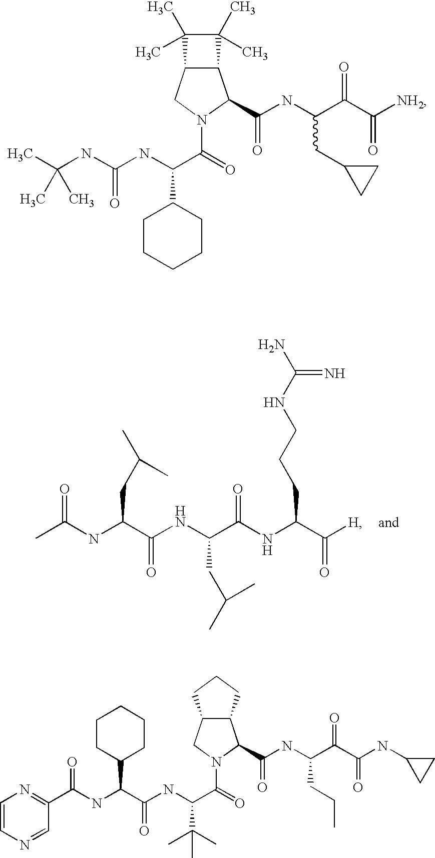
or a pharmaceutically acceptable salt, solvate or ester thereof.

In yet another embodiment, the “at least one compound” is: 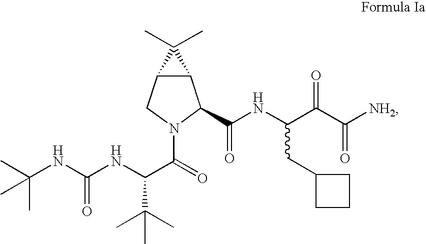
a pharmaceutically acceptable salt, solvate, or ester thereof, or a mixture of two or more thereof.

In still yet another embodiment, the “at least one compound” is: 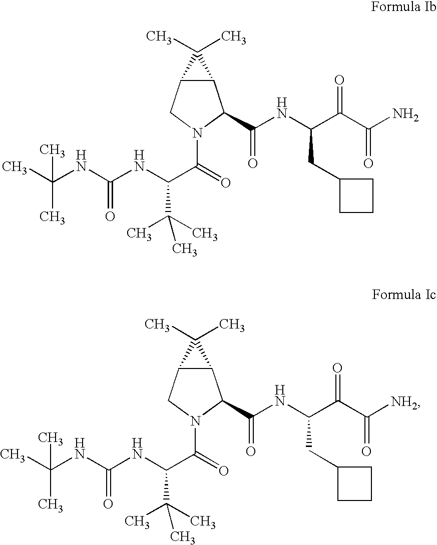
a pharmaceutically acceptable salt, solvate, or ester thereof, or a mixture of two or more thereof.

In a preferred embodiment, the “at least one compound” is administered in an amount of about 100 mg to about 3000 mg per day.

In one embodiment, at least one AKR competitor is an AKR substrate, an AKR inhibitor, or a mixture of two or more thereof. In one embodiment, the AKR substrate is a fibrate, a 5α-dihydroxytestosterone, dolasetron, doxorubicin, 17β-estradiol, a non-steroidal anti-inflammatory drug (NSAID), ketotifen, naltrexone, Z-10-oxo nortriptyline, oestrone, a S-1360 HIV integrase inhibitor, progesterone, prostaglandin, sorbinil, testosterone, tibolone, tolrestat, naringenin, or a mixture of two or more thereof. Preferably, the fibrate is benzafibrate, bezafibrate, binifibrate, ciprofibrate, clinofibrate, clofibrate, fenofibrate, gemfibrozil, lifibrol, or a mixture of two or more thereof. In another embodiment, the AKR inhibitor is an AKR1C1 AKR inhibitor, an AKR1C2 AKR inhibitor, an AKR1C3 AKR inhibitor, an AKR1C4 AKR inhibitor, naringenin, or a mixture of two or more thereof. In one preferred embodiment, the AKR inhibitor is a benzodiazepine, a cyclooxygenase (COX)2 inhibitor, a NSAID, testosterone, naringenin, or a mixture of two or more thereof. Preferably, the benzodiazepine is cloxazolam, diazepam, estazolam, flunitrazepam, nitrazepam, medazepam, or a mixture of two or more thereof. Preferably, the COX 2 inhibitor is celecoxib. Preferably, the NSAID is ibuprofen, diclofenac, diflunisal, flufenamic acid, indomethacin, mefenamic acid, naproxen, or a mixture of two or more thereof. In one preferred embodiment, at least one AKR competitor is diflusinal. Preferably, diflunisal is administered in an amount ranging from about 5 to about 499 mg per day.

In one embodiment, the medicament further comprises at least one other therapeutic agent. Preferably, at least one other therapeutic agent is ribavirin, levovirin, VP 50406, ISIS 14803, Heptazyme, VX 497, Thymosin, Maxamine, mycophenolate mofetil, interferon, an antibody specific to IL-10, or a mixture of two or more thereof. In one embodiment, at least one other therapeutic agent is an antibody specific to IL-10, preferably humanized 12G8. In another embodiment, interferon is interferon-alpha, PEG-interferon alpha conjugates, interferon alpha fusion polypeptides, consensus interferon, or a mixture of two or more thereof. In one embodiment, at least one other therapeutic agent is administered concurrently or consecutively with at least one compound and the AKR competitor. In one embodiment, the medicament further comprises at least one anti-cancer agent.

The present invention also provides a medicament comprising, separately or together:
(a) at least one AKR competitor; and
(b) a HCV protease inhibitor selected from the group consisting of a compound of Formula Ia, Ib, or Ic, or a pharmaceutically acceptable salt, solvate or ester thereof, or a mixture of two or more thereof, for concurrent or consecutive administration in treating or ameliorating one or more symptoms of HCV, or disorders associated with HCV in a subject in need thereof.

The present invention also provides a medicament comprising, separately or together:
(a) at least one AKR competitor; and
(b) a HCV protease inhibitor selected from the group consisting of a compound of Formula XXVII, or a pharmaceutically acceptable salt, solvate, or ester thereof, or a mixture of two or more thereof,
   for concurrent or consecutive administration in treating or ameliorating one or more symptoms of HCV, or disorders associated with HCV in a subject in need thereof.

In one embodiment of the medicament, at least one AKR competitor is diflunisal. Preferably, the diflunisal is administered at a unit dosage sufficient to increase the bioavailability of the HCV protease inhibitor. Preferably, the diflunisal is administered at a unit dosage of about 5 mg to about 499 mg per day. Preferably, the diflunisal is administered at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, or 499 mg per day.

The present invention also provides a pharmaceutical composition comprising a therapeutically effective amount of the medicament and a pharmaceutically acceptable carrier.

The present invention also provides pharmaceutical kits comprising the medicament, in separate unit dosage forms, said forms being suitable for administration of (a) and (b) in effective amounts, and instructions for administering (a) and (b).

In one embodiment of the pharmaceutical kit, at least one AKR competitor is diflunisal. Preferably, the diflunisal is administered at a unit dosage sufficient to increase the bioavailability of the HCV protease inhibitor. Preferably, the diflunisal is administered at a unit dosage of about 5 mg to about 499 mg per day. Preferably, the diflunisal is administered at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, or 499 mg per day.

The present invention also provides methods for treating or ameliorating one or more symptoms of HCV, or disorders associated with HCV in a subject in need thereof, comprising the step of administering to the subject an effective amount of the medicament.

The present invention also provides a composition comprising diflunisal at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, or 249 mg.

In one embodiment, the diflunisal unit dosage is sufficient to increase the bioavailability of a HCV protease inhibitor. In one preferred embodiment, the HCV protease inhibitor is selected from the group consisting of a compound of Formula Ia, Ib, or Ic, or a pharmaceutically acceptable salt, solvate or ester thereof, or a mixture of two or more thereof. In another preferred embodiment, the HCV protease inhibitor is selected from the group consisting of a compound of Formula XXVII, or a pharmaceutically acceptable salt, solvate, or ester thereof, or a mixture of two or more thereof.

The present invention also provides a pharmaceutical composition comprising an amount of the composition sufficient to increase the bioavailability of a HCV protease inhibitor and a pharmaceutically acceptable carrier.

The present invention also provides a kit comprising:

(a) diflunisal at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, or 499 mg; and

(b) instructions for administering (a).

In one embodiment, the kit further comprises at least one HCV protease inhibitor. In one preferred embodiment, at least one HCV protease inhibitor is selected from the group consisting of a compound of Formula Ia, Ib, or Ic, or a pharmaceutically acceptable salt, solvate or ester thereof, or a mixture of two or more thereof. In another preferred embodiment, at least one HCV protease inhibitor is selected from the group consisting of a compound of Formula XXVII, or a pharmaceutically acceptable salt, solvate, or ester thereof, or a mixture of two or more thereof. In yet another preferred embodiment, at least one HCV protease inhibitor is selected from the group consisting of a compound of Formula I to XXVII, or a pharmaceutically acceptable salt, solvate, or ester thereof, or a mixture of two or more thereof.

The present invention also provides a method of increasing the bioavailability of a drug metabolized by AKR comprising administering diflunisal at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, or 499 mg. Preferably, the diflunisal unit dosage is sufficient to increase the bioavailability of a HCV protease inhibitor. In one preferred embodiment, the HCV protease inhibitor is selected from the group consisting of a compound of Formula Ia, Ib, or Ic, or a pharmaceutically acceptable salt, solvate or ester thereof, or a mixture of two or more thereof. In another preferred embodiment, the HCV protease inhibitor is selected from the group consisting of a compound of Formula XXVII, or a pharmaceutically acceptable salt, solvate, or ester thereof, or a mixture of two or more thereof. In one embodiment, the method further comprises administering at least one HCV protease inhibitor concurrently or consecutively. In one preferred embodiment, at least one HCV protease inhibitor is selected from the group consisting of a compound of Formula Ia, Ib, or Ic, or a pharmaceutically acceptable salt, solvate, or ester thereof, or a mixture of two or more thereof. In another preferred embodiment, at least one HCV protease inhibitor is selected from the group consisting of a compound of Formula XXVII, or a pharmaceutically acceptable salt, solvate, or ester thereof, or a mixture of two or more thereof. In one preferred embodiment, the diflunisal unit dosage is sufficient to increase the blood level of a HCV protease inhibitor.

### BRIEF DESCRIPTION OF THE DRAWINGS

The foregoing summary, as well as the following detailed description, is better understood when read in conjunction with the appended drawings. In the drawings:

FIG. 1 is a radiometric profile of incubation of 14C-compound of Formula Ia with Human AKR1C2.

FIG. 2 is a radiometric profile of incubation of 14C-compound of Formula Ia with Human AKR1C3.

FIG. 3 is a radiometric profile of incubation of 14C-compound of Formula Ia with Human AKR1C4.

FIG. 4 is graph of the effect of ibuprofen on the formation on the compound of Formula Ia′ with human liver cytosol (60 minute incubation).

FIG. 5a depicts the AUC ratio of compound Formula Ia′ to compound Formula Ic in plasma levels of cynomolgus monkeys following administration of 200 mg Formula Ia and 0, 62.5, 125, or 250 mg diflunisal.

FIG. 5b depicts the AUC ratio of compound Formula Ia′ to compound Formula Ib in plasma levels of cynomolgus monkeys following administration of 200 mg Formula Ia and 0, 62.5, 125, or 250 mg diflunisal.

FIG. 5c depicts the AUC ratio of compound Formula Ia′ to compound Formula Ia in plasma levels of cynomolgus monkeys following administration of 200 mg Formula Ia and 0, 62.5, 125, or 250 mg diflunisal.

FIG. 6 is a schematic of the clinical study conducted to evaluate the effect of ibuprofen on the pharmacokinetics and metabolism of Formula I.

FIG. 7 is a schematic of Part 1 of the proposed clinical study to assess the pharmacokinetics, safety, and tolerability of 800 mg Formula Ia (QD) administered in combination with 0, 125, 250, or 500 mg diflunisal (BID for 2 days).

FIG. 8 is a schematic of Part 2 of the proposed clinical study to assess the pharmacokinetics, safety, and tolerability of 800 mg Formula Ia (QD) administered in combination with 0, 15, 45, or 125 mg diflunisal (BID for 2 days).

### DETAILED DESCRIPTION

The present invention provides medicaments, pharmaceutical compositions, pharmaceutical kits, and methods based on combinations comprising, separately or together: (a) at least one aldo-keto reductase (AKR) competitor; and (b) at least one HCV protease inhibitor, for concurrent or consecutive administration in treating HCV in an infected subject.

In one embodiment, the present invention is directed to medicaments, pharmaceutical compositions, pharmaceutical kits, and methods of treating, preventing, or ameliorating one or more symptoms of HCV, treating disorders associated with HCV, or inhibiting cathepsin activity in a subject using the same, comprising at least one (one or more) AKR competitors and at least one (one or more) compound of Formula I to XXVII above.

In one embodiment, at least one HCV protease inhibitor is selected from the group of HCV protease inhibitors referred to in the following documents (which are incorporated by reference herein): US20040048802A1, US20040043949A1, US20040001853A1, US20030008828A1, US20020182227A1, US20020177725A1, US20020150947A1, US20050267018A1, US20020034732A1, US20010034019A1, US20050153877A1, US20050074465A1, US20050053921A1, US20040253577A1, US20040229936A1, US20040229840A1, US20040077551 A1, EP1408031 A1, WO9837180A2, U.S. Pat. No. 6,696,281 B1, JP11137252A, WO01111089A1, U.S. Pat. No. 6,280,940B1, EP1106702A1, US20050118603A1, JP2000007645A, WO0053740A1, WO0020400A1, WO2004013349A2, WO2005027871A2, WO2002100900A2, WO155703A1, US20030125541A1, US20040039187A1, U.S. Pat. No. 6,608,027B1, US20030224977A1, WO2003010141 A2, WO2003007945A1, WO2002052015A2, WO0248375A2, WO0066623A2, WO0009543A2, WO9907734A2, U.S. Pat. No. 6,767,991 B1, US20030187018A1, US20030186895A1, WO2004087741A1, WO2004039970A1, WO2004039833A1, WO2004037855A1, WO2004030670A1, US20040229818A1, US20040224900A1, WO2005028501A1, WO2004103996A1, WO2004065367A1, WO2004064925A1, WO2004093915A1, WO2004009121A1, WO2003066103A1, WO2005034850A2, WO2004094452A2, WO2004015131A2, WO2003099316A1, WO2003099274A1, WO2003053349A2, WO2002060926A2, WO0040745A1, U.S. Pat. No. 6,586,615B1, WO2002061048A2, WO0248157A2, WO0248116A2, WO2005017125A2, WO0022160A1, US20060051745A1, WO2004021871A2, WO2004011647A1, WO9816657A1, U.S. Pat. No. 5,371,017A, WO9849190A2, U.S. Pat. No. 5,807,829A, WO0005243A2, WO0208251A2, WO2005067437A2, WO9918856A1, WO0004914A1, WO0212543A2, WO9845040A1, WO0140262A1, WO0102424A2, WO0196540A2, WO0164678A2, U.S. Pat. No. 5,512,391A, WO0218369A2, WO9846597A1, WO2005010029A1, WO2004113365A2, WO2004093798A2, WO2004072243A2, WO9822496A2, WO2004046159A1, JP11199509A, WO2005012288A1, WO2004108687A2, WO9740168A1, US20060110755A1, WO2002093519A2, U.S. Pat. No. 6,187,905B1, WO2003077729A2, WO9524414A1, WO2005009418A2, WO2004003000A2, US20050037018A1, WO9963998A1, WO0063444A2, WO9938888A2, WO9964442A1, WO0031129A1, WO0168818A2, WO9812308A1, WO9522985A1, WO0132691A1, WO9708304A2, WO2002079234A1, JP10298151A, JP09206076A, JP09009961A, JP2001103993A, JP11127861A, JP11124400A, JP11124398A, WO2003051910A2, WO2004021861A2, WO9800548A1, WO2004026896A2, WO0116379A1, U.S. Pat. No. 5,861,297A, WO2004007512A2, WO2004003138A2, WO2002057287A2, WO2004009020A2, WO2004000858A2, WO2003105770A2, WO0114517A1, WO9805333A1, U.S. Pat. No. 6,280,728B1, EP1443116A1, US20040063911A1, WO2003076466A1, WO2002087500A2, WO0190121A2, WO2004016222A2, WO9839030A1, WO9846630A1, WO0123331A1, WO9824766A1, U.S. Pat. No. 6,168,942B1, WO0188113A2, WO2005018330A1, WO2005003147A2, WO9115596A1, WO9719103A1, WO9708194A1, WO2002055693A2, WO2005030796A1, WO2005021584A2, WO2004113295A1, WO2004113294A1, WO2004113272A1, WO2003062228A1, WO0248172A2, WO0208198A2, WO0181325A2, WO0177113A2, WO0158929A1, WO9928482A2, WO9743310A1, WO9636702A2, WO9635806A1, WO9635717A2, U.S. Pat. No. 6,326,137B1, U.S. Pat. No. 6,251,583B1, U.S. Pat. No. 5,990,276A, U.S. Pat. No. 5,759,795A, U.S. Pat. No. 5,714,371A, U.S. Pat. No. 6,524,589B1, WO0208256A2, WO0208187A1, WO2003062265A2, U.S. Pat. No. 7,012,066B2, JP07184648A, JP06315377A, WO2002100851A2, WO2002100846A1, WO0039348A1, JP06319583A, JP 11292840A, JP08205893A, WO0075338A2, WO0075337A1, WO2003059384A1, WO2002063035A2, WO2002070752A1, U.S. Pat. No. 6,190,920B1, WO2002068933A2, WO0122984A1, JP04320693A, JP2003064094A, WO0179849A2, WO0006710A1, WO0001718A2, WO0238799A2, WO2005037860A2, WO2005035525A2, WO2005025517A2, WO2005007681A2, WO2003035060A1, WO2003006490A1, WO0174768A2, WO0107027A2, WO0024725A1, WO0012727A1, WO9950230A1, WO9909148A1, WO9817679A1, WO9811134A1, WO9634976A1, WO2003087092A2, WO2005028502A1, U.S. Pat. No. 5,837,464A, DE20201549U1, WO2003090674A2, WO9727334A1, WO0034308A2, U.S. Pat. No. 6,127,116A, US20030054000A1, JP2001019699A, U.S. Pat. No. 6,596,545B1, U.S. Pat. No. 6,329,209B1, IT1299179, CA2370400, KR2002007244, KR165708, KR2000074387, KR2000033010, KR2000033011, KR2001107178, KR2001107179, ES2143918, KR2002014283, KR149198, KR2001068676. Preferably, at least one HCV protease inhibitor is a compound selected from the group of compounds of Formula I to XXVII (described above).

Preferably, the HCV protease inhibitor is administered at a dosage range of about 100 to about 3000 mg per day (e.g., 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg per day). In one preferred embodiment, the HCV protease inhibitor is administered at a dosage range of about 400 mg to about 2500 mg per day. Note that the dosage of HCV protease inhibitor may be administered as a single dose (i.e., QD) or divided over 2-4 doses (i.e., BID, TID, or QID) per day. Preferably, the HCV protease inhibitor is administered orally.

In one embodiment, where the HCV protease inhibitor is selected from the group consisting of a compound of Formula Ia, Ib, or Ic, or a pharmaceutically acceptable salt, solvate, or ester thereof, or a mixture of two or more thereof, the preferred dosage range is about 400 mg to 2400 mg per day. In one preferred embodiment, where the HCV protease inhibitor is selected from the group consisting of a compound of Formula Ia, Ib, or Ic, or a pharmaceutically acceptable salt, solvate, or ester thereof, or a mixture of two or more thereof, the dosage is about 1200 mg per day administered as about 400 mg TID. In another preferred embodiment, where the HCV protease inhibitor is selected from the group consisting of a compound of Formula Ia, Ib, or Ic, or a pharmaceutically acceptable salt, solvate, or ester thereof, or a mixture of two or more thereof, the dosage is about 800 mg, 1600 mg, or 2400 mg per day administered as about 800 mg QD, BID, or TID, respectively.

In another embodiment, where the HCV protease inhibitor is selected from the group consisting of Formula XXVII, or a pharmaceutically acceptable salt, solvate, or ester thereof, or a mixture of two or more thereof, the preferred dosage range is about 1350 mg to about 2500 mg per day. In one preferred embodiment, where the HCV protease inhibitor is selected from the group consisting of Formula XXVII, or a pharmaceutically acceptable salt, solvate, or ester thereof, or a mixture of two or more thereof, the dosage is about 1350 mg, about 2250 mg, or about 2500 mg per day administered as about 450 mg TID, about 750 BID, or about 1250 BID, respectively.

In one embodiment, at least one AKR competitor is diflunisal, and at least one compound is Formula Ia, Ib, or Ic, or a pharmaceutically acceptable salt, solvate or ester thereof, or a mixture of two or more thereof.

In another embodiment, at least one AKR competitor is diflunisal, and at least one compound is Formula XXVII or a pharmaceutically acceptable salt, solvate or ester thereof, or a mixture of two or more thereof.

The aldo-keto reductases (AKRs) or aldehyde keto reductases are one of the carbonyl reductase enzyme superfamilies that perform oxidoreduction on a wide variety of natural and foreign substrates. There are four human AKR1C enzymes (also called hydroxysteroid dehydrogenases (HSDs)) and include AKR1C1 (20α-HSD), AKR1C2 (3α-HSD Type 3), AKR1C3 (3α-HSD Type 2, 17β-HSD, Type 5) and AKR1C4 (3α-HSD Type 1).

The above-described compounds of Formula I to XXVII each include a keto amide moiety: 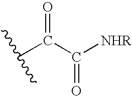
wherein R is any of the organic groups discussed in Formula I to XXVII above. The AKR enzyme can reduce the ketone moiety to create a new chiral center: 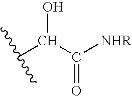
during metabolism of the compound.

For example, the compound of Formula Ia can be metabolized by the NADPH-dependent cytosolic human AKRs (AKR) AKR1C2 and AKR1C3 to yield a mixture of four stereoisomers that results from the reduction of the ketone moiety of the ketoamide moiety in Formula Ia to create a new chiral center.

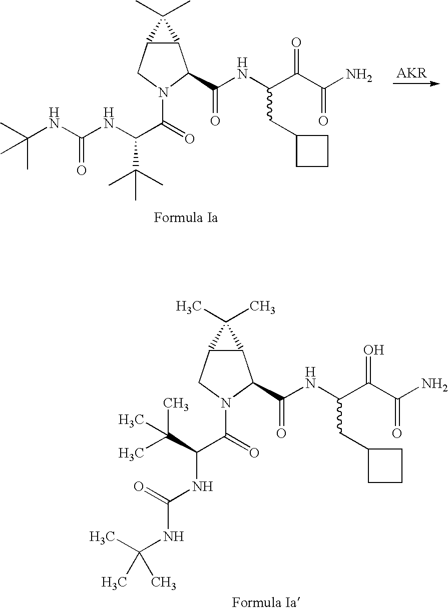

Coadministration of AKR competitor(s) (substrates or inhibitors of AKR) would be desirable to modify the pharmacokinetic behavior of the compounds of Formula I-XXVII, for example to slow or prevent reduction of the ketone moiety and thereby increase duration of action of the compounds.

Non-limiting examples of suitable AKR competitors include AKR substrates, AKR inhibitors, or a mixture of two or more thereof. Suitable AKR substrates include fibrates, 5α-dihydroxytestosterone, dolasetron (such as ANZEMET dolasetron mesylate which is commercially available from Aventis Pharmaceuticals), doxorubicin (such as DOXIL, ADRIMYCIN OR ONCOJET doxorubicin hydrochloride), 17β-estradiol, non-steroidal anti-inflammatory drugs (NSAIDS), ketotifen (such as is commercially available from Apotex), naltrexone (such as ReVia naltrexone hydrochloride opioid antagonist), Z-10-oxo nortriptyline (such as AVENTYL or PAMELOR nortriptyline), oestrone, S-1360 HIV integrase inhibitor, progesterone, prostaglandin, sorbinil, testosterone, tibolone, tolrestat, naringenin (available from grapefruit juice or from R&S Pharmchem, Hangzhou City, China) and a mixture of two or more thereof.

Fibrates (fibric acid derivatives) are peroxisome proliferator-activated receptor (PPAR) alpha activators. Non-limiting examples of suitable fibric acid derivatives include clofibrate (such as ethyl 2-(p-chlorophenoxy)-2-meth-yl-propionate, for example ATROMID-S capsules which are commercially available from Wyeth-Ayerst); gemfibrozil (such as 5-(2,5-dimethylphenoxy)-2,2-dimethylpentanoic acid, for example LOPID® tablets which are commercially available from Parke Davis); ciprofibrate (C.A.S. Registry No. 52214-84-3, see U.S. Pat. No. 3,948,973 which is incorporated herein by reference); benzafibrate, bezafibrate (C.A.S. Registry No.41859-67-0, see U.S. Pat. No. 3,781,328 which is incorporated herein by reference); clinofibrate (C.A.S. Registry No. 30299-08-2, see U.S. Pat. No. 3,716,583 which is incorporated herein by reference); binifibrate (C.A.S. Registry No. 69047-39-8, see BE 884722 which is incorporated herein by reference); lifibrol (C.A.S. Registry No. 96609-16-4); fenofibrate (such as TRICOR micronized fenofibrate (2-[4-(4-chlorobenzoyl)phenoxy]-2-methyl-propanoic acid, 1-methylethyl ester) which is commercially available from Abbott Laboratories or LIPANTHYL micronized fenofibrate which is commercially available from Labortoire Founier, France) and a mixture of two or more thereof. These compounds can be used in a variety of forms, including but not limited to acid form, salt form, racemates, enantiomers, zwitterions and tautomers.

Suitable NSAIDs include NSAIDS agents (e.g., cyclogenase-2 inhibitors such as Celecoxib (Celebrex®)), Diclofenac (Cataflam®, Voltaren®, Arthrotec®,) Diflunisal (Dolobid®, commercially available from Merck & Co), Etodolac (Lodine®), Fenoprofen (Nalfon®), Flurbirofen (Ansaid®), Ibuprofen (Motrin®, ADVIL®, NUPRIN®, Tab-Profen®, Vicoprofen®, Combunox®), Indornethacin (Indocin®, Indo-Lemmon®, Indornethagan®), Ketoprofen (Oruvail®), Ketorolac (Toradol®), Mefenamic acid (Ponstel®, commercially available from First Horizon Pharmaceutical), flufenamic acid ([N-(3-trifluoromethylphenyl)anthranilic acid]), Meloxicam (Mobic®), Naburnetone (Relafen®), Naproxen (Naprosyn®, ALEVE®, Anaprox®, Naprelan®, Naprapac®), Oxaprozin (Daypro®), Piroxicam (Feldene®), Sulindac (Clinoril®) and Tolmetin (Tolectin®)) and a mixture of two or more thereof. Preferably, the AKR competitor is Flufenamic acid ([N-(3-trifluoromethylphenyl)anthranilic acid]), Mefenamic acid (Ponstel®), Diclofenac (Cataflam®, Voltaren®, Arthrotec®,) Diflunisal (Dolobid®), or phenolphthalein. More preferably, the AKR competitor is Diflunisal (Dolobid®).

In one embodiment, at least one AKR competitor is an AKR1C1 AKR inhibitor, an AKR1C2 AKR inhibitor, an AKR1C3 AKR inhibitor, an AKR1C4 AKR inhibitor, or a mixture of two or more thereof.

Examples of suitable AKR inhibitors include benzodiazepines, cyclooxygenase (COX) 2 inhibitors, non-steroidal anti-inflammatory drugs (NSAIDS), testosterone, and a mixture of two or more thereof.

Examples of suitable benzodiazepines include cloxazolam, diazepam, estazolam, flunitrazepam, nitrazepam, medazepam, and a mixture of two or more thereof.

An example of a suitable cyclooxygenase (COX) 2 inhibitor is celecoxib.

Preferably, the AKR competitor is administered at a dosage range of about 5 to about 3200 mg per day (e.g., 5 mg, 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg per day). In one preferred embodiment, the AKR competitor is administered at a dosage range of about 5 mg to about 1500 mg per day.

In one embodiment, where the AKR competitor is diflunisal, the preferred dosage range is about 5 mg to about 1000 mg per day. In one embodiment, the preferred dosage range of diflunisal is about 5 mg to about 499 mg per day, preferably administered at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, or 499 mg per day; more preferably, at a unit dosage of 30 mg, 90 mg, or 250 mg per day. In another embodiment, the preferred dosage range of diflunisal is about 500 mg to about 1000 mg per day, preferably administered at a unit dosage of 500 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg per day; more preferably at a unit dosage of 250 mg, 500 mg or 1000 mg per day. Note that the dosage of AKR competitor may be administered as a single dose or divided over 2-4 doses per day. Preferably, the AKR competitor is administered orally or transdermally; more preferably, orally.

Preferably, the pharmaceutical composition of diflunisal is in a unit dosage form. In such form, the pharmaceutical composition of diflunisal is subdivided into suitably sized unit doses containing an amount of diflunisal effective to increase the bioavailability of a drug metabolized by AKR (e.g., a HCV protease inhibitor). An increase in bioavailability of a drug includes, but is not limited to, one or more of the following: an increase in half-life (t_{1/2}) of the drug, an increase in the time to peak plasma concentration (Cmax) of the drug, an increase in the area under the plasma concentration-time curve (AUC) of the drug, an increase in blood level of the drug.

In addition to the AKR competitor(s), the compositions, pharmaceutical compositions, therapeutic combinations, comprise at least one (one or more) compound of Formula I to XXVII above.

Suitable compounds of Formula I are disclosed in PCT International publication WO03/062265 published Jul. 31, 2003. Non-limiting examples of certain compounds disclosed in this publication include those listed at pages 48-75, incorporated herein by reference, or a pharmaceutically acceptable salt, solvate or ester thereof.

In one embodiment, at least one compound is: 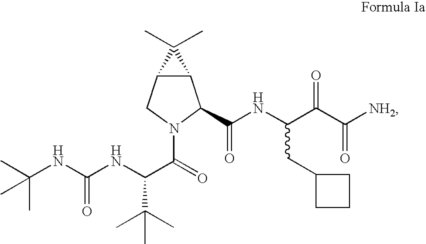
a pharmaceutically acceptable salt, solvate, or ester thereof, or a mixture of two or more thereof.

The compound of Formula Ia has recently been separated into its isomer/diastereomers of Formula Ib and Ic. In one embodiment, at least one compound is Formula Ic (a potent inhibitor of HCV NS3 serine protease), 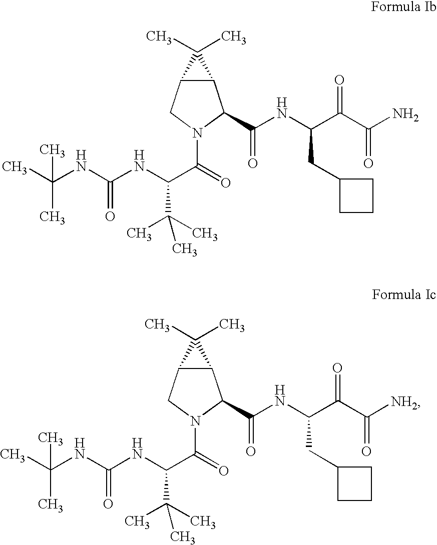
a pharmaceutically acceptable salt, solvate, or ester thereof, or a mixture of two or more thereof. The chemical name of the compound of Formula Ic is (1R,2S,5S)—N-[(1S)-3-amino-1-(cyclobutylmethyl)-2,3-dioxopropyl]-3-[(2S)-2-[[[(1,1-dimethylethyl)amino]carbonyl]amino]-3,3-dimethyl-1-oxobutyl]-6,6-dimethyl-3-azabicyclo[3.1.0]hexane-2-carboxamide.

Processes for making compounds of Formula I are disclosed in U.S. Patent Publication Nos. 2005/0059648, 2005/0020689 and 2005/0059800, incorporated by reference herein.

Non-limiting examples of suitable compounds of Formula II and methods of making the same are disclosed in WO02/08256 and in U.S. Pat. No. 6,800,434, at col. 5 through col. 247, incorporated herein by reference.

Non-limiting examples of suitable compounds of Formula III and methods of making the same are disclosed in International Patent Publication WO02/08187 and in U.S. Patent Publication 2002/0160962 at page 3, paragraph 22 through page 132, incorporated herein by reference.

Non-limiting examples of suitable compounds of Formula V1 and methods of making the same are disclosed in U.S. Patent Publication Ser. No. 2005/0085425 at page 3, paragraph 0023 through page 139, incorporated herein by reference.

Non-limiting examples of suitable compounds of Formula VIII and methods of making the same are disclosed in International Patent Publication WO2005/051980 and in U.S. Patent Publication 2005/0164921 at page 3, paragraph [0026] through page 113, paragraph [0271], incorporated herein by reference.

Non-limiting examples of suitable compounds of Formula X and methods of making the same are disclosed in International Patent Publication WO2005/085275 and in U.S. Patent Publication 2005/0267043 at page 4, paragraph [0026] through page 519, paragraph [0444], incorporated herein by reference.

Non-limiting examples of suitable compounds of Formula XI and methods of making the same are disclosed in International Patent Publication WO2005/087721 and in U.S. Patent Publication 2005/0288233 at page 3, paragraph [0026] through page 280, paragraph [0508], incorporated herein by reference.

Non-limiting examples of suitable compounds of Formula XII and methods of making the same are disclosed in International Patent Publication WO2005/087725 and in U.S. Patent Publication 2005/0245458 at page 4, paragraph [0026] through page 194, paragraph [0374], incorporated herein by reference.

Non-limiting examples of suitable compounds of Formula XIII and methods of making the same are disclosed in International Patent Publication WO2005/085242 and in U.S. Patent Publication 2005/0222047 at page 3, paragraph [0026] through page 209, paragraph [0460], incorporated herein by reference.

Non-limiting examples of suitable compounds of Formula XIV and methods of making the same are disclosed in International Patent Publication WO2005/087731 at page 8, line 20 through page 683, line 6, incorporated herein by reference.

Non-limiting examples of suitable compounds of Formula XV and methods of making the same are disclosed in International Patent Publication WO2005/058821 and in U.S. Patent Publication 2005/0153900 at page 4, paragraph [0028] through page 83, paragraph [0279], incorporated herein by reference.

Non-limiting examples of suitable compounds of Formula XVI and methods of making the same are disclosed in International Patent Publication WO2005/087730 and in U.S. Patent Publication 2005/0197301 at page 3, paragraph [0026] through page 156, paragraph [0312], incorporated herein by reference.

Non-limiting examples of suitable compounds of Formula XVII and methods of making the same are disclosed in International Patent Publication WO2005/085197 and in U.S. Patent Publication 2005/0209164 at page 3, paragraph [0026] through page 87, paragraph [0354], incorporated herein by reference.

Non-limiting examples of suitable compounds of Formula XIX and methods of making the same are disclosed in International Patent Publication WO2005/113581 and in U.S. Patent Publication 2005/0272663 at page 3, paragraph [0026] through page 76, incorporated herein by reference.

Non-limiting examples of suitable compounds of Formula XXIII and methods of making the same are disclosed in International Patent Publication WO02/18369 at page 4, line 4 through page 311, incorporated herein by reference.

Non-limiting examples of suitable compounds of Formula XXIV and methods of making the same are disclosed in U.S. Patent Publication No. 2002/0032175, 2004/0266731 and U.S. Pat. No. 6,265,380 at col. 3, line 35 through col. 121 and 6,617,309 at col. 3, line 40 through col. 121, each incorporated herein by reference.

Non-limiting examples of suitable compounds of Formula XXVI and methods of making the same are disclosed in U.S. Pat. No. 6,143,715 at col. 3, line 6 through col. 62, line 20, incorporated herein by reference.

Isomers of the various compounds of the present invention (where they exist), including enantiomers, stereoisomers, rotamers, tautomers and racemates are also contemplated as being part of this invention. The invention includes d and I isomers in both pure form and in admixture, including racemic mixtures. Isomers can be prepared using conventional techniques, either by reacting optically pure or optically enriched starting materials or by separating isomers of a compound of the present invention. Isomers may also include geometric isomers, e.g., when a double bond is present. Polymorphous forms of the compounds of the present invention, whether crystalline or amorphous, also are contemplated as being part of this invention. The (+) isomers of the present compounds are preferred compounds of the present invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon are also within the scope of this invention.

It is apparent to one skilled in the art that certain compounds of this invention may exist in alternative tautomeric forms. All such tautomeric forms of the present compounds are within the scope of the invention. Unless otherwise indicated, the representation of either tautomer is meant to include the other. For example, both isomers (1) and (2) are contemplated: 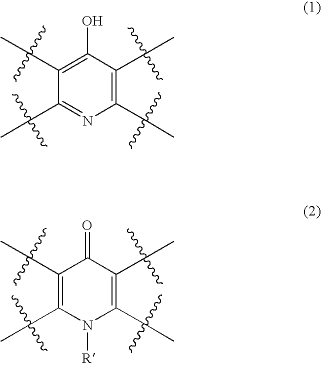
wherein R′ is H or C₁₋₆ unsubstituted alkyl.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula I or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula I or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl, and the like.

Similarly, if a compound of Formula I contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula I incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N- or di-N,N-(C₁-C₆)alkylaminoalkyl, —C(Y⁴)Y⁵ wherein Y⁴ is H or methyl and Y⁵ is mono-N— or di-N,N-(C₁-C₆)alkylamino morpholino, piperidin-1-yl or pyrrolidin-1-yl, and the like.

“Solvate” means a physical association of a compound of this invention with one or more solvent molecules. This physical association involves varying degrees of ionic and covalent bonding, including hydrogen bonding. In certain instances the solvate is capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may also exist as, or optionally converted to, a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving a compound in desired amounts of the desired solvent (organic or water or a mixture of two or more thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of a compound or a composition of the present invention effective in inhibiting HCV protease and/or cathepsins, and thus producing the desired therapeutic, ameliorative, inhibitory or preventative effect in a suitable subject.

Other than in the operating examples, or where otherwise indicated, all numbers expressing quantities of ingredients, reaction conditions, and so forth used in the specification and claims are to be understood as being modified in all instances by the term “about.”

The compounds of the present invention form salts that are also within the scope of this invention. Reference to a compound of the present invention herein is understood to include reference to salts, esters and solvates thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the various formulas of the present invention may be formed, for example, by reacting a compound of the present invention with an amount of acid or base, such as an equivalent amount, in a medium such as one in which the salt precipitates or in an aqueous medium followed by lyophilization. Acids (and bases) which are generally considered suitable for the formation of pharmaceutically useful salts from basic (or acidic) pharmaceutical compounds are discussed, for example, by S. Berge et al, Journal of Pharmaceutical Sciences (1977)66(1) 1-19; P. Gould, International J. of Pharmaceutics (1986) 33 201-217; Anderson et al, The Practice of Medicinal Chemistry (1996), Academic Press, New York; in The Orange Book (Food & Drug Administration, Washington, D.C. on their website); and P. Heinrich Stahl, Camille

G. Wermuth (Eds.), Handbook of Pharmaceutical Salts: Properties, Selection, and Use, (2002) Int'l. Union of Pure and Applied Chemistry, pp. 330-331. These disclosures are incorporated herein by reference thereto.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates,) undecanoates, and the like.

Exemplary basic salts include ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl)ethylenediamine), N-methyl-D-glucamines, N-methyl-D-glucamides, t-butyl amines, piperazine, phenylcyclohexylamine, choline, tromethamine, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention. All acid and base salts, as well as esters and solvates, are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

In such esters, unless otherwise specified, any alkyl moiety present preferably contains from 1 to 18 carbon atoms, particularly from 1 to 6 carbon atoms, more particularly from 1 to 4 carbon atoms. Any cycloalkyl moiety present in such esters preferably contains from 3 to 6 carbon atoms. Any aryl moiety present in such esters preferably comprises a phenyl group.

In another embodiment, this invention provides pharmaceutical compositions comprising the inventive peptides as an active ingredient. The pharmaceutical compositions generally additionally comprise a pharmaceutically acceptable carrier diluent, excipient or carrier (collectively referred to herein as carrier materials). Because of their HCV inhibitory activity, such pharmaceutical compositions possess utility in treating hepatitis C and related disorders.

Another embodiment of the invention discloses the use of the pharmaceutical compositions disclosed above for treatment of diseases such as, for example, HCV, inhibiting cathepsin activity and the like. The method comprises administering a therapeutically effective amount of the inventive pharmaceutical composition to a patient having such a disease or diseases and in need of such a treatment.

In yet another embodiment, the compounds of the invention may be used for the treatment of HCV in humans in monotherapy mode or in a combination therapy (e.g., dual combination, triple combination etc.) mode such as, for example, in combination with at least one other therapeutic agent (e.g., antiviral and/or immunomodulatory agents). Examples of other therapeutic agents include Ribavirin (from Schering-Plough Corporation, Madison, N.J.) and Levovirin™ (from ICN Pharmaceuticals, Costa Mesa, Calif.), VP 50406™ (from Viropharma, Incorporated, Exton, Pa.), ISIS 14803™ (from ISIS Pharmaceuticals, Carlsbad, Calif.), Heptazyme™ (from Ribozyme Pharmaceuticals, Boulder, Colo.), VX₄₉₇™ (from Vertex Pharmaceuticals, Cambridge, Mass.), Thymosin™ (from SciClone Pharmaceuticals, San Mateo, Calif.), Maxamine™ (Maxim Pharmaceuticals, San Diego, Calif.), mycophenolate mofetil (from Hoffman-LaRoche, Nutley, N.J.), interferon (such as, for example, interferon-alpha, PEG-interferon alpha conjugates), antibodies specific to IL-10 (such as those disclosed in US2005/0101770, paragraphs [0086] to [0104] incorporated herein by reference, e.g., humanized 12G8, a humanized monoclonal antibody against human IL-10, plasmids containing the nucleic acids encoding the humanized 12G8 light and heavy chains were deposited with the American Type Culture Collection (ATCC) as deposit numbers PTA-5923 and PTA-5922, respectively), and the like. “PEG-interferon alpha conjugates” are interferon alpha molecules covalently attached to a PEG molecule. Illustrative PEG-interferon alpha conjugates include interferon alpha-2a (Roferon™, from Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name PEG-Intron™), interferon alpha-2c (Berofor Alpha™, from Boehringer Ingelheim, Ingelheim, Germany), interferon alpha fusion polypeptides, or consensus interferon as defined by determination of a consensus sequence of naturally occurring interferon alphas (Infergen™, from Amgen, Thousand Oaks, Calif.).

The HCV protease inhibitor and AKR competitor can be administered in combination with interferon alpha, PEG-interferon alpha conjugates, interferon alpha fusion polypeptides, or consensus interferon concurrently or consecutively at recommended dosages for the duration of HCV treatment in accordance with the methods of the present invention. The commercially available forms of interferon alpha include interferon alpha 2a and interferon alpha 2b and also pegylated forms of both aforementioned interferon alphas. The recommended dosage of INTRON-A interferon alpha 2b (commercially available from Schering-Plough Corp.) as administered by subcutaneous injection at 3MIU(12 mcg)/0.5 mL/TIW is for 24 weeks or 48 weeks for first time treatment. The recommended dosage of PEG-INTRON interferon alpha 2b pegylated (commercially available from Schering-Plough Corp.) as administered by subcutaneous injection at 1.5 mcg/kg/week, within a range of 40 to 150 mcg/week, is for at least 24 weeks. The recommended dosage of ROFERON A inteferon alpha 2a (commercially available from Hoffmann-La Roche) as administered by subcutaneous or intramuscular injection at 3MIU(11.1 mcg/mL)/TIW is for at least 48 to 52 weeks, or alternatively 6MIU/TIW for 12 weeks followed by 3MIU/TIW for 36 weeks. The recommended dosage of PEGASUS interferon alpha 2a pegylated (commercially available from Hoffmann-La Roche) as administered by subcutaneous injection at 180 mcg/1 mL or 180 mcg/0.5 mL is once a week for at least 24 weeks. The recommended dosage of INFERGEN interferon alphacon-1 (commercially available from Amgen) as administered by subcutaneous injection at 9 mcg/TIW is for 24 weeks for first time treatment and up to 15 mcg/TIW for 24 weeks for non-responsive or relapse treatment. Optionally, Ribavirin, a synthetic nucleoside analogue with activity against a broad spectrum of viruses including HCV, can be included in combination with the interferon and the HCV protease inhibitor. The recommended dosage of ribavirin is in a range from 600 to 1400 mg per day for at least 24 weeks (commercially available as REBETOL ribavirin from Schering-Plough or COPEGUS ribavirin from Hoffmann-La Roche).

The compositions and combinations of the present invention can be useful for treating subjects of any hepatitis C virus (HCV) genotype. HCV types and subtypes may differ in their antigenicity, level of viremia, severity of disease produced, and response to interferon therapy. (Holland, J. et al., “Hepatitis C genotyping by direct sequencing of the product from the Roche Amplicor Test: methodology and application to a South Australian population,” Pathology, 30:192-195, 1998). The nomenclature of Simmonds, P. et al. (“Classification of hepatitis C virus into six major genotypes and a series of subtypes by phylogenetic analysis of the NS-5 region,” J. Gen. Virol., 74:2391-9, 1993) is widely used and classifies isolates into six major genotypes, 1 through 6, with two or more related subtypes, e.g., 1a, 1b. Additional genotypes 7-10 and 11 have been proposed, however the phylogenetic basis on which this classification is based has been questioned, and thus types 7, 8, 9 and 11 isolates have been reassigned as type 6, and type 10 isolates as type 3. (Lamballerie, X. et al., “Classification of hepatitis C variants in six major types based on analysis of the envelope 1 and nonstructural 5B genome r egions and complete polyprotein sequences,” J. Gen. Virol., 78:45-51, 1997). The major genotypes have been defined as having sequence similarities of between 55 and 72% (mean 64.5%), and subtypes within types as having 75%-86% similarity (mean 80%) when sequenced in the NS-5 region. (Simmonds, P. et al., “Identification of genotypes of hepatitis C by sequence comparisons in the core, E1 and NS-5 regions,” J. Gen. Virol., 75:1053-61, 1994).

In another embodiment, the compounds of the invention can be used to treat cellular proliferation diseases. Such cellular proliferation disease states which can be treated by the compounds, compositions and methods provided herein include, but are not limited to, cancer (further discussed below), hyperplasia, cardiac hypertrophy, autoimmune diseases, fungal disorders, arthritis, graft rejection, inflammatory bowel disease, immune disorders, inflammation, cellular proliferation induced after medical procedures, including, but not limited to, surgery, angioplasty, and the like. Treatment includes inhibiting cellular proliferation. It is appreciated that in some cases the cells may not be in a hyper- or hypoproliferation state (abnormal state) and still require treatment. For example, during wound healing, the cells may be proliferating “normally”, but proliferation enhancement may be desired. Thus, in one embodiment, the invention herein includes application to cells or subjects afflicted or subject to impending affliction with any one of these disorders or states.

The methods provided herein are particularly useful for the treatment of cancer including solid tumors such as skin, breast, brain, colon, gall bladder, thyroid, cervical carcinomas, testicular carcinomas, etc. More particularly, cancers that may be treated by the compounds, compositions and methods of the invention include, but are not limited to:

Cardiac: sarcoma (angiosarcoma, fibrosarcoma, rhabdomyosarcoma, liposarcoma), myxoma, rhabdomyoma, fibroma, lipoma and teratoma;

Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;

Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);

Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

Liver: hepatoma (hepatocellular carcinoma), cholangiocarcinoma, hepatoblastoma, angiosarcoma, hepatocellular adenoma, hemangioma;

Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;

Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma);

Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);

Hematologic: blood (myeloid leukemia (acute and chronic), acute lymphoblastic leukemia, acute and chronic lymphocytic leukemia, myeloproliferative diseases, multiple myeloma, myelodysplastic syndrome), Hodgkin's disease, non-Hodgkin's lymphoma (malignant lymphoma), B-cell lymphoma, T-cell lymphoma, hairy cell lymphoma, Burkett's lymphoma, promyelocytic leukemia;

Skin: malignant melanoma, basal cell carcinoma, squamous cell carcinoma, Karposi's sarcoma, moles dysplastic nevi, lipoma, angioma, dermatofibroma, keloids, psoriasis;

Adrenal glands: neuroblastoma; and

Other tumors: including xenoderoma pigmentosum, keratoctanthoma and thyroid follicular cancer.

As used herein, treatment of cancer includes treatment of cancerous cells, including cells afflicted by any one of the above-identified conditions.

The compounds of the present invention may also be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

The compounds of the present invention may also be useful in inhibiting tumor angiogenesis and metastasis.

The compounds of the present invention may also be useful as antifungal agents, by modulating the activity of the fungal members of the bimC kinesin subgroup, as is described in U.S. Pat. No. 6,284,480.

The present compounds are also useful in combination with one or more other known therapeutic agents and anti-cancer agents. Combinations of the present compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The present compounds are also useful when co-administered with radiation therapy.

The phrase “estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-I-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-ydrazone, aid SH646.

The phrase “androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

The phrase “retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, a difluoromethylornithine, ILX23-7553, trans-N-(4′-hydroxyphenyl) retinamide, and N-4-carboxyphenyl retinamide.

The phrase “cytotoxic/cytostatic agents” refer to compounds which cause cell death or inhibit cell proliferation primarily by interfering directly with the cell's functioning or inhibit or interfere with cell mycosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, haematopoietic growth factors, monoclonal antibody targeted therapeutic agents, monoclonal antibody therapeutics, topoisomerase inhibitors, proteasome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide (TEMODAR™ from Schering-Plough Corporation, Kenilworth, N.J.), cyclophosphamide, heptaplatin, estramustine, improsulfan tosilate, trofosfamide, nimustine, dibrospidium chloride, pumitepa, lobaplatin, satraplatin, profiromycin, cisplatin, doxorubicin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deansino-3′-morpholino-13-deoxo-10-hydroxycaminomycin, annamycin, galarubicin, elinafide, MEN10755, 4-demethoxy-3-deamino-3-aziridinyl-4-methylsulphonyl-daunombicin (see WO 00/50032), methoxtrexate, gemcitabine, and mixture thereof.

An example of a hypoxia activatable compound is tirapazamine.

Examples of proteasome inhibitors include, but are not limited to, lactacystin and bortezomib.

Examples of microtubule inhibitors/microtubule-stabilising agents include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyi-L-valyl-N-methyl-L-valyl-L-prolyl-L-proline-t-butylamide, TDX²⁵⁸, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino) ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro (3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino] benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2-(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, dimesna, and camptostar.

Other useful anti-cancer agents that can be used in combination with the present compounds include thymidilate synthase inhibitors, such as 5-fluorouracil.

In one embodiment, inhibitors of mitotic kinesins include, but are not limited to, inhibitors of KSP, inhibitors of MKLP1, inhibitors of CENP-E, inhibitors of MCAK, inhibitors of Kif14, inhibitors of Mphosphl and inhibitors of Rab6-KIFL.

The phrase “inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

The phrase “antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,1′-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

Examples of monoclonal antibody therapeutics useful for treating cancer include Erbitux (Cetuximab).

The phrase “HMG-CoA reductase inhibitors” refers to inhibitors of 3-hydroxy-3-methylglutaryl-CoA reductase. Examples of HMG-CoA reductase inhibitors that may be used include but are not limited to lovastatin, simvastatin (ZOCOR®), pravastatin (PRAVACHOL®), fluvastatin and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefore the use of such salts, esters, open acid and lactone forms is included in the scope of this invention.

The phrase “prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO, 97/30053, WO 97/44350, WO 98/02436, and U.S. Pat. No. 5,532,359. For an example of the role of a prenyl-protein transferase inhibitor on angiogenesis see European of Cancer, Vol. 35, No. 9, pp. 1394-1401 (1999).

Examples of farnesyl protein transferase inhibitors include SARASAR™ (4-[2-[4-[(11R)-3,10-dibromo-8-chloro-6,11-dihydro-5H-benzo[5,6]cyclohepta[1,2-b]pyridin-11-yl-]-1-piperidinyl]-2-oxoethyl]-1-piperidinecarboxamide from Schering-Plough Corporation, Kenilworth, N.J.), tipifarnib (Zarnestra® or R115777 from Janssen Pharmaceuticals), L778,123 (a farnesyl protein transferase inhibitor from Merck & Company, Whitehouse Station, N.J.), BMS 214662 (a farnesyl protein transferase inhibitor from Bristol-Myers Squibb Pharmaceuticals, Princeton, N.J.).

The phrase “angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α (for example Intron and Peg-Intron), interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin. Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin 11 antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). Examples of TAFIa inhibitors have been described in PCT Publication WO 03/013,526.

The phrase “agents that interfere with cell cycle checkpoints” refers to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

The phrase “inhibitors of cell proliferation and survival signaling pathway” refers to agents that inhibit cell surface receptors and signal transduction cascades downstream of those surface receptors. Such agents include inhibitors of EGFR (for example gefitinib and erlotinib), antibodies to EGFR (for example C225), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as described in WO 02/083064, WO 02/083139, WO 02/083140 and WO 02/083138), inhibitors of Raf kinase (for example BAY-43-9006), inhibitors of MEEK (for example CI-1040 and PD-098059), inhibitors of mTOR (for example Wyeth CCI-779), and inhibitors of C-abl kinase (for example GLEEVEC™, Novartis Pharmaceuticals). Such agents include small molecule inhibitor compounds and antibody antagonists.

The phrase “apoptosis inducing agents” includes activators of TNF receptor family members (including the TRAIL receptors).

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC50 for COX-2 over IC50 for COX-1 evaluated by cell or microsomal assays. Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5 pyridinyl)pyridine; or a pharmaceutically acceptable salt thereof.

Compounds that have been described as specific inhibitors of COX-2 and are therefore useful in the present invention include, but are not limited to, parecoxib, CELEBREX® and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RP14610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, ST1571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, ST1571A, N-4-chlorophenyl-4-(4-pyridylmethyl)-1-phthalazinamine, and EMD121974.

Combinations with compounds other than anti-cancer compounds are also encompassed in the instant methods. For example, combinations of the present compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999; 274:9116-9121; Invest. Opthalmol. Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid, and 2(R)-7-(3-(2-chloro-4-(4-fluorophenoxy)phenoxy)propoxy)-2-ethylchromane-2-carboxylic acid.

In one embodiment, useful anti-cancer (also known as anti-neoplastic) agents that can be used in combination with the present compounds include, but are not limited, to Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin (ELOXATIN™ from Sanofi-Synthelabo Pharmaeuticals, France), Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin (adriamycin), cyclophosphamide (cytoxan), gemcitabine, interferons, pegylated interferons, Erbitux, and a mixture of two or more thereof.

Another embodiment of the present invention is the use of the present compounds in combination with gene therapy for the treatment of cancer. For an overview of genetic strategies to treating cancer, see Hall et al (Am J Hum Genet 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J Immunol2000; 164:217-222).

The present compounds can also be administered in combination with one or more inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (vaispodar).

The present compounds can also be employed in conjunction with one or more anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with one or more other anti-emetic agents, especially neurokinin-1 receptor antagonists, 5HT3 receptor, antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or those as described in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In one embodiment, an anti-emesis agent selected from a neurokinin-1 receptor antagonist, a 5HT3 receptor antagonist and a corticosteroid is administered as an adjuvant for the treatment or prevention of emesis that may result upon administration of the present compounds.

Examples of neurokinin-1 receptor antagonists that can be used in conjunction with the present compounds are described in U.S. Pat. Nos. 5,162,339, 5,232,929, 5,242,930, 5,373,003, 5,387,595, 5,459,270, 5,494,926, 5,496,833, 5,637,699, and 5,719,147, content of which are incorporated herein by reference. In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the present invention may also be administered with one or more immunologic-enhancing drug, such as for example, levamisole, isoprinosine and Zadaxin.

Thus, the present invention encompasses the use of the present compounds (for example, for treating or preventing cellular proliferative diseases) in combination with a second compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug resistance, an anti-emetic agent, an immunologic-enhancing drug, an inhibitor of cell proliferation and survival signaling, an agent that interfers with a cell cycle checkpoint, and an apoptosis inducing agent.

In one embodiment, the present invention emcompasses the composition and use of the present compounds in combination with a second compound selected from: a cytostatic agent, a cytotoxic agent, taxanes, a topoisomerase II inhibitor, a topoisomerase I inhibitor, a tubulin interacting agent, hormonal agent, a thymidilate synthase inhibitors, anti-metabolites, an alkylating agent, a farnesyl protein transferase inhibitor, a signal transduction inhibitor, an EGFR kinase inhibitor, an antibody to EGFR, a C-abl kinase inhibitor, hormonal therapy combinations, and aromatase combinations.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In one embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MW (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-(O-chloroacetylcarbonyl)-fumagillol, thalidomide, angiostatin, troponin-1, or an antibody to VEGF. In an embodiment, the estrogen receptor modulator is tamoxifen or raloxifene.

Also included in the present invention are pharmaceutical kits comprising (a) at least one aldo-keto reductase (AKR) competitor; and (b) at least one compound selected from the group consisting of compounds of Formula I to XXVII described above, in separate dosage forms, said forms being suitable for administration of (a) and (b) in effective amounts, and instructions for administering (a) and (b).

Also included in the present invention is a method of treating cancer comprising administering a therapeutically effective amount of at least one compound of the present invention in combination with radiation therapy and at least one compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug resistance, an anti-emetic agent, an immunologic-enhancing drag, an inhibitor of cell proliferation and survival signaling, an agent that interfers with a cell cycle checkpoint, and an apoptosis inducing agent.

Yet another embodiment of the invention is a method of treating cancer comprising administering a therapeutically effective amount of at least one compound of the present invention in combination with paclitaxel or trastuzumab.

The present invention also includes a pharmaceutical composition useful for treating or preventing the various disease states mentioned herein cellular proliferation diseases (such as cancer, hyperplasia, cardiac hypertrophy, autoimmune diseases, fungal disorders, arthritis, graft rejection, inflammatory bowel disease, immune disorders, inflammation, and cellular proliferation induced after medical procedures) that comprises a therapeutically effective amount of at least one compound of the present invention and at least one compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of cell proliferation and survival signaling, an agent that interfers with a cell cycle checkpoint, and an apoptosis inducing agent.

Methods for treating, preventing, or ameliorating one or more symptoms of HCV, treating disorders associated with HCV, or inhibiting cathepsin activity in a subject, comprising the step of administering to a subject in need of such treatment an effective amount of the above medicaments, also are provided.

Examples of such cathepsin-associated disorders include proliferative diseases, such as cancer, autoimmune diseases, viral diseases, fungal diseases, neurological/neurodegenerative disorders, arthritis, inflammation, anti-proliferative (e.g., ocular retinopathy), neuronal, alopecia and cardiovascular disease. Many of these diseases and disorders are listed in U.S. Pat. No. 6,413,974, the disclosure of which is incorporated herein.

Other examples of diseases that can be treated include an inflammatory disease, such as organ transplant rejection, graft v. host disease, arthritis, rheumatoid arthritis, inflammatory bowel disease, atopic dermatitis, psoriasis, asthma, allergies, multiple sclerosis, fixed drug eruptions, cutaneous delayed-type hypersentitivity responses, tuberculoid leprosy, type I diabetes, and viral meningitis.

Another example of a disease that can be treated is a cardiovascular disease.

Other examples of diseases that can be treated include a central nervous system disease, such as depression, cognitive function disease, neurodegenerative disease such as Parkinson's disease, senile dementia such as Alzheimer's disease, and psychosis of organic origin.

Other examples of diseases that can be treated include diseases characterized by bone loss, such as osteoporosis; gingival diseases, such as gingivitis and periodontitis; and diseases characterized by excessive cartilage or matrix degradation, such as osteoarthritis and rheumatoid arthritis.

When the disease being treated by the cathepsin inhibitor compounds of the present invention is inflammatory disease, an embodiment of the present invention comprises administering: (a) a therapeutically effective amount of at least one compound of the present cathepsin inhibitors (e.g., a compound according to Formula I-XXVII) or a pharmaceutically acceptable salt, solvate or ester thereof concurrently or sequentially with (b) at least one medicament selected from the group consisting of: disease modifying antirheumatic drugs; nonsteroidal anti-inflammatory drugs; COX-2 selective inhibitors; COX-1 inhibitors; immunosuppressives (non-limiting examples include methotrexate, cyclosporin, FK506); steroids; PDE IV inhibitors, anti-TNF-α compounds, TNF-alpha-convertase inhibitors, cytokine inhibitors, MMP inhibitors, glucocorticoids, chemokine inhibitors, CB2-selective inhibitors, p38 inhibitors, biological response modifiers; anti-inflammatory agents and therapeutics.

Another embodiment of the present invention is directed to a method of inhibiting or blocking T-cell mediated chemotaxis in a patient in need of such treatment the method comprising administering to the patient a therapeutically effective amount of at least one compound of the present cathepsin inhibitors (e.g., a compound according to Formula I-XXVII) or a pharmaceutically acceptable salt, solvate or ester thereof.

Another embodiment of this invention is directed to a method of treating inflammatory bowel disease in a patient in need of such treatment comprising administering to the patient a therapeutically effective amount of at least one compound according to the present cathepsin inhibitors or a pharmaceutically acceptable salt, solvate or ester thereof.

Another embodiment of this invention is directed to a method of treating or preventing graft rejection in a patient in need of such treatment comprising administering to the patient a therapeutically effective amount of at least one compound according to the present cathepsin inhibitors, or a pharmaceutically acceptable salt, solvate or ester thereof.

Another embodiment of this invention is directed to a method comprising administering to the patient a therapeutically effective amount of: (a) at least one compound according to the present cathepsin inhibitors, or a pharmaceutically acceptable salt, solvate or ester thereof concurrently or sequentially with (b) at least one compound selected from the group consisting of: cyclosporine A, FK-506, FTY720, beta-Interferon, rapamycin, mycophenolate, prednisolone, azathioprine, cyclophosphamide and an antilymphocyte globulin.

Another embodiment of this invention is directed to a method of treating multiple sclerosis in a patient in need of such treatment the method comprising administering to the patient a therapeutically effective amount of: (a) at least one AKR inhibitor and at least one cathepsin inhibitor compound according to the present invention, or a pharmaceutically acceptable salt, solvate or ester thereof concurrently or sequentially with (b) at least one compound selected from the group consisting of: beta-interferon, glatiramer acetate, glucocorticoids, methotrexate, azothioprine, mitoxantrone, VLA-4 inhibitors and/or CB2-selective inhibitors.

Another embodiment of this invention is directed to a method of treating multiple sclerosis in a patient in need of such treatment the method comprising administering to the patient a therapeutically effective amount of: a) at least one AKR inhibitor and at least one cathepsin inhibitor compound according to the present invention, or a pharmaceutically acceptable salt, solvate or ester thereof concurrently or sequentially with (b) at least one compound selected from the group consisting of: methotrexate, cyclosporin, leflunimide, sulfasalazine, β-methasone, β-interferon, glatiramer acetate, prednisone, etonercept, and infliximab.

Another embodiment of this invention is directed to a method of treating rheumatoid arthritis in a patient in need of such treatment the method comprising administering to the patient a therapeutically effective amount of: (a) at least one AKR inhibitor and at least one cathepsin inhibitor compound according to the present invention or a pharmaceutically acceptable salt, solvate or ester thereof concurrently or sequentially with (b) at least one compound selected from the group consisting of: COX-2 inhibitors, COX inhibitors, immunosuppressives, steroids, PDE IV inhibitors, anti-TNF-α compounds, MMP inhibitors, glucocorticoids, chemokine inhibitors, CB2-selective inhibitors, caspase (ICE) inhibitors and other classes of compounds indicated for the treatment of rheumatoid arthritis.

Another embodiment of this invention is directed to a method of treating psoriasis in a patient in need of such treatment the method comprising administering to the patient a therapeutically effective amount of: a) at least one AKR inhibitor and at least one cathepsin inhibitor compound according to the present invention, or a pharmaceutically acceptable salt, solvate or ester thereof concurrently or sequentially with (b) at least one compound selected from the group consisting of: immunosuppressives, steroids, and anti-TNF-α compounds.

Another embodiment of this invention is directed to a method of treating a disease selected from the group consisting of: inflammatory disease, rheumatoid arthritis, multiple sclerosis, inflammatory bowel disease, graft rejection, psoriasis, fixed drug eruptions, cutaneous delayed-type hypersensitivity responses, tuberculoid leprosy, type I diabetes, viral meningitis and tumors in a patient in need of such treatment, such method comprising administering to the patient an effective amount of at least one AKR inhibitor and at least one cathepsin inhibitor compound according to the present invention, or a pharmaceutically acceptable salt, solvate or ester thereof.

Another embodiment of this invention is directed to a method of treating a disease selected from the group consisting of inflammatory disease, rheumatoid arthritis, multiple sclerosis, inflammatory bowel disease, graft rejection, psoriasis, fixed drug eruptions, cutaneous delayed-type hypersensitivity responses, tuberculoid leprosy and cancer in a patient in need of such treatment, such method comprising administering to the patient an effective amount of at least one AKR inhibitor and at least one cathepsin inhibitor compound according to the present invention, or a pharmaceutically acceptable salt, solvate or ester thereof.

Another embodiment of this invention is directed to a method of treating a disease selected from the group consisting of inflammatory disease, rheumatoid arthritis, multiple sclerosis, inflammatory bowel disease, graft rejection, psoriasis, fixed drug eruptions, cutaneous delayed-type hypersensitivity responses and tuberculoid leprosy, type I diabetes, viral meningitis and cancer in a patient in need of such treatment, such method comprising administering to the patient an effective amount of (a) at least one AKR inhibitor and at least one cathepsin inhibitor compound according to the present invention, or a pharmaceutically acceptable salt, solvate or ester thereof concurrently or sequentially with (b) at least one medicament selected from the group consisting of: disease modifying antirheumatic drugs; nonsteroidal anti-inflammatory drugs; COX-2 selective inhibitors; COX-1 inhibitors; immunosuppressives; steroids; PDE IV inhibitors, anti-TNF-α compounds, MMP inhibitors, glucocorticoids, chemokine inhibitors, CB2-selective inhibitors, biological response modifiers; anti-inflammatory agents and therapeutics.

When the present invention involves a method of treating a cardiovascular disease, in addition to administering at least one AKR inhibitor and at least one cathepsin inhibitor compound according to the present invention, the method further comprises administering to the subject in need one or more pharmacological or therapeutic agents or drugs such as cholesterol biosynthesis inhibitors and/or lipid-lowering agents discussed below.

Non-limiting examples of cholesterol biosynthesis inhibitors for use in the compositions, therapeutic combinations and methods of the present invention include competitive inhibitors of HMG CoA reductase, the rate-limiting step in cholesterol biosynthesis, squalene synthase inhibitors, squalene epoxidase inhibitors and a mixture of two or more thereof. Non-limiting examples of suitable HMG CoA reductase inhibitors include statins such as lovastatin (for example MEVACOR® which is available from Merck & Co.), pravastatin (for example PRAVACHOL® which is available from Bristol Meyers Squibb), fluvastatin, simvastatin (for example ZOCOR® which is available from Merck & Co.), atorvastatin, cerivastatin, rosuvastatin, rivastatin (sodium 7-(4-fluorophenyl)-2,6-diisopropyl-5-methoxymethylpyridin-3-yl)-3,5-dihydroxy-6-heptanoate, CI-981 and pitavastatin (such as NK-104 of Negma Kowa of Japan); HMG CoA synthetase inhibitors, for example L-659,699 ((E,E)-11-[3′R-(hydroxy-methyl)-4′-oxo-2′R-oxetanyl]-3,5,7R-trimethyl-2,4-undecadienoic acid); squalene synthesis inhibitors, for example squalestatin 1; and squalene epoxidase inhibitors, for example, NB-598 ((E)-N-ethyl-N-(6,6-dimethyl-2-hepten-4-ynyl)-3-[(3,3′-bithiophen-5-yl)methoxy]benzene-methanamine hydrochloride) and other sterol biosynthesis inhibitors such as DMP-565. Preferred HMG CoA reductase inhibitors include lovastatin, pravastatin and simvastatin.

In another embodiment, the method of treatment comprises administering at least one AKR inhibitor and at least one cathepsin inhibitor compound according to the present invention in combination with one or more cardiovascular agents and one or more cholesterol biosynthesis inhibitors.

In another alternative embodiment, the method treatment of the present invention can further comprise administering nicotinic acid (niacin) and/or derivatives thereof, optionally with the cardiovascular agent(s) and sterol absorption inhibitor(s) discussed above.

As used herein, “nicotinic acid derivative” means a compound comprising a pyridine-3-carboxylate structure or a pyrazine-2-carboxylate structure, including acid forms, salts, esters, zwitterions and tautomers, where available. Examples of nicotinic acid derivatives include niceritrol, nicofuranose and acipimox (5-methyl pyrazine-2-carboxylic acid 4-oxide). Nicotinic acid and its derivatives inhibit hepatic production of VLDL and its metabolite LDL and increases HDL and apo A-1 levels. An example of a suitable nicotinic acid product is NIASPAN® (niacin extended-release tablets) which are available from Kos.

In another alternative embodiment, the method of treatment of the present invention can further comprise administering one or more AcylCoA:Cholesterol O-acyltransferase (“ACAT”) Inhibitors, which can reduce LDL and VLDL levels, coadministered with or in combination with the cardiovascular agent(s) and sterol absorption inhibitor(s) discussed above. ACAT is an enzyme responsible for esterifying excess intracellular cholesterol and may reduce the synthesis of VLDL, which is a product of cholesterol esterification, and overproduction of apo B-100-containing lipoproteins.

Non-limiting examples of useful ACAT inhibitors include avasimibe ([[2,4,6-tris(1-methylethyl)phenyl]acetyl]sulfamic acid, 2,6-bis(1-methylethyl)phenyl ester, formerly known as CI-1011), HL-004, lecimibide (DuP-128) and CL-277082 (N-(2,4-difluorophenyl)-N-[[4-(2,2-dimethylpropyl)phenyl]methyl]-N-heptylurea). See P. Chang et al., “Current, New and Future Treatments in Dyslipidaemia and Atherosclerosis”, Drugs 2000 July; 60(1); 55-93, which is incorporated by reference herein.

In another alternative embodiment, the method of treatment of the present invention can further comprise administering probucol or derivatives thereof (such as AGI-1067 and other derivatives disclosed in U.S. Pat. Nos. 6,121,319 and 6,147,250), which can reduce LDL levels, coadministered with or in combination with the cardiovascular agent(s) and sterol absorption inhibitor(s) discussed above.

In another alternative embodiment, the method of treatment of the present invention can further comprise administering fish oil, which contains Omega 3 fatty acids (3-PUFA), which can reduce VLDL and triglyceride levels, coadministered with or in combination with the cardiovascular agent(s) and sterol absorption inhibitor(s) discussed above. Generally, a total daily dosage of fish oil or Omega 3 fatty acids can range from about 1 to about 30 grams per day in single or 2-4 divided doses.

In another alternative embodiment, the method of treatment of the present invention can further comprise administering natural water soluble fibers, such as psyllium, guar, oat and pectin, which can reduce cholesterol levels, coadministered with or in combination with the cardiovascular agent(s) and sterol absorption inhibitor(s) discussed above. Generally, a total daily dosage of natural water soluble fibers can range from about 0.1 to about 10 grams per day in single or 2-4 divided doses.

In another alternative embodiment, the method of treatment of the present invention can further comprise administering plant sterols, plant stanols and/or fatty acid esters of plant stanols, such as sitostanol ester used in BENECOL® margarine, which can reduce cholesterol levels, coadministered with or in combination with the cardiovascular agent(s) and sterol absorption inhibitor(s) discussed above. Generally, a total daily dosage of plant sterols, plant stanols and/or fatty acid esters of plant stanols can range from about 0.5 to about 20 grams per day in single or 2-4 divided doses.

In another alternative embodiment, the method of treatment of the present invention can further comprise administering antioxidants, such as probucol, tocopherol, ascorbic acid, β-carotene and selenium, or vitamins such as vitamin B₆ or vitamin B₁₂, coadministered with or in combination with at least one AKR inhibitor and at least one cathepsin inhibitor compound according to the present invention. Generally, a total daily dosage of antioxidants or vitamins can range from about 0.05 to about 10 grams per day in single or 2-4 divided doses.

In another alternative embodiment, the method of treatment of the present invention can further comprise administering one or more bile acid sequestrants (insoluble anion exchange resins), coadministered with or in combination with at least one AKR inhibitor and at least one cathepsin inhibitor compound according to the present invention.

Bile acid sequestrants bind bile acids in the intestine, interrupting the enterohepatic circulation of bile acids and causing an increase in the faecal excretion of steroids. Use of bile acid sequestrants is desirable because of their non-systemic mode of action. Bile acid sequestrants can lower intrahepatic cholesterol and promote the synthesis of apo B/E (LDL) receptors which bind LDL from plasma to further reduce cholesterol levels in the blood.

Non-limiting examples of suitable bile acid sequestrants include cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colestipol (a copolymer of diethylenetriamine and 1-chloro-2,3-epoxypropane, such as COLESTID® tablets which are available from Pharmacia), colesevelam hydrochloride (such as WelChol® Tablets (poly(allylamine hydrochloride) cross-linked with epichlorohydrin and alkylated with 1-bromodecane and (6-bromohexyl)-trimethylammonium bromide) which are available from Sankyo), water soluble derivatives such as 3,3-ioene, N-(cycloalkyl) alkylamines and poliglusam, insoluble quaternized polystyrenes, saponins and a mixture of two or more thereof. Other useful bile acid sequestrants are disclosed in PCT Patent Applications Nos. WO 97/11345 and WO 98/57652, and U.S. Pat. Nos. 3,692,895 and 5,703,188 which are incorporated herein by reference. Suitable inorganic cholesterol sequestrants include bismuth salicylate plus montmorillonite clay, aluminum hydroxide and calcium carbonate antacids.

Also useful with the present invention are methods of treatment that can further comprise administering at least one (one or more) activators for peroxisome proliferator-activated receptors (PPAR). These activators act as agonists for the peroxisome proliferator-activated receptors. Three subtypes of PPAR have been identified, and these are designated as peroxisome proliferator-activated receptor alpha (PPARα), peroxisome proliferator-activated receptor gamma (PPARγ) and peroxisome proliferator-activated receptor delta (PPARδ). It should be noted that PPARδ is also referred to in the literature as PPARβ and as NUC1, and each of these names refers to the same receptor.

PPARα regulates the metabolism of lipids. PPARα is activated by fibrates and a number of medium and long-chain fatty acids, and it is involved in stimulating β-oxidation of fatty acids. The PPARγ receptor subtypes are involved in activating the program of adipocyte differentiation and are not involved in stimulating peroxisome proliferation in the liver. PPARδ has been identified as being useful in increasing high density lipoprotein (HDL) levels in humans. See, e.g., WO 97/28149.

PPARα activator compounds are useful for, among other things, lowering triglycerides, moderately lowering LDL levels and increasing HDL levels. Useful examples of PPARα activators include the fibrates discussed above.

Other examples of PPARα activators useful with the practice of the present invention include suitable fluorophenyl compounds as disclosed in U.S. Pat. No. 6,028,109 which is incorporated herein by reference; certain substituted phenylpropionic compounds as disclosed in WO 00/75103 which is incorporated herein by reference; and PPARα activator compounds as disclosed in WO 98/43081 which is incorporated herein by reference.

Non-limiting examples of PPARγ activator include suitable derivatives of glitazones or thiazolidinediones, such as, troglitazone (such as REZULIN® troglitazone (-5-[[4-[3,4-dihydro-6-hydroxy-2,5,7,8-tetramethyl-2H-1-benzopyran-2-yl)methoxy]phenyl]methyl]-2,4-thiazolidinedione) commercially available from Parke-Davis); rosiglitazone (such as AVANDIA® rosiglitazone maleate (-5-[[4-[2-(methyl-2-pyridinylamino)ethoxy]phenyl]methyl]-2,4-thiazol idinedione, (Z)-2-butenedioate) (1:1) commercially available from SmithKline Beecham) and pioglitazone (such as ACTOS™ pioglitazone hydrochloride (5-[[4-[2-(5-ethyl-2-pyridinyl)ethoxy]phenyl]methyl]-2,4-] thiazolidinedione monohydrochloride) commercially available from Takeda Pharmaceuticals). Other useful thiazolidinediones include ciglitazone, englitazone, darglitazone and BRL 49653 as disclosed in WO 98/05331 which is incorporated herein by reference; PPARγ activator compounds disclosed in WO 00/76488 which is incorporated herein by reference; and PPARγ activator compounds disclosed in U.S. Pat. No. 5,994,554 which is incorporated herein by reference.

Other useful classes of PPARγ activator compounds include certain acetylphenols as disclosed in U.S. Pat. No. 5,859,051 which is incorporated herein by reference; certain quinoline phenyl compounds as disclosed in WO 99/20275 which is incorporated herein by reference; aryl compounds as disclosed by WO 99/38845 which is incorporated herein by reference; certain 1,4-disubstituted phenyl compounds as disclosed in WO 00/63161; certain aryl compounds as disclosed in WO 01/00579 which is incorporated herein by reference; benzoic acid compounds as disclosed in WO 01/12612 & WO 01/12187 which are incorporated herein by reference; and substituted 4-hydroxy-phenylalconic acid compounds as disclosed in WO 97/31907 which is incorporated herein by reference.

PPARδ compounds are useful for, among other things, lowering triglyceride levels or raising HDL levels. Non-limiting examples of PPARδ activators include suitable thiazole and oxazole derivates, such as C.A.S. Registry No. 317318-32-4, as disclosed in WO 01/00603 which is incorporated herein by reference); certain fluoro, chloro or thio phenoxy phenylacetic acids as disclosed in WO 97/28149 which is incorporated herein by reference; suitable non-β-oxidizable fatty acid analogues as disclosed in U.S. Pat. No. 5,093,365 which is incorporated herein by reference; and PPARδ compounds as disclosed in WO 99/04815 which is incorporated herein by reference.

Moreover, compounds that have multiple functionality for activating various combinations of PPARα, PPARγ and PPARδ are also useful with the practice of the present invention. Non-limiting examples include certain substituted aryl compounds as disclosed in U.S. Pat. No. 6,248,781; WO 00/23416; WO 00/23415; WO 00/23425; WO 00/23445; WO 00/23451; and WO 00/63153, all of which are incorporated herein by reference, are described as being useful PPARα and/or PPARγ activator compounds. Other non-limiting examples of useful PPARα and/or PPARγ activator compounds include activator compounds as disclosed in WO 97/25042 which is incorporated herein by reference; activator compounds as disclosed in WO 00/63190 which is incorporated herein by reference; activator compounds as disclosed in WO 01/21181 which is incorporated herein by reference; biaryl-oxa(thia)zole compounds as disclosed in WO 01/16120 which is incorporated herein by reference; compounds as disclosed in WO 00/63196 and WO 00/63209 which are incorporated herein by reference; substituted 5-aryl-2,4-thiazolidinediones compounds as disclosed in U.S. Pat. No. 6,008,237 which is incorporated herein by reference; arylthiazolidinedione and aryloxazolidinedione compounds as disclosed in WO 00/78312 and WO 00/78313G which are incorporated herein by reference; GW2331 or (2-(4-[difluorophenyl]-1 heptylureido)ethyl]phenoxy)-2-methylbutyric compounds as disclosed in WO 98/05331 which is incorporated herein by reference; aryl compounds as disclosed in U.S. Pat. No. 6,166,049 which is incorporated herein by reference; oxazole compounds as disclosed in WO 01/17994 which is incorporated herein by reference; and dithiolane compounds as disclosed in WO 01/25225 and WO 01/25226 which are incorporated herein by reference.

Other useful PPAR activator compounds include substituted benzylthiazolidine-2,4-dione compounds as disclosed in WO 01/14349, WO 01/14350 and WO/01/04351 which are incorporated herein by reference; mercaptocarboxylic compounds as disclosed in WO 00/50392 which is incorporated herein by reference; ascofuranone compounds as disclosed in WO 00/53563 which is incorporated herein by reference; carboxylic compounds as disclosed in WO 99/46232 which is incorporated herein by reference; compounds as disclosed in WO 99/12534 which is incorporated herein by reference; benzene compounds as disclosed in WO 99/15520 which is incorporated herein by reference; α-anisamide compounds as disclosed in WO 01/21578 which is incorporated herein by reference; and PPAR activator compounds as disclosed in WO 01/40192 which is incorporated herein by reference.

Also useful with the present invention are methods of treatment which further comprise administering hormone replacement agents and compositions. Useful hormone agents and compositions for hormone replacement therapy of the present invention include androgens, estrogens, progestins, their pharmaceutically acceptable salts and derivatives. Combinations of these agents and compositions are also useful.

The cathepsin inhibitors of the present invention are useful in the treatment of central nervous system diseases such as depression, cognitive function diseases and neurodegenerative diseases such as Parkinson's disease, senile dementia as in Alzheimer's disease, and psychoses of organic origin. In particular, the cathepsin inhibitors of the present invention can improve motor-impairment due to neurodegenerative diseases such as Parkinson's disease.

The other agents known to be useful in the treatment of Parkinson's disease which can be administered in combination with the cathepsin inhibitors of the present invention include: L-DOPA; dopaminergic agonists such as quinpirole, ropinirole, pramipexole, pergolide and bromocriptine; MAO-B inhibitors such as deprenyl and selegiline; DOPA decarboxylase inhibitors such as carbidopa and benserazide; and COMT inhibitors such as tolcapone and entacapone.

A preferred dosage for the administration of a compound of the present invention is about 0.001 to 500 mg/kg of body weight/day of a compound of the present invention or a pharmaceutically acceptable salt or ester thereof. An especially preferred dosage is about 0.01 to 25 mg/kg of body weight/day of a compound of the present invention or a pharmaceutically acceptable salt or ester thereof.

The phrases “effective amount” and “therapeutically effective amount” mean that amount of a compound of the present invention, and other pharmacological or therapeutic agents described herein, that will elicit a biological or medical response of a tissue, a system, or a subject (e.g., animal or human) that is being sought by the administrator (such as a researcher, doctor or veterinarian) which includes alleviation of the symptoms of the condition or disease being treated and the prevention, slowing or halting of progression of one or more of the presently claimed diseases. The formulations or compositions, combinations and treatments of the present invention can be administered by any suitable means which produce contact of these compounds with the site of action in the body of, for example, a mammal or human.

For administration of pharmaceutically acceptable salts of the above compounds, the weights indicated above refer to the weight of the acid equivalent or the base equivalent of the therapeutic compound derived from the salt.

As described above, this invention includes combinations comprising an amount of at least one AKR inhibitor and an amount of at least one HCV protease or cathepsin inhibitor compound or a pharmaceutically acceptable salt or ester thereof, and an amount of one or more additional therapeutic agents listed above (administered together or sequentially) wherein the amounts of the compounds/treatments result in desired therapeutic effect.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for illustration purposes, a compound of the present invention and an additional therapeutic agent may be present in fixed amounts (dosage amounts) in a single dosage unit (e.g., a capsule, a tablet and the like).

If formulated as a fixed dose, such combination products employ the compounds of this invention within the dosage range described herein and the other pharmaceutically active agent or treatment within its dosage range. Compounds of the present invention may also be administered sequentially with known therapeutic agents when a combination formulation is inappropriate. The invention is not limited in the sequence of administration; compounds of the present invention may be administered either prior to or after administration of the known therapeutic agent. Such techniques are within the skills of persons skilled in the art as well as attending physicians.

The pharmacological properties of the compounds of this invention may be confirmed by a number of pharmacological assays for measuring HCV viral activity or cathepsin activity, such as are well know to those skilled in the art.

While it is possible for the active ingredient to be administered alone, it is preferable to present it as a pharmaceutical composition. The compositions of the present invention comprise at least one active ingredient, as defined above, together with one or more acceptable carriers, adjuvants or vehicles thereof and optionally other therapeutic agents. Each carrier, adjuvant or vehicle must be acceptable in the sense of being compatible with the other ingredients of the composition and not injurious to the mammal in need of treatment.

Accordingly, this invention also relates to pharmaceutical compositions comprising at least one compound utilized in the presently claimed methods, or a pharmaceutically acceptable salt or ester thereof and at least one pharmaceutically acceptable carrier, adjuvant or vehicle.

In yet another embodiment, the present invention discloses methods for preparing pharmaceutical compositions comprising the inventive compounds as an active ingredient. In the pharmaceutical compositions and methods of the present invention, the active ingredients will typically be administered in admixture with suitable carrier materials suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent inventive composition. Surfactants may be present in the pharmaceutical formulations of the present invention in an amount of about 0.1 to about 10% by weight or about 1 to about 5% by weight. Acidifying agents may be present in the pharmaceutical formulations of the present invention in a total amount of about 0.1 to about 10% by weight or about 1 to 5% by weight.

Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like.

Sweetening and flavoring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate controlled release of any one or more of the components or active ingredients to optimize the therapeutic effects, i.e. HCV inhibitory activity or cathepsin inhibitory activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injections or addition of sweeteners and pacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions may take the form of creams, lotions, aerosols and/or emulsions and can be included in a transdermal patch of the matrix or reservoir type as are conventional in the art for this purpose. For example, DiffusiMAX® (available from Maxima Pharmaceuticals) can be used for transdermal delivery of compounds.

Preferably the compound is administered orally, intravenously, subcutaneously, or transdermally.

Preferably, the pharmaceutical preparation is in a unit dosage form. In such form, the preparation is subdivided into suitably sized unit doses containing appropriate quantities of the active components, e.g., an effective amount to achieve the desired purpose.

Some useful terms are described below:

Capsule—refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet—refers to a compressed or molded solid dosage form containing the active ingredients with suitable diluents. The tablet can be prepared by compression of mixtures or granulations obtained by wet granulation, dry granulation or by compaction.

Oral gel—refers to the active ingredients dispersed or solubilized in a hydrophillic semi-solid matrix.

Powder for constitution refers to powder blends containing the active ingredients and suitable diluents which can be suspended in water or juices.

Diluent—refers to substances that usually make up the major portion of the composition or dosage form. Suitable diluents include sugars such as lactose, sucrose, mannitol and sorbitol; starches derived from wheat, corn, rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

Disintegrant—refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

Binder—refers to substances that bind or “glue” powders together and make them cohesive by forming granules, thus serving as the “adhesive” in the tion. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant—refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d′l-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glident—material that prevents caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

Coloring agents—excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

Bioavailability—refers to the rate and extent to which the active drug ingredient or therapeutic moiety is absorbed into the systemic circulation from an administered dosage form as compared to a standard or control.

Conventional methods for preparing tablets are known. Such methods include dry methods such as direct compression and compression of granulation produced by compaction, or wet methods or other special procedures. Conventional methods for making other forms for administration such as, for example, capsules, suppositories and the like are also well known.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

The term pharmaceutical composition is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a subject by administering a pharmaceutical composition of the present invention is also intended to encompass the administration of the afore-said bulk composition and individual dosage units.

Additionally, the compositions of the present invention may be formulated in sustained release form to provide the rate controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the present invention and/or the pharmaceutically acceptable salts or esters thereof is regulated according to the judgment of the attending clinician considering such factors as age, condition and size of the patient as well as severity of the symptoms being treated.

The compounds of the present invention preferably are administered in an amount effective to reduce the concentration of HCV RNA per milliliter of plasma to a level of less than about 29 IU/mL. The term “concentration of less than 29 International Units of HCV RNA per milliliter of plasma (29 IU/mL)” in the context of the present invention means that there are fewer than 29 IU/ml of HCV RNA, which translates into fewer than 100 copies of HCV-RNA per ml of plasma of the patient as measured by quantitative, multi-cycle reverse transcriptase PCR methodology. HCV-RNA is preferably measured in the present invention by research-based RT-PCR methodology well known to the skilled clinician. This methodology is referred to herein as HCV-RNA/qPCR. The lower limit of detection of HCV-RNA is 29 IU/ml or 100 copies/ml. Serum HCV-RNA/qPCR testing and HCV genotype testing is performed by a central laboratory. See also J. G. McHutchinson et al. (N. Engl. J. Med., 1998, 339:1485-1492), and G. L. Davis et al. (N. Engl. J. Med. 339:1493-1499).

The Following Experimental Section Applies for the Preparation of the Compounds of Formula XII:

Abbreviations which are used in the descriptions of the schemes, preparations and the examples that follow are:

THF: Tetrahydrofuran

DMF: N,N-Dimethylformamide

EtOAc: Ethyl acetate

AcOH: Acetic acid

HOOBt: 3-Hydroxy-1,2,3-benzotriazin-4(3H)-one

EDCl: 1-(3-dimethylaminopropyl)-3-ethylcarbodiimide hydrochloride

NMM: N-Methylmorpholine

ADDP: 1,1′-(Azodicarbobyl)dipiperidine

DEAD: Diethylazodicarboxylate

MeOH: Methanol

EtOH: Ethanol

Et₂O: Diethyl ether

DMSO: Dimethylsulfoxide

HOBt: N-Hydroxybenzotriazole

PyBrOP: Bromo-tris-pyrrolidinophosphonium hexafluorophosphate

DCM: Dichloromethane

DCC: 1,3-Dicyclohexylcarbodiimide

TEMPO: 2,2,6,6-Tetramethyl-1-piperidinyloxy

Phg: Phenylglycine

Chg: Cyclohexylglycine

Bn: Benzyl

Bzl: Benzyl

Et: Ethyl

Ph: Phenyl

iBoc: isobutoxycarbonyl

iPr: isopropyl

^{t}Bu or Bu^{t}: tert-Butyl

Boc: tert-Butyloxycarbonyl

Cbz: Benzyloxycarbonyl

Cp: Cylcopentyldienyl

Ts: p-toluenesulfonyl

Me: Methyl

HATU: O-(7-azabenzotriazol-1-yl)-1,1,3,3-tetramethyluronium hexafluorophosphate

DMAP: 4-N,N-Dimethylaminopyridine

BOP: Benzotriazol-1-yl-oxy-tris(dimethylamino)hexafluorophosphate

PCC: Pyridiniumchlorochromate

General Schemes for Preparation of Target Compounds

Compounds of the present invention were synthesized using the general schemes (Methods A-E) described below.

Method A:

Deprotection of the N-Boc functionality of 1.01 under acidic conditions provided the hydrochloride salt 1.02 which was subsequently coupled with N-Boc-tert-leucine under peptide coupling methodology to afford 1.03. N-Boc deprotection followed by treatment with appropriate isocyanate gave the urea 1.05. Hydrolysis of the methyl ester provided the acid 1.06. Peptide coupling of the acid 1.06 with the appropriate P₁-P′ primary amide moiety afforded the hydroxylamide 1.07. Oxidation (Moffatt or related process—T. T. Tidwell, Synthesis, 1990, 857; or Dess-Martin's—J. Org. Chem., 1983, 48, 4155) resulted in the target compound 1.08. 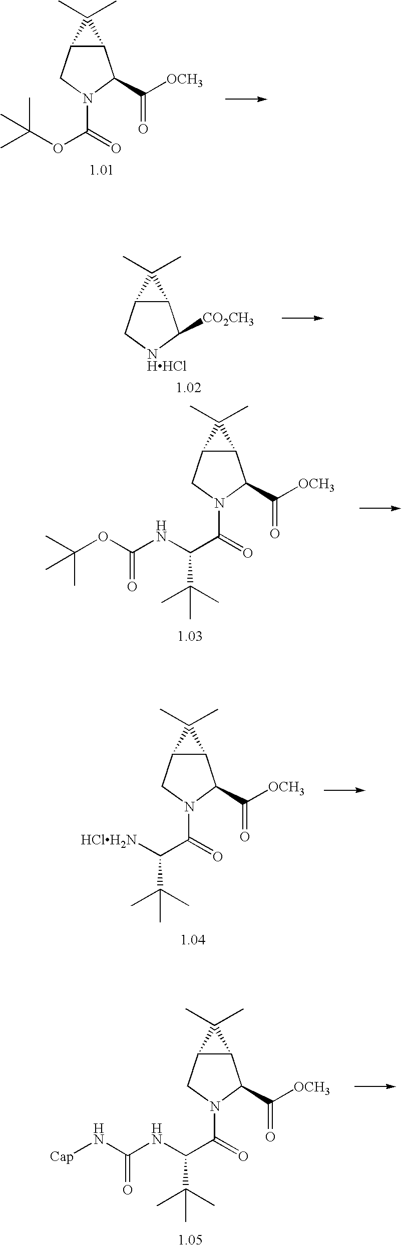 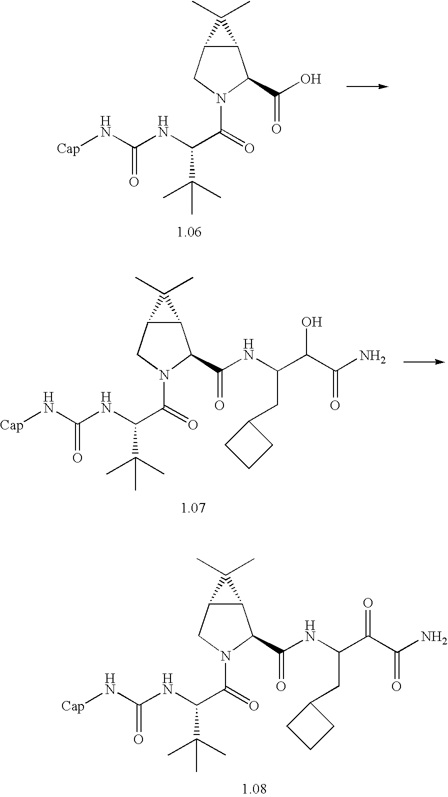
Method B
Peptide coupling of the acid 1.06 with the appropriate P₁-P′ secondary amide moiety afforded the hydroxylamide 1.09. Oxidation (Moffatt or Dess-Martin's) resulted in the target compound 1.10. 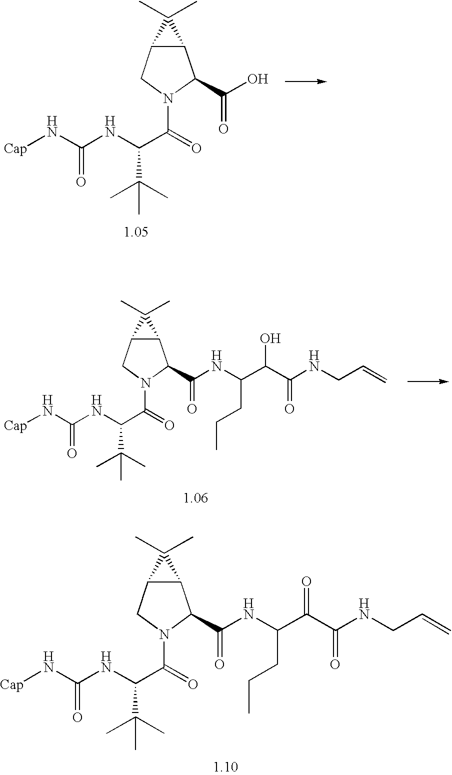
Method C
In another variation, peptide coupling of the N-Boc-P₂-P₃-acid 1.17 with the appropriate P₁-P′ amide moiety afforded the hydroxylamide 1.11. Oxidation (Moffatt or Dess-Martin's) resulted in the keto amide 1.12. Deprotection of the N-Boc functionality gave the hydrochloride salt 1.13. Treatment with a suitable isocyanate (or isocyanate equivalent) resulted in the target compound 1.14. 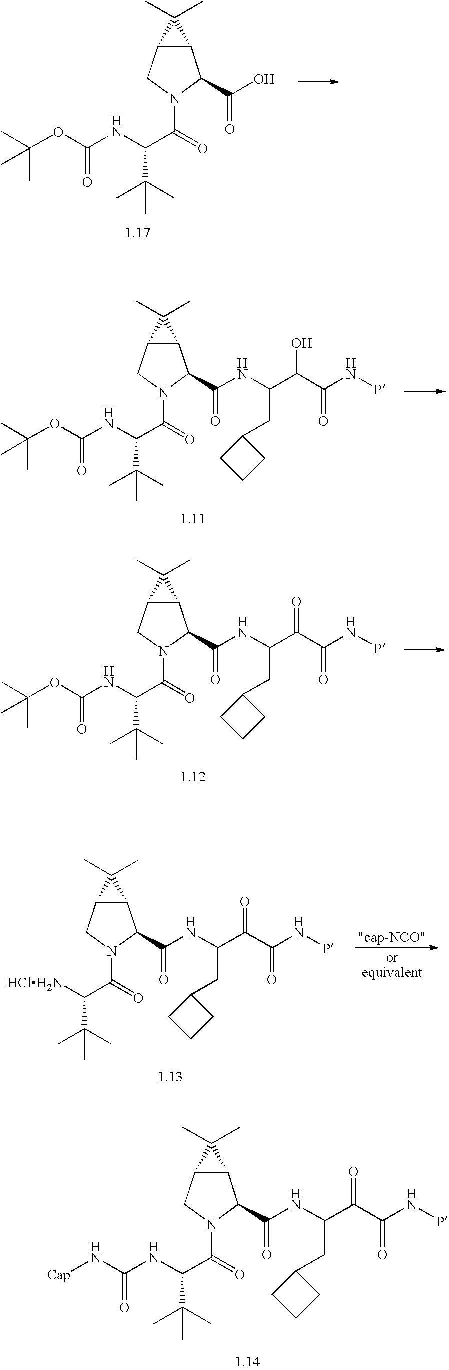
Method D
In yet another variation, the hydrochloride salt 1.13 was converted to the 4-nitrophenyl carbamate 1.15 by reaction with 4-nitrophenyl chloroformate. Subsequent treatment with an amine (or amine hydrochloride salt) of choice provided the target compound 1.14. 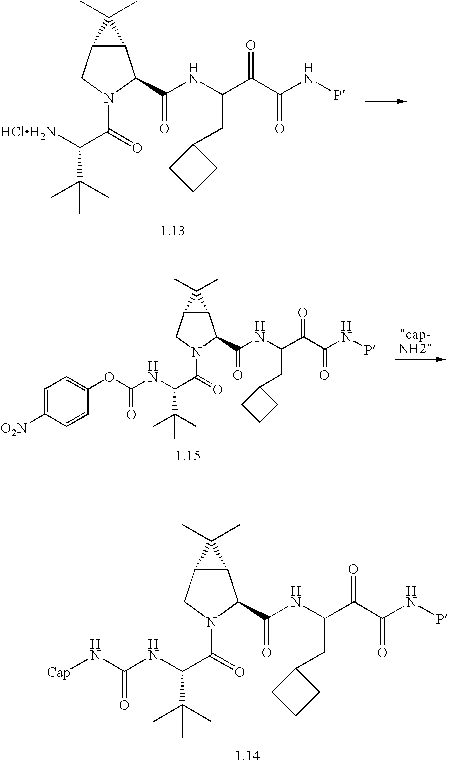
Method E
In yet another variation, the dipeptide hydrochloride salt 1.03 was converted to the 4-nitrophenyl carbamate as described above. Treatment with an amine (or amine hydrochloride salt) of choice provided the urea derivative 1.05. Hydrolysis and further elaboration as described in Methods A/B provided the target compounds 1.14.

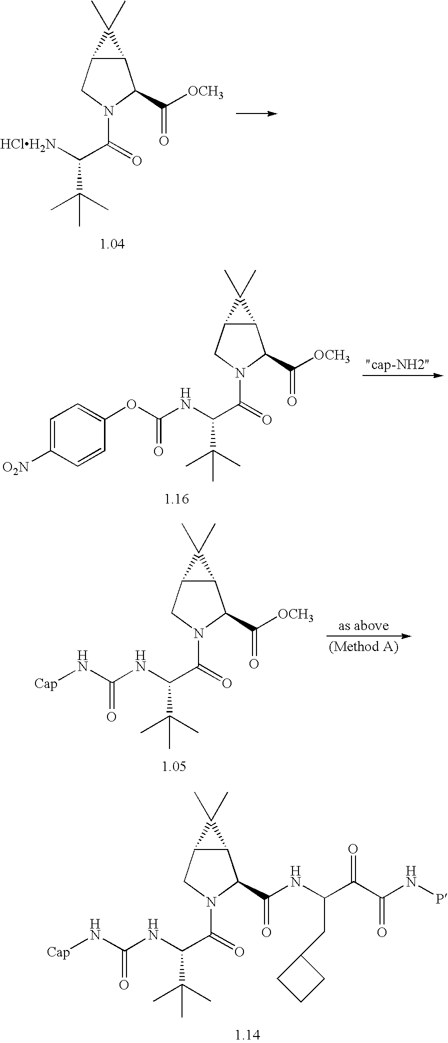

The following experimental section applies for the preparation of the compounds of Formula XIII: Abbreviations which are used in the descriptions of the schemes, preparations and the examples that follow are:

THF: Tetrahydrofuran

DMF: N,N-Dimethylformamide

EtOAc: Ethyl acetate

AcOH: Acetic acid

HOOBt: 3-Hydroxy-1,2,3-benzotriazin-4(3H)-one

EDCl: 1-(3-dimethylaminopropyl)-3-ethylcarbodiimide hydrochloride

NMM: N-Methylmorpholine

ADDP: 1,1′-(Azodicarbobyl)dipiperidine

DEAD: Diethylazodicarboxylate

DIAD: Diisopropylazodicarboxylate

MeOH: Methanol

EtOH: Ethanol

Et₂O: Diethyl ether

DMSO: Dimethylsulfoxide

HOBt: N-Hydroxybenzotriazole

PyBrOP: Bromo-tris-pyrrolidinophosphonium hexafluorophosphate

DCM: Dichloromethane

DCC: 1,3-Dicyclohexylcarbodiimide

TEMPO: 2,2,6,6-Tetramethyl-1-piperidinyloxy

Phg: Phenylglycine

Chg: Cyclohexylglycine

Bn: Benzyl

Bz: Benzyl

Et: Ethyl

Ph: Phenyl

iBoc: isobutoxycarbonyl

iPr: isopropyl

^{t}Bu or Bu^{t}: tert-Butyl

Boc: tert-Butyloxycarbonyl

Cbz: Benzyloxycarbonyl

Cp: Cylcopentyldienyl

Ts: p-toluenesulfonyl

Me: Methyl

Ms or Mesyl: Methane sulfonyl

HATU: O-(7-azabenzotriazol-1-yl)-1,1,3,3-tetramethyluronium hexafluorophosphate

DMAP: 4-N,N-Dimethylaminopyridine

Bop: Benzotriazol-1-yl-oxy-tris(dimethylamino)hexafluorophosphate

PCC: Pyridiniumchlorochromate

DIBAL-H: diisopropyl aluminum hydride

rt or RT: Room temperature

quant.: Quantitative yield

h or hr: hour

min: minute

TFA: Trifluoroacetic acid

General Schemes for Preparation of Target Compounds

Compounds of the present invention were synthesized using the general schemes (Methods A-E) described below.

Method A

Deprotection of the N-Boc functionality of 1.01 under acidic conditions provided the hydrochloride salt 1.02 which was subsequently coupled with N-Boc-tert-leucine under peptide coupling methodology to afford 1.03. N-Boc deprotection followed by treatment with appropriate isocyanate gave the urea 1.05. Hydrolysis of the methyl ester provided the acid 1.06. Peptide coupling of the acid 1.06 with the appropriate P₁-P′ primary amide moiety afforded the hydroxylamide 1.07. Oxidation (Moffatt or related process—T. T. Tidwell, Synthesis, 1990, 857; or Dess-Martin's periodinane (J. Org. Chem., 1983, 48, 4155) resulted in the target compound 1.08. 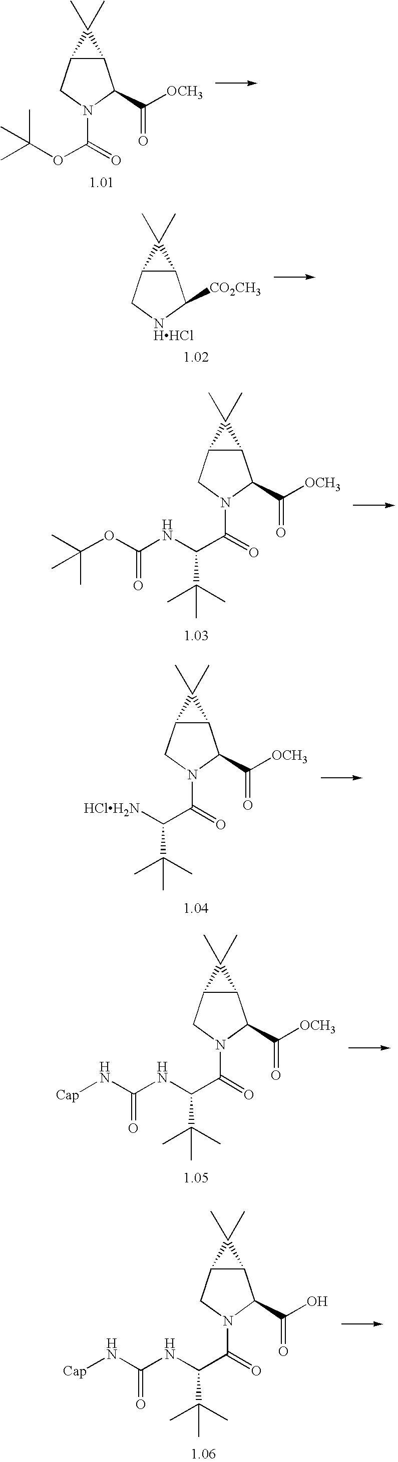 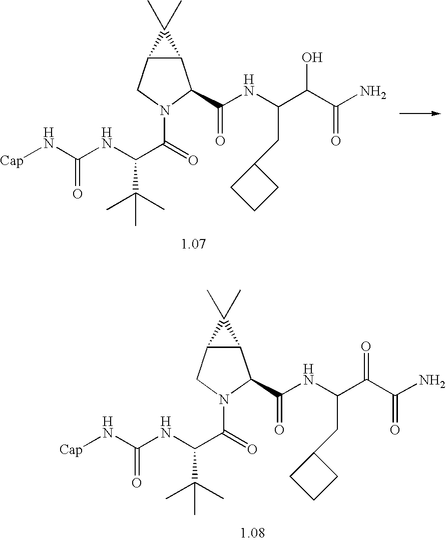
Method B
Peptide coupling of the acid 1.06 with the appropriate P₁-P′ secondary amide moiety afforded the hydroxylamide 1.09. Oxidation (Moffatt or Dess-Martin's) resulted in the target compound 1.10. 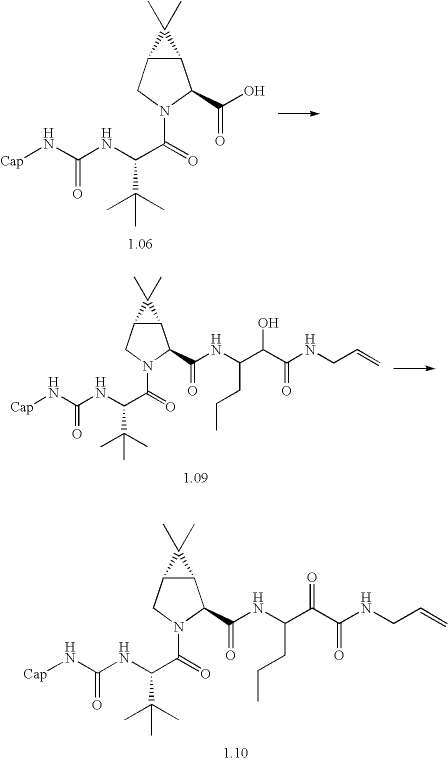
Method C

In another variation, peptide coupling of the N-Boc-P₂—P₃-acid 1.17 with the appropriate P₁-P′ amide moiety afforded the hydroxylamide 1.11. Oxidation (Moffatt or Dess-Martin's) resulted in the keto amide 1.12. Deprotection of the N-Boc functionality gave the hydrochloride salt 1.13. Treatment with a suitable isocyanate (or isocyanate equivalent) resulted in the target compound 1.14. 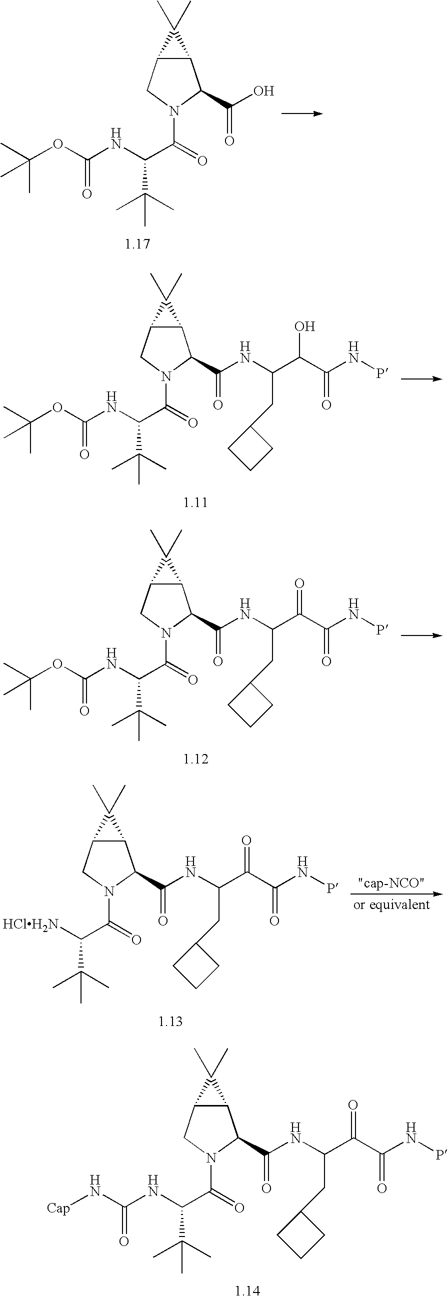
Method D

In yet another variation, the hydrochloride salt 1.13 was converted to the 4-nitrophenyl carbamate 1.15 by reaction with 4-nitrophenyl chloroformate. Subsequent treatment with an amine (or amine hydrochloride salt) of choice provided the target compound 1.14. 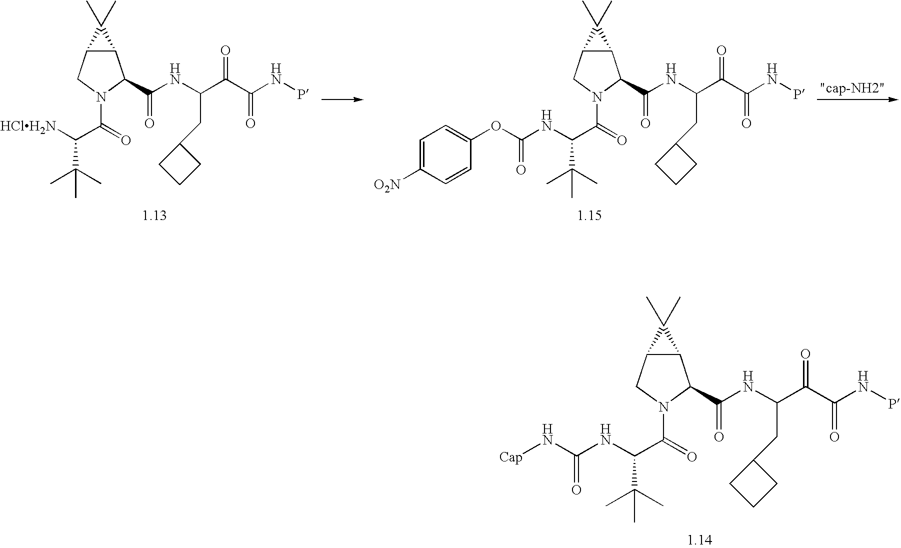
Method E
In yet another variation, the dipeptide hydrochloride salt 1.03 was converted to the 4-nitrophenyl carbamate as described above. Treatment with an amine (or amine hydrochloride salt) of choice provided the urea derivative 1.05. Hydrolysis and further elaboration as described in Methods A/B provided the target compounds 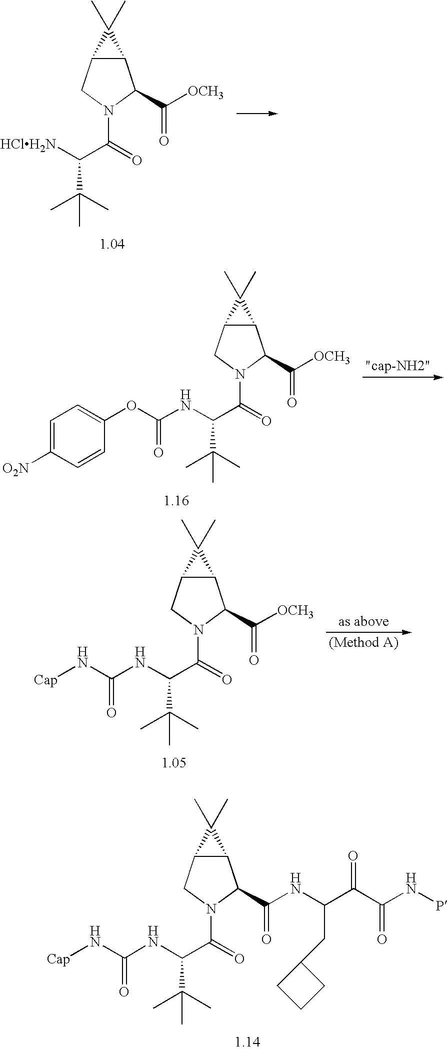
The Following Experimental Section Applies for the Preparation of the Compounds of Formula XIV:

For the procedures described below, the following abbreviations are used:

THF: Tetrahydrofuran

DMF: N,N-Dimethylformamide

EtOAc: Ethyl acetate

AcOH: Acetic acid

HOOBt: 3-Hydroxy-1,2,3-benzotriazin-4(3H)-one

EDCl: 1-(3-dimethylaminopropyl)-3-ethylcarbodiimide hydrochloride

NMM: N-Methylmorpholine

ADDP: 1,1′-(Azodicarbobyl)dipiperidine

DEAD: Diethylazodicarboxylate

MeOH: Methanol

EtOH: Ethanol

Et2O: Diethyl ether

DMSO: Dimethylsulfoxide

HOBt: N-Hydroxybenzotriazole

PyBrOP: Bromo-tris-pyrrolidinophosphonium hexafluorophosphate

DCM: Dichloromethane

DCC: 1,3-Dicyclohexylcarbodiimide

TEMPO: 2,2,6,6-Tetramethyl-1-piperidinyloxy

Phg: Phenylglycine

Chg: Cyclohexylglycine

Bn: Benzyl

Bzl: Benzyl

Et: Ethyl

Ph: Phenyl

DMF-DMA: N,N-Dimethylformamide-dimethylacetal

iBoc: isobutoxycarbonyl

iPr: isopropyl

^{t}Bu or Bu^{t}: tert-Butyl

Boc: tert-Butyloxycarbonyl

Cbz: Benzyloxycarbonyl

Cp: Cylcopentyldienyl

Ts: p-toluenesulfonyl

Me: Methyl

HATU: O-(7-azabenzotriazol-1-yl)-1,1,3,3-tetramethyluronium hexafluorophosphate

DMAP: 4-N,N-Dimethylaminopyridine

BOP: Benzotriazol-1-yl-oxy-tris(dimethylamino)hexafluorophosphate

PCC: Pyridiniumchlorochromate

KHMDS: Potassium Hexamethyldisilazide or Potassium bis(trimethylsilylamide)

NaHMDS: Sodium Hexamethyldisilazide or Sodium bis(trimethylsilylamide)

LiHMDS: Lithium Hexamethyldisilazide or Lithium bis(trimethylsilylamide)

10% Pd/C: 10% Palladium on carbon (by weight).

TG: Thioglycerol

General Schemes for Preparation of Target Compounds

Compounds of the present invention were synthesized using the general schemes (Methods A-E) described below.

Method A

Deprotection of the N-Boc functionality of 1.01 under acidic conditions provided the hydrochloride salt 1.02 which was subsequently coupled with N-Boc-tert-leucine under peptide coupling methodology to afford 1.03. N-Boc deprotection followed by treatment with appropriate isocyanate gave the urea 1.05. Hydrolysis of the methyl ester provided the acid 1.06. Peptide coupling of the acid 1.06 with the appropriate P₁-P′ primary amide moiety afforded the hydroxylamide 1.07. Oxidation (Moffatt oxidation or related process—see, T. T. Tidwell, Synthesis, 1990, 857), or Dess-Martin Periodinane—J. Org. Chem., (1983) 48, 4155) resulted in the target compound 1.08. 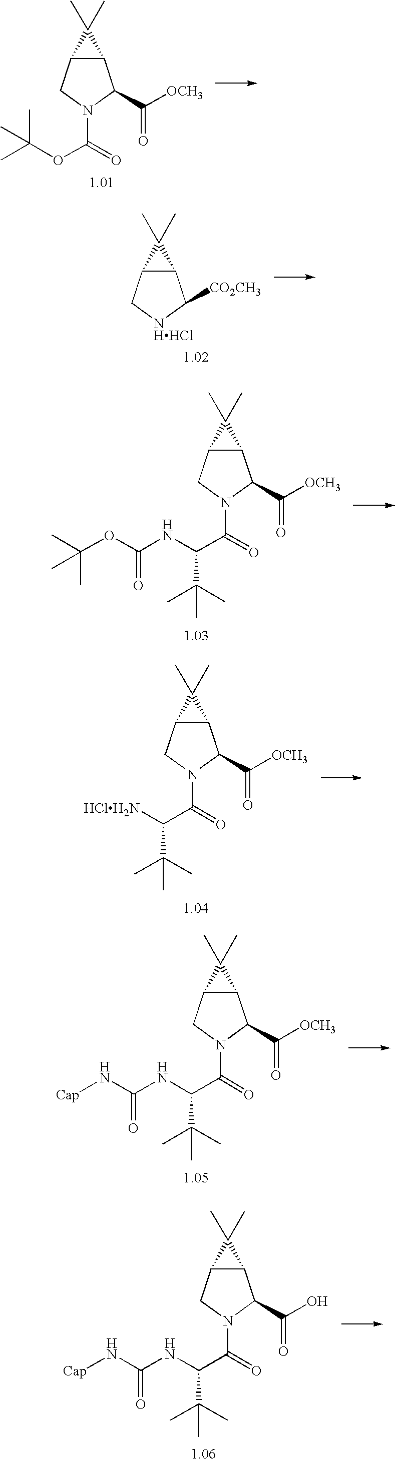 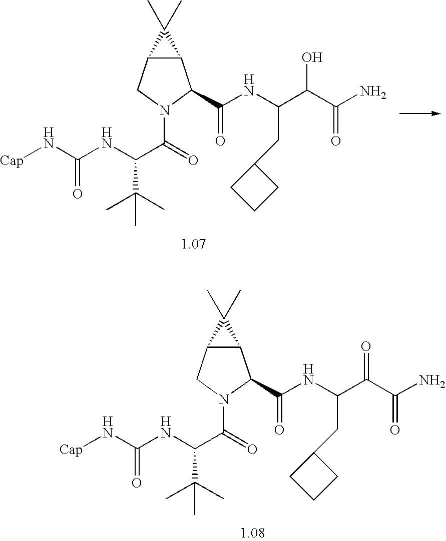
Method B

Peptide coupling of the acid 1.06 with the appropriate P₁-P′ secondary amide moiety afforded the hydroxylamide 1.09. Oxidation (Moffatt or Dess-Martin's) resulted in the target compound 1.10 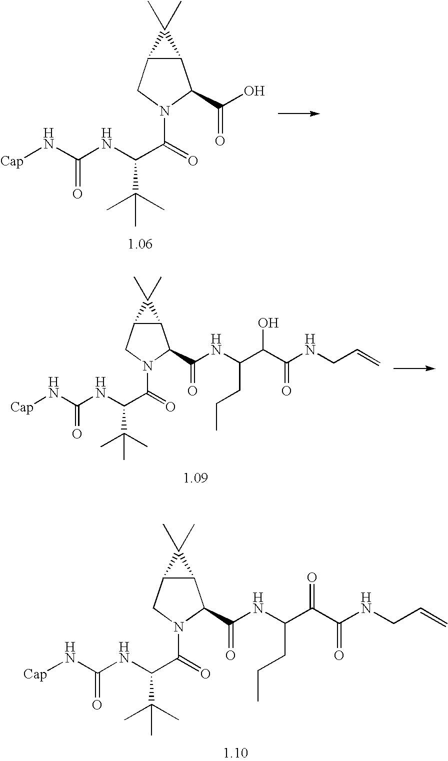
Method C

In another variation, peptide coupling of the N-Boc-P2-P₃-acid 1.17 with the appropriate P₁-P′ amide moiety afforded the hydroxylamide 1.11. Oxidation (Moffatt or Dess-Martin Periodinane) resulted in the keto amide 1.12. Deprotection of the N-Boc functionality gave the hydrochloride salt 1.13. Treatment with a suitable isocyanate (or isocyanate equivalent) resulted in the target compound 1.14. 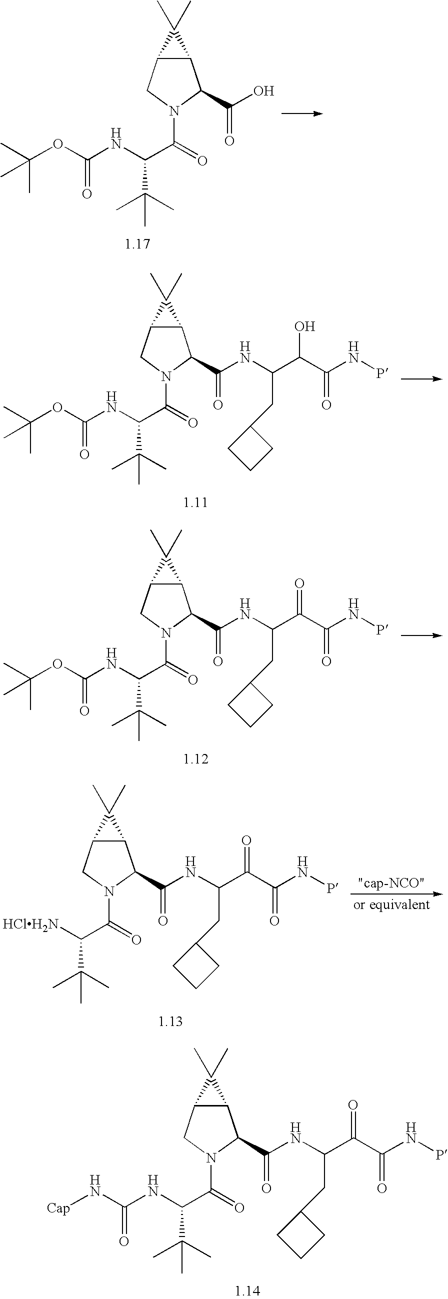
Method D

In yet another variation, the hydrochloride salt 1.13 was converted to the 4-nitrophenyl carbamate 1.15 by reaction with 4-nitrophenyl chloroformate. Subsequent treatment with an amine (or amine hydrochloride salt) of choice provided the target compound 1.14. 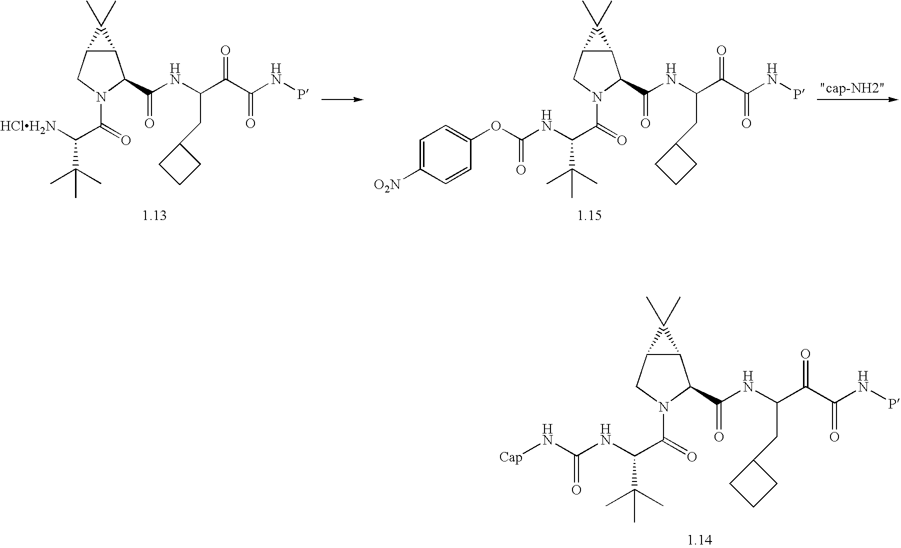
Method E

In yet another variation, the dipeptide hydrochloride salt 1.03 was converted to the 4-nitrophenyl carbamate as described above. Treatment with an amine (or amine hydrochloride salt) of choice provided the urea derivative 1.05. Hydrolysis and further elaboration as described in Methods A/B provided the target compounds 1.14. 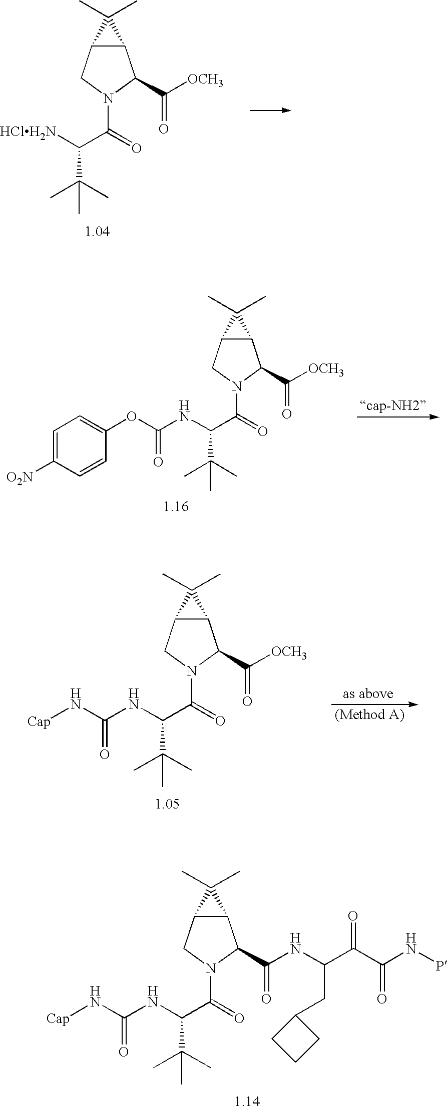
The Following Experimental Section Applies for the Preparation of the Compounds of Formula XV:

For the procedures described below, the following abbreviations are used:

THF: Tetrahydrofuran

DMF: N,N-Dimethylformamide

EtOAc: Ethyl acetate

AcOH: Acetic acid

HOOBt: 3-Hydroxy-1,2,3-benzotriazin-4(3H)-one

EDCl: 1-(3-dimethylaminopropyl)-3-ethylcarbodiimide hydrochloride

NMM: N-Methylmorpholine

ADDP: 1,1′-(Azodicarbobyl)dipiperidine

DEAD: Diethylazodicarboxylate

MeOH: Methanol

EtOH: Ethanol

Et2O: Diethyl ether

DMSO: Dimethylsulfoxide

HOBt: N-Hydroxybenzotriazole

PyBrOP: Bromo-tris-pyrrolidinophosphonium hexafluorophosphate

DCM: Dichloromethane

DCC: 1,3-Dicyclohexylcarbodiimide

TEMPO: 2,2,6,6-Tetramethyl-1-piperidinyloxy

Phg: Phenylglycine

Chg: Cyclohexylglycine

Bn: Benzyl

Bzl: Benzyl

Et: Ethyl

Ph: Phenyl

iBoc: isobutoxycarbonyl

iPr: isopropyl

^{t}Bu or Bu^{t}: tert-Butyl

Boc: tert-Butyloxycarbonyl

Cbz: Benzyloxycarbonyl

Cp: Cylcopentyldienyl

Ts: p-toluenesulfonyl

Me: Methyl

HATU: O-(7-azabenzotriazol-1-yl)-1,1,3,3-tetramethyluronium hexafluorophosphate

DMAP: 4-N,N-Dimethylaminopyridine

BOP: Benzotriazol-1-yl-oxy-tris(dimethylamino)hexafluorophosphate

PCC: Pyridiniumchlorochromate

KHMDS: Potassium Hexamethyldisilazide or Potassium bis(trimethylsilylamide)

NaHMDS: Sodium Hexamethyldisilazide or Sodium bis(trimethylsilylamide)

LiHMDS: Lithium Hexamethyldisilazide or Lithium bis(trimethylsilylamide) 10% Pd/C: 10% Palladium on carbon (by weight).

### PREPARATIVE EXAMPLE 1

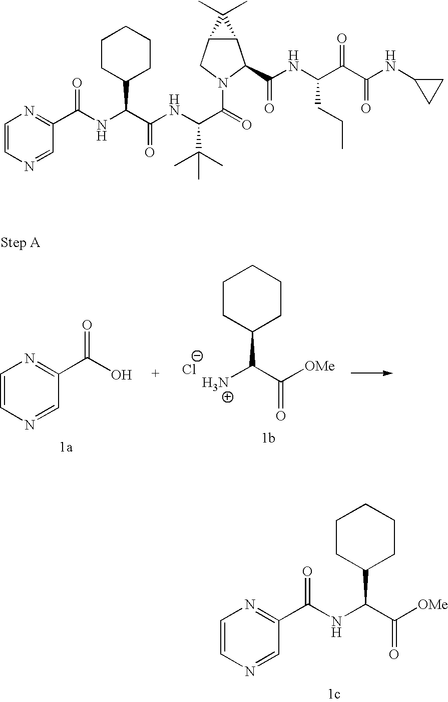

A solution of pyrazinecarboxylic acid 1a (3 g) in 150 mL of dry dichloromethane and 150 mL of dry DMF was stirred at 0° C. and treated with HATU (1.4 eq, 6.03 g). L-cyclohexylglycine hydrochloride lb (1.2 eq, 6.03 g) was added in small portions. Then, N-methylmorpholine (4 eq, 10 mL, d 0.920) was added dropwise. The reaction mixture was gradually warmed to room temperature and stirred for 20 h. All the volatiles were removed under vacuum and the residue was dissolved in 500 mL of ethyl acetate. The organic layer washed with water (100 mL), aqueous 1N HCl (100 mL), aqueous saturated sodium bicarbonate solution (100 mL), and brine (100 mL). The organic layer was dried over magnesium sulfate, filtered and concentrated under reduced pressure. The residue was chromatographed on silica gel (gradient: acetone/hexanes; 5:95 to 3:7) to afford the product 1c as a white solid.

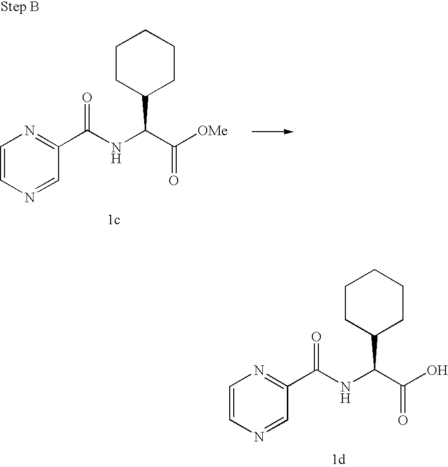

A solution of methyl ester 1c (6.5 g) in 270 mL of a 1:1:1 mixture of THF/MeOH/water was cooled to 0° C. and treated with lithium hydroxide monohydrate (2.5 eq, 2.45 g). The mixture was stirred and monitored by TLC (acetone/hexanes; 2:8). When all the starting material had been consumed, the reaction mixture was treated with 100 mL of aqueous 1N HCl and the mixture was concentrated on the rotavap. Dichloromethane (250 mL) was added and layers separated. The aqueous layer was extracted with dichloromethane (3×80 mL). The combined organic layers were dried over magnesium sulfate, filtered, and concentrated to afford the product 1d as a white solid.

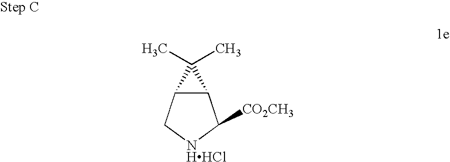

The amino ester 1e was prepared following the method of R. Zhang and J. S. Madalengoitia (J. Org. Chem. 1999, 64, 330), with the exception that the Boc group was cleaved by the reaction of the Boc-protected amino acid with methanolic HCl (4M HCl in dioxane was also employed for the deprotection).
(Note: In a variation of the reported synthesis, the sulfonium ylide was replaced with the corresponding phosphonium ylide).

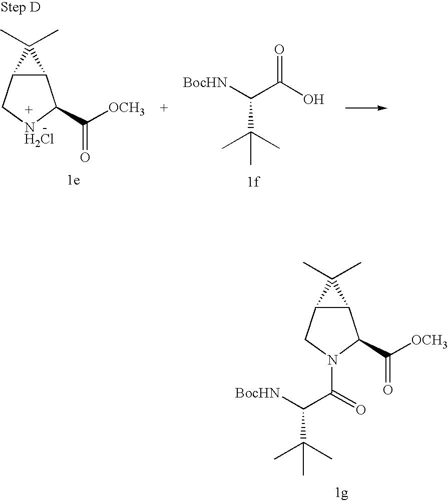

A solution of Boc-tert-Leu 1f (Fluka, 5.0 g, 21.6 mmol) in dry CH₂Cl₂/DMF (50 mL, 1:1) was cooled to 0° C. and treated with the amine hydrochloride 1e (5.3 g, 25.7 mmol), NMM (6.5 g, 64.8 mmol) and BOP reagent (11.6 g, 25.7 mmol). The reaction was stirred at rt. for 24 h, diluted with aqueous HCl (1 M) and extracted with CH₂Cl₂. The combined organic layers were washed with aqueous 1 M HCl, saturated NaHCO₃, brine, dried (MgSO₄), filtered and concentrated in vacuo and purified by chromatography (SiO₂, Acetone/Hexane 1:5) to yield 1g as a colorless solid.

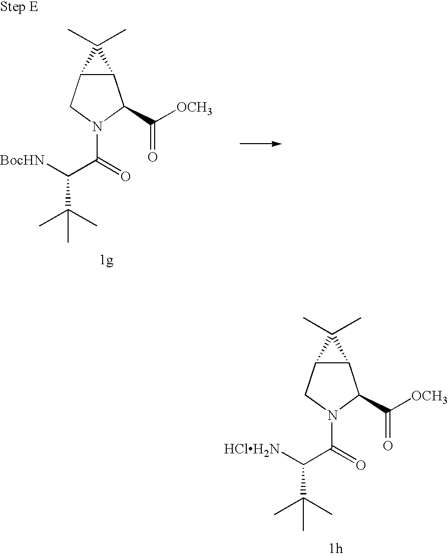

A solution of methyl ester 1g (4.0 g, 10.46 mmol) was dissolved in 4M HCl in dioxane and stirred at rt. for 3 h. The reaction mixture was concentrated in vacuo to obtain the amine hydrochloride salt, 1 h which was used without purification.

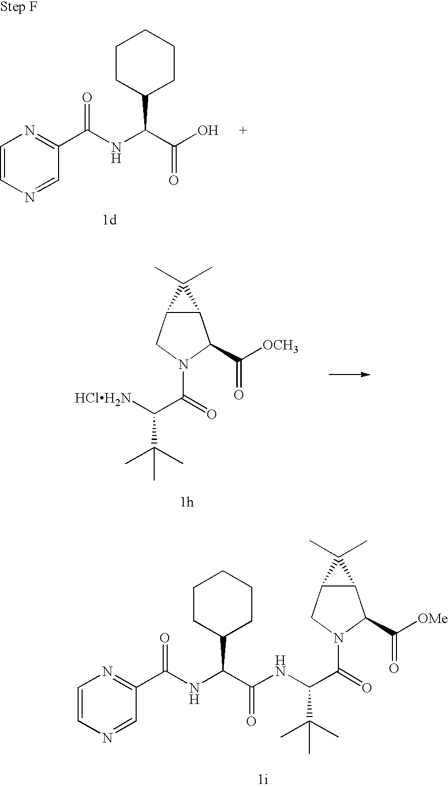

A solution of acid 1d (100 mg) in 5 mL of dry dichloromethane and 5 mL of dry DMF was stirred at 0° C. and treated with HATU (1.4 eq, 202 mg). The amine hydrochloride 1 h (1.2 eq, 146 mg) was added. Then, N-methylmorpholine (4 eq, 0.17 mL, d 0.920) was also added. The reaction mixture was stirred at 0° C. overnight. All the volatiles were removed under vacuum and the residue was dissolved in 80 mL of ethyl acetate. The organic layer washed with water (10 mL), aqueous 1N HCl (10 mL), aqueous saturated sodium bicarbonate solution (10 mL), and brine (10 mL). The organic layer was dried over magnesium sulfate, filtered and concentrated under reduced pressure. The residue was chromatographed on silica gel (gradient: acetone/hexanes; 1:9 to 4:6) to afford the product 1i as a white solid.

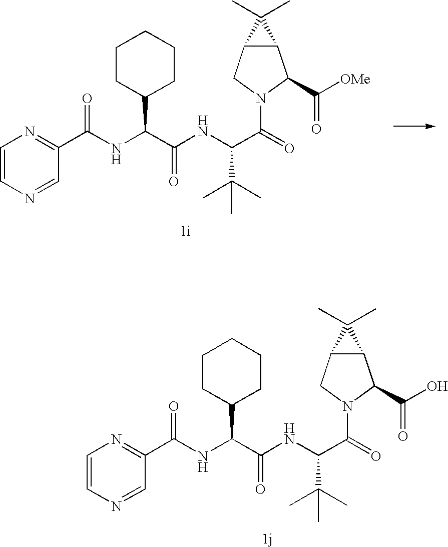

A solution of methyl ester 11 (180 mg) in 9 mL of a 1:1:1 mixture of THF/MeOH/water was cooled to 0° C. and treated with lithium hydroxide monohydrate (2.5 eq, 35 mg). The mixture was stirred and monitored by TLC (acetone/hexanes; 3:7). When all the starting material had been consumed, the reaction mixture was treated with 50 mL of aqueous 1N HCl and the mixture was concentrated on the rotavap. Dichloromethane (80 mL) was added and layers separated. The aqueous layer was extracted with dichloromethane (3×50 mL). The combined organic layers were dried over magnesium sulfate, filtered, and concentrated to afford the product 1j as a white solid.

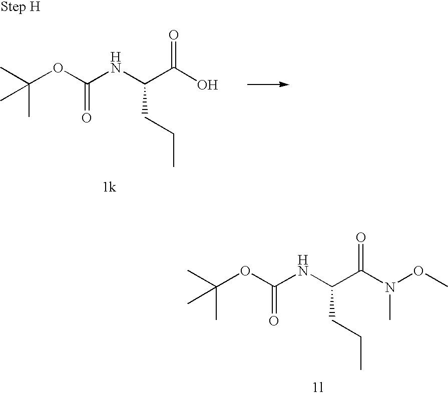

A solution of acid 1k (2 g) in 100 mL of dry dichloromethane and 5 mL of DMF was treated with N,O-dimethylhydroxylamine hydrochloride (1.1 eq, 986 mg), BOP reagent (1.1 eq, 4.47 g), and N-methylmorpholine (3.3 eq, 3.3 mL, d 0.920) in that order. The mixture was heated to 50° C. overnight. The reaction mixture was concentrated to half its volume and diluted with 400 mL of ethyl acetate. The organic layer washed with water (80 mL), aqueous 1M HCl (80 mL), aqueous saturated sodium bicarbonate solution (80 mL), and brine (80 mL). The organic layer was dried over magnesium sulfate, filtered, and concentrated under reduced pressure. The residue was chromatographed on silica gel (gradient: acetone/hexanes; 5:95 to 3:7) to afford the product II as a clear oil.

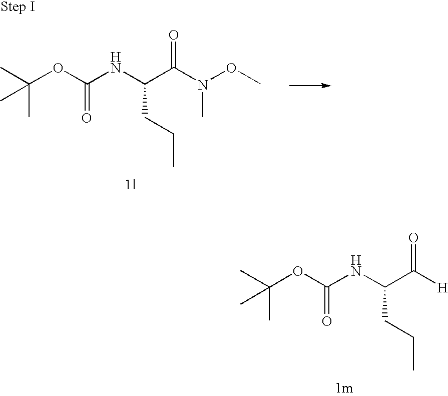

A solution of amide 1I (2.2 g) in 100 mL of dry THF was cooled to ° C. Lithium aluminum hydride solution (1.3 eq) was added dropwise. The cooling bath was removed after 5 min and the mixture was allowed to reach room temperature. TLC analysis (ethyl acetate/hexanes; 2:8) showed that all the starting material had been consumed. The excess LAH was carefully quenched by addition of drops of aqueous saturated sodium hydrogen sulfate. The mixture was diluted with 200 mL of ether and aqueous saturated sodium hydrogen sulfate was added in small portions until a white solid precipitated. The mixture was filtered thru celite and the filtrate washed with 50 mL of brine. The organic layer was dried over magnesium sulfate, filtered and concentrated. The residue was chromatographed on silica gel (gradient: ethyl acetate/hexanes; 5:95 to 4:6) to afford the aldehyde product 1m as a colorless oil.
Step J

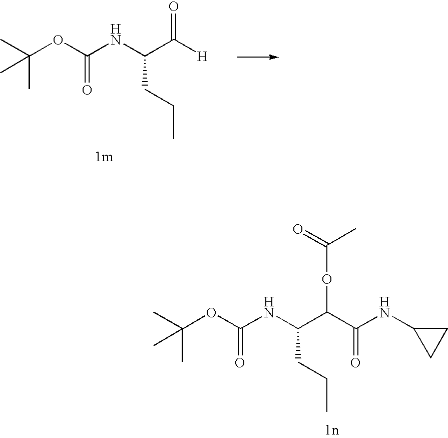

A solution of aldehyde 1m (1.8 g) in 100 mL of dry dichloromethane was treated with isonitrile (1.1 eq, 680 mg) and acetic acid (2 eq, 1.02 mL, d 1.0149). The mixture was stirred overnight. All the volatiles were removed under vacuum and the residue was chromatographed on silica gel (gradient: ethyl acetate/hexanes; 2:8 to 6:4) to afford the product 1 n as a white solid.
Step K

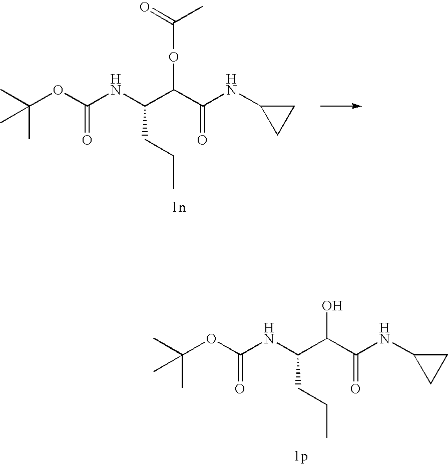

A solution of acetate 1n (1.6 g) in 60 mL of a 1:1:1 mixture of THF/MeOH/water was treated with lithium hydroxide monohydrate and stirred for approximately 1 h until all the starting material had been consumed as determined by TLC analysis (ethyl acetate/hexanes; 1:1). The volatiles were removed in rotavap and the residue was diluted with dichloromethane (150 mL). The layers were separated and the aqueous layer was diluted with 30 mL of aqueous saturated sodium bicarbonate solution and extracted with dichloromethane (3×80 mL). The combined organic layers were dried over magnesium sulfate, filtered and concentrated to afford the product 1p as a white solid.
Step L

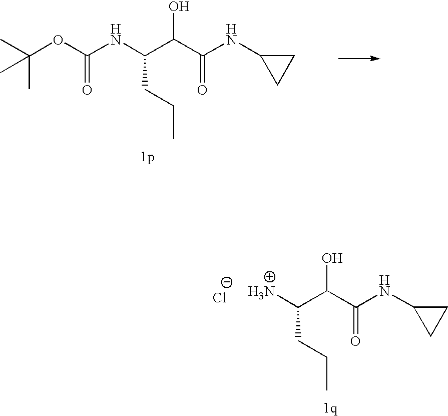

The N-Boc protected amine 1p (1.5 g) was dissolved in 20 mL of 4M HCl in dioxane. The reaction mixture was stirred for about 1 h until all the starting material had been consumed. All the volatiles were removed under vacuum to afford the product 1q as a white solid.
Step M

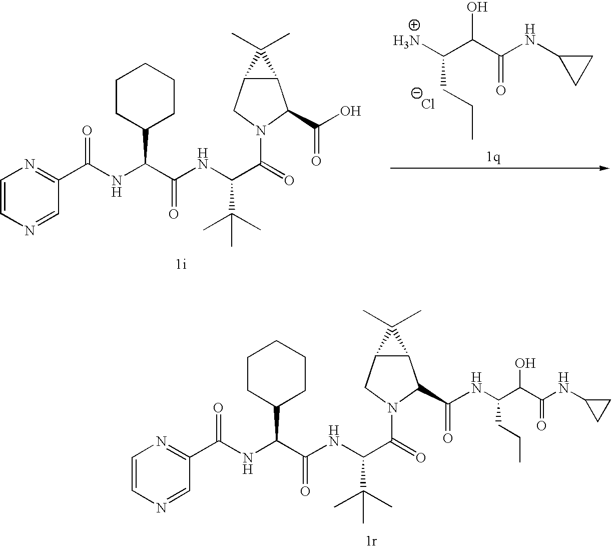

A solution of acid 1j (50 mg) in 2 mL of dry dichloromethane and 2 mL of dry DMF was stirred at 0° C. and treated with HATU (1.4 eq, 52 mg). The amine hydrochloride 1q (1.2 eq, 26 mg) was added. Then, N-methylmorpholine (4 eq, 0.042 mL, d 0.920) was also added. The reaction mixture was stirred at 0° C. overnight. All the volatiles were removed under vacuum and the residue was dissolved in 80 mL of ethyl acetate. The organic layer washed with water (10 mL), aqueous 1N HCl (10 mL), aqueous saturated sodium bicarbonate solution (10 mL), and brine (10 mL). The organic layer was dried over magnesium sulfate, filtered and concentrated under reduced pressure. The product Ir was used without further purification.
Step N

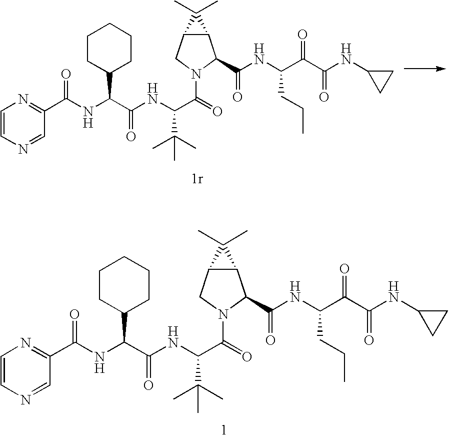

A solution of alcohol 1r (65 mg) in 5 mL of dry dichloromethane was treated with Dess-Martin periodinane (3 eq, 121 mg). Reaction mixture was stirred at room temperature for 45 min. The mixture was treated with aqueous 1M sodium thiosulfate solution (10 mL) and aqueous saturated sodium bicarbonate solution (10 mL) and stirred for 15 min. The mixture was extracted with dichloromethane (3×20 mL). The combined organic layers were dried over magnesium sulfate, filtered, and concentrated. The residue was chromatographed on silica gel (gradient: acetone/hexanes; 2:8 to 5:5) to afford the product I as a white solid.

One skilled in the art would understand that other suitable compounds of Formula XV can be prepared in a similar manner to that disclosed above.

The Following Experimental Section Applies for the Preparation of the Compounds of Formula XVI:

### PREPARATIVE EXAMPLE A

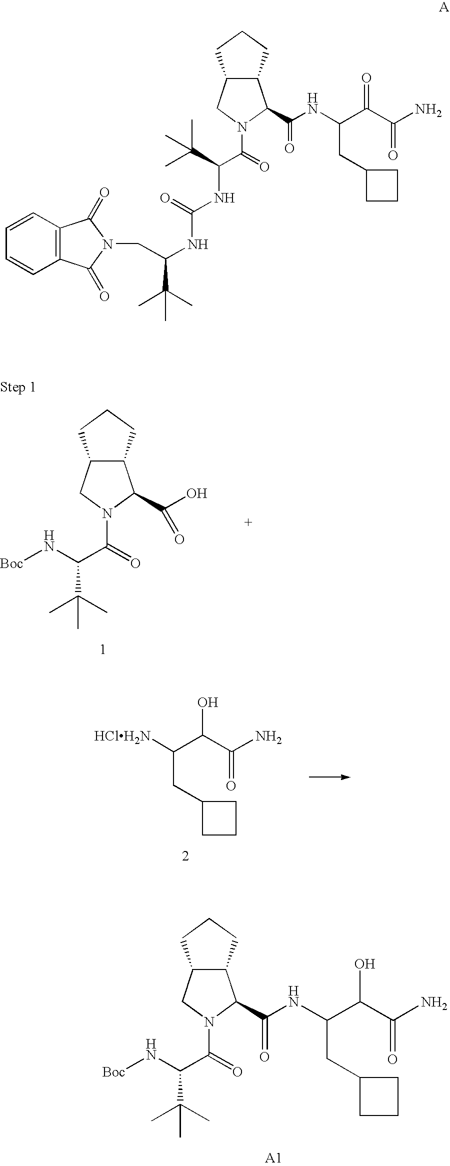

A solution of acid 1 (255 mg) in 5 mL of dry dichloromethane and 5 mL of dry DMF was stirred at 0° C. and treated with HATU (368 mg). The amine hydrochloride 2 (201 mg) was added followed by addition of N-methylmorpholine (0.42 mL). The reaction mixture was gradually warmed to room temperature and stirred overnight.

All the volatiles were removed under vacuum and the residue was taken into 100 mL of ethyl acetate. The organic layer washed with aqueous 1 N HCl (15 mL), aqueous saturated NaHCO3 (15 mL), water (15 mL), brine (15 mL), dried over MgSO4, filtered, and concentrated under reduced pressure to afford the desired product A1. No further purification was carried out for the product.
Step 2

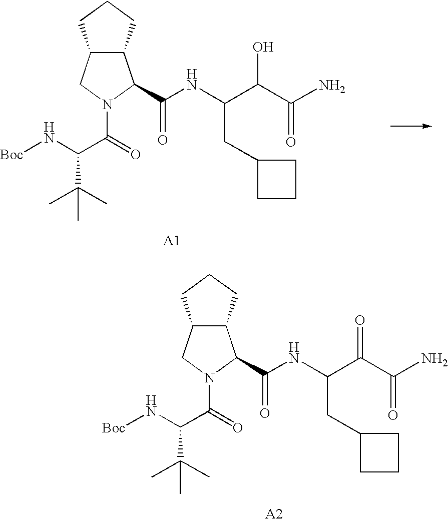

A solution of A1 (360 mg) in 20 mL of a 1:1 mixture of toluene/DMSO was treated with EDCl (1.3 g) and dichloroacetic acid (0.42 mL, d 1.563). Reaction mixture was stirred at room temperature for about 3 h. The reaction mixture was diluted with dichloromethane (100 mL) and washed with aqueous saturated NaHCO₃ (15 mL), aqueous 1N HCl (15 mL), and brine (15 mL). The organic layer was dried over magnesium sulfate, filtrated, and concentrated under reduced pressure. The residue was chromatographed on silica gel (gradient: acetone/hexanes; 2:8 to 5:5) to afford the product A2 in 84% yield.
Step 3

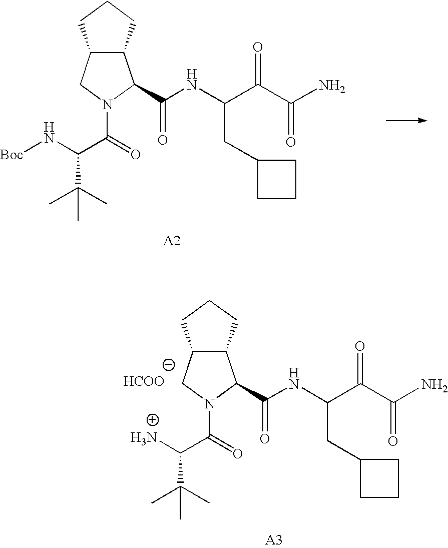

The N-Boc protected amine A2 was treated with 10 mL of formic acid. The resulting solution was stirred for 2 h. All the volatiles were removed under reduced pressure. No further purification was done for the product A3.
Step 4

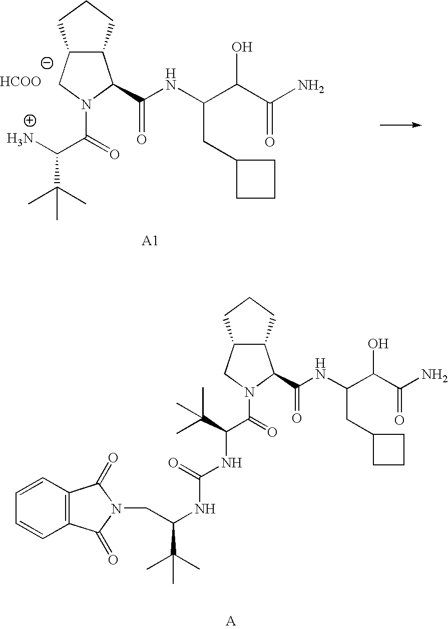

To a solution of the amine salt A3 in 1 mL of dry methylene chloride was added N-methylmorpholine (0.037 mL, d 0.920). The resulting solution was cooled in an ice-water bath and a solution of isocyanate in toluene (2.5 mL of a 0.135M soln) was slowly added. The mixture was stirred for 2 h (temp 0 to 25° C.). The reaction mixture was diluted with 60 mL of dichloromethane and washed with 15 mL of aqueous 1N HCl. Aqueous layer was back extracted with dichloromethane (2×20 mL). Combined organic layers were dried over magnesium sulfate, filtered and concentrated under reduced pressure. The residue was chromatographed on Silica gel (gradient: acetone/hexanes; 1:9 to 6:4) to give the product A (15 mg) as a white solid in 20% yield. HRMS (FAB) calcd for C₃₇H₅₃N₆O₇ [M+H] 693.3976; found 693.3987.

One skilled in the art would understand that other suitable compounds of Formula XVI can be prepared in a similar manner to that disclosed above.

The Following Experimental Section Applies for the Preparation of the Compounds of Formula XVII:

Abbreviations which are used in the descriptions of the schemes, preparations and the examples that follow are:

THF: Tetrahydrofuran

DMF: N,N-Dimethylformamide

EtOAc: Ethyl acetate

AcOH: Acetic acid

HOOBt: 3-Hydroxy-1,2,3-benzotriazin-4(3H)-one

EDCl:1-(3-dimethylaminopropyl)-3-ethylcarbodiimide hydrochloride

NMM: N-Methylmorpholine

ADDP: 1,1′-(Azodicarbobyl)dipiperidine

DEAD: Diethylazodicarboxylate

MeOH: Methanol

EtOH: Ethanol

Et2O: Diethyl ether

DMSO: Dimethylsulfoxide

HOBt: N-Hydroxybenzotriazole

PyBrOP: Bromo-tris-pyrrolidinophosphonium hexafluorophosphate

DCM: Dichloromethane

DCC: 1,3-Dicyclohexylcarbodiimide

TEMPO: 2,2,6,6-Tetramethyl-1-piperidinyloxy

Phg: Phenylglycine

Chg: Cyclohexylglycine

Bn: Benzyl

Bzl: Benzyl

Et: Ethyl

Ph: Phenyl

iBoc: isobutoxycarbonyl iPr: isopropyl

^{t}Bu or Bu^{t}: tert-Butyl

Boc: tert-Butyloxycarbonyl

Cbz: Benzyloxycarbonyl

Cp: Cylcopentyldienyl

Ts: p-toluenesulfonyl

Me: Methyl

HATU: O-(7-azabenzotriazol-1-yl)-1,1,3,3-tetramethyluronium hexafluorophosphate

DMAP: 4-N,N-Dimethylaminopyridine

BOP: Benzotriazol-1-yl-oxy-tris(dimethylamino)hexafluorophosphate

PCC: Pyridiniumchlorochromate

KHMDS: Potassium Hexamethyldisilazide or Potassium bis(trimethylsilylamide)

NaHMDS: Sodium Hexamethyldisilazide or Sodium bis(trimethylsilylamide)

LiHMDS: Lithium Hexamethyldisilazide or Lithium bis(trimethylsilylamide)

10% Pd/C: 10% Palladium on carbon (by weight).

TG: Thioglycerol

General Schemes for Preparation of Target Compounds

Compounds of the present invention were synthesized using the general schemes (Methods A-E) described below.

Method A

Deprotection of the N-Boc functionality of 1.01 under acidic conditions provided the hydrochloride salt 1.02 which was subsequently coupled with N-Boc-tert-leucine under peptide coupling methodology to afford 1.03. N-Boc deprotection followed by treatment with appropriate isocyanate gave the urea 1.05. Hydrolysis of the methyl ester provided the acid 1.06. Peptide coupling of the acid 1.06 with the appropriate P₁-P′ primary amide moiety afforded the hydroxylamide 1.07. Oxidation (Moffatt oxidation or related process—see, T. T. Tidwell, Synthesis, 1990, 857), or Dess-Martin Periodinane—J. Org. Chem., (1983) 48, 4155) resulted in the target compound 1.08. 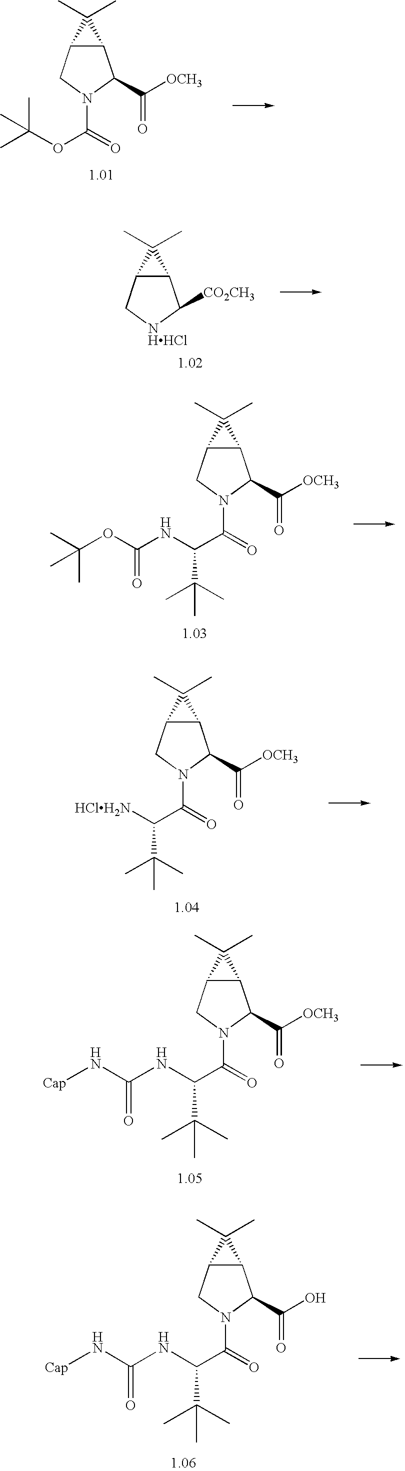 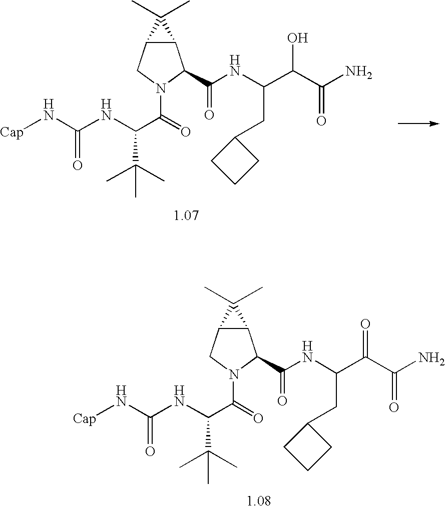
Method B

Peptide coupling of the acid 1.06 with the appropriate P₁-P′ secondary amide moiety afforded the hydroxylamide 1.09. Oxidation (Moffatt or Dess-Martin's) resulted in the target compound 1.10. 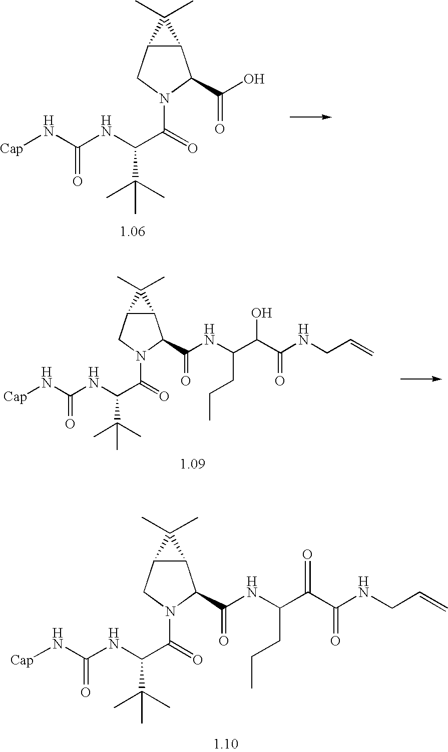
Method C

In another variation, peptide coupling of the N-Boc-P2-P₃-acid 1.17 with the appropriate P₁-P′ amide moiety afforded the hydroxylamide 1.11. Oxidation (Moffatt or Dess-Martin Periodinane) resulted in the keto amide 1.12. Deprotection of the N-Boc functionality gave the hydrochloride salt 1.13. Treatment with a suitable isocyanate (or isocyanate equivalent) resulted in the target compound 1.14. 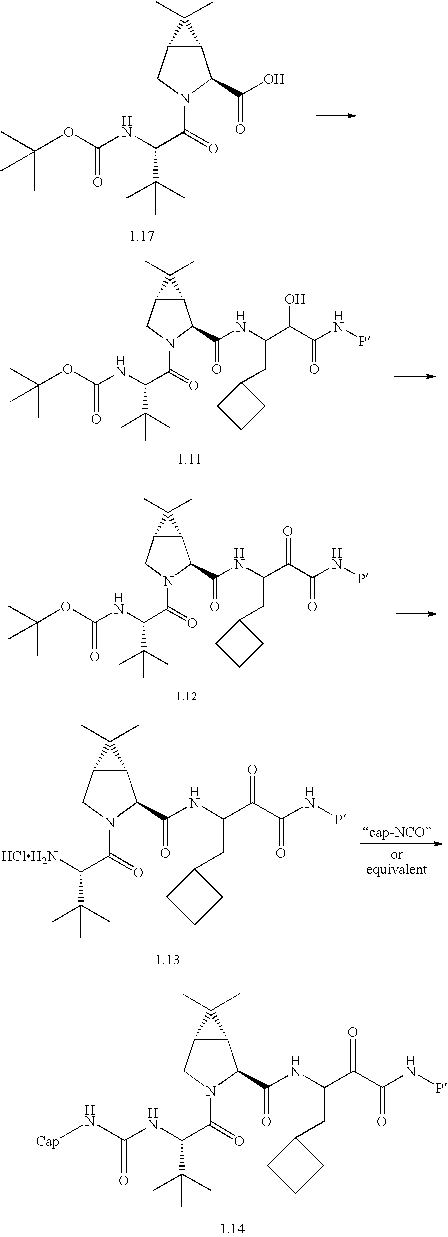
Method D

In yet another variation, the hydrochloride salt 1.13 was converted to the 4-nitrophenyl carbamate 1.15 by reaction with 4-nitrophenyl chloroformate. Subsequent treatment with an amine (or amine hydrochloride salt) of choice provided the target compound 1.14. 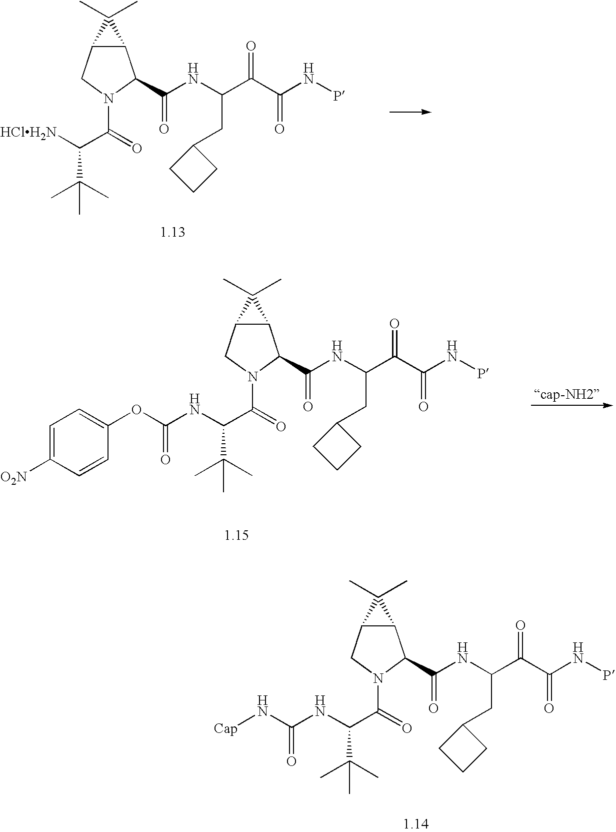
Method E

In yet another variation, the dipeptide hydrochloride salt 1.03 was converted to the 4-nitrophenyl carbamate as described above. Treatment with an amine (or amine hydrochloride salt) of choice provided the urea derivative 1.05. Hydrolysis and further elaboration as described in Methods A/B provided the target compounds 1.14. 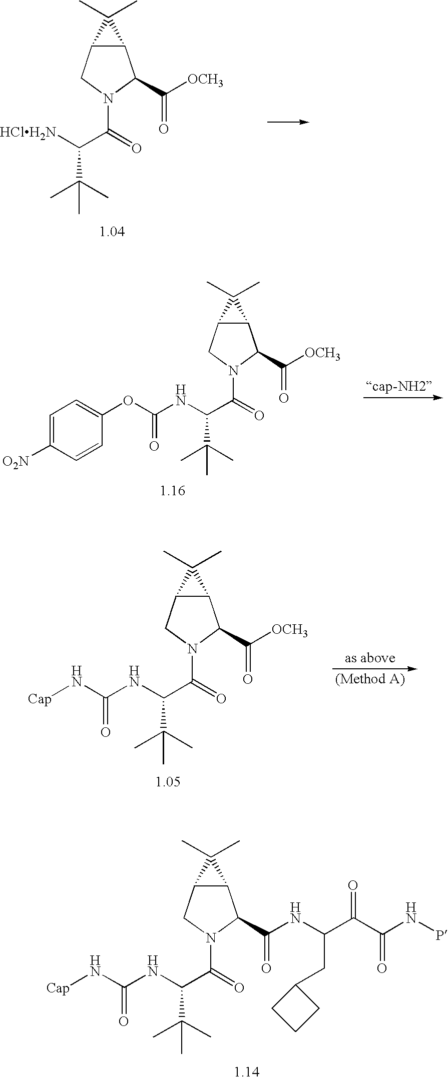
The Following Experimental Section Applies for the Preparation of the compounds of Formula XIX:
Synthesis of Preparative Examples
Synthesis of Example 101
Step 1

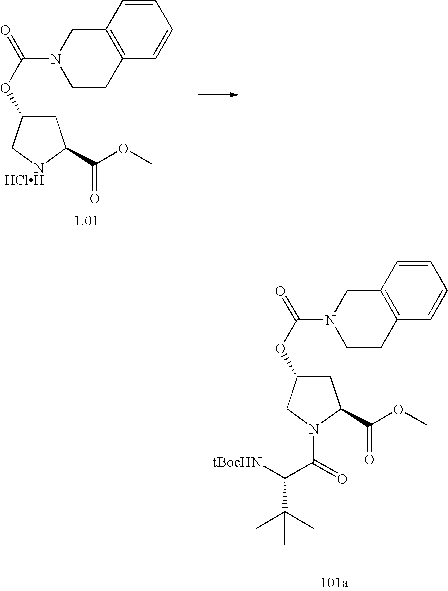

To a stirred solution of the proline derivative 1.01 (3.66 mmol, prepared as described above) in dichloromethane (20 mL) and DMF (15 mL) at 0° C. was added L-boc-tert-leucine (930 mg, 4.03 mmol), DIPEA (2.02 mL, 10.98 mmol) and HATU (1.8 g, 4.76 mmol). After 15 minutes at that temperature, the reaction flask was stored in the freezer (−20° C.), overnight (16 hr). The reaction mixture was diluted with dichloromethane (80 mL) and washed with saturated sodium bicarbonate solution (80 mL), 10% aq. citric acid solution (80 mL), brine (80 mL), dried (Na₂SO₄), filtered and concentrated. The crude material was purified by silica chromatography using 25/75 to 50/50 EtOAc/hexanes to provide 1.77 g of the required material, 101a. LC-MS: 518.1 (M+H)⁺.
Step 2

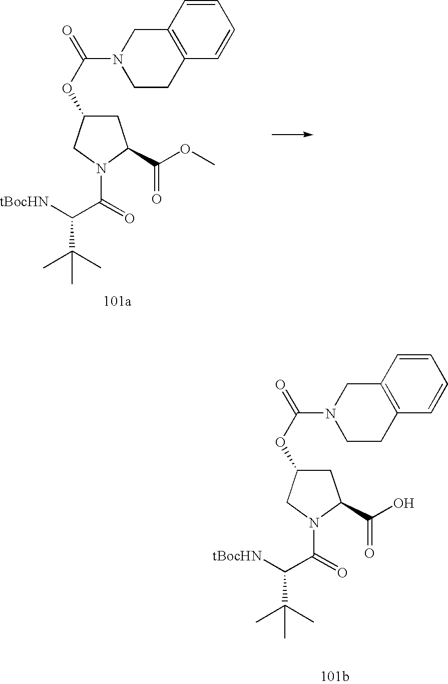

To a solution of the methyl ester 101a (1.21 g, 2.34 mmol) in THF (10 mL) and MeOH (5 mL) was added aq. 1 M LiOH solution (5 mL). The reaction mixture was stirred at RT for 4 h. It was then concentrated, diluted with water (50 mL) and acidified with solid citric acid (pH approximately 3) when white solid material crashed out. This solid was filtered off, washed with water and dried in vacuo to afford 970 mg of 101b. LC-MS: 504.1 (M+H)⁺.
Step 3

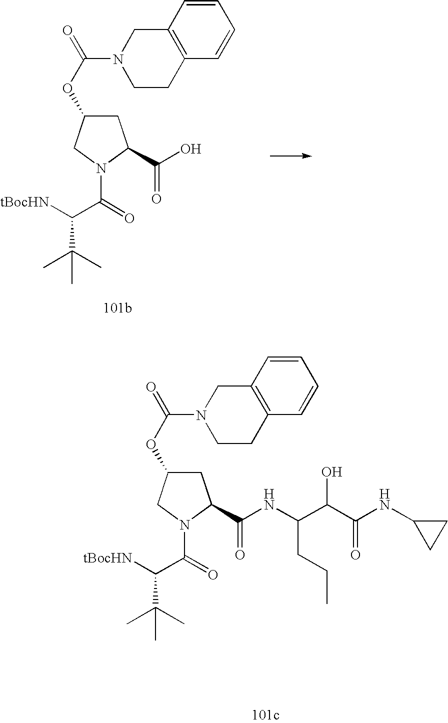

The acid 101b (503 mg, 1 mmol) was coupled with intermediate 13.06 (334 mg, 1.5 mmol) using essentially procedure described above (Step 1, preparation of 101a) to provide 101c which was used without purification. MS: 672.37 (M+H)⁺.
Step 4

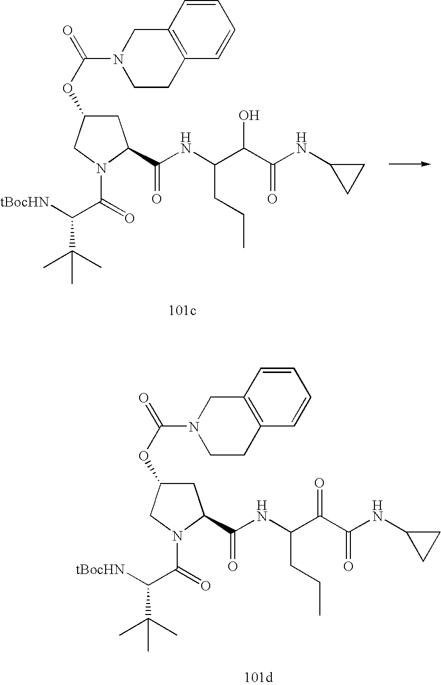

To a solution of the hydroxyl compound 101c from above in dichloromethane (15 mL) was added Dess-Martin's periodinane (848 mg, 2 mmol) and the reaction mixture was stirred at RT for 5 h. At this time, the reaction mixture was diluted with dichloromethane (30 mL) and washed with 1:1 mixture of aq. 10% sodium thiosulfate solution and saturated sodium bicarbonate solution (2×25 mL each), brine (50 mL), dried (Na₂SO₄), filtered and concentrated. The crude material was purified by silica chromatography using 15/85 to 50/50 acetone/hexanes to provide 410 mg of the required material, 101d. LC-MS: 670.2 (M+H)⁺.
Step 5

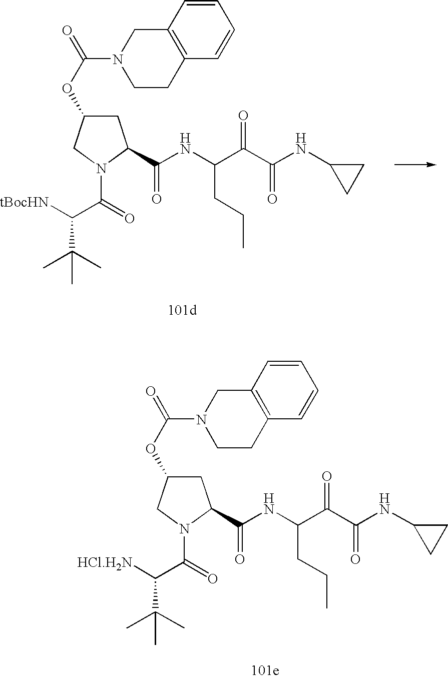

Deprotection of the N-boc functionality of 101d to provide the required material 101e was carried out as described for intermediate 1.01, Step 3 (reaction time=2 h). LC-MS: 570.1 (M+H)⁺.
Step 6

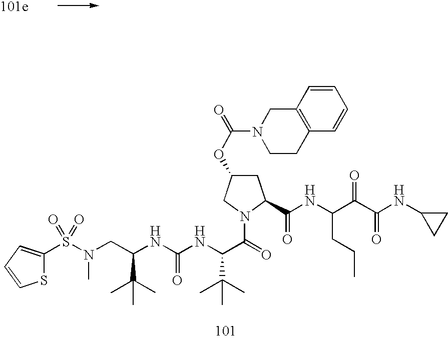

To a solution of the amine salt 101e (60 mg, 0.1 mmol) in dichloromethane (2 mL) at 0° C. was added DIPEA (0.06 mL, 0.3 mmol) followed by the isocyanate intermediate 65.01 (0.25 M solution in toluene, 0.8 mL, 0.2 mmol). After 15 minutes at that temperature, the reaction flask was stored in the freezer (−20° C.), overnight (16 hr). The reaction mixture was diluted with dichloromethane (20 mL) and washed with saturated ammonium chloride solution (20 mL), brine (20 mL), dried (Na₂SO₄), filtered and concentrated. The crude material was purified by silica chromatography using 15/85 to 50/50 acetone/hexanes to provide the required compound 101 (53 mg); LC-MS: 872.2 (M+H)⁺.

One skilled in the art would understand that other suitable compounds of Formula XIX can be prepared in a similar manner to that disclosed above.

The Following Experimental Section Applies for the Preparation of the Compounds of Formula Ia, Ib and Ic:

Abbreviations:

Abbreviations which are used in the descriptions of the schemes, preparations and the examples that follow are:

THF: Tetrahydrofuran

DMF: N,N-Dimethylformamide

EtOAc: Ethyl acetate

AcOH: Acetic acid

HOOBt: 3-Hydroxy-1,2,3-benzotriazin-4(3H)-one

EDCl: 1-(3-dimethylaminopropyl)-3-ethylcarbodiimide hydrochloride

NMM: N-Methylmorpholine

MeOH: Methanol

EtOH: Ethanol

Et2O: Diethyl ether

DMSO: Dimethylsulfoxide

K^{t}BuO: Potassium tert-butoxide

DCM: Dichloromethane

Chg: Cyclohexylglycine

Bn: Benzyl

Et: Ethyl

Ph: Phenyl

iPr: isopropyl

^{t}Bu or Bu^{t}: tert-Butyl

Boc: tert-Butyloxycarbonyl

Cbz: Benzyloxycarbonyl

HATU: O-(7-azabenzotriazol-1-yl)-1,1,3,3-tetramethyluronium hexafluorophosphate

BOP: Benzotriazol-1-yl-oxy-tris(dimethylamino)hexafluorophosphate

10% Pd/C: 10% Palladium on carbon (by weight).

### EXAMPLE

### Synthesis of (1R,5S)—N-[3-Amino-1-(Cyclobutylmethyl)-2,3-Dioxopropyl]-3-[2(S)-[[[(1,1-Dimethylethyl)Amino]Carbonyl]Amino]-3,3-Dimethyl-1-Oxobutyl]-6,6-Dimethyl-3-Azabicyclo[3.1.0]Hexan-2(S)-Carboxamide (Structure 1a):

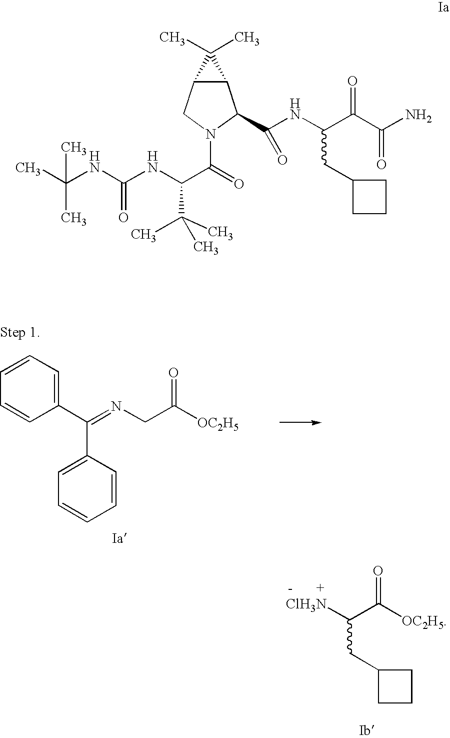

A stirred solution of the ketimime 1a′ (50 g, 187.1 mmol, available from Aldrich Chemical Company, Milwaukee, Wis.) under N₂ in dry THF (400 mL) was cooled to −78° C. and treated with 1 M solution of K-^{t}BuO (220 mL, 1.15 equiv.) in THF. The reaction mixture was warmed to 0° C. and stirred for 1 h and treated with bromomethylcyclobutane (28 mL, 249 mmol). The reaction mixture was stirred at room temperature for 48 h and concentrated in vacuo. The residue was dissolved in Et₂O (300 mL) and treated with aq. HCl (2 M, 300 mL) The resulting solution was stirred at room temperature for 5 h and extracted with Et₂O (1 L). The aqueous layer was made basic to pH ˜12-14 with aq. NaOH (50%) and extracted with CH₂Cl₂ (3×300 mL). The combined organic layers were dried (MgSO₄), filtered, and concentrated to give pure amine (1b′, 18 g) as a colorless oil.
Step 2.

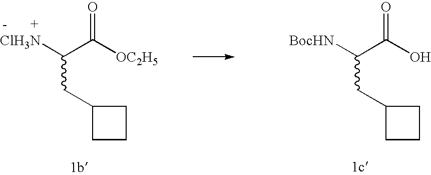

A solution of the amine 1b′ (18 g, 105.2 mmol) at 0° C. in CH₂Cl₂ (350 mL) was treated with di-tert-butyldicarbonate (23 g, 105.4 mmol) and stirred at rt. for 12 h. After the completion of the reaction (TLC), the reaction mixture was concentrated in vacuo and the residue was dissolved in THF/H₂O (200 ml, 1:1) and treated with LiOH.H₂O (6.5 g, 158.5 mmol) and stirred at room temperature for 3 h. The reaction mixture was concentrated and the basic aqueous layer was extracted with Et₂O. The aqueous layer was acidified with conc. HCl to pH˜1-2 and extracted with CH₂Cl₂. The combined organic layers were dried (MgSO₄), filtered, and concentrated in vacuo to yield 1c′ as a colorless viscous oil which was used for next step without any further purification.
Step 3.

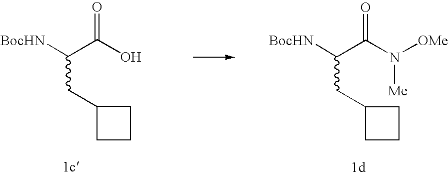

A solution of the acid 1c′ (15.0 g, 62 mmol) in CH₂Cl₂ (250 mL) was treated with BOP reagent (41.1 g, 93 mmol), N-methylmorpholine (27 mL), N,O-dimethyl hydroxylamine hydrochloride (9.07 g, 93 mmol) and stirred overnight at rt. The reaction mixture was diluted with 1N aq. HCl (250 mL), and the layers were separated and the aqueous layer was extracted with CH₂Cl₂ (3×300 ml). The combined organic layers were dried (MgSO₄), filtered, concentrated in vacuo and purified by chromatography (SiO₂, EtOAc/Hex 2:3) to yield the amide 1d (15.0 g) as a colorless solid.
Step 4.

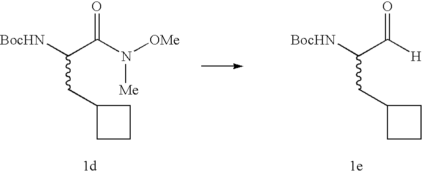

A solution of the amide 1d (15 g, 52.1 mmol) in dry THF (200 mL) was treated dropwise with a solution of LiAlH₄ (1M, 93 mL, 93 mmol) at 0° C. The reaction mixture was stirred at room temperature for 1 h and carefully quenched at 0° C. with a solution of KHSO₄ (10% aq.) and stirred for 0.5 h. The reaction mixture was diluted with aq. HCl (1M, 150 mL) and extracted with CH₂Cl₂ (3×200 mL), The combined organic layers were washed with aq. HCl (1 M), saturated NaHCO₃, brine, and dried (MgSO₄). The mixture was filtered and concentrated in vacuo to yield 1e as viscous colorless oil (14 g).
Step 5.

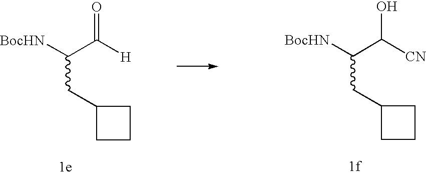

A solution of the aldehyde 1e (14 g, 61.6 mmol) in CH₂Cl₂ (50 mL), was treated with Et₃N (10.73 mL, 74.4 mmol), and acetone cyanohydrin (10.86 g, 127.57 mmol) and stirred at room temperature for 24 hrs. The reaction mixture was concentrated in vacuo and diluted with aq. HCl (1 M, 200 mL) and extracted into CH₂Cl₂ (3×200 mL). The combined organic layer were washed with H₂O, brine, dried (MgSO₄), filtered, concentrated in vacuo and purified by chromatography (SiO₂, EtOAc/Hex 1:4) to yield 1f (10.3 g) as a colorless liquid as a mixture of diastereomers.
Step 6.

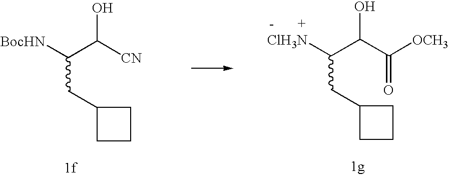

Methanol saturated with HCl*, prepared by bubbling HCl gas to CH₃OH (700 ml) at 0° C., was treated with cyanohydrin 1f and heated to reflux for 24 h. The reaction was concentrated in vacuo to yield 1 g, which was used in the next step without purification.

* Alternatively 6M HCl prepared by addition of AcCl to dry methanol can also be used.

Step 7.

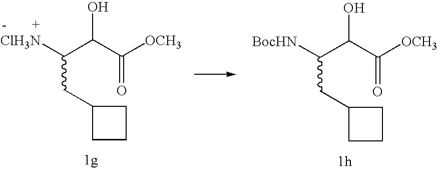

A solution of the amine hydrochloride 1g in CH₂Cl₂ (200 mL) was treated with Et₃N (45.0 mL, 315 mmol) and Boc₂O (45.7 g, 209 mmol) at −78° C. The reaction mixture was then stirred at room temperature overnight and diluted with HCl (2 M, 200 mL) and extracted into CH₂Cl₂. The combined organic layers were dried (MgSO₄) filtered, concentrated in vacuo and purified by chromatography (EtOAc/Hex 1:4) to yield hydroxy ester 1 h.
Step 8.

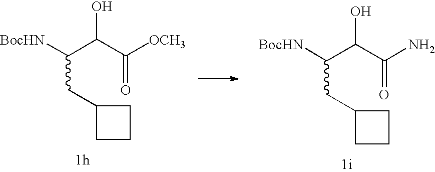

A solution of methyl ester 1h (3 g, 10.5 mmol) in THF/H₂O (1:1) was treated with LiOH.H₂O (645 mg, 15.75 mmol) and stirred at rt. for 2 h. The reaction mixture was acidified with aq HCl (1 M, 15 mL) and concentrated in vacuo. The residue was dried in vacuum.

A solution of the acid in CH₂Cl₂ (50 mL) and DMF (25 mL) was treated with NH₄Cl (2.94 g, 5.5 mmol), EDCl (3.15 g, 16.5 mmol), HOOBt (2.69 g, 16.5 mmol), and NMM (4.4 g, 44 mmol). The reaction mixture was stirred at room temperature for 3 d. The solvents were removed under vacuo and the residue was diluted with aq. HCl (250 mL) and extracted with CH₂Cl₂. The combined organic layers were washed with aq. saturated NaHCO₃, dried (MgSO₄) filtered concentrated in vacuo to obtain 1i, which was used as it is in the following steps. (Alternatively 1i can also be obtained directly by the reaction of 1f (4.5 g, 17.7 mmol) with aq. H₂O₂ (10 mL), LiOH.H₂O (820 mg, 20.8 mmol) at 0° C. in 50 mL of CH₃OH for 0.5 h.)
Step 9.

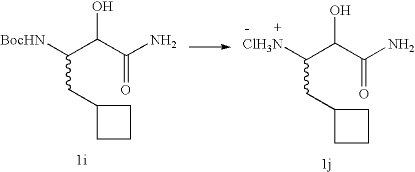

A solution of 1i obtained in the previous step was dissolved in 4 N HCl in dioxane and stirred at rt. for 2 h. The reaction mixture was concentrated in vacuo to give 1j as a solid, which was used without further purification.
Step 10.

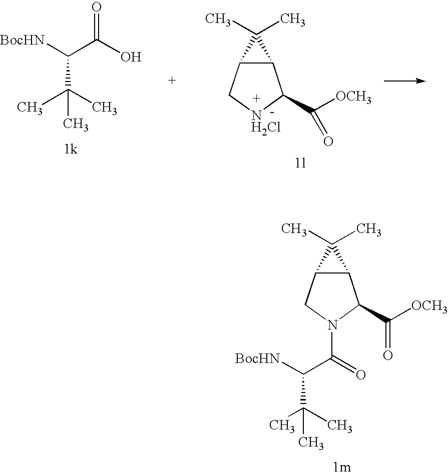

The amino ester 1l was prepared following the method of R. Zhang and J. S. Madalengoitia (J. Org. Chem. 1999, 64, 330), with the exception that the Boc group was cleaved by the reaction of the Boc-protected amino acid with methanolic HCl.

A solution of Boc-tert-Lue 1k (Fluka, 5.0 g 21.6 mmol) in dry CH₂Cl₂/DMF (50 mL, 1:1) was cooled to 0° C. and treated with the amine 11 (5.3 g, 25.7 mmol), NMM (6.5 g, 64.8 mmol) and BOP reagent (11.6 g, 25.7 mmol). The reaction was stirred at rt. for 24 hrs, diluted with aq. HCl (1 M) and extracted with CH₂Cl₂. The combined organic layers were washed with HCl (aq, 1 M), saturated NaHCO₃, brine, dried (MgSO₄), filtered and concentrated in vacuo and purified by chromatography (SiO₂, acetone/hexane 1:5) to yield 1m as a colorless solid.
Step 11.

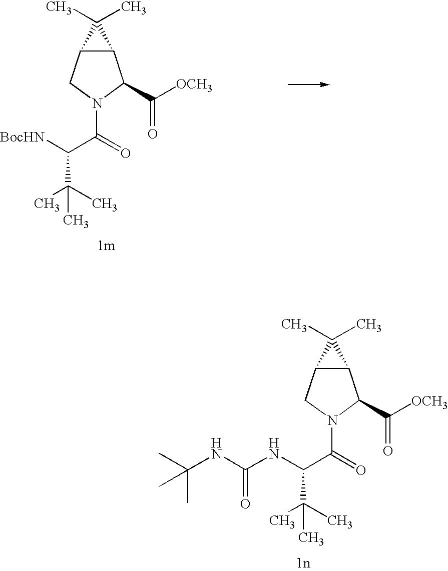

A solution of methyl ester 1m (4.0 g, 10.46 mmol) was dissolved in HCl (4 M solution in dioxane) and stirred at rt. for 3 h. The reaction mixture was concentrated in vacuo to obtain the amine hydrochloride salt used in the next step without further purification.

A solution of the amine hydrochloride salt (397 mg, 1.24 mmol) in CH₂Cl₂ (10 mL) was cooled to −78° C. and treated with tert-butyl isocyanate (250 mg, 2.5 mmol) and stirred at rt. overnight. The reaction mixture was concentrated in vacuo and the residue was diluted with aq. HCl (1 M) and extracted with CH₂Cl₂. The combined organic layers were washed with aq. HCl (1 M), saturated NaHCO₃ and brine. The organic layers were dried, filtered and concentrated in vacuo and the residue was purified by chromatography (SiO₂, acetone/Hex 1:4) to yield 1n as a colorless solid.
Step 12.

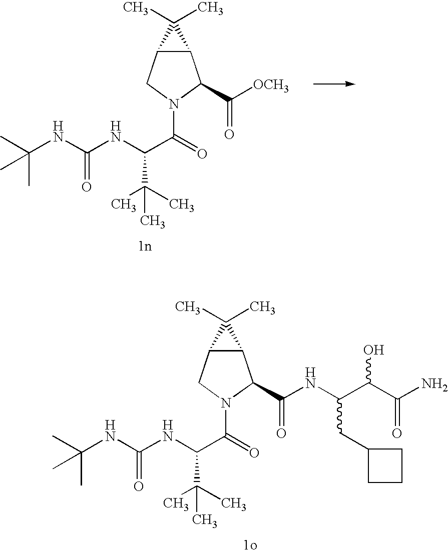

A solution of methyl ester 1n (381 mg, 1.0 mmol) in THF/H₂O (1:1, 5 mL) was treated with LiOH.H₂O (62 mg, 1.5 mmol) and stirred at rt. for 3 h. The reaction mixture was acidified with aq. HCl and concentrated in vacuo to obtain the free acid.

A solution of acid (254.9 mg, 0.69 mmol) in DMF/CH₂Cl₂ (1:1, 5.0 mL) was treated with amine 1j (159 mg, 0.763 mmol), EDCl (199 mg, 1.04 mmol), HOOBt (169.5 mg, 1.04 mmol) and NMM (280 mg, 2.77 mmol) at −20° C. The reaction mixture was stirred at −20° C. for 48 h and concentrated in vacuo. The residue was diluted with aq. 1M HCl and extracted with EtOAc, The combined organic layers were extracted with aq. NaHCO₃, aq. HCl, brine, dried (MgSO₄) filtered, concentrated in vacuo to obtain 1o (470 mg) as a tan colored solid that was used in the next reaction without further purification.
Step 13.

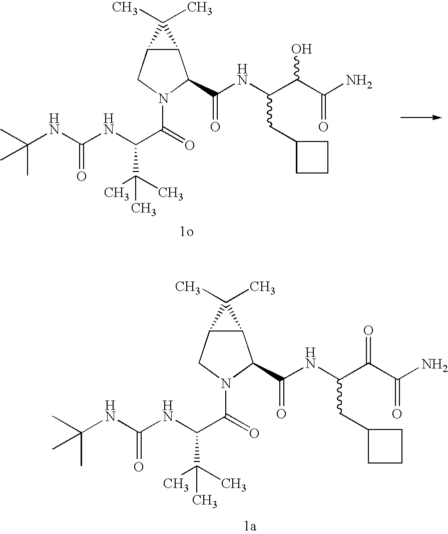

A solution of amide 1o (470 mg, 0.9 mmol) in toluene and DMSO (1:1 20 mL) at 0° C. was treated with EDCl (1.72 g, 9.0 mmol) and dichloroacetic acid (0.37 mL, 4.5 mmol) and stirred at 0° C. for 4 hrs. The reaction mixture was diluted with CH₂Cl₂, and washed with saturated NaHCO₃, and brine. The organic layer was dried (MgSO₄), filtered, concentrated, in vacuo and purified by chromatography (SiO₂, acetone/hexanes 3:7) to yield 1a as a colorless solid.
Separation of the Compound of Formula 1 into Diastereomers of Formula Ib and Ic:

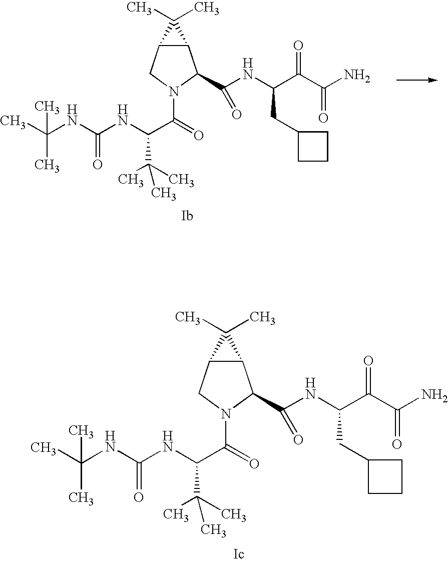

Preparative HPLC Condition for Separation

| | |
|---|---|
| COLUMN USED: | NORMAL PHASE YMC DIOL-NP COLUMN |
| | 120 Å, S-10/20; 50 mm × 500 mm I.D/length |
| SOLVENT A: | Hexanes |
| SOLVENT B: | To make 4 L of solvent (1.7 L Isopropanol + |
| | 300 mL of CH₃CN + 2 L of CH₂Cl₂) |
| HPLC CONDITIONS: | 12% of Solvent B/88% of Solvent A |
| FLOW: | 120 mL/min |

Procedure: 1 g of compound 1a was dissolved in 10 mL of CH₂Cl₂/25 mL of Hexanes and injected into the column. It was eluted with 120 mL/min and two peaks were independently collected and concentrated. The solid residue was further dried in high vacuum and analyzed by analytical HPLC. Since the polar (second isomer) contained 2.6% of nonpolar diastereomer (First isomer), it was purified once more to isolate the pure diastereomers.

Analytical Conditions for Analysis of Diastereomeric Purity

| | |
|---|---|
| COLUMN USED: | NORMAL PHASE YMC DIOL-NP COLUMN |
| | 200 Å, S-5 □M; 150 mm × 3 mm length/I.D |
| SOLVENT A: | Hexanes |
| SOLVENT B: | To make 4 L of solvent (1.7 L Isopropanol + |
| | 300 mL of CH₃CN + 2 L of CH₂Cl₂) |
| HPLC CONDITIONS: | 8.5% of Solvent B/91.5% of Solvent A |
| FLOW: | 0.7 mL/min |
| Rt | Nonpolar isomer (compound lb) = 13.2 min |
| | Polar isomer (compound lc) = 16.1 min |

2.5 mg of compound in 1 mL was used and 20 μL was injected and analyzed with a U.V detector at λ=254 nm.

Analytical Data for Compounds 2 and 3.

Compound 3 [Polar Diastereomer]

¹H NMR (d₆-dmso, 500 MHz): δ 8.26 (d, 1H, J=7.0 Hz), 8.00 (s, 1H), 7.75 (s, 1H), 5.96 (s, 1H), 5.84 (d, 1H, J=10 Hz), 4.96 (m, 1H), 4.28 (s, 1H), 4.11 (d, 1H, J=11 Hz), 3.94 (d, 1H, J=10 Hz), 3.73 (dd, 1H, J=10 & 5 Hz), 2.48 (m, 1H), 1.95 (m, 2H), 1.61 (m, 1H), 1.59 (m, 1H), 1.77 (m, 1H), 1.57 (m, 1H), 1.74 (m, 2H), 1.42 (dd, 1H, J=7.5 & 5 Hz), 1.28 (d, 1H, J=7.5 Hz), 1.17 (s, 9H), 1.01 (s, 3H), 0.90 (s, 9H), 0.85 (s, 3H). ¹³C NMR (d₆-dmso, 125 MHz): δ 197.8, 170.9, 170.8, 162.8, 157.4, 59.1, 56.8, 51.8, 48.9, 47.4, 36.7, 34.0, 32.0, 30.6, 29.1, 27.8, 27.3, 27.1, 26.4, 26.1, 18.5, 17.7, 12.5. MS [FAB] 520 (55), 421 (100), 308 (75), 213 (90).

HRMS calcd for C₂₇H₄₆O₅N₅ [M+1]⁺ 520.3499; observed: 520.3505.

Compound 2 [Non-polar Diastereomer]

¹H NMR (d₆-dmso, 500 MHz): δ 8.15 (d, 1H, J=7.0 Hz), 7.96 (s, 1H), 7.74 (s, 1H), 5.96 (s, 1H), 5.86 (d, 1H, J=10 Hz), 4.85 (m, 1H), 4.27 (s, 1H), 4.13 (d, 1H, J=11.0 Hz), 3.97 (d, 1H, J=10 Hz), 3.76 (dd, 1H, J=10 & 5 Hz), 2.36 (m, 1H), 1.97 (m, 2H), 1.60 (m, 2H), 1.78 (m, 1H), 1.64 (m, 1H), 1.75 (m, 2H), 1.44 (dd, 1H, J=7.5 & 5 Hz), 1.27 (d, 1H, J=7.5 Hz), 1.17 (s, 9H), 1.00 (s, 3H), 0.89 (s, 9H), 0.82 (s, 3H). ¹³C NMR (d₆-dmso125 MHz): δ 197.1, 171.1, 170.7, 163.0, 157.3, 59.4, 56.9, 52.1, 48.9, 47.4, 36.6, 34.0, 32.1, 30.5, 29.1, 27.9, 27.4, 26.8, 26.4, 26.1, 18.5, 17.8, 12.4. MS [FAB] 520 (40), 421 (100), 308 (60), 213 (65). HRMS calcd. for C₂₇H₄₆O₅N₅ [M+1]+520.3499; observed: 520.3514.

A preferred formulation of HCV protease inhibitor Formula I is illustrated below.

| Constituent | Concentration (mg/capsule) |
|---|---|
| Precipitate of Compound of Formula I | 200 |
| Microcrystalline Cellulose | 40 |
| Lactose Monohydrate | 56 |
| Croscarmellose Sodium | 24 |
| Pregelatinized Starch | 60 |
| Sodium Lauryl Sulfate | 12 |
| Magnesium Stearate | 8 |
| Purified Water | (—) |
| Capsule Net Fill Weight | 400 |
| Hard Gelatin Capsule | 1 each |

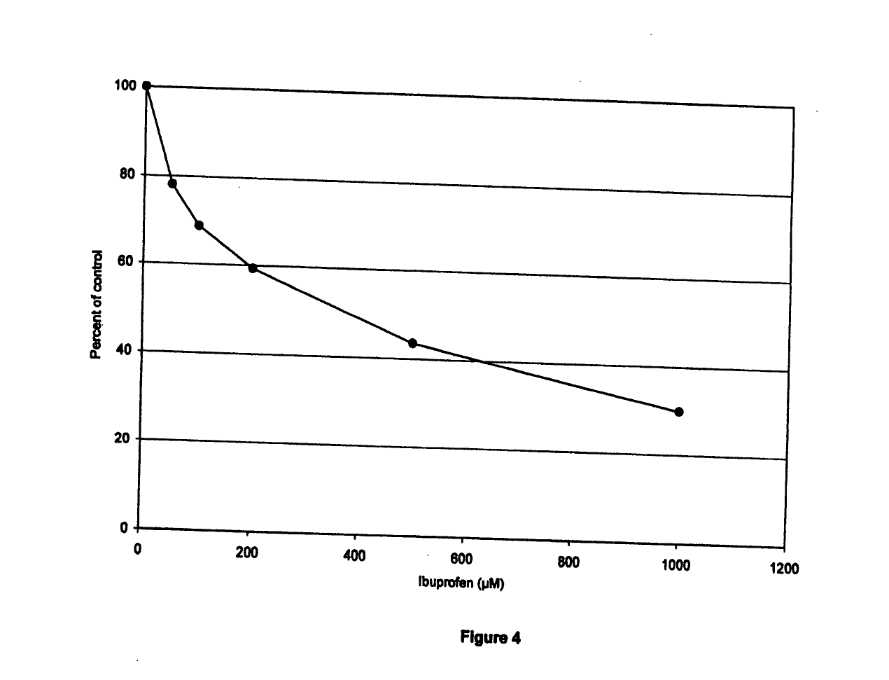

The method of making this preferred formulation is detailed in U.S. Patent Application No. 60/796,490 and U.S. Patent Application Ser. No. 60/796,717 (e.g., see, U.S. Patent Application Ser. No. 60/796,717, Example III). In addition, the preferred purification process for Formula I is detailed in U.S. Patent Application No. 60/796,490 and U.S. Patent Application Ser. No. 60/796,717 (e.g., see, U.S. Patent Application Ser. No. 60/796,717, FIG. 4 and pages 9-21), incorporated herein by reference.

### EXAMPLES

Inhibition Studies with Selective Inhibitors of Cytosolic Enzymes

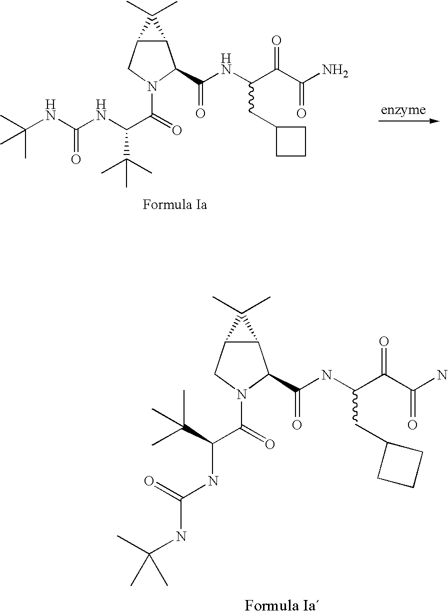

Inhibition of metabolism of ¹⁴C-radiolabeled compound of Formula Ia to compound of Formula Ia′ was evaluated using the following selective chemical inhibitors of cytosolic enzymes: bis(4-nitrophenyl)-phosphate (BNPP) for carboxylesterase/amidase, quercetin for carbonyl reductase, menadione for aldehyde oxidase and carbonyl reductase, allopurinol for xanthine oxidase, and flufenamic acid for AKR (see Table 1). Human liver S9, cytosol or mitochondria (1.6 mg protein/mL) were pre-incubated separately with the selected inhibitors for 15 min at room temperature followed by the addition of buffer, cofactor and substrate (20 μM ¹⁴C-radiolabeled compound of Formula Ia). All incubations contained 3 mM magnesium chloride and NADPH-generating system in 0.5 mL of 50 mM potassium phosphate buffer, pH 7.4. Prior to the addition of drug, incubation mixtures were preincubated for 2 min at 37° C. Reactions were initiated by addition of drug, allowed to proceed for 120 min at 37° C. and then terminated by the addition of 0.5 mL of ice-cold methanol. The incubation mixtures were vortexed and centrifuged (˜10,000 g) at 4° C. for 10 min; supernatants were analyzed by HPLC coupled with radiometric detector. Heat activated S9 cofactor or mitochondria were used as control. For LC-MS analysis, supernatants were concentrated in SpeedVac for 3 hrs. Incubation volumes were 0.5 mL and the final concentration of the organic solvents in the incubation system was less than 1% (v/v).

The sample analysis was performed on a Waters Alliance HPLC system (Alliance Model 2690; Waters Corp., Milford, Mass.), equipped with Model 996 Photodiode Array Detector (Waters Corp.), Model 500TR Radioactivity Detector (PerkinElmer Life & Analytical Sciences, Boston, Mass.) and a 5-μm Varian Polaris C18-A, 250×4.6 mm analytical column (ANSYS Technologies, Lake Forrest, Calif.). The analytical column was maintained at 40° C. and the guard column (MetaGuard polaris C18-A from ANSYS Technologies). The mobile phase consisted of 10 mM ammonium acetate adjusted to pH 7.0 with 1% ammonium hydroxide (A) and 100% methanol (B). The flow rate was maintained at 1 mL/min and the metabolite was detected at 254 nm. Gradient elution of metabolites was achieved using programmed changes in mobile phase composition as summarized in the following table.

| Time (min) | % A | % B |
|---|---|---|
| 0.00 | 95 | 5 |
| 5.00 | 95 | 5 |
| 7.00 | 46 | 54 |
| 26.00 | 46 | 54 |
| 43.00 | 35 | 65 |
| 46.00 | 5 | 95 |
| 49.00 | 5 | 95 |
| 50.00 | 95 | 5 |
| 60.00 | 95 | 5 |

The results of the chemical inhibition studies showed that at 100 μM menadione (CBR and aldehyde oxidase inhibitor) inhibited formation of the compound of Formula Ia′ by 30 and 18% in cytosol and S9, respectively. Similarly, at 100 μM, quercetin (CBR inhibitor) inhibited formation of the compound of Formula Ia′ by 33.4 and 9.3% in cytosol and S9, respectively. BNPP, carboxylase/amidase inhibitor, inhibited formation of the compound of Formula Ia′ by 63.4 and 57.4% from cytosol and S9, respectively. However, amidase is not NADPH-dependent suggesting that its involvement, if any, is minimal. Pargyline (MAO-A and MAO-B inhibitor) and allopurinol (xanthine oxidase inhibitor) showed no inhibition. Flufenamic acid (AKR inhibitor) and phenolphthalein inhibited formation of the compound of Formula Ia′ by 80.3 and 86.1%, respectively, implicating the involvement of AKR.

TABLE 1
Inhibitors of cytosolic enzymes.

| Cytosolic Enzymes | Inhibitors |
|---|---|
| Carbonyl reductase | Menadione |
| Aldehyde oxidase | Menadione |
| Carbonyl reductase | Quercetin |
| AKR | Flufenamic acid |
| Xanthine oxidase | Allopurinol |
| Carboxylesterase | bis (4-nitrophenyl) phosphate (BNPP) |
| Amidase | |
| MAO (A and B) | Pargyline |

Incubation of Compound Formula Ia with Recombinant Human AKRs

Recombinant human AKRs (AKR1C2, AKR1C3 and AKR1C4) were grown, sonicated and centrifuged at ˜10,000 g to obtain S9 fractions. Incubations of ¹⁴C-radiolabeled compound of Formula Ia, ¹⁴C-radiolabeled compound of Formula Ib and ¹⁴C-radiolabeled compound of Formula Ic (all at 20 μM) with S9-fractions from three recombinant human AKRs (2.5 mg protein/mL) were conducted as described above.

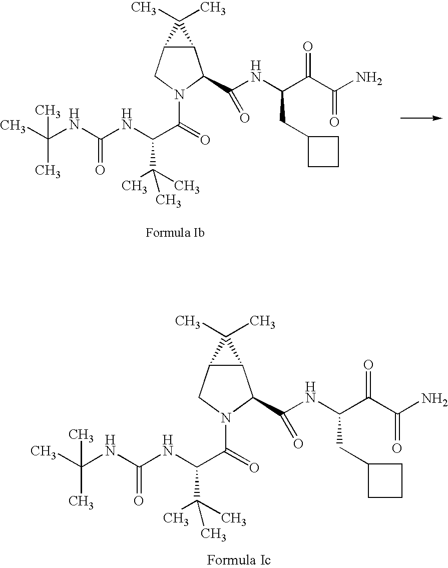

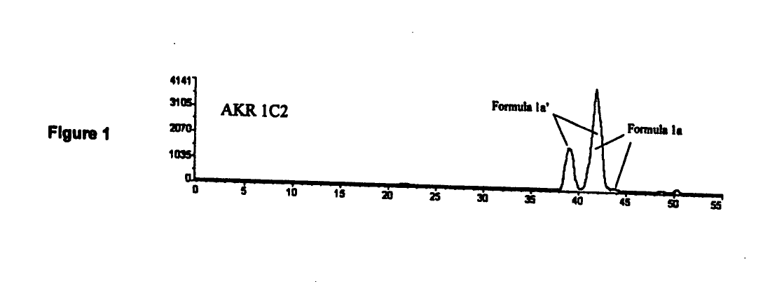

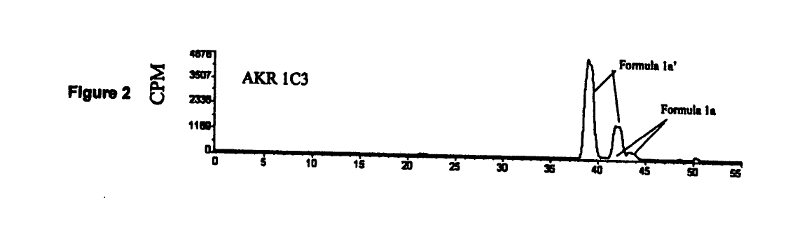

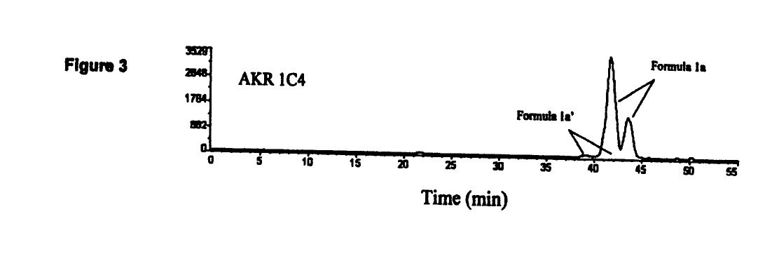

The samples were analyzed by HPLC-coupled with radiometric detector and confirmed by LC-MS. As shown in FIGS. 1-3, incubation of ¹⁴C-radiolabeled compound of Formula Ia with AKR1C2, AKR1C3 and AKR1C4 in the presence of NADPH showed that AKR1C2 and AKR1C3 yielded compound of Formula Ia′. AKR1C3 preferentially metabolized ¹⁴C-radiolabeled compound of Formula Ib, while AKR1C2 preferentially metabolized ¹⁴C-radiolabeled compound of Formula Ic.

Inhibition Studies with AKR Competitors

Inhibition studies were also conducted using some of the AKR competitors listed in Table 2.

TABLE 2
AKR competitors (i.e., AKR substrate or inhibitor).

| | Inhibitor | AKR1C3- |
|---|---|---|
| Substrate | AKR1C2/3 | specific |
| Benzafibrate | Diazepam | Cloxazolam |
| Clinofibrate | Estazolam | |
| Clofibric acid | Flunitrazepam | |
| DHT (5α-dihydroxytestosterone | Medazepam | |
| Dolasetron (5-HT3 receptor anatagonist) | Nitrazepam | |
| Doxorubicin | Celecoxib | |
| 17β-Estradiol | Naproxen | |
| Ibuprofen | Ibuprofen | |
| Flufenamic acid (NSAID) | Testosterone | |
| Indomethacin | 5beta-cholanic | |
| | acid 3alpha, | |
| | 7-alpha-diol | |
| Mefenamic acid | | |
| Ketofifen | | |
| Naltrexone (oploid antagonist) | | |
| Naproxen | | |
| Z-10-oxo nortriptyline | | |
| Oestrone | | |
| S-1360 (HIV integrase inhibitor) | | |
| Progesterone | | |
| Prostaglandin | | |
| Sorbinil | | |
| Testosterone | | |
| Tibolone | | |
| Tolrestat | | |

Note that AKR1C2/3 inhibitor 5beta-cholanic acid 3alpha, 7-alpha-diol, a bile acid displayed IC50s of 0.21 uM and 74.4 uM for AKR1C2 and AKR1C3, respectively, using tibolone as a substrate (see, Steckelbroeck et al., J Pharmacol Exp Ther, 316(3): 1300-1309 (2006).

In vitro results of pooled human liver cytosol (1.6 mg/mL) incubated with ¹⁴C-radiolabeled compound of Formula Ia (20 μM) are presented in FIG. 4 as well as Tables 3, 4, and 5 below. Of note, diazepam (100 μM) inhibited metabolism of ¹⁴C-radiolabeled compound of Formula Ia by 75% while midazolam and flunitrazepam inhibited 37 and 51%, respectively. Ibuprofen is capable of inhibiting metabolism of ¹⁴C-radiolabeled compound of Formula Ia by 70%.

TABLE 3
Effect of AKR competitors on metabolite formation of
compound Formula Ia.

| | AKR | % Inhibition on Metabolite |
|---|---|---|
| | Competitor | Formation of Compound |
| AKR Competitor | Conc (μM) | Formula Ia |
| Diazepam | 2 | 15.7 |
| | 10 | 38.6 |
| | 100 | 75.1 |
| Ibuprofen | 50 | 31.3 |
| | 100 | 33.4 |
| | 200 | 43.3 |
| | 1000 | 70 |
| Diazepam | 10 | 38.6 |
| Ibuprofen^{a} | 100 | 25.9 |
| Diazepam + Ibuprofen^{a} | 10 + 100 | 43.7 |
| Midazolam | 60 | 37 |
| Flunitrazepam | 60 | 51 |
| Nitrazepam | 50 | 24 |
| Celecoxib | 2 | 8.42 |
| | 10 | 9.18 |
| | 50 | 20.5 |
| Ribavirin | 10 | 1.76 |
| | 30 | 0 |
| Phenolphthalein | 100 | 86.1 |
| Naproxen | 100 | 44.7 |
| Indomethacin | 100 | 19 |
| Gemfibrozil | 100 | 27.4 |
| Phenobarbital | 100 | 0 |
| Testosterone | 40 | 48.2 |

^{a}Incubated on same day
Average of duplicate determinations

TABLE 4
Effect of Inhibitors of Cytosolic Enzymes on metabolite formation of
compound Formula Ia.

| | Cytosolic | |
|---|---|---|
| Inhibitors of | Enzyme | % Inhibition on Metabolite |
| Cytosolic | Inhibitor | Formation of Compound |
| Enzymes | Conc (μM) | Formula Ia |
| Flufenamic acid^{c} | 100 | 80.3 |
| Menadione^{c} | 100 | 29.7 |
| BNPP^{c} | 1000 | 63.4 |
| Pargyline^{c} | 1 | 0 |
| Quercetin^{c} | 100 | 33.4 |
| Allopurinol^{c} | 100 | 2 |
| Indomethacin | 100 | 19 |
| Phenobarbital | 100 | 0 |
| Naproxen | 100 | 44.7 |
| Gemfibrozil | 100 | 27.4 |
| Testosterone | 40 | 48.2 |
| Ibuprofen | 50 | 21.9 |
| | 100 | 31.4 |
| | 200 | 40.7 |
| | 500 | 56.6 |
| | 1000 | 70 |

^{c}data collected following 2 hr incubation.

TABLE 5
Effect of AKR competitors (NSAID) and other compounds on
metabolite formation of compound Formula Ia.

| | | | |
|---|---|---|---|
| | | | % of Inhibition on |
| | | | Metabolite Formation |
| Generic | | | of Compound Formula |
| Name | Trade Name | Conc (μM) | Ia (IC50) |

NSAID

| | | | |
|---|---|---|---|
| Celecoxib | Celebrex | 50 | 20.5 |
| Diclofenac | Voltaren, | 7 | 57.3 (3.43 μM) |
| | Cataflam, | | |
| | Arthrotec | | |
| Diflunisal | Dolobid | 200 | 89.4 (4.58 μM)^{a} |
| Etodolac | Lodine | 100 | 3.74 |
| Fenoprofen | Nalfon | 80 | 14.2 |
| Flurbirofen | Ansaid | 50 | 31.6 |
| Ibuprofen | Motrin, Advil | 200 | 43.3 (411.8 μM) |
| Indomethacin | Indocin | 100 | 19 |
| Ketoprofen | Oruvail | 10 | 0 |
| Ketorolac | Toradol | 4 | 0 |
| Mefenamic | Ponstel | 1 | 14.6 |
| acid | | 10 | 51.1 (6.06 μM) |
| Meloxicam | Mobic | 6 | 2.46 |
| Nabumetone | Relafen | 200 | 42.4 |
| Naproxen | Naprosyn, Alleve | 100 | 44.7 (267.9 μM) |
| Oxaprozin | Daypro | 500 | 76.1 |
| Sulindac | Clinoril | 30 | 19 |
| Tolmetin | Tolectin | 125 | 2.14 |

Other compounds

| | | | |
|---|---|---|---|
| Naringenin | — | 500 | 86.7 (21.7 μM) |
| Bergamottin | — | 10 | 10 |

Note:
data collected following 2 hr incubation using human liver cytosol.
^{a}IC50 in monkey liver cytosol = 11 μM

Incubation studies of compound Formula Ia or compound Formula XXVII with AKR Competitor

Pooled human liver microsomes (1 nmol P450/mL) and cytosol (1.6 mg/mL) were incubated with 1 and 20 μM Formula XXVII for 30 and 60 min respectively, in the presence of an NADPH-generating system (1 mM NADP, 5 mM glucose-6-phosphate and 1.5 units/mL glucose-6-phosphate dehydrogenase) and 3 mM magnesium chloride in 0.5 mL of 100 mM potassium phosphate buffer, pH 7.4. Prior to the addition of drug, the incubation mixture was preincubated for 2 min at 37° C. Reactions were initiated by addition of drug, allowed to proceed for up to 30 or 60 min at 37° C., and then terminated by the addition of 0.5 mL of ice-cold acetonitrile with 1% acetic acid. The incubation mixture was vortexed and centrifuged (˜10,000 g) at 4° C. for 15 min and supernatants were analyzed by LC-MS. Human liver microsomes and cytosol without NADPH served as negative controls. Parallel incubations with the compound of Formula Ia were used as positive controls.

Inhibition of Formula XXVII metabolism was evaluated using selective chemical inhibitors of aldo-keto reductase (100 μM flufenamic acid, 50 μM mefenamic acid, 200 μM diflunisal and 100 μM phenolphthalein). Human liver cytosol (1.6 mg protein/mL) was pre-incubated separately with various inhibitors for 15 min at room temperature followed by the addition of buffer, cofactor and substrate (20 μM). All incubations were performed as described previously for human liver cytosols. Incubation volumes were 0.5 mL and the final concentration of the organic solvents in the incubation system was less than or equal to 1% (v/v). Reactions were initiated by addition of substrate, allowed to proceed for 60 min at 37° C., and then terminated by the addition of 0.5 mL of ice-cold acetonitrile with 1% acetic acid. The incubation mixture was vortexed and centrifuged (˜10,000 g) at 4° C. for 10 min; supernatants were analyzed by LC-MS. Parallel incubations with the compound of Formula Ia were used as positive controls.

Following incubation of Formula XXVII with human liver (HL) cytosol, an ‘M+2’ metabolite (m/z=680) was formed apparently by a metabolic pathway similar to that for the formation of the ‘M+2’ metabolite (m/z=522) from the compound of Formula Ia following similar incubations. Formation of the ‘M+2’ metabolite from Formula XXVII was inhibited 2- to 4-fold following incubations of Formula XXVII in human liver cytosol in presence of AKR inhibitors such as flufenamic acid, mefenamic acid, diflunisal, and phenolphthalein (see Table 6). Formation of the ‘M+2’ metabolite from the compound of Formula Ia following similar incubations was inhibited 3- to 8-fold.

Metabolic inhibition of liver cytosolic enzymes (including AKRs) can be used clinically for improving the pharmacokinetics (PK) and/or pharmacodynamics (PD)/therapeutic outcome of Formula XXVII and the compound of Formula Ia resulting in either lower doses and/or decrease in dosing frequency.

Additional metabolic inhibition can be obtained clinically by concomitant inhibition of alternate metabolic pathways for the metabolism of Formula XXVII and/or the compound of Formula Ia. Concomitant use of inhibitors of parallel metabolic/transport pathways other than the AKR pathway would allow inhibition of these pathways that would otherwise be involved from the diversion of metabolism resulting from inhibition of the AKR pathway for example.

TABLE 6
Incubation of compound Formula Ia or compound Formula XXVII with AKR competitor.

| | | | | % M + 2/ | |
|---|---|---|---|---|---|
| | | 1^{ST} PARENT | 1^{ST} M + 2 | PARENT | FOLD |
| COMPOUND | MATRICES | PEAK AREA | PEAK AREA | INITIAL | INHIBITION |
| Formula Ia | HL Cytosol w/o | 7.41E+07 | 1.93E+06 | 2.60 | |
| | NADPH | | | | |
| Formula | HL Cytosol w/o | 3.03E+08 | 0.00E+00 | 0.00 | |
| XXVII | NADPH | | | | |
| Formula Ia | HL Cytosol w/ | 3.95E+07 | 6.78E+07 | 91.49 | |
| | NADPH Vehicle | | | | |
| | Control | | | | |
| Formula | HL Cytosol w/ | 3.03E+08 | 2.09E+07 | 6.90 | |
| XXVII | NADPH Vehicle | | | | |
| | Control | | | | |
| Formula Ia | HL Cytosol w/ | 6.33E+07 | 1.75E+07 | 23.57 | 4 |
| | NADPH + 100 uM | | | | |
| | Flufenamic acid | | | | |
| Formula | HL Cytosol w/ | 3.08E+08 | 7.82E+06 | 2.58 | 3 |
| XXVII | NADPH + 100 uM | | | | |
| | Flufenamic acid | | | | |
| Formula Ia | HL Cytosol w/ | 6.19E+07 | 2.13E+07 | 28.68 | 3 |
| | NADPH + 50 uM | | | | |
| | Mefenamic acid | | | | |
| Formula | HL Cytosol w/ | 2.92E+08 | 9.48E+06 | 3.13 | 2 |
| XXVII | NADPH + 50 uM | | | | |
| | Mefenamic acid | | | | |
| Formula Ia | HL Cytosol w/ | 6.10E+07 | 9.02E+06 | 12.18 | 8 |
| | NADPH + 200 uM | | | | |
| | Diflunisal | | | | |
| Formula | HL Cytosol w/ | 2.88E+08 | 6.55E+06 | 2.16 | 3 |
| XXVII | NADPH + 200 uM | | | | |
| | Diflunisal | | | | |
| Formula Ia | HL Cytosol w/ | 6.23E+07 | 1.18E+07 | 15.90 | 6 |
| | NADPH + 100 uM | | | | |
| | Phenolphthalein | | | | |
| Formula | HL Cytosol w/ | 2.86E+08 | 4.89E+06 | 1.61 | 4 |
| XXVII | NADPH + 100 uM | | | | |
| | Phenolphthalein | | | | |

In Vivo Inhibition Studies of Compound Formula Ia with AKR Competitor Diflunisal

An in vivo study was conducted in cynomolgus monkeys where 200 mg Formula Ia and 0 (control), 62.5, 125, or 250 mg diflunisal (Dolobid) was administered as illustrated below. All six monkeys were first dosed with Formula Ia with blood samples collected over a 12 hr period for plasma pharmacokinetics (PK) of Formula Ia prior to administration of diflunisal (dose-escalation). Four doses of diflunisal were subsequently administered every 12 hr with Formula Ia administered at each 4^{th} diflunisal dose after which blood samples were collected for PK assessment of Formula Ia. The following chart summarizes the timing of Formula Ia and diflunisal administration.

| | Amount of Compound | Amount of |
|---|---|---|
| Time | Formula Ia | Diflunisal |
| (hr) | Administered | Administered |
| 0 | 200 mg Formula Ia | |
| 12 hr | | 62.5 mg Diflunisal |
| 24 hr | | 62.5 mg Diflunisal |
| 36 hr | | 62.5 mg Diflunisal |
| 48 hr | 200 mg Formula Ia | 62.5 mg Diflunisal |
| 60 hr | | 125 mg Diflunisal |
| 72 hr | | 125 mg Diflunisal |
| 84 hr | | 125 mg Diflunisal |
| 96 hr | 200 mg Formula Ia | 125 mg Diflunisal |
| 108 hr | | 250 mg Diflunisal |
| 120 hr | | 250 mg Diflunisal |
| 132 hr | | 250 mg Diflunisal |
| 144 hr | 200 mg Formula Ia | 250 mg Diflunisal |

Comparisons of PK parameters demonstrated the following for the diflunisal-dosed monkeys compared to controls:

1. 1.3- to 2,4-fold increase in Cₘₐₓ and 1.5- to 2,3-fold increase in AUC_{(0-12 hr)} of Formula Ia as a function of diflunisal dose (similar increases were also noted for Formula Ib and Formula Ic).

2. 2.0- to 5,7-fold increase in the concentration of Formula Ia at 8 hr post-dose (similar increases were also noted for Formula Ib and Formula Ic).

3. 1.1- to 4,6-fold increase in the concentration of Formula Ia at 12 hr post-dose (similar increases were also noted for Formula Ib and Formula Ic).

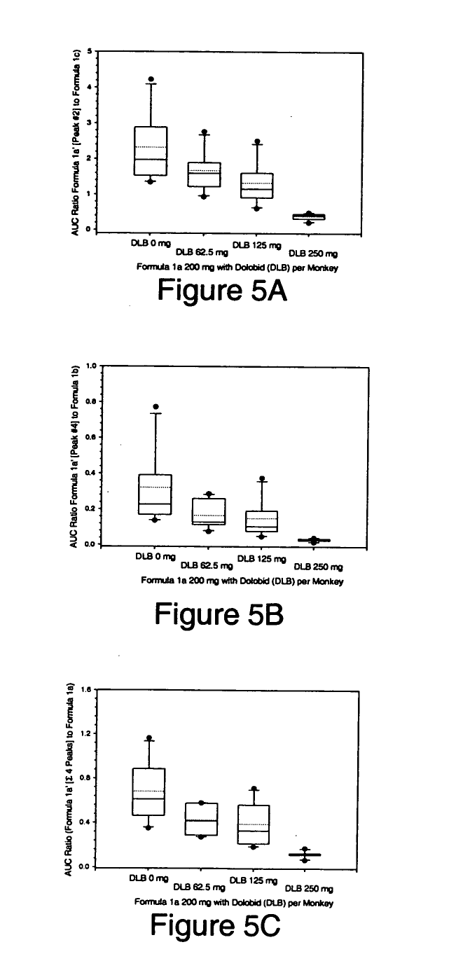

4. decline in the AUC ratio (Formula Ia′ to AUC for Formula Ia, Formula Ib, or Formula Ic; FIGS. 5A, 5B, and 5C, respectively) as a function of diflunisal suggest that the degree of inhibition of the formation of the Formula Ia′ is a function of diflunisal dose.

Clinical Study to Evaluate the Effect of AKR Substrate (Ibuprofen) on the Pharmacokinetics and Metabolism of Formula Ia

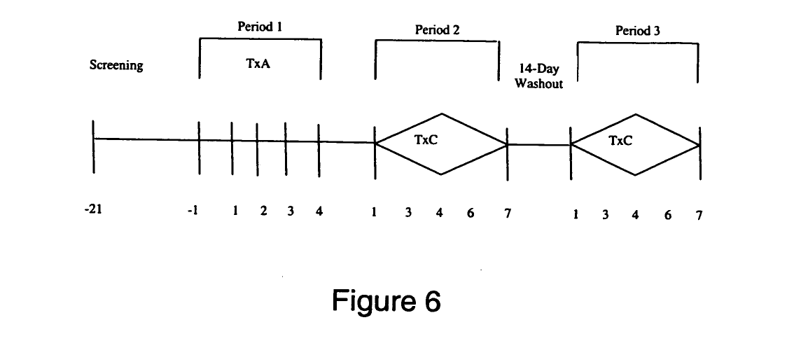

The study was conducted in an open-label, randomized, 3-period, 2-sequence crossover manner (FIG. 6). During Period 1, all 12 subjects were administered a single 400 mg dose of Formula Ia. During Periods 2 and 3, subjects received multiple doses of ibuprofen (600 mg TID) in a randomized sequence. The ibuprofen was administered beginning on Day 1 (3 days prior to Formula Ia administration) and continued through Day 6. A single dose of Formula Ia was administered on Day 4 (2 hours after administration of the AM dose of ibuprofen). Plasma samples for pharmacokinetic and metabolite analyses of Formula Ia was collected at predose (0 hour), 0.5, 1, 1.5, 2, 2.5, 3, 4, 5, 6, 7, 8, 9, 10, 12, 24, 36, 48, and 72 hour postdose for each period. (The 48 and 72 hour postdose samples for Period 1 was collected in an outpatient setting). In Periods 2 and 3, additional blood samples were collected immediately prior to dosing of the Formula Ia on Day 4 and two hours post ibuprofen administration on Day 5 for determination of ibuprofen concentration.

Treatment A: Formula Ia (4×100 mg capsules); single dose, PO following an overnight fast, administered on Day 1 or Period 1.

Treatment C: Ibuprofen 600 mg; PO, TID from Day 1 to Day 6 Formula Ia (4×100 mg capsules); single dose, PO following an overnight fast, administered on Day 4 (2 hours after the AM ibuprofen dose).

Subjects received a single dose of Formula Ia on Day 1 of Period 1. In Period 2 and Period 3, subjects were treated for 6 days with ibuprofen and received a single dose of Formula Ia on Day 4 of each period. There were at least 7 days between administration of Formula Ia in Period 1 and Period 2 and at least 14 days between administration of Formula Ia in Period 2 and 3.

The proportion of subjects with plasma concentrations above the in vitro IC₅₀ and IC₉₀ for the HCV replicon at each time point was determined. This plasma concentration data was used to estimate the following primary pharmacokinetic variables for the determination of bioavailability comparisons:
AUC—Area under the plasma concentration-time.
Cₘₐₓ—Maximum observed plasma concentration.
Tₘₐₓ—Time to maximum observed plasma concentration.
t_{1/2}—Terminal phase half-life.

The relative bioavailabilities of Formula Ia administered in the presence of the ibuprofen compared to Formula Ia administered alone are shown in Table 7.

TABLE 7
Comparison between Formula Ia treatment alone and Formula Ia
co-administered with ibuprofen for major PK parameters.
Mean (% CV) PK Parameters

| | Formula Ia | Formula Ia + ibuprofen |
|---|---|---|
| Cₘₐₓ | 571 (45) | 642 (87) |
| AUC₍ₗₐₛₜ₎ | 2001 (59) | 2013 (47) |
| AUC₍ₐₗₗ₎ | 2044 (58) | 2055 (45) |
| AUC₍ₗ₎ | 2067 (57) | 2090 (44) |
| C₈ | 48.0 (38) | 54.3 (65) |
| t_{1/2} | 9.11 (59) | 8.02 (51) |
| MRT₍ₗ₎ | 6.57 (30) | 6.91 (28) |
| t_{1/2}eff | 3.3 (26) | 4.16 (35) |
| Tₘₐₓ (median) | 1.75 | 2.00 |

A comparison between Formula Ia treatment alone and Formula Ia co-administered with ibuprofen for several PK parameters is displayed in Table 8.

TABLE 8
Comparison between Formula Ia treatment alone and Formula Ia
co-administered with ibuprofen for several PK parameters.

Formula Ia + ibuprofen

| Parameter | Ratio (%) | 90% Cl |
|---|---|---|
| Cₘₐₓ | 94 | 65-136 |
| AUC₍ₗₐₛₜ₎ | 104 | 90-121 |
| AUC₍ₗ₎ | 104 | 90-120 |
| C₈ | 118 | 82-169 |

A greater increase in the bioavailability of HCV protease inhibitor is expected with an AKR competitor having a higher IC₅₀ value than that of ibuprofen.

Clinical Study to Evaluate the Effect of AKR Substrate (Diflunisal) on the Pharmacokinetics and Metabolism of Formula Ia

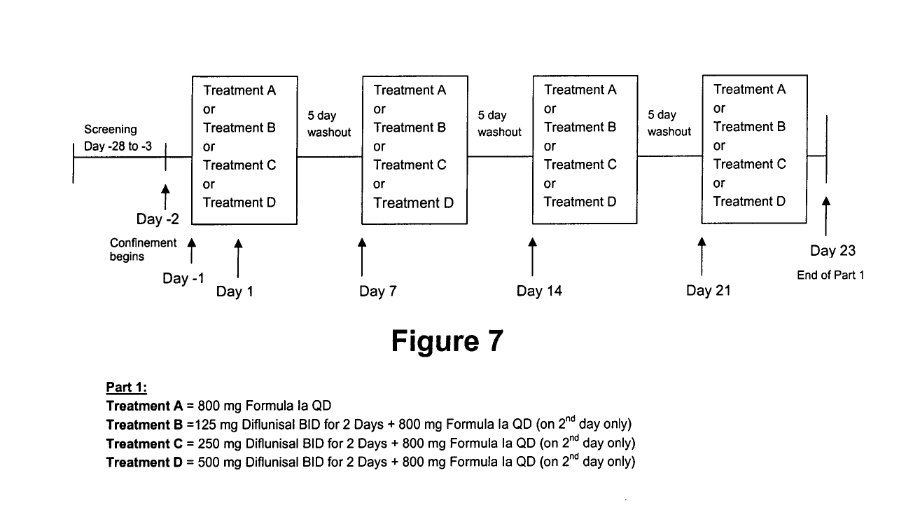

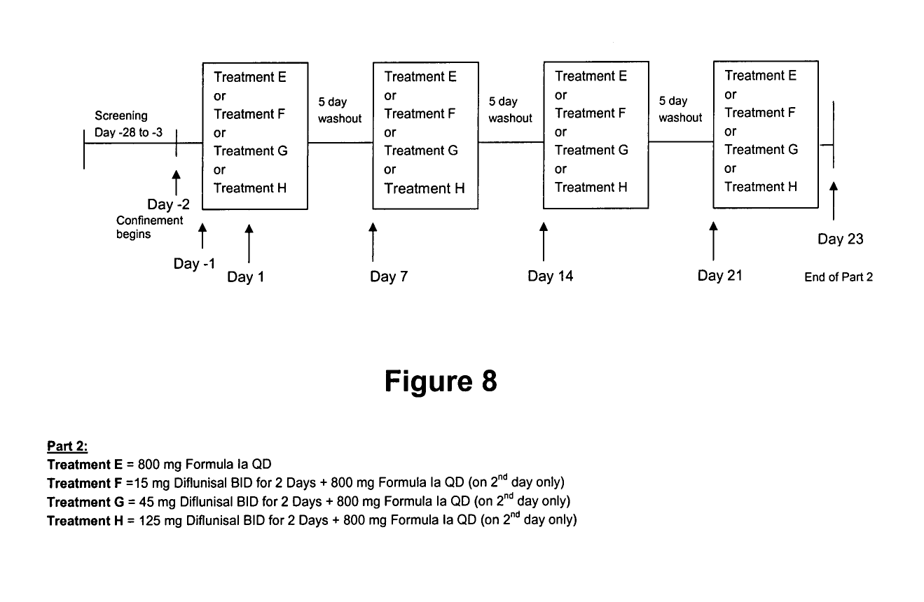

This study is an open-label, randomized, multi-part, multiple dose study in health volunteers where the pharmacokinetics and metabolism of Formula Ia administered in combination with diflunisal is assessed. In particular, the pharmacokinetic profile parameters (e.g., AUC, Cₘₐₓ, Cₘᵢₙ, Tₘₐₓ, T_{1/2}) of Formula Ia after a single dose and in combination with diflunisal are determined as well as the pharmacokinetic profile parameters of diflunisal. The study is divided into two parts based on the dose of diflunisal administered. In Part 1 (illustrated in FIG. 7), the dose of diflunisal administered is 0, 125, 250, or 500 mg. In Part 2 (illustrated in FIG. 8), the dose of diflunisal administered is 0, 15, 45, or 125 mg. Notably, diflunisal administered at a a dosage of 500 mg (BID) has a Cₘᵢₙ at steady state of 190 μg/ml where the protein binding equals about 99% and Vdss equals 0.1 L/kg. The in vitro IC₅₀ value of diflunisal measured using human cytosol equals 1 μg/ml and the Kᵢ equals 0.5 μg/ml (assuming the liver to plasma ratio equals 1). The I/Kᵢ ratio (corrected for protein binding) equals 1.9/0.5 (about 4, i.e., 3.8). Consequently, a sub-therapeutic dose (<250 mg) of diflunisal administered to humans should produce about 50% inhibition of AKR.

In both Parts 1 and 2, the medications are administered in a crossover manner. After a screening period of up to 28 days, subjects are confined at least 12 hours prior to start of any drug for baseline evaluation. Treatment days are for 2 days followed by a 5 day washout period between doses of Formula Ia. All dosing occurs with subjects confined to the study site. During the washout period subjects are furloughed form the site. All dosing occurs following a meal or snack that consist of at least 120 calories.

Part 1 - (4 way crossover):

Treatment A = 800 mg Formula Ia QD
Treatment B = 125 mg Diflunisal BID for 2 Days + 800 mg Formula
Ia QD (on 2^{nd} day only)
Treatment C = 250 mg Diflunisal BID for 2 Days + 800 mg Formula
Ia QD (on 2^{nd} day only)
Treatment D = 500 mg Diflunisal BID for 2 Days + 800 mg Formula
Ia QD (on 2^{nd} day only)
For Part 1 of the study, 8 subjects are randomized to four (4) different
treatment sequences. Subjects receive diflunisal Q12° (BID) for 2 days
and an 800 mg dose of Formula Ia (QD) on the morning of the second
day. For Treatment A, subjects receive an 800 mg dose of Formula Ia
(QD) on the morning on Days 1, 8, 15, and 22. Diflunisal doses start
on Days - 1, 7, 14, and 21 and vary according to treatment group;
Treatment B is 125 mg diflunisal, Treatment C is 250 mg Diflunisal and
Treatment D is 500 mg diflunisal. Diflunisal is administered as a tablet.
There is a 5 day washout period after all treatments.
Serial pharmacokinetic samples for Formula Ia and diflunisal are taken
0, 0.5, 1, 2, 3, 4, 5, 6, 7, 8, 12, 16, and 24 hours on Day 1, Day 8,
Day 15, and Day 22.
Formula Ia pharmacokinetic samples are analyzed for the following:
Formula Ia′, Formula Ib, and Formula Ic.

Part 2 - (4 way crossover):

Treatment E = 800 mg Formula Ia QD
Treatment F = 15 mg Diflunisal BID for 2 Days + 800 mg Formula
Ia QD (on 2^{nd} day only)
Treatment G = 45 mg Diflunisal BID for 2 Days + 800 mg Formula
Ia QD (on 2^{nd} day only)
Treatment H = 125 mg Diflunisal BID for 2 Days + 800 mg Formula
Ia QD (on 2^{nd} day only)
For Part 2 of the study, 8 subjects is randomized to four (4) different
treatment sequences. Subjects will receive diflunisal Q12° (BID)
for 2 days and an 800 mg dose of Formula Ia (QD) on the morning of
the second day. For Treatment E, subjects will receive an 800 mg dose
of Formula Ia (QD) on the morning on Days 1, 8, 15, and 22. Diflunisal
doses will start on Days - 1, 7, 14, and 21 and will vary according to
treatment group; Treatment F is 15 mg diflunisal, Treatment G is 45 mg
diflunisal and Treatment H is 125 mg diflunisal. Diflunisal is administered
as a solution. There is a 5 day washout period after all treatments.
Serial pharmacokinetic samples for Formula Ia and diflunisal is taken
0, 0.5, 1, 2, 3, 4, 5, 6, 7, 8, 12, 16, and 24 hours on Day 1, Day 8,
Day 15, and Day 22. Formula Ia pharmacokinetic samples is
analyzed for the following: Formula Ia′, Formula Ib,
and Formula Ic.

Test Product, Dose, Mode of Administration:

Formula Ia administered orally as a 200 mg capsules containing 3% SLS.

Diflunisal administered orally as a 250 mg tablet.

Diflunisal administered orally as a 500 mg tablet.

Diflunisal administered orally as a solution, to be prepared by the site.

All dosing administered after a meal or snack.

Reference Therapy, Dose, Mode of Administration: For Part 1 and 2: Formula Ia 800 mg (4×200 mg capsules containing 3% SLS), PO, ×1, following a meal or snack.

Duration of Treatment: For Part 1, subjects will receive a single 800 mg dose of Formula Ia four times; 125 mg BID of diflunisal for 2 days; 250 mg BID of diflunisal for 2 days; 500 mg BID of diflunisal for 2 days. For Part 2, subjects will receive a single 800 mg dose of Formula Ia four times; 15 mg BID of diflunisal for 2 days; 45 mg BID of diflunisal for 2 days; 125 mg BID of diflunisal for 2 days.

Safety and Tolerability: The overall safety and tolerability evaluation will include all safety data (safety labs, ECGs, AEs, and vital signs).

Pharmacokinetics: The trough levels (C₁₂ and C₂₄) after a single dose of Formula Ia alone and the trough levels after a single dose of Formula Ia with a range of diflunisal doses (BID×2 days) is compared. The following parameters of Formula Ib (active diastereomer), Formula Ic (less active diastereomer) and Formula Ia′ (metabolite) is determined: AUC, Cₘₐₓ, Cₘᵢₙ, and Tₘₐₓ.

Safety: Adverse events is tabulated by treatment. ECG parameters is summarized by treatment using descriptive statistics as well as the safety laboratory tests and vital signs.

Pharmacokinetics: Single dose plasma Formula Ia′, Formula Ib, and Formula Ic concentrations and pharmacokinetic parameters is listed and summarized by parts and treatment groups using descriptive statistics.

For Part 1 and 2, the primary pharmacokinetic parameters for Formula Ia′, Formula Ib, and Formula Ic are trough concentrations at hour 12 and 24 on the first day of Formula Ia dosing and secondary pharmacokinetic parameters are AUC and Cₘₐₓ. Log transformed pharmacokinetic parameters is analyzed for each part separately using ANOVA model for a four-way crossover extracting the effects due to treatment, sequence, period, and subject. Ratio estimate along with 90% confidence interval is calculated for the relative bioavailability of Treatment B, C or D vs. A for Part 1; Treatment F, G or H vs. E for Part 2. Additionally secondary comparisons may be made if warranted.

Preliminary analysis will include examining the pharmacokinetic parameters for extreme values by reviewing the standardized ranges of deviations from the expected value derived from the model to see if any value exceeds 3. The impact of any outlier on the results of the analyses is evaluated.

The inventors believe that the aforementioned study will demonstrate that an AKR competitor (illustrated by diflunisal) will increase the bioavailability of a HCV protease inhibitor (including, but not limited to, a compound of Formula Ia, Ib, or Ic, or a pharmaceutically acceptable salt, solvate or ester thereof).

It is appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

Each document (including granted patents, published patent applications, and nonpatent publications such as journal articles) referred to in this application is incorporated in its entirety by reference for all purposes.

What is claimed is:

1. A medicament comprising, separately or together:
(a) at least one aldo-keto reductase (AKR) competitor; and
(b) at least one hepatitis C virus (HCV) protease inhibitor;
or a mixture of two or more thereof for concurrent or consecutive administration in treating or ameliorating one or more symptoms of HCV, or disorders associated with HCV in a subject in need thereof.

2. A medicament comprising, separately or together:
(a) at least one AKR competitor; and
(b) at least one compound is Formula I to XXVII below: 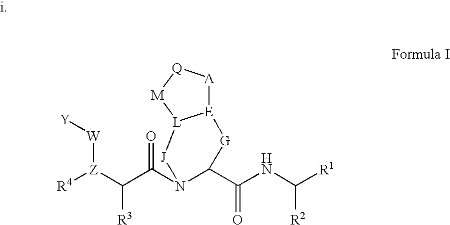
or a pharmaceutically acceptable salt, solvate or ester thereof;
wherein:
Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;
R¹ is COR⁵, wherein R⁵ is COR⁷ wherein R⁷ is NHR⁹, wherein R⁹ is selected from the group consisting of H, alkyl, aryl, heteroalkyl, heteroaryl, cycloalkyl, cycloalkyl, arylalkyl, heteroarylalkyl, [CH(R¹′)]ₚCOOR¹¹, [CH(R¹′)]ₚCONR¹²R¹³, [CH(R¹′)]ₚSO₂R¹¹, [CH(R¹′)]ₚCOR¹¹, [CH(R¹′) ]ₚCH(OH)R¹¹, CH(R¹′)CONHCH(R²)COOR¹¹, CH(R¹′)CONHCH(R²′)CONR¹²R¹³, CH(R¹′)CONHCH(R²)R′, CH(R¹′)CONHCH(R²′)CONHCH(R³′)COOR¹¹, CH(R¹′)CONHCH(R²′)CONHCH(R³′)CONR¹²R¹³, CH(R¹′)CONHCH(R²′)CONHCH(R³′)CONHCH(R⁴′)COOR¹¹, CH(R¹′)CONHCH(R²′)CONHCH(R³′)CONHCH(R⁴′)CONR¹²R¹³, CH(R¹′)CONHCH(R²′)CONHCH(R³′)CONHCH(R⁴′)CONHCH(R⁵′)COOR¹¹ and CH(R¹′)CONHCH(R²′)CONHCH(R³′)CONHCH(R⁴′)CONHCH(R⁵′) CONR¹²R¹³, wherein R¹′, R²′, R³′, R⁴′, R⁵′, R¹¹, R¹², R¹³, and R′ are independently selected from the group consisting of H, alkyl, aryl, heteroalkyl, heteroaryl, cycloalkyl, alkyl-aryl, alkyl-heteroaryl, aryl-alkyl and heteroaralkyl;
Z is selected from O, N, CH or CR;
W maybe present or absent, and if W is present, W is selected from C═O, C═S, C(═N—CN), or SO₂;
Q maybe present or absent, and when Q is present, Q is CH, N, P, (CH₂)ₚ, (CHR)ₚ, (CRR′)ₚ, O, NR, S, or SO₂; and when Q is absent, M may be present or absent; when Q and M are absent, A is directly linked to L;
A is O, CH₂, (CHR)ₚ, (CHR—CHR′)ₚ, (CRR′)ₚ, NR, S, SO₂ or a bond;
E is CH, N, CR, or a double bond towards A, L or G;
G may be present or absent, and when G is present, G is (CH₂)ₚ, (CHR)ₚ, or (CRR′)ₚ; and when G is absent, J is present and E is directly connected to the carbon atom in Formula I as G is linked to;
J may be present or absent, and when J is present, J is (CH₂)ₚ, (CHR)ₚ, or (CRR′)ₚ, SO₂, NH, NR or O; and when J is absent, G is present and E is directly linked to N shown in Formula I as linked to J;
L may be present or absent, and when L is present, L is CH, CR, O, S or NR; and when L is absent, then M may be present or absent; and if M is present with L being absent, then M is directly and independently linked to E, and J is directly and independently linked to E;
M may be present or absent, and when M is present, M is O, NR, S, SO₂, (CH₂)ₚ, (CHR)ₚ (CHR—CHR′)ₚ, or (CRR′)ₚ;
p is a number from 0 to 6; and
R, R′, R², R³ and R⁴ are independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen;
(cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally and chemically-suitably substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
further wherein said unit N-C-G-E-L-J-N represents a five-membered or six-membered cyclic ring structure with the proviso that when said unit N-C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of the cyclic ring; 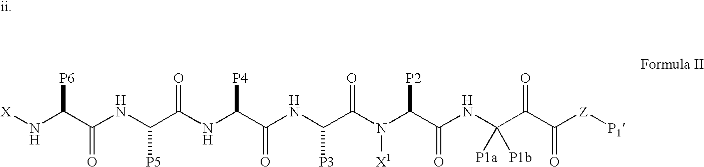
or a pharmaceutically acceptable salt, solvate or ester thereof;
wherein:
Z is NH;
X is alkylsulfonyl, heterocyclylsulfonyl, heterocyclylalkylsulfonyl, arylsulfonyl, heteroarylsulfonyl, alkylcarbonyl, heterocyclylcarbonyl, heterocyclylalkylcarbonyl, arylcarbonyl, heteroarylcarbonyl, alkoxycarbonyl, heterocyclyloxycarbonyl, aryloxycarbonyl, heteroaryloxycarbonyl, alkyaminocarbonyl, heterocyclylaminocarbonyl, arylaminocarbonyl, or heteroarylaminocarbonyl moiety, with the proviso that X may be additionally optionally substituted with R¹² or R¹³;
X¹ is H; C₁-C₄ straight chain alkyl; C₁-C₄ branched alkyl or; CH₂-aryl (substituted or unsubstituted);
R¹² is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that R¹² may be additionally optionally substituted with R¹³.
R¹³ is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro moiety, with the proviso that the alkyl, alkoxy, and aryl may be additionally optionally substituted with moieties independently selected from R¹³.
P1a, P1b, P2, P3, P4, P5, and P6 are independently: H; C1-C10 straight or branched chain alkyl; C2-C10 straight or branched chain alkenyl; C3-C8 cycloalkyl, C3-C8 heterocyclic; (cycloalkyl)alkyl or (heterocyclyl)alkyl, wherein said cycloalkyl is made up of 3 to 8 carbon atoms, and zero to 6 oxygen, nitrogen, sulfur, or phosphorus atoms, and said alkyl is of 1 to 6 carbon atoms;
aryl, heteroaryl, arylalkyl, or heteroarylalkyl, wherein said alkyl is of 1 to 6 carbon atoms;
wherein said alkyl, alkenyl, cycloalkyl, heterocyclyl; (cycloalkyl)alkyl and (heterocyclyl)alkyl moieties may be optionally substituted with R¹³, and further wherein said P1a and P1b may optionally be joined to each other to form a spirocyclic or spiroheterocyclic ring, with said spirocyclic or spiroheterocyclic ring containing zero to six oxygen, nitrogen, sulfur, or phosphorus atoms, and may be additionally optionally substituted with R¹³; and
P1′ is H, alkyl, alkenyl, alkynyl, cycloalkyl, cycloalkyl-alkyl, heterocyclyl, heterocyclyl-alkyl, aryl, aryl-alkyl, heteroaryl, or heteroaryl-alkyl; with the proviso that said P1′ may be additionally optionally substituted with R¹³; 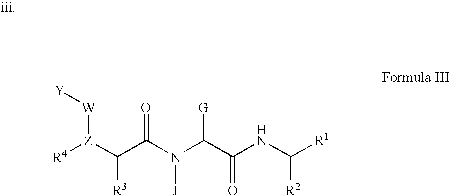
or a pharmaceutically acceptable salt, solvate or ester thereof;
wherein in Formula III:
G is carbonyl;
J and Y may be the same or different and are independently selected from the group consisting of the moieties: H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe additionally optionally substituted with X¹¹ or X¹²;
X¹¹ is selected from the group consisting of alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl moiety, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;
R¹ is COR⁵ or B(OR)₂, wherein R⁵ is selected from the group consisting of H, OH, OR⁸, NR⁹R¹⁰, CF₃, C₂F₅, C₃F₇, CF₂R⁶, R⁶ and COR⁷ wherein R⁷ is selected from the group consisting of H, OH, OR⁸, CHR⁹R¹⁰, and NR⁹R¹⁰, wherein R⁶, R⁸, R⁹ and R¹⁰ may be the same or different and are independently selected from the group consisting of H, alkyl, aryl, heteroalkyl, heteroaryl, cycloalkyl, cycloalkyl, arylalkyl, heteroarylalkyl, CH(R¹′)COOR¹¹, CH(R¹′)CONR¹²R¹³, CH(R¹′)CONHCH(R²′)COOR¹¹, CH(R¹′)CONHCH(R²′)CONR¹²R¹³, CH(R¹′)CONHCH(R²′)R′, CH(R¹′)CONHCH(R²′)CONHCH(R³′)COOR¹¹, CH(R¹′)CONHCH(R²′)CONHCH(R³′)CONR¹²R¹³, CH(R¹′)CONHCH(R²′)CONHCH(R³′)CONHCH(R⁴′)COOR¹¹, CH(R¹′)CONHCH(R²′)CONHCH(R³′)CONHCH(R^{4′})CONR¹²R¹³, CH(R¹′)CONHCH(R²′)CONHCH(R³′)CONHCH(R⁴′)CONHCH(R⁵′)COO R¹¹, and CH(R¹′)CONHCH(R²′)CONHCH(R³′)CONHCH(R⁴′)CONHCH(R⁵′)CONR¹²R¹³,
wherein R¹′, R²′, R³′, R⁴′, R⁵′, R¹¹, R¹², R¹³, and R′ may be the same or different and are independently selected from a group consisting of H, alkyl, aryl, heteroalkyl, heteroaryl, cycloalkyl, alkyl-aryl, alkyl-heteroaryl, aryl-alkyl and heteroaralkyl;
Z is selected from O, N, or CH;
W maybe present or absent, and if W is present, W is selected from C═O, C═S, or SO₂; and
R, R′, R², R³ and R⁴ are independently selected from the group consisting of H; C1-C10 alkyl; C2-C10 alkenyl; C3-C8 cycloalkyl; C3-C8 heterocycloalkyl, alkoxy, aryloxy, alkylthio, arylthio, amino, amido, ester, carboxylic acid, carbamate, urea, ketone, aldehyde, cyano, nitro; oxygen, nitrogen, sulfur, or phosphorus atoms (with said oxygen, nitrogen, sulfur, or phosphorus atoms numbering zero to six); (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamide, sulfoxide, sulfone, sulfonylurea, hydrazide, and hydroxamate; 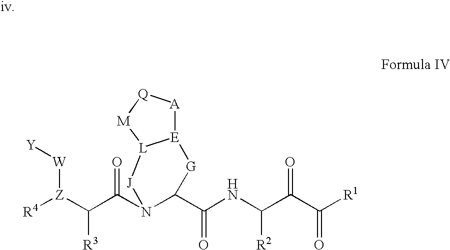
or a pharmaceutically acceptable salt, solvate or ester thereof;
wherein in Formula IV:
Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y maybe optionally substituted with X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X¹¹ may be additionally optionally substituted with X¹²;
X¹² is hydroxy, alkoxy, aryloxy, thio, alkylthio, arylthio, amino, alkylamino, arylamino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X¹²;
R¹ is selected from the following structures: 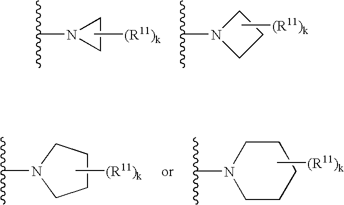
wherein k is a number from 0 to 5, which can be the same or different, R¹¹ denotes optional substituents, with each of said substituents being independently selected from the group consisting of alkyl, alkenyl, alkynyl, aryl, cycloalkyl, alkyl-aryl, heteroalkyl, heteroaryl, aryl-heteroaryl, alkyl-heteroaryl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino, heterocycloalkylamino, hydroxy, thio, alkylthio, arylthio, amino, alkylsulfonyl, arylsulfonyl, alkylsulfonamido, arylsulfonamido, carboxyl, carbalkoxy, carboxamido, alkoxycarbonylamino, alkoxycarbonyloxy, alkylureido, arylureido, halogen, cyano, and nitro, with the proviso that R¹¹ (when R¹¹ ≠H) maybe optionally substituted with X¹¹ or X¹²;
Z is selected from O, N, CH or CR;
W may be present or absent, and if W is present, W is selected from C═O, C═S, C(═N—CN), or S(O₂);
Q may be present or absent, and when Q is present, Q is CH, N, P, (CH₂)ₚ, (CHR)ₚ, (CRR′)ₚ, O, N(R), S, or S(O₂); and when Q is absent, M may be present or absent; when Q and M are absent, A is directly linked to L;
A is O, CH₂, (CHR)ₚ, (CHR—CHR′) p, (CRR′) p, N(R), S, S(O₂) or a bond;
E is CH, N, CR, or a double bond towards A, L or G;
G may be present or absent, and when G is present, G is (CH₂)ₚ, (CHR)ₚ, or (CRR′)ₚ; and when G is absent, J is present and E is directly connected to the carbon atom in Formula I as G is linked to;
J may be present or absent, and when J is present, J is (CH₂)ₚ, (CHR)ₚ, or (CRR′)ₚ, S(O₂), NH, N(R) or O; and when J is absent, G is present and E is directly linked to N shown in Formula I as linked to J;
L may be present or absent, and when L is present, L is CH, C(R), O, S or N(R);
and when L is absent, then M may be present or absent; and if M is present with L being absent, then M is directly and independently linked to E, and J is directly and independently linked to E;
M may be present or absent, and when M is present, M is O, N(R), S, S(O₂), (CH₂)ₚ, (CHR)ₚ (CHR—CHR′)ₚ, or (CRR′) p;
p is a number from 0 to 6; and
R, R′, R², R³ and R⁴ can be the same or different, each being independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen, (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl;
wherein said alkyl, heteroalkyl, alkenyl, heteroalkenyl, aryl, heteroaryl, cycloalkyl and heterocycloalkyl moieties may be optionally substituted, with said term “substituted” referring to substitution with one or more moieties which can be the same or different, each being independently selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate;
further wherein said unit N-C-G-E-L-J-N represents a five-membered cyclic ring structure or six-membered cyclic ring structure with the proviso that when said unit N-C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic ring structure lacks a carbonyl group as part of said five-membered cyclic ring; 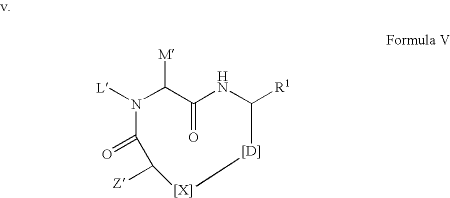
or a pharmaceutically acceptable salt, solvate or ester thereof;
wherein in Formula V:
(1) R¹ is —C(O)R⁵ or —B(OR)₂;
(2) R⁵ is H, —OH, —OR⁸, —NR⁹R¹⁰, —C(O)OR⁸, —C(O)NR⁹R¹⁰, —CF₃, —C₂F₅, C₃F₇, —CF₂R⁶, —R⁶, —C(O)R⁷ or NR⁷SO₂R⁸;
(3) R⁷ is H, —OH, —OR⁸, or —CHR⁹R¹⁰;
(4) R⁶, R⁸, R⁹ and R¹⁰ are independently selected from the group consisting of H: alkyl, alkenyl, aryl, heteroalkyl, heteroaryl, cycloalkyl, arylalkyl, heteroarylalkyl, R¹⁴, —CH(R¹′)CH(R¹′)C(O)OR¹¹, [CH(R¹′)]ₚC(O)OR¹¹, —[CH(R¹′)]ₚC(O)NR¹²R¹³, —[CH(R¹′)]ₚS(O₂)R¹¹, —[CH(R¹′)]ₚC(O)R¹¹, —[CH(R¹′)]ₚS(O₂)NR¹²R¹³, CH(R¹′)C(O)N(H)CH(R²′)(R′), CH(R¹′)CH(R¹′)C(O)NR¹²R¹³, —CH(R¹′)CH(R¹′)S(O₂)R¹′, —CH(R¹′)CH(R¹′)S(O₂)NR¹²R¹³, —CH(R¹′)CH(R¹′)C(O)R¹¹, —[CH(R¹′)]ₚCH(OH)R¹¹, —CH(R¹′)C(O)N(H)CH(R²′)C(O)OR¹¹, C(O)N(H)CH(R²′)C(O)OR¹¹, —C(O)N(H)CH(R²′)C(O)R¹′, CH(R¹′)C(O)N(H)CH(R²′) C(O)NR¹²R¹³, —CH(R¹′)C(O)N(H)CH(R²′)R′, CH(R¹′)C(O)N(H)CH(R²′)C(O)N(H) CH(R³′)C(O)OR¹¹, CH(R¹′)C(O)N(H)CH(R²′)C(O)CH(R³′)NR¹²R¹³, CH(R¹′)C(O)N(H)C H(R²′)C(O)N(H)CH(R³′)C(O)NR¹²R¹³, CH(R¹′)C(O)N(H)CH(R²′)C(O)N(H)CH(R³′)C(O)N(H)CH(R⁴′)C(O)OR¹¹, H(R¹′)C(O)N(H)CH(R²′)C(O)N(H)CH(R³′)C(O)N(H)CH(R⁴′)C(O)NR¹²R¹³, CH(R¹′)C(O)N(H)CH(R²′)C(O)N(H)CH(R³′)C(O)N(H)CH(R⁴′)C(O)N(H)CH(R⁵′)C(O)OR¹¹, and CH(R¹′)C(O)N(H)CH(R²′)C(O)N(H)CH(R³′)C(O)N(H)CH(R⁴′)C(O)N(H)CH(R⁵′) C(O)NR¹²R¹³;
wherein R¹′, R²′, R³′, R⁴′, R⁵′, R¹¹, R¹² and R¹³ can be the same or different, each being independently selected from the group consisting of: H, halogen, alkyl, aryl, heteroalkyl, heteroaryl, cycloalkyl, alkoxy, aryloxy, alkenyl, alkynyl, alkyl-aryl, alkyl-heteroaryl, heterocycloalkyl, aryl-alkyl and heteroaralkyl;
or
R¹² and R¹³ are linked together wherein the combination is cycloalkyl, heterocycloalkyl, ary or heteroaryl;
R¹⁴ is present or not and if present is selected from the group consisting of: H, alkyl, aryl, heteroalkyl, heteroaryl, cycloalkyl, alkyl-aryl, allyl, alkyl-heteroaryl, alkoxy, aryl-alkyl, alkenyl, alkynyl and heteroaralkyl;
(5) R and R′ are present or not and if present can be the same or different, each being independently selected from the group consisting of: H, OH, C₁-C₁₀ alkyl, C₂-C₁₀ alkenyl, C₃-C₈ cycloalkyl, C₃-C₈ heterocycloalkyl, alkoxy, aryloxy, alkylthio, arylthio, alkylamino, arylamino, amino, amido, arylthioamino, arylcarbonylamino, arylaminocarboxy, alkylaminocarboxy, heteroalkyl, alkenyl, alkynyl, (aryl)alkyl, heteroarylalkyl, ester, carboxylic acid, carbamate, urea, ketone, aldehyde, cyano, nitro, halogen, (cycloalkyl)alkyl, aryl, heteroaryl, (alkyl)aryl, alkylheteroaryl, alkyl-heteroaryl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms;
(6) L′ is H, OH, alkyl, heteroalkyl, aryl, heteroaryl, cycloalkyl, or heterocyclyl;
(7) M′ is H, alkyl, heteroalkyl, aryl, heteroaryl, cycloalkyl, arylalkyl, heterocyclyl or an amino acid side chain;
or L′ and M′ are linked together to form a ring structure wherein the portion of structural Formula 1 represented by 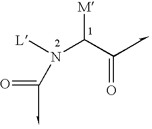
is represented by structural Formula 2: 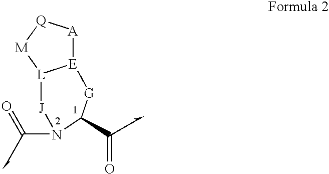
wherein in Formula 2:
E is present or absent and if present is C, CH, N or C(R);
J is present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;
p is a number from 0 to 6;
L is present or absent, and when L is present, L is C(H) or C(R); when L is absent, M is present or absent; if M is present with L being absent, then M is directly and independently linked to E, and J is directly and independently linked to E;
G is present or absent, and when G is present, G is (CH₂)ₚ, (CHR)ₚ, (CHR—CHR′)ₚ or (CRR′)ₚ; when G is absent, J is present and E is directly connected to the carbon atom marked position 1;
Q is present or absent, and when Q is present, Q is NR, PR, (CR═CR),
(CH₂)ₚ, (CHR)ₚ, (CRR′)ₚ, (CHR—CHR′)ₚ, O, NR, S, SO, or SO₂; when Q is absent, M is (i) either directly linked to A or (ii) an independent substituent on L, said independent substituent bing selected from —OR, —CH(R)(R′), S(O)₀₋₂R or —NRR′ or (iii) absent; when both Q and M are absent, A is either directly linked to L, or A is an independent substituent on E, said independent substituent being selected from —OR, —CH(R)(R′), S(O)₀₋₂R or —NRR′ or A is absent;
A is present or absent and if present A is O, O(R), (CH₂)ₚ, (CHR)ₚ, (CHR—CHR′)ₚ, (CRR′)ₚ, N(R), NRR′, S, S(O₂), —OR, CH(R)(R′) or NRR′; or A is linked to M to form an alicyclic, aliphatic or heteroalicyclic bridge;
M is present or absent, and when M is present, M is halogen, O, OR, N(R), S, S(O₂), (CH₂)ₚ, (CHR)ₚ(CHR—CHR′)ₚ, or (CRR′)ₚ; or M is linked to A to form an alicyclic, aliphatic or heteroalicyclic bridge;
(8) Z′ is represented by the structural Formula 3: 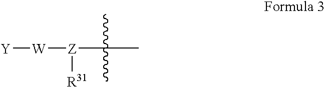
wherein in Formula 3:
Y is selected from the group consisting of: H, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, heteroalkyl-heterocycloalkyl, cycloalkyloxy, alkylamino, arylamino, alkyl-arylamino, arylamino, heteroarylamino, cycloalkylamino and heterocycloalkylamino, and Y is unsubstituted or optionally substituted with one or two substituents which are the same or different and are independently selected from X¹¹ or X¹²;
X¹¹ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹¹ is unsubstituted or optionally substituted with one or more of X¹² moieties which are the same or different and are independently selected;
X¹² is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, sulfonylurea, cycloalkylsulfonamido, heteroaryl-cycloalkylsulfonamido, heteroaryl-sulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;
Z is O, N, C(H) or C(R);
R³¹ is H, hydroxyl, aryl, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, heteroalkyl-heteroaryl, cycloalkyloxy, alkylamino, arylamino, alkyl-arylamino, arylamino, heteroarylamino, cycloalkylamino or heterocycloalkylamino, and R³¹ is unsubstituted or optionally substituted with one or two substituents which are the same or different and are independently selected from X¹³ or X¹⁴;
X¹³ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, and X¹³ is unsubstituted or optionally substituted with one or more of X¹⁴ moieties which are the same or different and are independently selected;
X¹⁴ is hydroxy, alkoxy, alkyl, alkenyl, alkynyl, aryl, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkylcarbonyl, arylcarbonyl, heteroalkylcarbonyl, heteroarylcarbonyl, cycloalkylsulfonamido, heteroaryl-cycloalkylsulfonamido, heteroarylsulfonamido, alkoxycarbonylamino, alkoxycarbonyloxy, alkylureido, arylureido, halogen, cyano, or nitro, and said alkyl, alkoxy, and aryl are unsubstituted or optionally independently substituted with one or more moieties which are the same or different and are independently selected from alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl;
W may be present or absent, and if W is present, W is C(═O), C(═S), C(═N—CN), or S(O₂);
(9) X is represented by structural Formula 4: 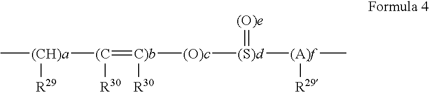
wherein in Formula 4:
a is 2, 3, 4, 5, 6, 7, 8 or 9;
b, c, d, e and f are 0, 1, 2, 3, 4 or 5;
A is C, N, S or O;
R²⁹ and R²⁹′ are independently present or absent and if present can be the same or different, each being independently one or two substituents independently selected from the group consisting of: H, halo, alkyl, aryl, cycloalkyl, cycloalkylamino, cycloalkylaminocarbonyl, cyano, hydroxy, alkoxy, alkylthio, amino, —NH(alkyl), —NH(cycloalkyl), —N(alkyl)₂, carboxyl, C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or
R²⁹ and R²⁹′ are linked together such that the combination is an aliphatic or heteroaliphatic chain of 0 to 6 carbons;
R³⁰ is present or absent and if present is one or two substituents independently selected from the group consisting of: H, alkyl, aryl, heteroaryl and cylcoalkyl;
(10) D is represented by structural Formula 5: 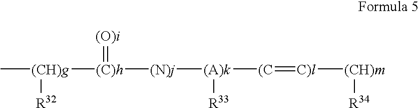
wherein in Formula 5:
R³², R³³ and R³⁴ are present or absent and if present are independently one or two substituents independently selected from the group consisting of: H, halo, alkyl, aryl, cycloalkyl, cycloalkylamino, spiroalkyl, cycloalkylaminocarbonyl, cyano, hydroxy, alkoxy, alkylthio, amino, —NH(alkyl), —NH(cycloalkyl), —N(alkyl)₂, carboxyl, —C(O)O-alkyl, heteroaryl, aralkyl, alkylaryl, aralkenyl, heteroaralkyl, alkylheteroaryl, heteroaralkenyl, hydroxyalkyl, aryloxy, aralkoxy, acyl, aroyl, nitro, aryloxycarbonyl, aralkoxycarbonyl, alkylsulfonyl, arylsulfonyl, heteroarylsulfonyl, alkylsulfinyl, arylsulfinyl, heteroarylsulfinyl, arylthio, heteroarylthio, aralkylthio, heteroaralkylthio, cycloalkenyl, heterocyclyl, heterocyclenyl, Y₁Y₂N-alkyl-, Y₁Y₂NC(O)— and Y₁Y₂NSO₂—, wherein Y₁ and Y₂ can be the same or different and are independently selected from the group consisting of hydrogen, alkyl, aryl, and aralkyl; or
R³² and R³⁴ are linked together such that the combination forms a portion of a cycloalkyl group;
g is 1,2,3,4,5,6,7,8 or 9;
h, i, j, k, l and m are 0, 1, 2, 3, 4 or 5; and
A is C, N, S or O,
(11) provided that when structural Formula 2: 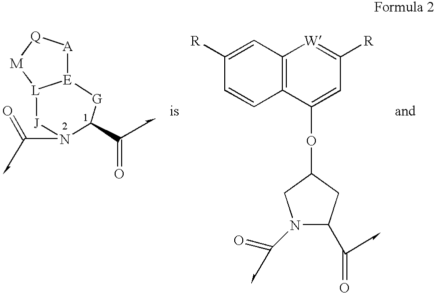
W′ is CH or N, both the following conditional exclusions (i) and (ii) apply: conditional exclusion (i): Z′ is not —NH—R³⁶, wherein R³⁶ is H, C_{6 or 10} aryl, heteroaryl, —C(O)—R³⁷, —C(O)—OR³⁷ or —C(O)—NHR³⁷, wherein R³⁷ is C₁₋₆ alkyl or C₃₋₆cycloalkyl; and
conditional exclusion (ii): R¹ is not —C(O)OH, a pharmaceutically acceptable salt of —C(O)OH, an ester of —C(O)OH or —C(O)NHR³⁸ wherein R³⁸ is selected from the group consisting of C₁₋₈ alkyl, C₃₋₆ cycloalkyl, C_{6 to 10} aryl or C₇₋₁₆ aralkyl; 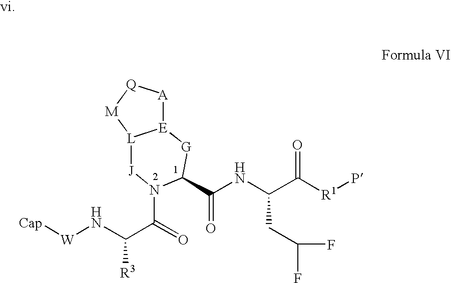
or a pharmaceutically acceptable salt, solvate or ester thereof;
wherein in Formula V1:
Cap is H, alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arlylalkyloxy or heterocyclylamino, wherein each of said alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, arlylalkyloxy or heterocyclylamino can be unsubstituted or optionally independently substituted with one or two substituents which can be the same or different and are independently selected from X¹ and X²;
P′ is —NHR;
X¹ is alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl, or heteroarylalkyl, and X¹ can be unsubstituted or optionally independently substituted with one or more of X² moieties which can be the same or different and are independently selected;
X² is hydroxy, alkyl, aryl, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, keto, ester or nitro, wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, arylalkyl, arylheteroaryl, heteroaryl, heterocyclylamino, alkylheteroaryl and heteroarylalkyl;
W may be present or absent, and when W is present W is C(═O), C(═S), C(═NH), C(═N—OH), C(═N—CN), S(O) or S(O₂);
Q maybe present or absent, and when Q is present, Q is N(R), P(R), CR═CR′, (CH₂)ₚ, (CHR)ₚ, (CRR′)ₚ, (CHR—CHR′)ₚ, O, S, S(O) or S(O₂); when Q is absent, M is (i) either directly linked to A or (ii) M is an independent substituent on L and A is an independent substituent on E, with said independent substituent being selected from —OR, —CH(R′), S(O)₀₋₂R or —NRR′; when both Q and M are absent, A is either directly linked to L, or A is an independent substituent on E, selected from —OR, CH(R)(R′), —S(O)₀₋₂R or —NRR′;
A is present or absent and if present A is —O—, —O(R) CH₂—, —(CHR)ₚ—, —(CHR—CHR′)ₚ—, (CRR′)ₚ, N(R), NRR′, S, or S(O₂), and when Q is absent, A is —OR, —CH(R)(R′) or —NRR′; and when A is absent, either Q and E are connected by a bond or Q is an independent substituent on M;
E is present or absent and if present E is CH, N, C(R);
G may be present or absent, and when G is present, G is (CH₂)ₚ, (CHR)ₚ, or (CRR′)ₚ; when G is absent, J is present and E is directly connected to the carbon atom marked position 1;
J may be present or absent, and when J is present, J is (CH₂)ₚ, (CHR—CHR′)ₚ, (CHR)ₚ, (CRR′)ₚ, S(O₂), N(H), N(R) or O; when J is absent and G is present, L is directly linked to the nitrogen atom marked position 2;
L may be present or absent, and when L is present, L is CH, N, or CR; when L is absent, M is present or absent; if M is present with L being absent, then M is directly and independently linked to E, and J is directly and independently linked to E;
M may be present or absent, and when M is present, M is O, N(R), S, S(O₂), (CH₂)ₚ, (CHR)ₚ, (CHR—CHR′)ₚ, or (CRR′)ₚ;
p is a number from 0 to 6;
R, R′ and R³ can be the same or different, each being independently selected from the group consisting of: H, C₁-C₁₀ alkyl, C₂-C₁₀ alkenyl, C₃-C₈ cycloalkyl, C₃-C₈ heterocyclyl, alkoxy, aryloxy, alkylthio, arylthio, amino, amido, arylthioamino, arylcarbonylamino, arylaminocarboxy, alkylaminocarboxy, heteroalkyl, heteroalkenyl, alkenyl, alkynyl, aryl-alkyl, heteroarylalkyl, ester, carboxylic acid, carbamate, urea, ketone, aldehyde, cyano, nitro, halogen, (cycloalkyl)alkyl, aryl, heteroaryl, alkyl-aryl, alkylheteroaryl, alkyl-heteroaryl and (heterocyclyl)alkyl;
R and R′ in (CRR′) can be linked together such that the combination forms a cycloalkyl or heterocyclyl moiety; and
R¹ is carbonyl; 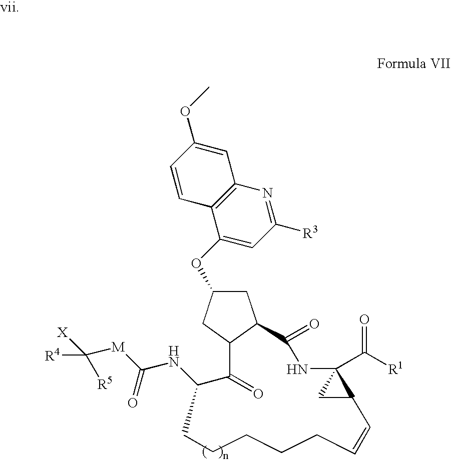
or a pharmaceutically acceptable salt, solvate or ester thereof;
wherein in Formula VII:
M is O, N(H), or CH₂;
n is 0-4;
R¹ is —OR⁶, —NR⁶R⁷ or 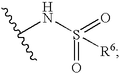
where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 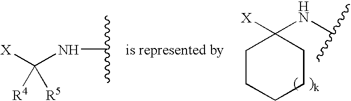
where k is 0 to 2;
X is selected from the group consisting of: 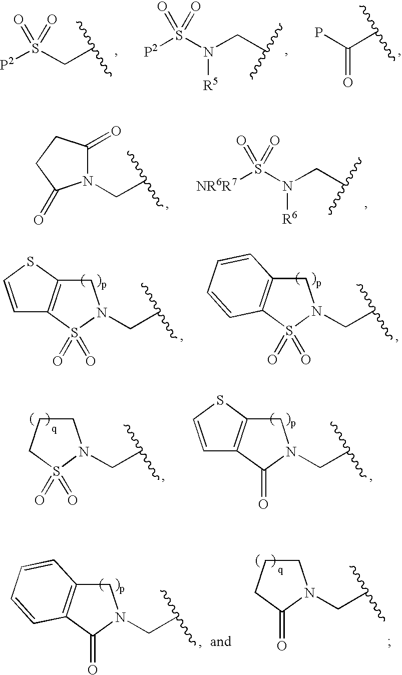
where p is 1 to 2, q is 1-3 and P² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 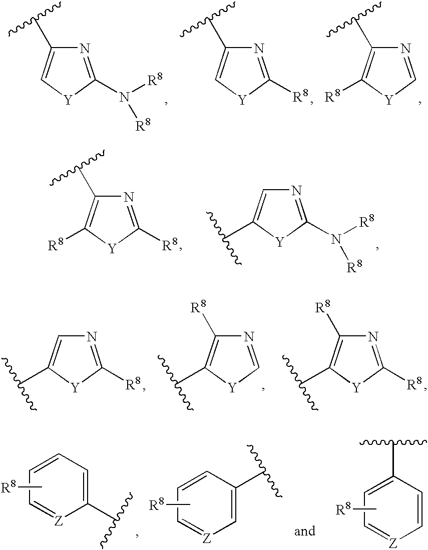
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy; 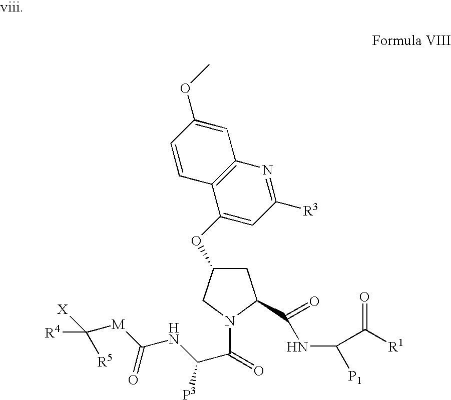
or a pharmaceutically acceptable salt, solvate or ester thereof;
wherein in Formula VIII:
M is O, N(H), or CH₂;
R¹ is —C(O)NHR⁶, where R⁶ is hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino or alkylamino;
P₁ is selected from the group consisting of alkyl, alkenyl, alkynyl, cycloalkyl haloalkyl;
P₃ is selected from the group consisting of alkyl, cycloalkyl, aryl and cycloalkyl fused with aryl;
R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 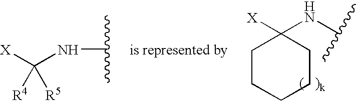
where k is 0 to 2;
X is selected from the group consisting of: 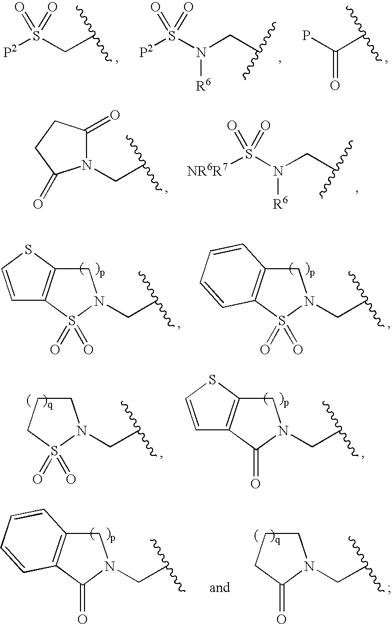
where p is 1 to 2, q is 1 to 3 and P² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 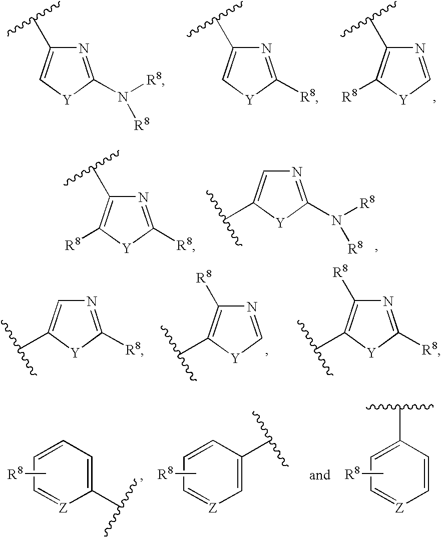
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy; 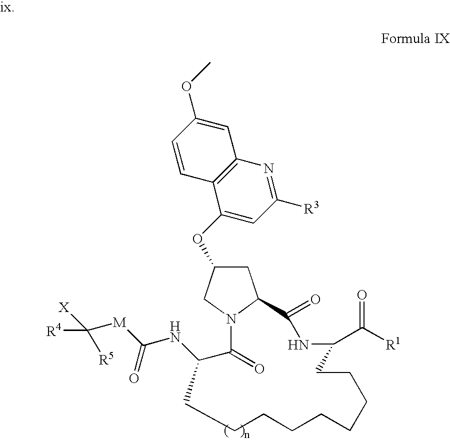
or a pharmaceutically acceptable salt, solvate or ester thereof;
wherein in Formula IX:
M is O, N(H), or CH₂;
n is 0-4;
R¹ is —OR⁶, —NR⁶R⁷ or 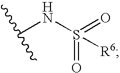
where R⁶ and R⁷ can be the same or different, each being independently selected from the group consisting of hydrogen, alkyl, alkenyl, alkynyl, heteroalkyl, cycloalkyl, cycloalkylalkyl, aryl, arylalkyl, heteroaryl, heteroarylalkyl, heterocyclyl, heterocyclylalkyl, hydroxyl, amino, arylamino and alkylamino;
R⁴ and R⁵ can be the same or different, each being independently selected from the group consisting of H, alkyl, aryl and cycloalkyl; or alternatively R⁴ and R⁵ together form part of a cyclic 5- to 7-membered ring such that the moiety 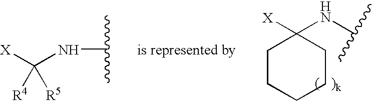
where k is 0 to 2;
X is selected from the group consisting of: 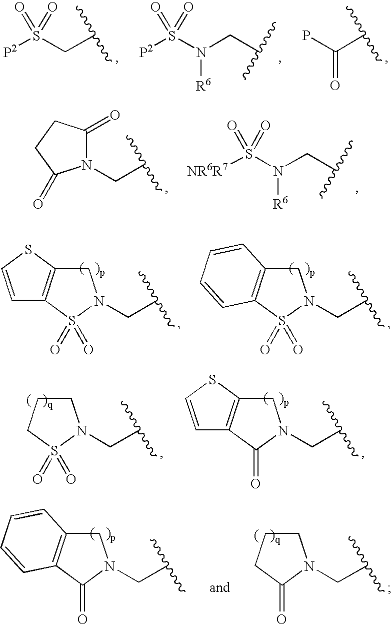
where p is 1 to 2, q is 1 to 3 and P² is alkyl, aryl, heteroaryl, heteroalkyl, cycloalkyl, dialkylamino, alkylamino, arylamino or cycloalkylamino; and
R³ is selected from the group consisting of: aryl, heterocyclyl, heteroaryl, 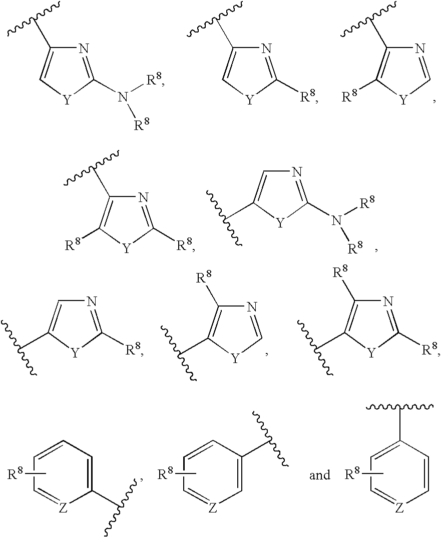
where Y is O, S or NH, and Z is CH or N, and the R⁸ moieties can be the same or different, each R⁸ being independently selected from the group consisting of hydrogen, alkyl, heteroalkyl, cycloalkyl, aryl, heteroaryl, heterocyclyl, hydroxyl, amino, arylamino, alkylamino, dialkylamino, halo, alkylthio, arylthio and alkyloxy; 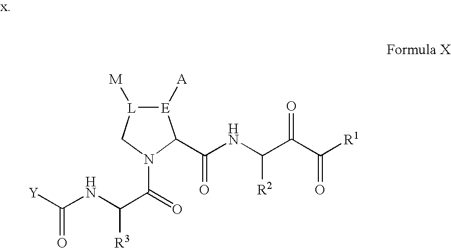
or a pharmaceutically acceptable salt, solvate or ester thereof;
wherein in Formula X:
R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;
A and M can be the same or different, each being independently selected from R, OR, NHR, NRR′, SR, SO₂R, and halo; or A and M are connected to each other such that the moiety: 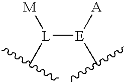
shown above in Formula I forms either a three, four, six, seven or eight-membered cycloalkyl, a four to eight-membered heterocyclyl, a six to ten-membered aryl, or a five to ten-membered heteroaryl;
E is C(H) or C═;
L is C(H), C═, CH₂C═, or C═CH₂;
R, R′, R², and R³ can be the same or different, each being independently selected from the group consisting of H, alkyl-, alkenyl-, alkynyl-, cycloalkyl-, heteroalkyl-,heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
and Y is selected from the following moieties: 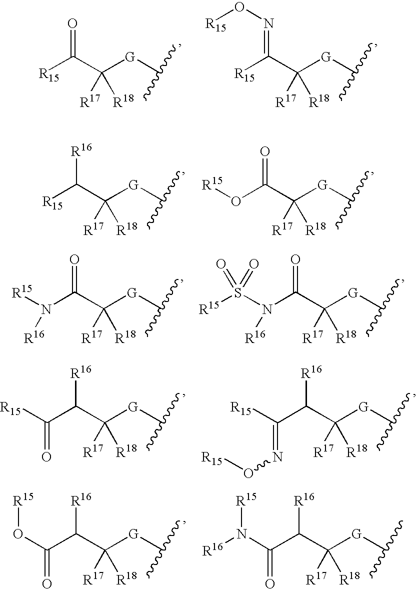
wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁵ and R¹⁶ are connected to each other to form a four to eight-membered cycloalkyl, heteroaryl or heterocyclyl structure, and likewise, independently R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro; 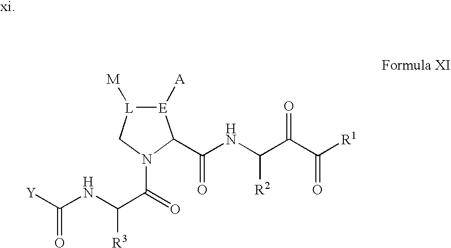
or a pharmaceutically acceptable salt, solvate or ester thereof;
wherein in Formula XI:
R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;
A and M can be the same or different, each being independently selected from R, NR⁹R¹⁰, SR, SO₂R, and halo; or A and M are connected to each other (in other words, A-E-L-M taken together) such that the moiety: 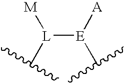
shown above in Formula I forms either a three, four, six, seven or eight-membered cycloalkyl, a four to eight-membered heterocyclyl, a six to ten-membered aryl, or a five to ten-membered heteroaryl;
E is C(H) or C═;
L is C(H), C═, CH₂C═, or C═CH₂;
R, R′, R², and R³ can be the same or different, each being independently selected from the group consisting of H, alkyl-, alkenyl-, alkynyl-, cycloalkyl-, heteroalkyl-,heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NR⁹R¹⁰ forms a four to eight-membered heterocyclyl;
Y is selected from the following moieties: 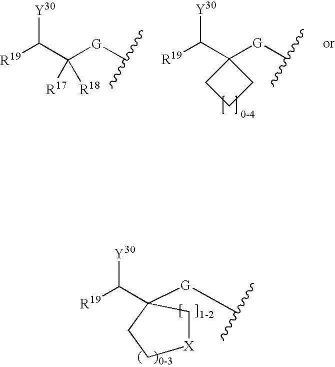
wherein Y³⁰ and Y³¹ are selected from 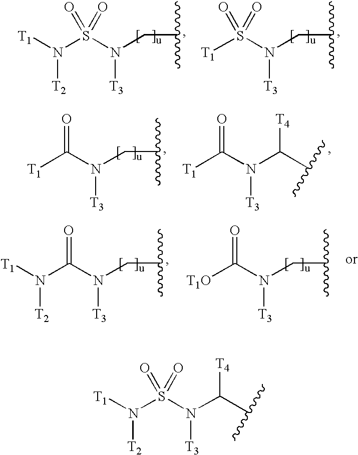
where u is a number 0-6;
X is selected from O, NR¹⁵, NC(O)R¹⁶, S, S(O) and SO₂;
G is NH or O; and
R¹⁵, R¹⁶, R¹⁷, R¹⁸, R¹⁹, T₁, T₂, T₃ and T₄ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl; wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro; 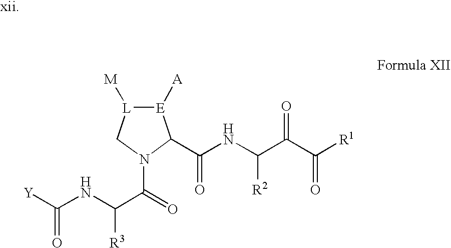
or a pharmaceutically acceptable salt, solvate or ester thereof;
wherein in Formula XII:
R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;
A and M can be the same or different, each being independently selected from R, OR, NHR, NRR′, SR, SO₂R, and halo; or A and M are connected to each other such that the moiety: 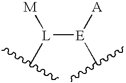
shown above in Formula I forms either a three, four, six, seven or eight-membered cycloalkyl, a four to eight-membered heterocyclyl, a six to ten-membered aryl, or a five to ten-membered heteroaryl;
E is C(H) or C═;
L is C(H), C═, CH₂C═, or C═CH₂;
R, R′, R², and R³ can be the same or different, each being independently selected from the group consisting of H, alkyl-, alkenyl-, alkynyl-, cycloalkyl-, heteroalkyl-,heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
and Y is selected from the following moieties: 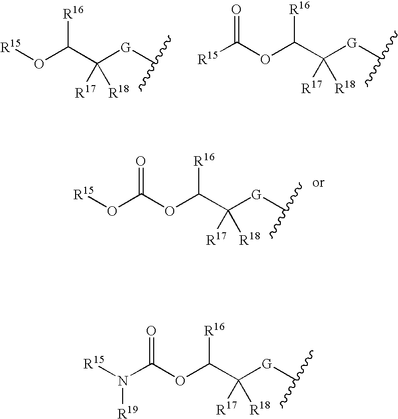
wherein G is NH or O; and R¹⁵, R¹⁶, R¹⁷, R¹⁸, and R¹⁹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, (i) either R¹⁵ and R¹⁶ are connected to each other to form a four to eight-membered cyclic structure, or R¹⁵ and R¹⁹ are connected to each other to form a four to eight-membered cyclic structure, and (ii) likewise, independently, R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: □ulfonam, alkoxy, aryloxy, thio, alkylthio, arylthio, amino, amido, alkylamino, arylamino, alkylsulfonyl, arylsulfonyl, □ulfonamide, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro; 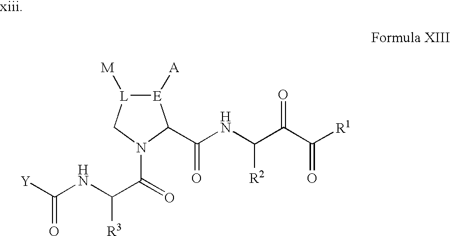
or a pharmaceutically acceptable salt, solvate or ester thereof;
wherein in Formula XIII:
R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;
A and M can be the same or different, each being independently selected from R, OR, NHR, NRR′, SR, SO₂R, and halo; or A and M are connected to each other (in other words, A-E-L-M taken together) such that the moiety: 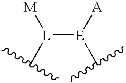
shown above in Formula I forms either a three, four, six, seven or eight-membered cycloalkyl, a four to eight-membered heterocyclyl, a six to ten-membered aryl, or a five to ten-membered heteroaryl;
E is C(H) or C═;
L is C(H), C═, CH₂C═, or C═CH₂;
R, R′, R², and R³ can be the same or different, each being independently selected from the group consisting of H, alkyl-, alkenyl-, alkynyl-, cycloalkyl-, heteroalkyl-,heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
and Y is selected from the following moieties: 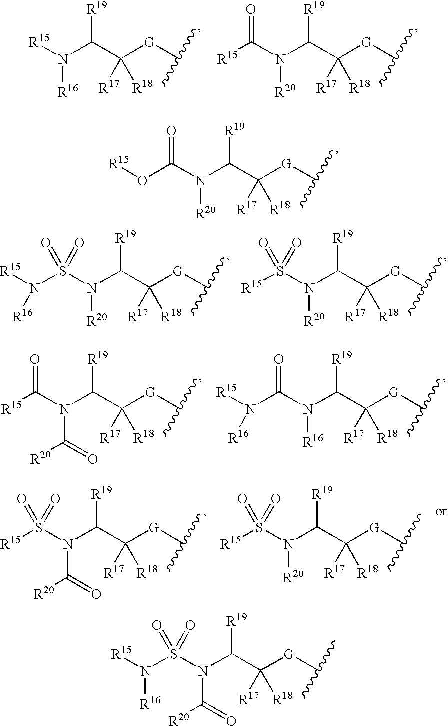
wherein G is NH or O, and R¹⁵, R¹⁶, R¹⁷, R¹′, R¹⁹ and R²⁰ can be the same or different, each being independently selected from the group consisting of H, C₁-C₁₀ alkyl, C₁-C₁₀ heteroalkyl, C₂-C₁₀ alkenyl, C₂-C₁₀ heteroalkenyl, C₂-C₁₀ alkynyl, C₂-C₁₀ heteroalkynyl, C₃-C₈ cycloalkyl, C₃-C₈ heterocyclyl, aryl, heteroaryl, or alternately: (i) either R¹⁵ and R¹⁶ can be connected to each other to form a four to eight-membered cycloalkyl or heterocyclyl, or R¹⁵ and R¹⁹ are connected to each other to form a five to eight-membered cycloalkyl or heterocyclyl, or R¹⁵ and R²⁰ are connected to each other to form a five to eight-membered cycloalkyl or heterocyclyl, and (ii) likewise, independently, R¹⁷ and R¹⁸ are connected to each other to form a three to eight-membered cycloalkyl or heterocyclyl,
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, keto, carboxy, carbalkoxy, carboxamido, alkoxycarbonylamino, alkoxycarbonyloxy, alkylureido, arylureido, halo, cyano, and nitro; 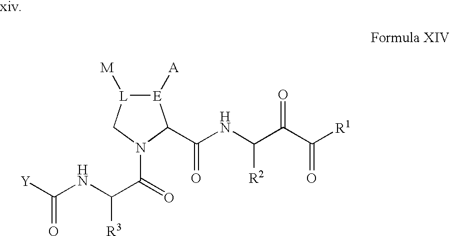
or a pharmaceutically acceptable salt, solvate or ester thereof;
wherein in Formula XIV:
R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;
A and M can be the same or different, each being independently selected from R, OR, NHR, NRR′, SR, SO₂R, and halo;
or A and M are connected to each other such that the moiety: 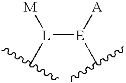
shown above in Formula I forms either a three, four, six, seven or eight-membered cycloalkyl, a four to eight-membered heterocyclyl, a six to ten-membered aryl, or a five to ten-membered heteroaryl;
E is C(H) or C═;
L is C(H), C═, CH₂C═, or C═CH₂;
R, R′, R², and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately R and R′ in NRR′ are connected to each other such that NRR′ forms a four to eight-membered heterocyclyl;
and Y is selected from the following moieties: 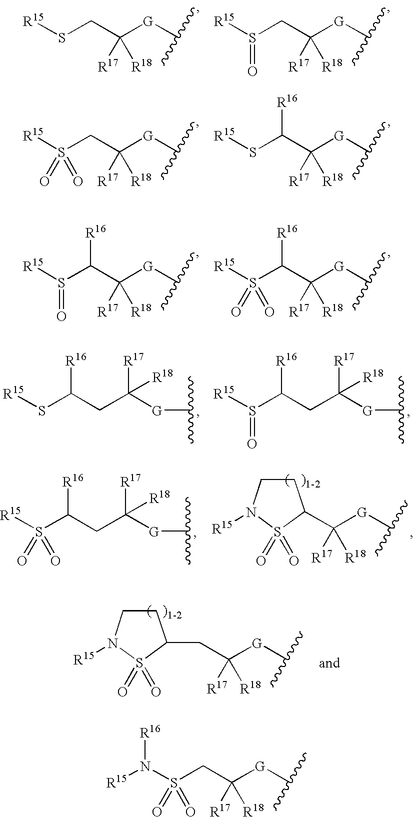
wherein G is NH or O; and R¹⁵, R¹⁶, R¹⁷ and R¹⁸ can be the same or different, each being independently selected from the group consisting of H, alkyl, heteroalkyl, alkenyl, heteroalkenyl, alkynyl, heteroalkynyl, cycloalkyl, heterocyclyl, aryl, and heteroaryl, or alternately, (i) R¹⁵ and R¹⁶ are connected to each other to form a four to eight-membered cyclic structure, and (ii) likewise, independently R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkylsulfonamido, arylsulfonamido, alkyl, aryl, heteroaryl, keto, carboxy, carbalkoxy, carboxamido, alkoxycarbonylamino, alkoxycarbonyloxy, alkylureido, arylureido, halo, cyano, and nitro; 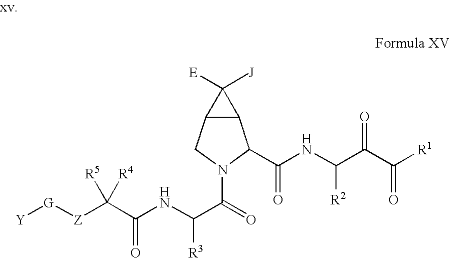
or a pharmaceutically acceptable salt, solvate or ester thereof;
wherein in Formula XV:
R¹ is NHR⁹, wherein R⁹ is H, alkyl-, aryl-, heteroalkyl-, heteroaryl-, cycloalkyl-, cycloalkyl-, arylalkyl-, or heteroarylalkyl;
E and J can be the same or different, each being independently selected from the group consisting of R, OR, NHR, NRR⁷, SR, halo, and S(O₂)R, or E and J can be directly connected to each other to form either a three to eight-membered cycloalkyl, or a three to eight-membered heterocyclyl moiety;
Z is N(H), N═, or O, with the proviso that when Z is O, G is present or absent and if G is present with Z being O, then G is C(═O);
G maybe present or absent, and if G is present, G is C(═O) or S(O₂), and
when G is absent, Z is directly connected to Y;
Y is selected from the group consisting of: 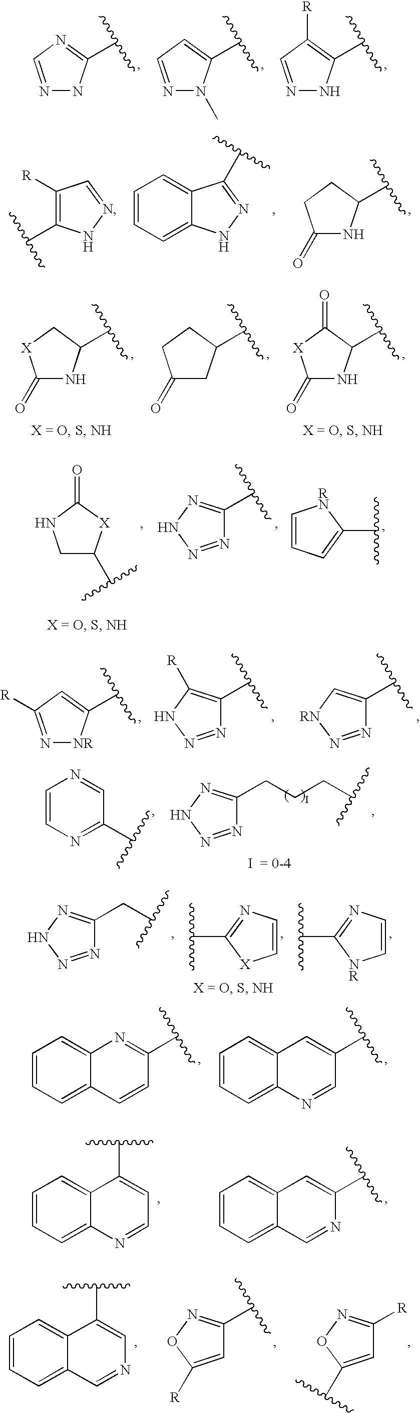 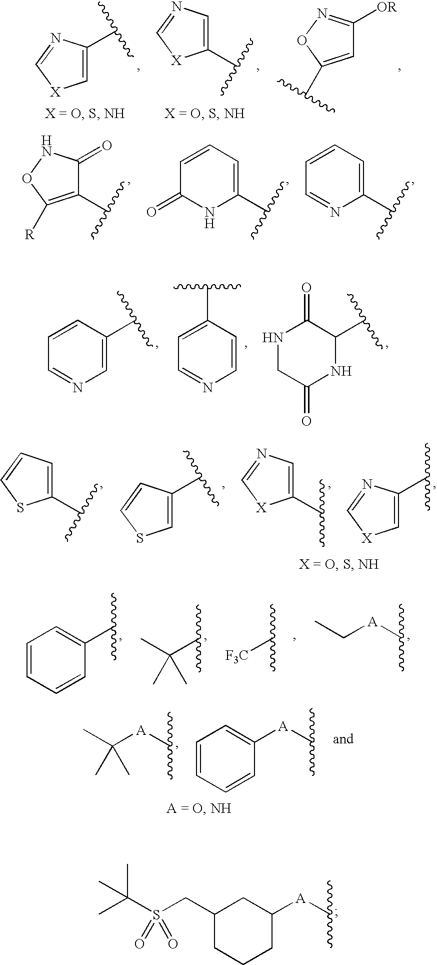
R, R⁷, R², R³, R⁴ and R⁵ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-, wherein each of said heteroalkyl, heteroaryl and heterocyclyl independently has one to six oxygen, nitrogen, sulfur, or phosphorus atoms;
wherein each of said alkyl, heteroalkyl, alkenyl, alkynyl, aryl, heteroaryl, cycloalkyl and heterocyclyl moieties can be unsubstituted or optionally independently substituted with one or more moieties selected from the group consisting of alkyl, alkenyl, alkynyl, aryl, aralkyl, cycloalkyl, heterocyclyl, halo, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate; 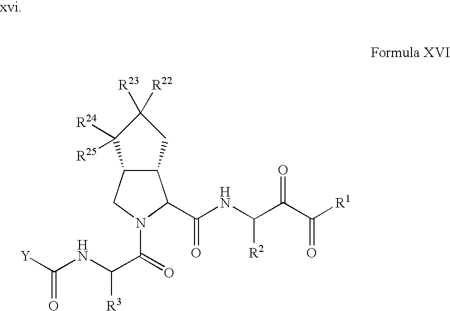
or a pharmaceutically acceptable salt, solvate or ester thereof;
wherein in Formula XVI:
R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;
R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;
Y is selected from the following moieties: 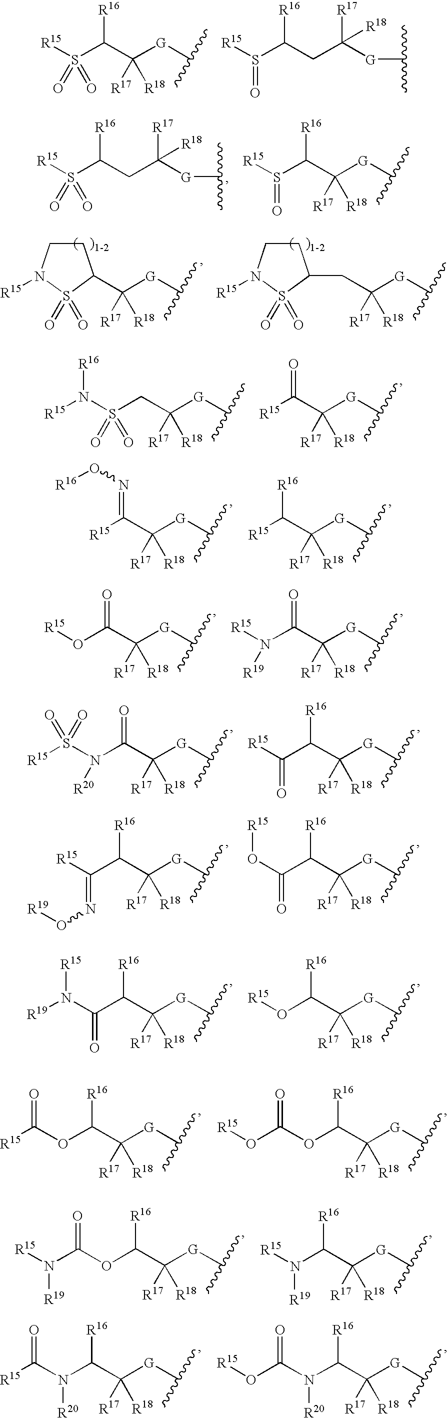 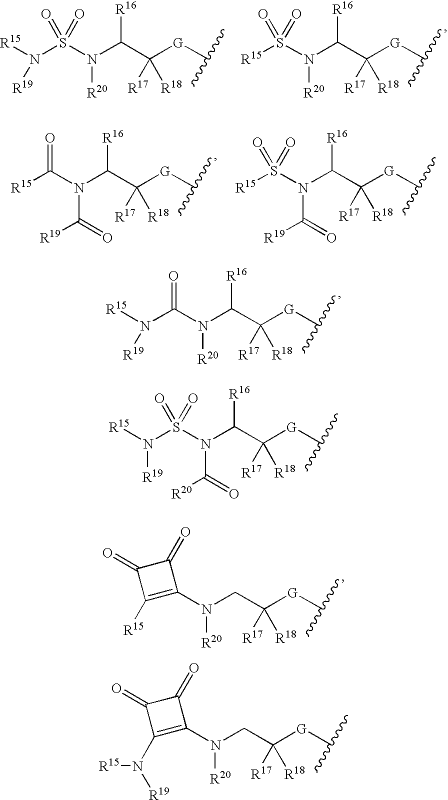
wherein G is NH or O; and R¹⁵, R¹⁶, R¹⁷, R¹⁸, R¹⁹, R²⁰R²¹, R²², R²³, R²⁴ and R²⁵ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; (iv) likewise independently R¹⁵ and R²⁰ are connected to each other to form a four to eight-membered heterocyclyl; (v) likewise independently R²² and R²³ are connected to each other to form a three to eight-membered cycloalkyl or a four to eight-membered heterocyclyl; and (vi) likewise independently R²⁴ and R²⁵ are connected to each other to form a three to eight-membered cycloalkyl or a four to eight-membered heterocyclyl; wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro; 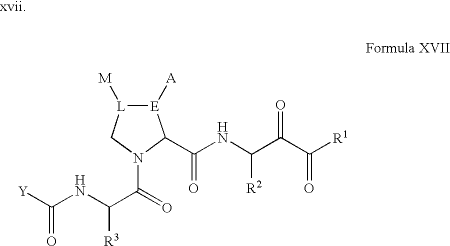
or a pharmaceutically acceptable salt, solvate or ester thereof;
wherein in Formula XVII:
R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;
A and M can be the same or different, each being independently selected from R, OR, NHR, NRR′, SR, SO₂R, and halo; or A and M are connected to each other such that the moiety: 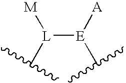
shown above in Formula I forms either a three, four, six, seven or eight-membered cycloalkyl, a four to eight-membered heterocyclyl, a six to ten-membered aryl, or a five to ten-membered heteroaryl;
E is C(H) or C═;
L is C(H), C═, CH₂C═, or C═CH₂;
R, R′, R², and R³ can be the same or different, each being independently selected from the group consisting of H, alkyl-, alkenyl-, alkynyl-, cycloalkyl-, heteroalkyl-,heterocyclyl-, aryl-, heteroaryl-, (cycloalkyl)alkyl-, (heterocyclyl)alkyl-, aryl-alkyl-, and heteroaryl-alkyl-; or alternately R and R′ in NRR′ are connected to each other such that NRR′ forms a four to eight-membered heterocyclyl;
Y is selected from the following moieties: 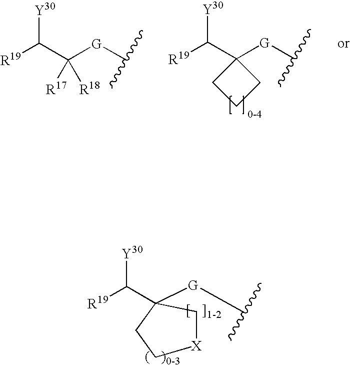
wherein Y³⁰ is selected from 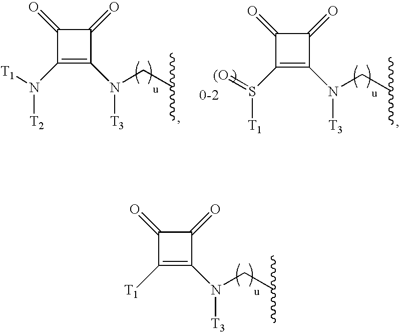
where u is a number 0-1;
X is selected from O, NR¹⁵, NC(O)R¹⁶, S, S(O) and SO₂;
G is NH or O; and
R¹⁵, R¹⁶, R¹⁷, R¹⁸, R¹⁹, T₁, T₂, and T₃ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately, R¹⁷ and R¹⁸ are connected to each other to form a three to eight-membered cycloalkyl or heterocyclyl;
wherein each of said alkyl, aryl, heteroaryl, cycloalkyl or heterocyclyl can be unsubstituted or optionally independently substituted with one or more moieties selected from the group consisting of: hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro; 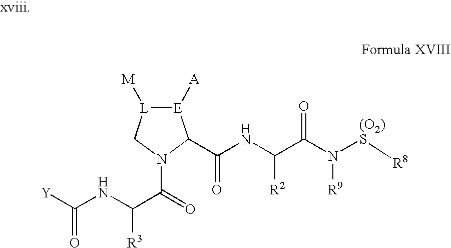
or a pharmaceutically acceptable salt, solvate or ester thereof;
wherein in Formula XVIII:
R⁸ is selected from the group consisting of alkyl-, aryl-, heteroalkyl-, heteroaryl-, cycloalkyl-, heterocyclyl-, arylalkyl-, heteroarylalkyl-, and heterocyclylalkyl;
R⁹ is selected from the group consisting of H, alkyl, alkenyl, alkynyl, aryl and cycloalkyl;
A and M can be the same or different, each being independently selected from R, OR, N(H)R, N(RR′), SR, S(O₂)R, and halo; or A and M are connected to each other (in other words, A-E-L-M taken together) such that the moiety: 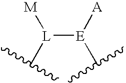
shown above in Formula I forms either a three, four, five, six, seven or eight-membered cycloalkyl, a four to eight-membered heterocyclyl, a six to ten-membered aryl, or a five to ten-membered heteroaryl;
E is C(H) or C(R);
L is C(H), C(R), CH₂C(R), or C(R)CH₂;
R and R′ can be the same or different, each being independently selected from the group consisting of H, alkyl-, alkenyl-, alkynyl-, cycloalkyl-, heteroalkyl-, heterocyclyl-, aryl-, heteroaryl-, (cycloalkyl)alkyl-, (heterocyclyl)alkyl-, aryl-alkyl-, and heteroaryl-alkyl-; or alternately R and R′ in N(RR′) are connected to each other such that N(RR′) forms a four to eight-membered heterocyclyl;
R² and R³ can be the same or different, each being independently selected from the group consisting of H, alkyl, heteroalkyl, alkenyl, heteroalkenyl, alkynyl, heteroalkynyl, cycloalkyl, spiro-linked cycloalkyl, heterocyclyl, aryl, arylalkyl, heteroaryl, and heteroarylalkyl;
Y is selected from the following moieties: 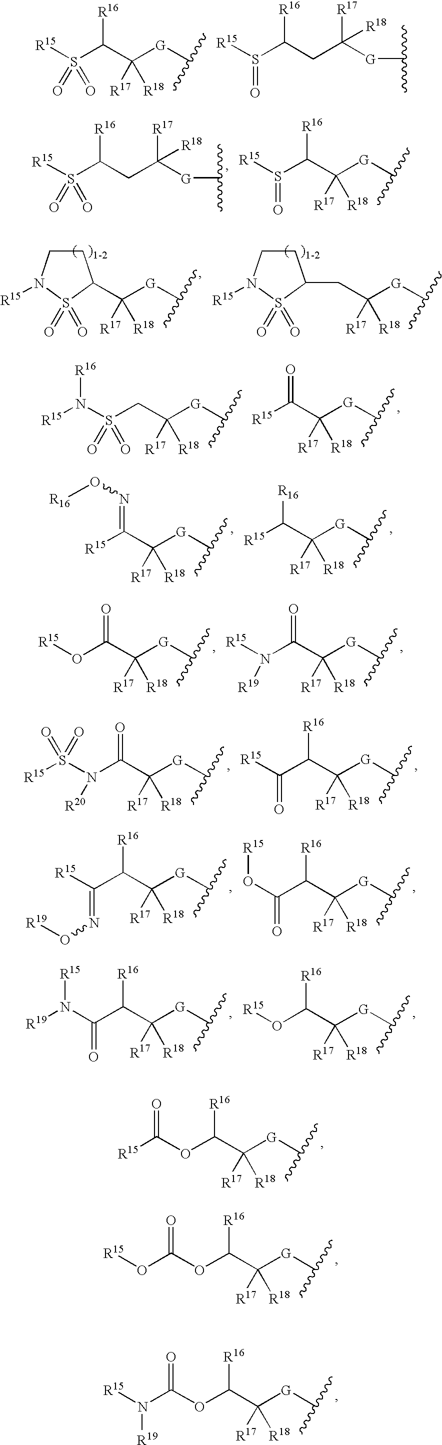 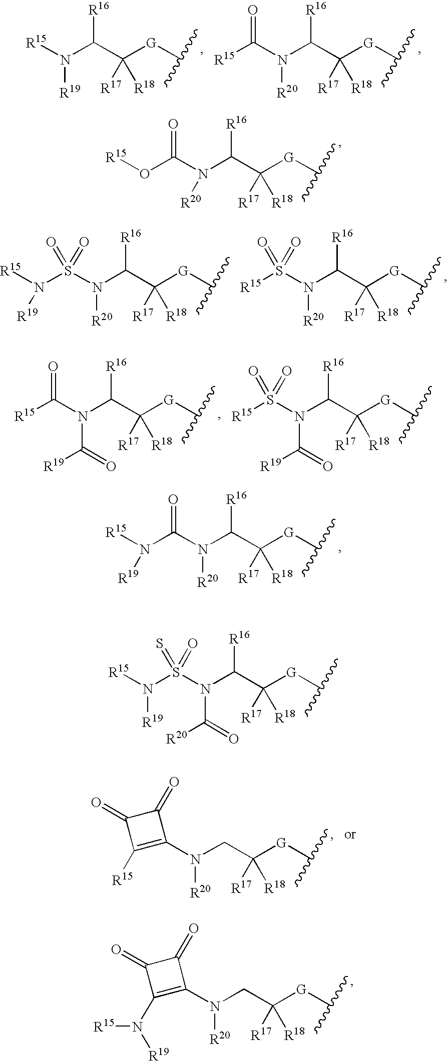
wherein G is NH or O; and R¹⁵, R¹⁶, R¹⁷, R¹⁸, R¹⁹ and R²⁰ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl; wherein each of said alkyl, aryl, heteroaryl, cycloalkyl, spiro-linked cycloalkyl, and heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, alkenyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro; 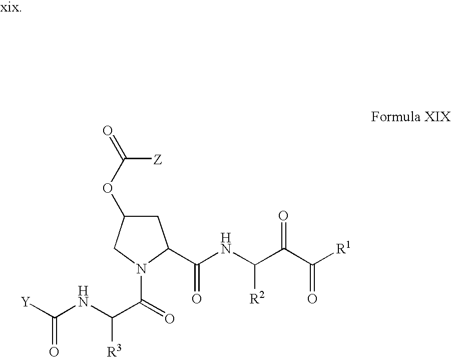
or a pharmaceutically acceptable salt, solvate or ester thereof;
wherein in Formula XIX:
Z is selected from the group consisting of a heterocyclyl moiety, N(H)(alkyl), —N(alkyl)₂, —N(H)(cycloalkyl), —N(cycloalkyl)₂, —N(H)(aryl, —N(aryl)₂, —N(H)(heterocyclyl), —N(heterocyclyl)₂, —N(H)(heteroaryl), and —N (heteroaryl)₂;
R¹ is NHR⁹, wherein R⁹ is H, alkyl-, alkenyl-, alkynyl-, aryl-, heteroalkyl-, heteroaryl-, cycloalkyl-, heterocyclyl-, arylalkyl-, or heteroarylalkyl;
R² and R³ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl;
Y is selected from the following moieties: 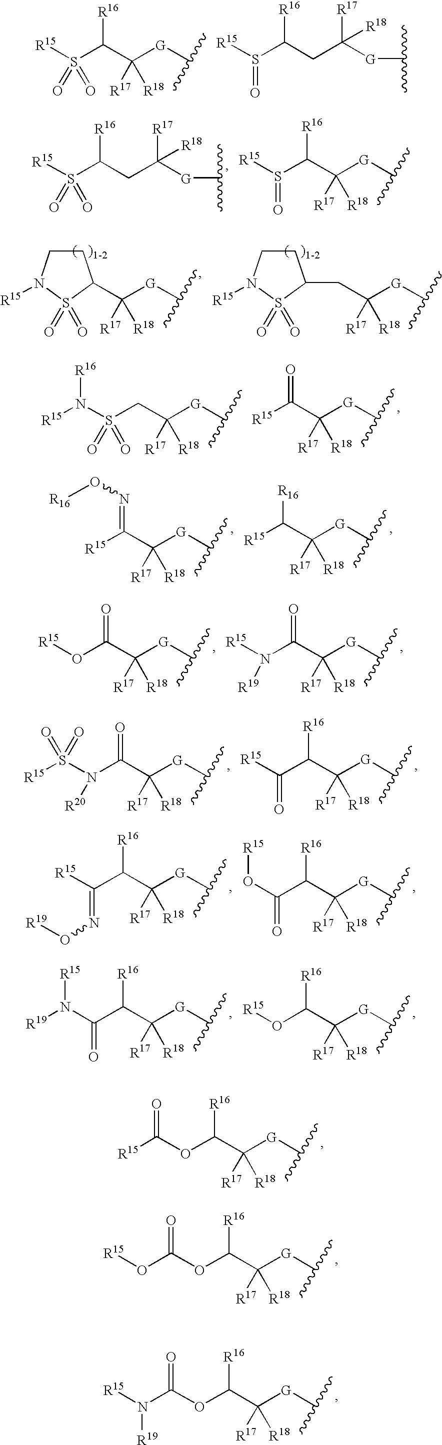 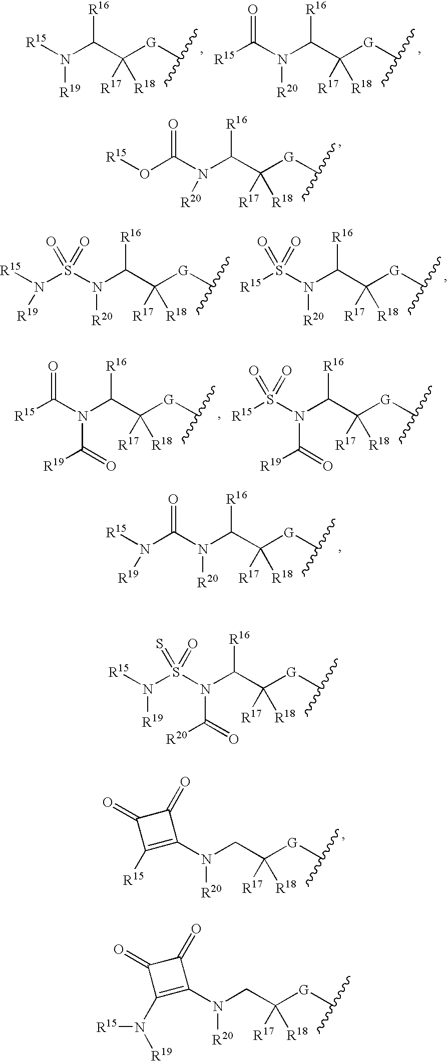
wherein G is NH or O; and R¹⁵, R¹⁶, R¹⁷, R¹⁸, R¹⁹, R²⁰ and R²¹ can be the same or different, each being independently selected from the group consisting of H, alkyl, heteroalkyl, alkenyl, heteroalkenyl, alkynyl, heteroalkynyl, cycloalkyl, heterocyclyl, aryl, arylalkyl, heteroaryl, and heteroarylalkyl, or alternately (i) R¹⁷ and R¹⁸ are independently connected to each other to form a three to eight-membered cycloalkyl or heterocyclyl; (ii) likewise independently R¹⁵ and R¹⁹ are connected to each other to form a four to eight-membered heterocyclyl; (iii) likewise independently R¹⁵ and R¹⁶ are connected to each other to form a four to eight-membered heterocyclyl; and (iv) likewise independently R¹⁵ and R²⁰ are connected to each other to form a four to eight-membered heterocyclyl;
wherein each of said alkyl, aryl, heteroaryl, cycloalkyl or heterocyclyl can be unsubstituted or optionally independently substituted with one or more moieties selected from the group consisting of hydroxy, alkoxy, aryloxy, thio, alkylthio, arylthio, amino, amido, alkylamino, arylamino, alkylsulfonyl, arylsulfonyl, sulfonamido, alkyl, aryl, heteroaryl, alkylsulfonamido, arylsulfonamido, keto, carboxy, carbalkoxy, carboxamido, alkoxycarbonylamino, alkoxycarbonyloxy, alkylureido, arylureido, halo, cyano, and nitro; 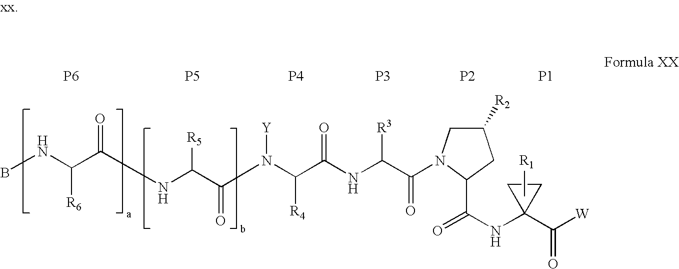
or a pharmaceutically acceptable salt, solvate or ester thereof;
wherein in Formula XX:
a is 0 or 1; b is 0 or 1; Y is H or C₁₋₆alkyl;
B is H, an acyl derivative of formula R₇—C(O)— or a sulfonyl of formula R₇—SO2 wherein
R7 is (i) C₁₋₁₀ alkyl optionally substituted with carboxyl, C₁₋₆ alkanoyloxy or C₁₋₆ alkoxy;
(ii) C₃₋₇ cycloalkyl optionally substituted with carboxyl, (C₁₋₆ alkoxy)carbonyl or phenylmethoxycarbonyl;
(iii) C₆ or C₁₀ aryl or C₇₋₁₆ aralkyl optionally substituted with C₁₋₆ alkyl, hydroxy, or amino optionally substituted with C₁₋₆ alkyl; or
(iv) Het optionally substituted with C₁₋₆ alkyl, hydroxy, amino optionally substituted with C₁₋₆ alkyl, or amido optionally substituted with C₁₋₆ alkyl;
R₆, when present, is C₁₋₆ alkyl substituted with carboxyl;
R₅, when present, is C₁₋₆ alkyl optionally substituted with carboxyl;
R₄ is C₁₋₁₀ alkyl, C₃₋₇ cycloalkyl or C₄₋₁₀ (alkylcycloalkyl);
R₃ is C₁₋₁₀ alkyl, C₃₋₇ cycloalkyl or C₄₋₁₀ (alkylcycloalkyl);
R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkyl cycloalkyl) being optionally mono-, di- or tri-substituted with R₂₁, or R₂₀ is a C₆ or C₁₀ aryl or C₇₋₁₆ aralkyl optionally mono-, di- or tri-substituted with R₂₁,
or R₂₀ is Het or (lower alkyl)-Het optionally mono-, di- or tri-substituted with R₂₁, wherein each R₂₁ is independently C₁₋₆ alkyl; C₁₋₆alkoxy; amino optionally mono- or di-substituted with C₁₋₆ alkyl; sulfonyl; N0₂; OH; SH; halo; haloalkyl; amido optionally mono-substituted with C₁₋₆ alkyl, C₆ or C₁₀ aryl, C₇₋₁₆ aralkyl, Het or (lower alkyl)-Het; carboxyl; carboxy(lower alkyl); C₆ or C₁₀ aryl, C₇₋₁₆ aralkyl or Het, said aryl, aralkyl or Het being optionally substituted with R₂₂;
wherein R₂₂ is C₁₋₆alkyl; C₁₋₆ alkoxy; amino optionally mono- or di-substituted with C₁₋₆ alkyl; sulfonyl; NO₂; OH; SH; halo; haloalkyl; carboxyl; amide or (lower alkyl)amide;
R₁ is C₁₋₆ alkyl or C₂₋₆ alkenyl optionally substituted with halogen; and
W is hydroxy or a N-substituted amino.
In the above-shown structure of the compound of Formula XX, the terms P6, P5, P4, P3, P2 and P1 denote the respective amino acid moieties as is conventionally known to those skilled in the art; 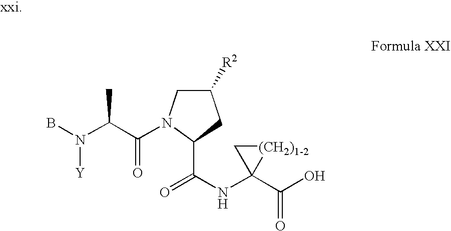
or a pharmaceutically acceptable salt, solvate or ester thereof;
wherein in Formula XXI:
B is H, a C₆ or C₁₀ aryl, C₇₋₁₆ aralkyl; Het or (lower alkyl)-Het, all of which optionally substituted with C₁₋₆ alkyl; C₁₋₆ alkoxy; C₁₋₆ alkanoyl; hydroxy; hydroxyalkyl; halo; haloalkyl; nitro; cyano; cyanoalkyl; amino optionally substituted with C₁₋₆ alkyl; amido; or (lower alkyl)amide; or B is an acyl derivative of formula R₄—C(O)—; a carboxyl of formula R₄—O—C(O)—; an amide of formula R₄—N(R₅)—C(O)—; a thioamide of formula R₄—N(R₅)—C(S)—; or a sulfonyl of formula R₄—SO₂ wherein
R₄ is (i) C₁₋₁₀ alkyl optionally substituted with carboxyl, C₁₋₆ alkanoyl, hydroxy, C₁₋₆ alkoxy, amino optionally mono- or di-substituted with C₁₋₆ alkyl, amido, or (lower alkyl) amide;
(ii) C₃₋₇ cycloalkyl, C₃₋₇ cycloalkoxy, or C₄₋₁₀ alkylcycloalkyl, all optionally substituted with hydroxy, carboxyl, (C₁₋₆ alkoxy)carbonyl, amino optionally mono- or di-substituted with C₁₋₆ alkyl, amido, or (lower alkyl) amide;
(iii) amino optionally mono- or di-substituted with C₁₋₆ alkyl; amido; or (lower alkyl)amide;
(iv) C₆ or C₁₀ aryl or C₇₋₁₆ aralkyl, all optionally substituted with C₁₋₆ alkyl, hydroxy, amido, (lower alkyl)amide, or amino optionally mono- or di-substituted with C₁₋₆ alkyl; or
(v) Het or (lower alkyl)-Het, both optionally substituted with C₁₋₆ alkyl, hydroxy, amido, (lower alkyl) amide, or amino optionally mono- or di-substituted with C₁₋₆ alkyl;
R₅ is H or C₁₋₆ alkyl;
with the proviso that when R₄ is an amide or a thioamide, R₄ is not (ii) a cycloalkoxy;
Y is H or C₁₋₆ alkyl;
R₃ is C₁₋₆alkyl, C₃₋₇ cycloalkyl, or C₄₋₁₀ alkylcycloalkyl, all optionally substituted with hydroxy, C₁₋₆ alkoxy, C₁₋₆ thioalkyl, amido, (lower alkyl)amido, C₆ or C₁₀ aryl, or C₇₋₁₆ aralkyl;
R₂ is CH₂—R₂₀, NH—R₂₀, O—R₂₀ or S—R₂₀, wherein R₂₀ is a saturated or unsaturated C₃₋₇ cycloalkyl or C₄₋₁₀ (alkylcycloalkyl), all of which being optionally mono-, di- or tri-substituted with R₂₁, or R₂₀ is a C₆ or C₁₀ aryl or C₇₋₁₄ aralkyl, all optionally mono-, di- or tri-substituted with R₂₁,
or R₂₀ is Het or (lower alkyl)-Het, both optionally mono-, di- or tri-substituted with R₂₁,
wherein each R₂₁ is independently C₁₋₆ alkyl; C₁₋₆ alkoxy; lower thioalkyl; sulfonyl; N0₂; OH; SH; halo; haloalkyl; amino optionally mono- or di-substituted with C₁₋₆ alkyl, C₆ or C₁₀ aryl, C₇₋₁₄ aralkyl, Het or (lower alkyl)-Het; amido optionally mono-substituted with C₁₋₆ alkyl, C₆ or C₁₀ aryl, C₇₋₁₄ aralkyl, Het or (lower alkyl)-Het; carboxyl; carboxy(lower alkyl); C₆ or C₁₀ aryl, C₇₋₁₄ aralkyl or Het, said aryl, aralkyl or Het being optionally substituted with R₂₂;
wherein R₂₂ is C₁₋₆ alkyl; C₃₋₇ cycloalkyl; C₁₋₆ alkoxy; amino optionally mono- or di-substituted with C₁₋₆ alkyl; sulfonyl; (lower alkyl)sulfonyl; N0₂; OH; SH; halo; haloalkyl; carboxyl; amide; (lower alkyl)amide; or Het optionally substituted with C₁₋₆ alkyl;
R¹ is H; C₁₋₆ alkyl, C₃₋₇ cycloalkyl, C₂₋₆ alkenyl, or C₂₋₆ alkynyl, all optionally substituted with halogen; 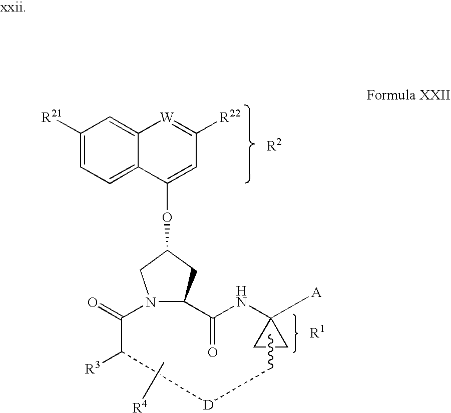
or a pharmaceutically acceptable salt, solvate or ester thereof;
wherein in Formula XXII:
W is CH or N,
R²¹ is H, halo, C₁₋₆ alkyl, C₃₋₆ cycloalkyl, C₁₋₆ haloalkyl, C₁₋₆ alkoxy, C₃₋₆ cycloalkoxy, hydroxy, or N(R²³)₂, wherein each R²³ is independently H, C₁₋₆ alkyl or C₃₋₆ cycloalkyl;
R²² is H, halo, C₁₋₆ alkyl, C₃₋₆ cycloalkyl, C₁₋₆ haloalkyl, C₁₋₆ thioalkyl, C₁₋₆ alkoxy, C₃₋₆ cycloalkoxy, C₂₋₇ alkoxyalkyl, C₃₋₆ cycloalkyl, C_{6 or 10} aryl or Het, wherein Het is a five-, six-, or seven-membered saturated or unsaturated heterocycle containing from one to four heteroatoms selected from nitrogen, oxygen and sulfur;
said cycloalkyl, aryl or Het being substituted with R²⁴, wherein R²⁴ is H, halo, C₁₋₆ alkyl, C₃₋₆ cycloalkyl, C₁₋₆ alkoxy, C₃₋₆ cycloalkoxy, NO₂, N(R²⁵)₂, NH—C(O)—R²⁵ or NH—C(O)—NH—R²⁵, wherein each R²⁵ is independently: H, C₁₋₆ alkyl or C₃₋₆ cycloalkyl;
or R²⁴ is NH—C(O)—OR²⁶ wherein R²⁶ is C₁₋₆ alkyl or C₃₋₆ cycloalkyl;
R³ is hydroxy, NH₂, or a group of formula —NH—R³¹, wherein R³¹ is C₆ or 10 aryl, heteroaryl, —C(O)—R³², —C(O)—NHR³² or —C(O)—OR³², wherein R³² is C₁₋₆ alkyl or C₃₋₆ cycloalkyl;
D is a 5 to 10-atom saturated or unsaturated alkylene chain optionally containing one to three heteroatoms independently selected from: O, S, or N—R⁴¹, wherein R⁴¹ is H, C₁₋₆ alkyl, C₃₋₆ cycloalkyl or —C(O)—R⁴², wherein R⁴² is C₁₋₆ alkyl, C₃₋₆ cycloalkyl or C₆ or 10 aryl; R⁴ is H or from one to three substituents at any carbon atom of said chain D, said substituent independently selected from the group consisting of: C₁₋₆ alkyl, C₁₋₆ haloalkyl, C₁₋₆ alkoxy, hydroxy, halo, amino, oxo, thio and C₁₋₆ thioalkyl, and
A is an amide of formula —C(O)—NH—R⁵, wherein R⁵¹S selected from the group consisting of: C₁₋₈ alkyl, C₃₋₆ cycloalkyl, C_{6 or 10} aryl and C₇₋₁₆ aralkyl;
or A is a carboxylic acid; 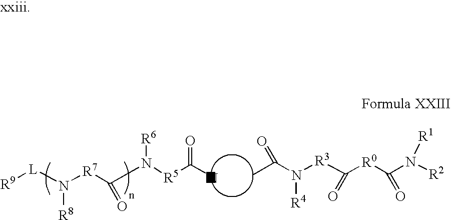
or a pharmaceutically acceptable salt, solvate or ester thereof;
wherein in Formula XXIII:
R⁰ is a bond or difluoromethylene;
R¹ is hydrogen;
R² and R⁹ are each independently optionally substituted aliphatic group, optionally substituted cyclic group or optionally substituted aromatic group;
R³, R⁵ and R⁷ are each independently:
optionally substituted (1,1- or 1,2-)cycloalkylene; or
optionally substituted (1,1- or 1,2-) heterocyclylene; or
methylene or ethylene), substituted with one substituent selected from the group consisting of an optionally substituted aliphatic group, an optionally substituted cyclic group or an optionally substituted aromatic group, and wherein the methylene or ethylene is further optionally substituted with an aliphatic group substituent; or;
R4, R6, R8 and R¹⁰ are each independently hydrogen or optionally substituted aliphatic group; 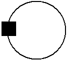
is substituted monocyclic azaheterocyclyl or optionally substituted multicyclic azaheterocyclyl, or optionally substituted multicyclic azaheterocyclenyl wherein the unsaturatation is in the ring distal to the ring bearing the R⁹—L-(N(R⁸)—R⁷—C(O)—)ₙN(R⁶)—R⁵—C(O)—N moiety and to which the —C(O)—N(R⁴)—R³—C(O)C(O)NR²R¹ moiety is attached;
L is —C(O)—, —OC(O)—, —NR¹⁰C(O)—, —S(0)₂—, or —NR¹⁰S(0)₂—; and
n is 0 or 1,
provided
when 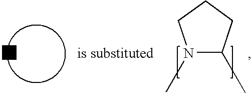
then L is —OC(O)— and R⁹ is optionally substituted aliphatic; or at least one of R³, R⁵ and R⁷ is ethylene, substituted with one substituent selected from the group consisting of an optionally substituted aliphatic group, an optionally substituted cyclic group or an optionally substituted aromatic group and wherein the ethylene is further optionally substituted with an aliphatic group substituent; or R⁴ is optionally substituted aliphatic; 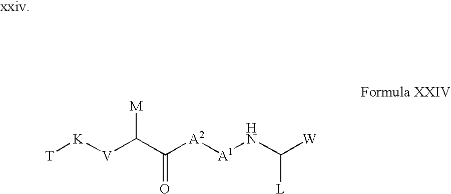
or a pharmaceutically acceptable salt, solvate or ester thereof;
wherein in Formula XXIV:
W is: 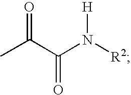
m is 0 or 1;
R² is independently hydrogen, alkyl, alkenyl, aryl, aralkyl, aralkenyl, cycloalkyl, cycloalkylalkyl, cycloalkenyl, cycloalkenylalkyl, heterocyclyl, heterocyclylalkyl, heterocyclylalkenyl, heteroaryl, or heteroaralkyl, wherein any R² carbon atom is optionally substituted with J;
J is alkyl, aryl, aralkyl, alkoxy, aryloxy, aralkoxy, cycloalkyl, cycloalkoxy, heterocyclyl, heterocyclyloxy, heterocyclylalkyl, keto, hydroxy, amino, alkylamino, alkanoylamino, aroylamino, aralkanoylamino, carboxy, carboxyalkyl, carboxamidoalkyl, halo, cyano, nitro, formyl, acyl, sulfonyl, or sulfonamido and is optionally substituted with 1-3 J¹ groups;
J¹ is alkyl, aryl, aralkyl, alkoxy, aryloxy, heterocyclyl, heterocyclyloxy, keto, hydroxy, amino, alkanoylamino, aroylamino, carboxy, carboxyalkyl, carboxamidoalkyl, halo, cyano, nitro, formyl, sulfonyl, or sulfonamido;
L is alkyl, alkenyl, or alkynyl, wherein any hydrogen is optionally substituted with halogen, and wherein any hydrogen or halogen atom bound to any terminal carbon atom is optionally substituted with sulfhydryl or hydroxy;
A¹ is a bond;
R⁴ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;
R⁵ and R⁶ are independently hydrogen, alkyl, alkenyl, aryl, aralkyl, aralkenyl, cycloalkyl, cycloalkylalkyl, cycloalkenyl, heterocyclyl, heterocyclylalkyl, heteroaryl, or heteroaralkyl, and is optionally substituted with 1-3 J groups;
X is a bond, —C(H)(R7)—, -0-, —S—, or —N(R8)—;
R⁷ is hydrogen, alkyl, alkenyl, aryl, aralkyl, heterocyclyl, heterocyclylalkyl, heteroaryl, or heteroaralkyl, and is optionally substituted with 1-3 J groups;
R⁸ is hydrogen alkyl, aryl, aralkyl, heterocyclyl, heterocyclylalkyl, heteroaryl, heteroaralkyl, aralkanoyl, heterocyclanoyl, heteroaralkanoyl, —C(O)R¹⁴, —SO₂R¹⁴, or carboxamido, and is optionally substititued with 1-3 J groups; or R⁸ and Z, together with the atoms to which they are bound, form a nitrogen containing mono- or bicyclic ring system optionally substituted with 1-3 J groups;
R¹⁴ is alkyl, aryl, aralkyl, heterocyclyl, heterocyclyalkyl, heteroaryl, or heteroaralkyl;
Y is a bond, —CH₂—, —C(O)—, —C(O)C(O)—, —S(O)—, —S(O)₂—, or —S(O)(NR⁷)—,
wherein R⁷ is as defined above;
Z is alkyl, aryl, aralkyl, cycloalkyl, cycloalkylalkyl, heterocyclyl, heterocyclylalkyl, heteroaryl, heteroaralkyl, —OR², or —N(R²)₂, wherein any carbon atom is optionally substituted with J, wherein R² is as defined above;
A² is a bond or 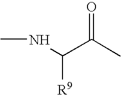
R⁹ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups;
M is alkyl, cycloalkyl, aryl, aralkyl, heterocyclyl, heterocyclylalkyl, heteroaryl, or heteroaralkyl, optionally substituted by 1-3 J groups, wherein any alkyl carbon atom may be replaced by a heteroatom;
V is a bond, —CH₂—, —C(H)(R¹¹)—, -0-, —S—, or —N(R¹¹)—;
R¹¹ is hydrogen or C₁₋₃ alkyl;
K is a bond, -0-, —S—, —C(O)—, —S(O)—, —S(O)₂—, or —S(O)(NR¹¹)—, wherein R¹¹ is as defined above;
T is —R¹², -alkyl-R², -alkenyl-R¹², -alkynyl-R², —OR¹², —N(R¹²)₂, —C(O)R¹², —C(═NOalkyl)R¹², or 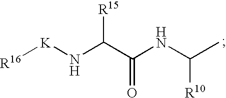
R¹² is hydrogen, aryl, heteroaryl, cycloalkyl, heterocyclyl, cycloalkylidenyl, or heterocycloalkylidenyl, and is optionally substituted with 1-3 J groups, or a first R¹² and a second R¹², together with the nitrogen to which they are bound, form a mono- or bicyclic ring system optionally substituted by 1-3 J groups;
R¹⁰ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 hydrogens J groups;
R¹⁵ is alkyl, cycloalkyl, aryl, aralkyl, heterocyclyl, heterocyclylalkyl, heteroaryl, heteroaralkyl, carboxyalkyl, or carboxamidoalkyl, and is optionally substituted with 1-3 J groups; and
R¹⁶ is hydrogen, alkyl, aryl, heteroaryl, cycloalkyl, or heterocyclyl; and 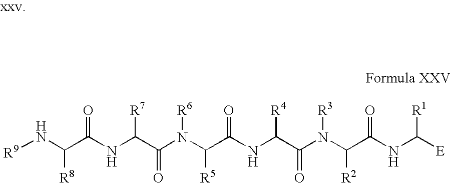
or a pharmaceutically acceptable salt, solvate or ester thereof;
wherein in Formula XXV:
E represents CHO or B(OH)₂;
R¹ represents lower alkyl, halo-lower alkyl, cyano-lower alkyl, lower alkylthio-lower alkyl, aryl-lower alkylthio-lower alkyl, aryl-lower alkyl, heteroaryllower alkyl, lower alkenyl or lower alkynyl;
R² represents lower alkyl, hydroxy-lower alkyl, carboxylower alkyl, aryl-lower alkyl, aminocarbonyl-lower alkyl or lower cycloalkyl-lower alkyl; and
R³ represents hydrogen or lower alkyl;
or R² and R³ together represent di- or trimethylene optionally substituted by hydroxy;
R⁴ represents lower alkyl, hydroxy-lower alkyl, lower cycloalkyl-lower alkyl, carboxy-lower alkyl, aryllower alkyl, lower alkylthio-lower alkyl, cyano-lower alkylthio-lower alkyl, aryl-lower alkylthio-lower alkyl, lower alkenyl, aryl or lower cycloalkyl;
R⁵ represents lower alkyl, hydroxy-lower alkyl, lower alkylthio-lower alkyl, aryl-lower alkyl, aryl-lower alkylthio-lower alkyl, cyano-lower alkylthio-lower alkyl or lower cycloalkyl;
R⁶ represents hydrogen or lower alkyl;
R⁷ represent lower alkyl, hydroxydower alkyl, carboxylower alkyl, aryl-lower alkyl, lower cycloalkyl-lower alkyl or lower cycloalkyl;
R⁸ represents lower alkyl, hydroxy-lower alkyl, carboxylower alkyl or aryl-lower alkyl; and
R⁹ represents lower alkylcarbonyl, carboxy-lower alkylcarbonyl, arylcarbonyl, lower alkylsulphonyl, arylsulphonyl, lower alkoxycarbonyl or aryl-lower alkoxycarbonyl; 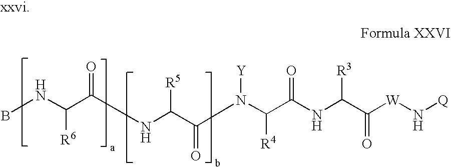
or a pharmaceutically acceptable salt, solvate or ester thereof;
wherein in Formula XXVI:
B is an acyl derivative of formula R₁₁—C(O)— wherein R₁₁ is Cl-10 alkyl optionally substituted with carboxyl; or R₁₁ is C₆ or C₁₀ aryl or C₇₋₁₆ aralkyl optionally substituted with a C₁₋₆ alkyl;
a is 0 or 1;
R₆, when present, is carboxy(lower)alkyl;
b is 0 or 1;
R₅, when present, is C₁₋₆ alkyl, or carboxy(lower)alkyl;
Y is H or C₁₋₆alkyl;
R₄ is C₁₋₁₀ alkyl; C₃₋₁₀ cycloalkyl;
R₃ is C₁₋₁₀ alkyl; C₃₋₁₀ cycloalkyl;
W is a group of formula: 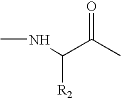
wherein R₂ is C₁₋₁₀ alkyl or C₃₋₇ cycloalkyl optionally substituted with carboxyl; C₆ or C₁₀ aryl; or C₇₋₁₆ aralkyl; or
W is a group of formula: 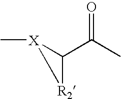
wherein X is CH or N; and
R₂′ is C₃₋₄ alkylene that joins X to form a 5- or 6-membered ring, said ring optionally substituted with OH; SH; NH2; carboxyl; R₁₂; OR₁₂, SR₁₂, NHR₁₂ or NR₁₂R₁₂′ wherein R₁₂ and R₁₂′ are independently:
cyclic C₃₋₁₆ alkyl or acyclic C₁₋₁₆ alkyl or cyclic C₃₋₁₆ alkenyl or acyclic C₂₋₁₆ alkenyl, said alkyl or alkenyl optionally substituted with NH₂, OH, SH, halo, or carboxyl; said alkyl or alkenyl optionally containing at least one heteroatom selected independently from the group consisting of: 0, S, and N; or
R₁₂ and R₁₂′ are independently C₆ or C₁₀ aryl or C₇₋₁₆ aralkyl optionally substituted with C₁₋₆ alkyl, NH₂, OH, SH, halo, carboxyl or carboxy(lower)alkyl; said aryl or aralkyl optionally containing at least one heteroatom selected independently from the group consisting of: 0, S, and N;
said cyclic alkyl, cyclic alkenyl, aryl or aralkyl being optionally fused with a second 5-, 6-, or 7-membered ring to form a cyclic system or heterocycle, said second ring being optionally substituted with NH₂, OH, SH, halo, carboxyl or carboxy(lower)alkyl; C₆ or C₁₀ aryl, or heterocycle; said second ring optionally containing at least one heteroatom selected independently from the group consisting of: O, S, and N;
Q is a group of the formula: 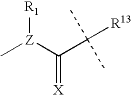
wherein Z is CH;
X is O or S;
R₁ is H, C₁₋₆ alkyl or C₁₋₆ alkenyl both optionally substituted with thio or halo; and
R₁₃ is C0—NH—R₁₄ wherein R₁₄ is hydrogen, cyclic C₃₋₁₀ alkyl or acyclic C₁₋₁₀ alkyl or cyclic C₃₋₁₀ alkenyl or acyclic C₂₋₁₀ alkenyl, said alkyl or alkenyl optionally substituted with NH₂, OH, SH, halo or carboxyl; said alkyl or alkenyl optionally containing at least one heteroatom selected independently from the group consisting of: O, S, and N; or
R₁₄ is C₆ or C₁₀ aryl or C₇₋₁₆ aralkyl optionally substituted with C₁₋₆ alkyl, NH₂, OH, SH, halo, carboxyl or carboxy(lower)alkyl or substituted with a further C₃₋₇ cycloalkyl, C₆ or C₁₀ aryl, or heterocycle; said aryl or aralkyl optionally containing at least one heteroatom selected independently from the group consisting of: O, S, and N;
said cyclic alkyl, cyclic alkenyl, aryl or aralkyl being optionally fused with a second 5-, 6-, or 7-membered ring to form a cyclic system or heterocycle, said second ring being optionally substituted with NH₂, OH, SH, halo, carboxyl or carboxy(lower)alkyl or substituted with a further C₃₋₇ cycloalkyl, C₆ or C₁₀ aryl, or heterocycle; said second ring optionally containing at least one heteroatom selected independently from the group consisting of: 0, S, and N;
with the proviso that when Z is CH, then R₁₃ is not an α-amino acid or an ester thereof;
Q is a phosphonate group of the formula: 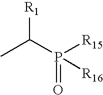
wherein R₁₅ and R₁₆ are independently C₆₋₂₀ aryloxy; and R₁, is as defined above; 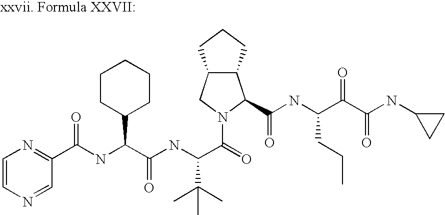
or a pharmaceutically acceptable salt, solvate or ester thereof;
or a mixture of two or more thereof for concurrent or consecutive administration in treating or ameliorating one or more symptoms of HCV, or disorders associated with HCV in a subject in need thereof.

3. The medicament according to claim 1 or 2, wherein at least one AKR competitor is an AKR substrate, an AKR inhibitor, or a mixture of two or more thereof.

4. The medicament of claim 3, wherein the AKR substrate is a fibrate, a 5α-dihydroxytestosterone, dolasetron, doxorubicin, 17β-estradiol, a non-steroidal anti-inflammatory drug (NSAID), ketotifen, naltrexone, Z-10-oxo nortriptyline, oestrone, a S-1360 HIV integrase inhibitor, progesterone, prostaglandin, sorbinil, testosterone, tibolone, tolrestat, naringenin, or a mixture of two or more thereof.

5. The medicament of claim 4, wherein the fibrate is benzafibrate, bezafibrate, binifibrate, ciprofibrate, clinofibrate, clofibrate, fenofibrate, gemfibrozil, lifibrol, or a mixture of two or more thereof.

6. The medicament of claim 4, wherein the NSAID is ibuprofen, diclofenac, diflunisal, flufenamic acid, indomethacin, mefenamic acid, naproxen, or a mixture of two or more thereof.

7. The medicament of claim 3, wherein the AKR inhibitor is an AKR1C1 AKR inhibitor, an AKR1C2 AKR inhibitor, an AKR1C3 AKR inhibitor, an AKR1C4 AKR inhibitor, naringenin, or a mixture of two or more thereof.

8. The medicament of claim 3, wherein the AKR inhibitor is a benzodiazepine, a cyclooxygenase (COX) 2 inhibitor, a NSAID, testosterone, naringenin, or a mixture of two or more thereof.

9. The medicament of claim 8, wherein the benzodiazepine is cloxazolam, diazepam, estazolam, flunitrazepam, nitrazepam, medazepam, or a mixture of two or more thereof.

10. The medicament of claim 8, wherein the COX 2 inhibitor is celecoxib.

11. The medicament of claim 8, wherein the NSAID is diclofenac, diflunisal, flufenamic acid, ibuprofen, indomethacin, mefenamic acid, naproxen, or a mixture of two or more thereof.

12. The medicament of claim 1 or 2, wherein at least one AKR competitor is diflusinal.

13. The medicament according to claim 12, wherein diflunisal is administered in an amount ranging from about 5 to about 499 mg per day.

14. The medicament according to claim 2, wherein at least one compound is: 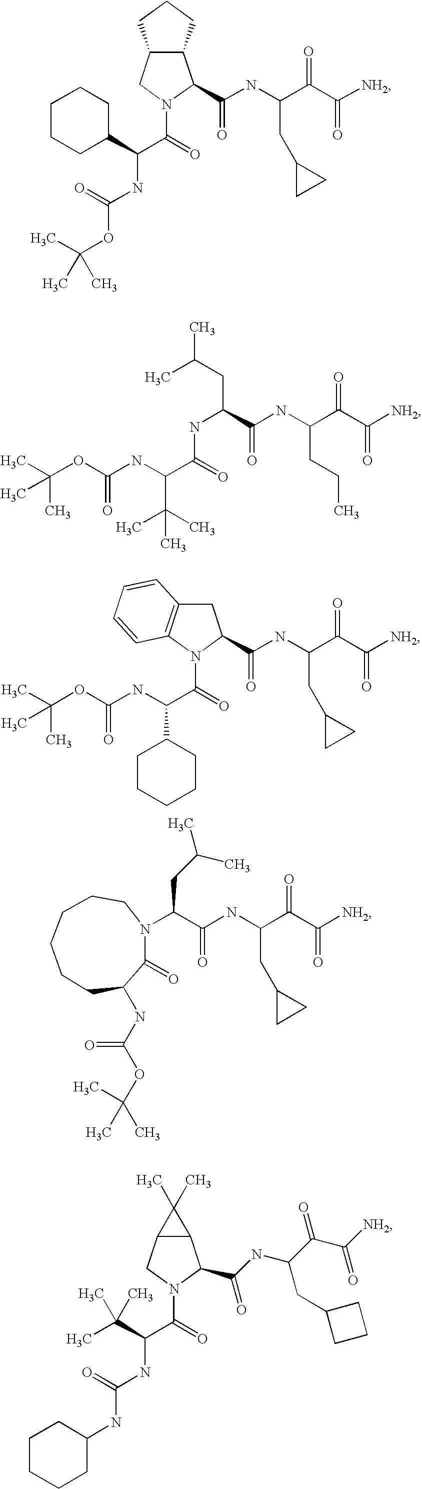 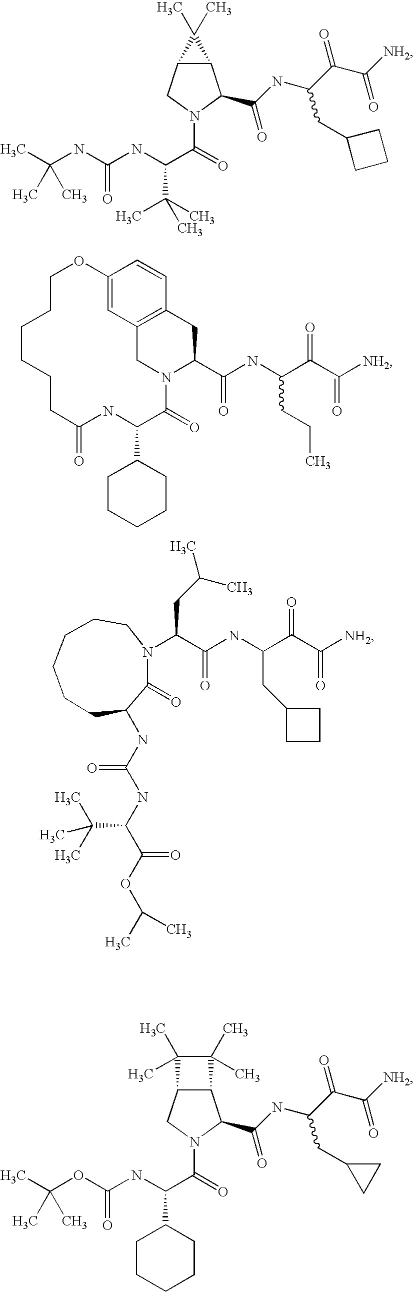 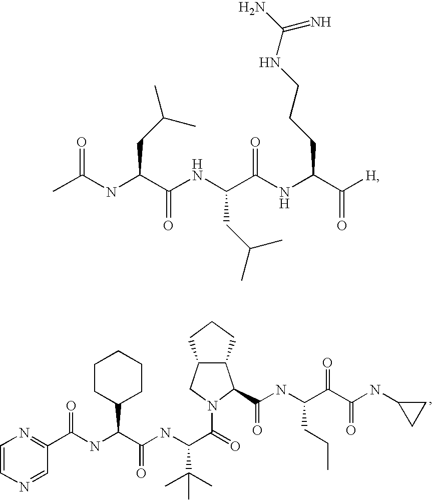
or a pharmaceutically acceptable salt, solvate or ester thereof, or a mixture of two or more thereof.

15. The medicament according to claim 2, wherein at least one compound is: 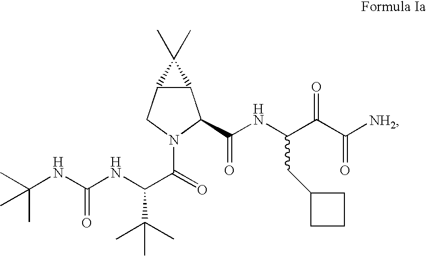
a pharmaceutically acceptable salt, solvate, or ester thereof, or a mixture of two or more thereof.

16. The medicament according to claim 2, wherein at least one compound is: 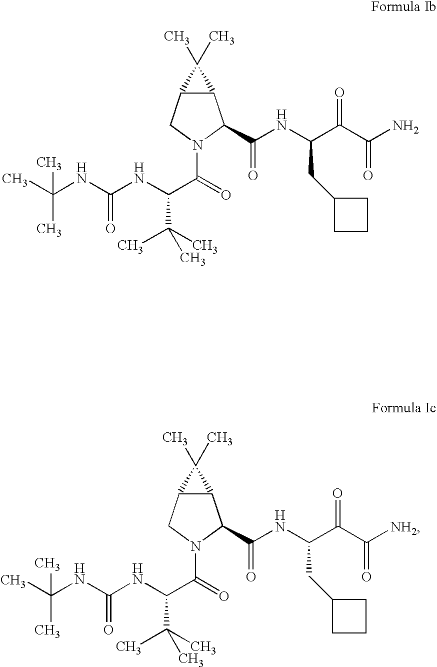
a pharmaceutically acceptable salt, solvate, or ester thereof, or a mixture of two or more thereof.

17. The medicament according to claim 2, wherein at least one compound is administered in an amount of about 100 mg to about 3000 mg per day.

18. The medicament according to claim 1 or 2, further comprising at least one other therapeutic agent.

19. The medicament according to claim 18, wherein at least one other therapeutic agent is ribavirin, levovirin, VP 50406, ISIS14803, Heptazyme, VX 497, Thymosin, Maxamine, mycophenolate mofetil, interferon, an antibody specific to IL-10, or a mixture of two or more thereof.

20. The medicament according to claim 18, wherein at least one other therapeutic agent is an antibody specific to IL-10.

21. The medicament according to claim 20, wherein the antibody specific to IL-10 is humanized 12G8.

22. The medicament according to claim 19, wherein interferon is interferon-alpha, PEG-interferon alpha conjugates, interferon alpha fusion polypeptides, consensus interferon, or a mixture of two or more thereof.

23. The medicament of claim 22, wherein at least one other therapeutic agent is administered concurrently or consecutively with at least one compound and the AKR competitor.

24. The medicament according to claim 1 or 2, further comprising at least one anti-cancer agent.

25. A pharmaceutical composition comprising a therapeutically effective amount of the medicament according to claim 1 or 2, and a pharmaceutically acceptable carrier.

26. A pharmaceutical kit comprising (a) as defined in claim 2, and (b) as defined in claim 2, in separate unit dosage forms, said forms being suitable for administration of (a) and (b) in effective amounts, and instructions for administering (a) and (b).

27. A method for treating or ameliorating one or more symptoms of HCV, or disorders associated with HCV in a subject in need thereof, comprising the step of administering to the subject an effective amount of the medicament of claim 2.

28. A medicament comprising, separately or together:
(a) at least one AKR competitor; and
(b) a HCV protease inhibitor selected from the group consisting of a compound of Formula Ia, Ib, or Ic, or a pharmaceutically acceptable salt, solvate or ester thereof, or a mixture of two or more thereof,
for concurrent or consecutive administration in treating or ameliorating one or more symptoms of HCV, or disorders associated with HCV in a subject in need thereof.

29. The medicament of claim 28, wherein at least one AKR competitor is diflunisal.

30. The medicament of claim 29, wherein the diflunisal is administered at a unit dosage sufficient to increase the bioavailability of the HCV protease inhibitor.

31. The medicament of claim 29, wherein the diflunisal is administered at a unit dosage of about 5 mg to about 499 mg per day.

32. The medicament of claim 29, wherein the diflunisal is administered at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, or 499 mg per day.

33. A pharmaceutical composition comprising a therapeutically effective amount of the medicament according to claim 28 and a pharmaceutically acceptable carrier.

34. A pharmaceutical kit comprising (a) as defined in claim 28, and (b) as defined in claim 28, in separate unit dosage forms, said forms being suitable for administration of (a) and (b) in effective amounts, and instructions for administering (a) and (b).

35. The pharmaceutical kit of claim 34, wherein at least one AKR competitor is diflunisal.

36. The pharmaceutical kit of claim 35, wherein the diflunisal is administered at a unit dosage sufficient to increase the bioavailability of the HCV protease inhibitor.

37. The pharmaceutical kit of claim 35, wherein the diflunisal is administered at a unit dosage of about 5 mg to about 499 mg per day.

38. The pharmaceutical kit of claim 35, wherein the diflunisal is administered at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, or 499 mg per day.

39. A method for treating or ameliorating one or more symptoms of HCV, or disorders associated with HCV in a subject in need thereof, comprising the step of administering to the subject an effective amount of the medicament of claim 28.

40. A medicament comprising, separately or together:
(a) at least one AKR competitor; and
(b) a HCV protease inhibitor selected from the group consisting of a compound of Formula XXVII, or a pharmaceutically acceptable salt, solvate, or ester thereof, or a mixture of two or more thereof, for concurrent or consecutive administration in treating or ameliorating one or more symptoms of HCV, or disorders associated with HCV in a subject in need thereof.

41. The medicament of claim 40, wherein at least one AKR competitor is diflunisal.

42. The medicament of claim 41, wherein the diflunisal is administered at a unit dosage sufficient to increase the bioavailability of the HCV protease inhibitor.

43. The medicament of claim 41, wherein the diflunisal is administered at a unit dosage of about 5 mg to about 499 mg per day.

44. The medicament of claim 41, wherein the diflunisal is administered at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, or 499 mg per day.

45. A pharmaceutical composition comprising a therapeutically effective amount of the medicament according to claim 40 and a pharmaceutically acceptable carrier.

46. A pharmaceutical kit comprising (a) as defined in claim 40, and (b) as defined in claim 40, in separate unit dosage forms, said forms being suitable for administration of (a) and (b) in effective amounts, and instructions for administering (a) and (b).

47. The pharmaceutical kit of claim 46, wherein at least one AKR competitor is diflunisal.

48. The pharmaceutical kit of claim 47, wherein the diflunisal is administered at a unit dosage sufficient to increase the bioavailability of the HCV protease inhibitor.

49. The pharmaceutical kit of claim 47, wherein the diflunisal is administered at a unit dosage of about 5 mg to about 499 mg per day.

50. The pharmaceutical kit of claim 47, wherein the diflunisal is administered at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, or 499 mg per day.

51. A method for treating or ameliorating one or more symptoms of HCV, or disorders associated with HCV in a subject in need thereof, comprising the step of administering to the subject an effective amount of the medicament of claim 40.

52. A composition comprising diflunisal at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, or 249 mg.

53. The composition of claim 52, wherein the diflunisal unit dosage is sufficient to increase the bioavailability of a HCV protease inhibitor.

54. The composition of claim 53, wherein the HCV protease inhibitor is selected from the group consisting of a compound of Formula Ia, Ib, or Ic, or a pharmaceutically acceptable salt, solvate or ester thereof, or a mixture of two or more thereof.

55. The composition of claim 53, wherein the HCV protease inhibitor is selected from the group consisting of a compound of Formula XXVII, or a pharmaceutically acceptable salt, solvate, or ester thereof, or a mixture of two or more thereof.

56. A pharmaceutical composition comprising an amount of the composition according to claim 52 sufficient to increase the bioavailability of a HCV protease inhibitor and a pharmaceutically acceptable carrier.

57. A kit comprising:
(a) diflunisal at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, or 499 mg; and
(b) instructions for administering (a).

58. The kit of claim 57 further comprising at least one HCV protease inhibitor.

59. The kit of claim 58, wherein at least one HCV protease inhibitor is selected from the group consisting of a compound of Formula Ia, Ib, or Ic, or a pharmaceutically acceptable salt, solvate or ester thereof, or a mixture of two or more thereof.

60. The kit of claim 58, wherein at least one HCV protease inhibitor is selected from the group consisting of a compound of Formula XXVII, or a pharmaceutically acceptable salt, solvate, or ester thereof, or a mixture of two or more thereof.

61. The kit of claim 58, wherein at least one HCV protease inhibitor is selected from the group consisting of a compound of Formula I to XXVII, or a pharmaceutically acceptable salt, solvate, or ester thereof, or a mixture of two or more thereof.

62. A method of increasing the bioavailability of a drug metabolized by AKR comprising administering diflunisal at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, or 499 mg.

63. The method of claim 62, wherein the diflunisal unit dosage is sufficient to increase the bioavailability of a HCV protease inhibitor.

64. The method of claim 63, wherein the HCV protease inhibitor is selected from the group consisting of a compound of Formula Ia, Ib, or Ic, or a pharmaceutically acceptable salt, solvate or ester thereof, or a mixture of two or more thereof.

65. The method of claim 63, wherein the HCV protease inhibitor is selected from the group consisting of a compound of Formula XXVII, or a pharmaceutically acceptable salt, solvate, or ester thereof, or a mixture of two or more thereof.

66. The method of claim 62 further comprising administering at least one HCV protease inhibitor concurrently or consecutively.

67. The method of claim 66 wherein at least one HCV protease inhibitor is selected from the group consisting of a compound of Formula Ia, Ib, or Ic, or a pharmaceutically acceptable salt, solvate, or ester thereof, or a mixture of two or more thereof.

68. The method of claim 66 wherein at least one HCV protease inhibitor is selected from the group consisting of a compound of Formula XXVII, or a pharmaceutically acceptable salt, solvate, or ester thereof, or a mixture of two or more thereof.

69. The method of claim 62, wherein the diflunisal unit dosage is sufficient to increase the blood level of a HCV protease inhibitor.
